(19) United States
(12) Patent Application Publication
(10) Pub. No.: US 2024/0409935 A1
(43) Pub. Date: Dec. 12, 2024

(54) Atg8ylation coordinates stress granule formation and mTOR inactivation in response to lysosomal damage

(71) Applicant: UNM RAINFOREST INNOVATIONS, Albuquerque, NM (US)

(72) Inventors: Vojo P. Deretic, Placitas, NM (US); Jingyue Jia Cassano, Albuquerque, NM (US)

(21) Appl. No.: 18/732,755

(22) Filed: Jun. 4, 2024

Related U.S. Application Data
(60) Provisional application No. 63/471,060, filed on Jun. 5, 2023.

(51) Int. Cl. C12N 15/113; A61K 31/133; A61K 31/16; A61K 31/167; A61K 31/4164; A61K 31/438; A61K 31/4706; A61K 31/496; A61K 31/5415; A61K 31/55; A61K 38/05

(52) U.S. Cl. CPC C12N 15/113; A61K 31/133; A61K 31/16; A61K 31/167; A61K 31/4164; A61K 31/438; A61K 31/4706; A61K 31/496; A61K 31/5415; A61K 31/55; A61K 38/05; C12N 15/1137; C12N 2310/14; C12N 2310/351; C12N 2310/3513; C12N 2320/31

(57) ABSTRACT

The present invention relates to the discovery that lysosomal damage is an inducer of stress granule (SG) formation and that SG formation and mTOR inactivation are coordinated through a noncanonical function of mammalian Atg8 proteins (mAtg8s), primarily associated with autophagosomes but also modifying other stressed membranes in a process referred to as Atg8ylation. Proteomics indicated recruitment to damaged lysosomes of the core SG proteins NUFIP2 and G3BP1 along with the GABARAP subset of mAtg8s. GABARAPs interacted with NUFIP2 and G3BP1 and lipidation of mAtg8s was needed for their recruitment to damaged lysosomes whereupon NUFIP2 contributed to mTOR inactivation via the Ragulator-RagA/B complex. The shared function of NUFIP2 and G3BP1 in SG formation and mTOR inactivation was reflected in competition between their roles in SGs and mTOR regulation. Thus, cells employ Atg8ylation to control and coordinate SG and mTOR responses to lysosomal damage. Methods of treating autophagy modulated disease states and/or conditions are described, particularly cancer using inhibitors of NUFIP2 and/or G3BP1, alone or in combination with lysosomotropic agents.

### RELATED APPLICATIONS AND GRANT SUPPORT

This application claims the benefit of priority of provisional application Ser. No. 63/471,060, filed 5 Jun. 2023, the entire contents of which are incorporated by reference here.

This invention was made with government support under grant nos. R37AI042999, no. R01AI111935 and P20GM 121176 awarded by the National Institutes of Health (NIIH). The government has certain rights in the invention.

### FIELD OF THE INVENTION

The present invention relates to the discovery that lysosomal damage is an inducer of stress granule (SG) formation and that SG formation and mTOR inactivation are coordinated through a noncanonical function of mammalian Atg8 proteins (mAtg8s), primarily associated with autophagosomes but also modifying other stressed membranes in a process referred to as Atg8ylation. SGs were induced by lysosome-damaging biochemical agents SARS-CoV-2^{ORF3a}, Mycobacterium tuberculosis and tau. Proteomics indicated recruitment to damaged lysosomes of the core SG proteins NUFIP2 and G3BP1 along with the GABARAP subset of mAtg8s. GABARAPs interacted with NUFIP2 and G3BP1 and lipidation of mAtg8s was needed for their recruitment to damaged lysosomes whereupon NUFIP2 contributed to mTOR inactivation via the Ragulator-RagA/B complex. The shared function of NUFIP2 and G3BP1 in SG formation and mTOR inactivation was reflected in competition between their roles in SGs and mTOR regulation. Thus, cells employ Atg8ylation to control and coordinate SG and mTOR responses to lysosomal damage. These results lead to the use of certain inhibitors of NUFIP2 and/or G3BP1 for treating disease states and/or conditions, especially cancer.

### BACKGROUND AND OVERVIEW OF THE INVENTION

The mammalian ATG factors participate in a number of processes including canonical (Levine and Kroemer, 2019; Morishita and Mizushima, 2019) and noncanonical autophagy variations, such as LC3-associated phagocytosis (LAP) and additional phenomena (Galluzzi and Green, 2019), and have been implicated in numerous diseases and physiological conditions (Deretic, 2021; Klionsky et al., 2021; Levine and Kroemer, 2019). ATG factors also participate in non-autophagic processes, including the role of ULK1 (mammalian ortholog of yeast Atg1) in glycolysis (Li et al., 2016) and disassembly of stress granules (SGs) (Wang et al., 2019), whereas ATG9A works in plasma membrane damage repair (Claude-Taupin et al., 2021). Several modules containing subsets of ATGs are necessary for canonical autophagy whereas their subsets but not all modules play roles in noncanonical functions (Galluzzi and Green, 2019; Kumar et al., 2021b). A number of these processes are under the control by metabolic regulators, mTOR and AMPK (Deretic, 2021; Kim et al., 2011; Levine and Kroemer, 2019), with signals transduced to the ATG apparatus through a module consisting of FIP200-ULK1-ATG13-ATG101, via phosphorylation by mTOR of several targets including Ser757 in ULK1 (Jia et al., 2018; Kim et al., 2011). In other cases, ATGs can play unique roles such as the mammalian Atg8 proteins (mAtg8s) (Gu et al., 2019a; Kumar et al., 2018), which can act even upstream of the lysosomally positioned regulators such as mTOR or TFEB, as evident during starvation or lysosomal damage (Kumar et al., 2020; Nakamura et al., 2020). These diverse responses, of which autophagy and LAP are only two subsets, include mAtg8s' conjugation to various stressed or remodeling membranes via mAtg8 lipidation (Kumar et al., 2021 b) or protein modifications (Carosi et al., 2021), collectively termed Atg8ylation.

Lysosomal damage elicits a complex set of responses (Ellison et al., 2020; Jia et al., 2018; Maejima et al., 2013; Papadopoulos et al., 2017; Radulovic et al., 2018; Skowyra et al., 2018; Thurston et al., 2012; Yoshida et al., 2017). This includes mobilization of ESCRT membrane repair systems (Jia et al., 2020b; Radulovic et al., 2018; Skowyra et al., 2018), mTOR inactivation (Eapen et al., 2021; Jia et al., 2018; Koerver et al., 2019), the translocation of TFEB (transcriptional regulator of lysosomal genes; (Napolitano and Ballabio, 2016)) from lysosomes to the nucleus (Chauhan et al., 2016), ubiquitination response (Jia et al., 2020a; Papadopoulos et al., 2017) and AMPK activation (Jia et al., 2020a), autophagy of lysosomes (lysophagy) (Maejima et al., 2013), and phospholipid and sphingolipid changes (Durgan et al., 2021; Ellison et al., 2020). Since inactivation of the cardinal regulator of cellular anabolic processes, mTOR (Betz and Hall, 2013; Shin and Zoncu, 2020), impacts multiple downstream processes such as autophagy and protein translation, it is of interest to consider global changes in transcription and translation, which is yet to be pursued.

A morphologically tractable process of stress granule (SG) formation has received recent attention as a part of global modulation of protein translation (Ivanov et al., 2019; Riggs et al., 2020; Yang et al., 2020). SGs (Collier et al., 1988; Collier and Schlesinger, 1986; Ivanov et al., 2019; Kedersha et al., 1999; Nover et al., 1983) are cytoplasmic, membraneless liquid-liquid phase separated biomolecular condensates (Alberti et al., 2019; Han et al., 2012; Kato et al., 2012; Weber and Brangwynne, 2012) containing ribonucleoprotein particles (RNP), translational factors, the 40S small ribosomal subunit (Cotto and Morimoto, 1999; Gallouzi et al., 2000; Kedersha et al., 2002; Kedersha et al., 1999), and a multitude of specialized components including proteins G3BP1, TIA1, and NUFIP2 (Ivanov et al., 2019; Jain et al., 2016; Yang et al., 2020). Canonical SG formation depends on phosphorylation of eukaryotic translation initiation factor 2α (eIF2α), which blocks the assembly of productive translation preinitiation complexes (Kedersha et al., 1999). Heat shock, oxidative stress, hypoxia, and viral infections, are the known stressors that trigger SG formation and translational arrest in mammalian cells (Anderson and Kedersha, 2002). Mammalian eIF2α is phosphorylated by four upstream kinases transducing the stress signals. This includes PKR, a double-stranded RNA-dependent kinase activated by viral infection (Srivastava et al., 1998), PERK, a resident ER protein activated by ER stress (Patil and Walter, 2001), GCN2, activated by amino acid deprivation (Kimball, 2001), and HRI activated by oxidative stress or heat shock (McEwen et al., 2005).

SGs are dynamic structures that can progress to P-bodies (Kedersha et al., 2005) leading to mRNA degradation (Ivanov et al., 2019; Kedersha and Anderson, 2007; Riggs et al., 2020), be disassembled (Wang et al., 2019), or subjected to autophagic degradation (Buchan et al., 2013; Chitiprolu et al., 2018). Furthermore, functional interactions and reciprocal regulation between SGs or SG proteins with mTORC1 components have been reported during heat shock or oxidative and osmotic stress (Sfakianos et al., 2018; Takahara and Maeda, 2012; Thedieck et al., 2013; Wippich et al., 2013) or during insulin-dependent reactivation of mTOR following amino acid starvation (Prentzell et al., 2021). Here we show that lysosomal damage, in part via PKR, induces formation of mRNA containing SGs, as a previously unrecognized stressor eliciting canonical SG formation. Lysosomal damage stress induced translation changes evidenced by enhanced selective translation of ATF4, which is a part of integrated stress response (ISR) (Vattern and Wek, 2004)(Lu et al., 2004)(Costa-Mattioli and Walter, 2020). The inventors furthermore report that Atg8ylation, a process that modifies stressed or remodeling membranes by lipid (Kumar et al., 2021 b) or protein (Carosi et al., 2021) conjugation is concomitantly elicited by lysosomal damage and that it competes with SG formation. Based on unbiased proteomic approaches, the inventors report that the key components of the SGs, NUFIP2 and G3BP1, which interact with mAtg8s, are recruited to lysosomes where they inactivate mTOR specifically via the Ragulator-Rag system. These processes are elicited under a spectrum of lysosomal damaging agents including protopathic tau, Mycobacterium tuberculosis, and SARS-CoV-2^{ORF3a}.

Lysosomes are acidic hydrolase-rich membrane-bound organelles that play a vital role in cellular degradation and signaling (Ballabio et al., 2020; Lamming et al., 2019; Lawrence et al., 2019; C. Yang et al., 2021). Damage to lysosomes can be triggered by numerous physiological and pathological conditions (Nakamura et al., 2021; C. Papadopoulos et al., 2017; H. Yang et al., 2023). These include microbial pathogens (Ghosh et al., 2020; Montespan et al., 2017; Thurston et al., 2012), environmental pollutants (Hornung et al., 2008; Mossman et al., 1998; J. Wang et al., 2017), toxic protein aggregates (Flavin et al., 2017; C. Papadopoulos et al., 2017), endogenous crystals (Hui et al., 2012; Maejima et al., 2013), and many lysosomotropic drugs (Marceau et al., 2012; Pisonero-Vaquero et al., 2017). These agents, along with various others, damage lysosomes, leading to the leakage of acidic contents and the disruption of cellular functions, thereby threatening cell survival (Patra et al., 2023; Saftig et al., 2021; Fengjuan Wang et al., 2018). Lysosomal damage is strongly linked to various human diseases, e.g., cancer, infectious, and neurodegenerative diseases (Amaral et al., 2023; Ballabio et al., 2020; Bonam et al., 2019; Fehrenbacher et al., 2005). Although lysosomal damage is of physiological importance and pathological relevance, understanding of how cells respond to this damage remains largely unknown (C. Papadopoulos et al., 2017).

Cells can detect lysosomal damage through several mechanisms, including the identification of calcium leakage or the exposure of luminal glycan (Aits et al., 2015; Radulovic et al., 2018; Skowyra et al., 2018). Minorly damaged lysosomes can be repaired through multiple cellular systems, including annexins (Ebstrup et al., 2023; Yim et al., 2022), sphingomyelin turnover (Niekamp et al., 2022), microautophagy (Ogura et al., 2023), ER-lysosome lipid transfer (Tan et al., 2022) as The well as ESCRT (the endosomal sorting complexes required for transport) machinery (Radulovic et al., 2018; Skowyra et al., 2018). Notably, the protein ALIX (ALG-2-Interacting Protein X), a key ESCRT component, can detect lysosomal damage by sensing calcium release, a function it performs alongside its partner, ALG2 (Apoptosis-Linked Gene-2)(W. Chen et al., 2024; Maki et al., 2016; Sun et al., 2015). Upon detecting such damage, ALIX facilitates the recruitment of other ESCRT components to the site of damage for repair (W. Chen et al., 2024; Radulovic et al., 2018; Skowyra et al., 2018). Severely damaged lysosomes can be removed by selective autophagy (Chauhan et al., 2016; Maejima et al., 2013), noncanonical autophagy (Boyle et al., 2023; Kaur et al., 2023), or lysosomal exocytosis (Wang et al., 2023). Master regulators mTORC1 (mechanistic target of rapamycin complex 1) and AMPK (AMP-activated protein kinase), located on lysosomes (Sancak et al., 2010; C.-S. Zhang et al., 2014), are finely tuned respond to lysosomal damage, subsequently activating downstream processes e.g., autophagy and lysosomal biogenesis (Jia et al., 2018; Jia et al., 2020a, b; Jia et al., 2020c). These mechanisms collectively safeguard lysosomal quality, maintaining cellular homeostasis (Jia et al., 2020d).

Recently, the inventors reported that lysosomal damage induces the formation of stress granules (SGs) (Jia et al., 2022). SGs are membrane-less organelles identified as ribonucleoprotein condensates that are believed to serve as protective responses in cells under adverse conditions (Ivanov et al., 2019; McCormick et al., 2017; Riggs et al., 2020). Consequently, dysfunctional SGs have been implicated in various human diseases e.g., neurodegenerative and infectious diseases (Advani et al., 2020; Protter et al., 2016; Fei Wang et al., 2020). SG formation is triggered by specific kinases, such as PKR (Protein Kinase R), that sense various stress stimuli, leading to the phosphorylation of eIF2α (eukaryotic translation initiation factor 2) (N. L. Kedersha et al., 1999; Srivastava et al., 1998). Phosphorylated eIF2α (p-eIF2α) halts global translation, resulting in the accumulation of untranslated mRNA (Jackson et al., 2010). Simultaneously, it promotes the selective expression of stress response proteins, a process known as the integrated stress response (Costa-Mattioli et al., 2020; Pakos-Zebrucka et al., 2016). SG formation can also occur through mTORC1-mediated translational shutdown, independent of p-eIF2α (Emara et al., 2012; Fujimura et al., 2012; McCormick et al., 2017). RNA-binding proteins G3BP1/2 (GAP SH3 Domain-Binding Protein 1/2) detect untranslated mRNA and collectively initiate SG formation through an RNA-protein network, driven by liquid-liquid phase separation (Hyman et al., 2014; Ivanov et al., 2019).

Despite the extensive knowledge of SG composition and dynamics, an understanding of the functional consequences of SG formation remains limited (Riggs et al., 2020). Significantly, SG formation has often been investigated under non-physiological conditions such as arsenic stress or heat shock (Jain et al., 2016; Sidrauski et al., 2015; Turakhiya et al., 2018; Verma et al., 2021; P. Yang et al., 2020). Our study (Jia et al., 2022) which originally revealed lysosomal damage as a critical internal physiological trigger for SGs, underscores the need to better understand the nature of SG formation in disease contexts. Additionally, this new connection between damaged lysosomes and SGs provides a novel perspective on the interaction between membrane-bound and membrane-less organelles (Zhao et al., 2020). For example, recent research suggests that SGs have the ability to plug and stabilize damaged lysosomes (Bussi et al., 2023). However, the precise regulation of SG formation in response to lysosomal damage and its consequential impact on cell fate remains largely unexplored.

In the present application, the inventors employed unbiased approaches to investigate how lysosomal damage signals are transduced to induce stress granule formation and to elucidate the cytoprotective role of SG formation in promoting cell survival against lysosomal damage. The findings revealed a novel function of ALIX, which senses calcium release from damaged lysosomes, in controlling the phosphorylation of eIF2α through PKR and its activator on damaged lysosomes, thereby initiating SG formation. This process is critical for cell survival in response to lysosomal damage caused by microbiolgical, pathological and environmental agents including SARS-CoV-2 ORF3a, adenovirus, Malaria hemozoin, proteopathic tau and silica. In conclusion, the present study uncovers a calcium-dependent signaling mechanism that transmits lysosomal damage signals to induce SG formation and reveals the cytoprotective role of SG formation in response to lysosomal damage caused by diverse stresses.

### BRIEF DESCRIPTION OF THE INVENTION

The invention relates to the discovery that lysomal damage is an inducer of stress granule (SG) formation and that SG formation and mTOR inactivation are coordinated through a noncanonical function of mammalian Atg8 proteins (mAtg8s), primarily associated with autophagosomes but also modifying other stressed membranes in a process referred to as Atg8ylation. SGs were induced by lysosome-damaging biochemical agents SARS-CoV-2^{ORF3a}, Mycobacterium tuberculosis and tau. Proteomics indicated recruitment to damaged lysosomes of the core SG proteins NUFIP2 and G3BP1 along with the GABARAP subset of mAtg8s. GABARAPs interacted with NUFIP2 and G3BP1 and lipidation of mAtg8s was needed for their recruitment to damaged lysosomes whereupon NUFIP2 contributed to mTOR inactivation via the Ragulator-RagA/B complex.

Accordingly, pursuant to the present invention it has been discovered by the present inventors that modulators of NUFIP2 and/or G3BP1, which may be agonists or especially antagonists of NUFIP2 or G3BP1 (such as SJ-19-0043) or alternatively agents which upregulate or downregulate NUFIP2 or G3BP1 may be used to treat autophagy mediated disease states and/or conditions as described herein. In an embodiment, the disease state is cancer and the modulators of NUFIP2 and/or G3BP1 are inhibitory siRNAs (small interfering RNAs, aka short interfering RNAs) of SEQ ID Nos: 3-10 as set forth below:

SEQ ID NO: 3
AGGAAAGCUAGGCGCAAUA;
SEQ ID NO: 4
GGGUGAUAUGCUUCGGAAA;
SEQ ID NO: 5
AAUUAAGCCCUGCGAGAAU;
SEQ ID NO: 6
AUGGUGAACUAAACGGUAA;
SEQ ID NO: 7
GUGGUGGAGUUGCGCAUUA;
SEQ ID NO: 8
AGACAUAGCUCAGACAGUA;
SEQ ID NO: 9
GAAGGCGACCGACGAGAUA;
SEQ ID NO: 10
GCGAGAACAACGAAUAAAU.

SiRNAs corresponding to SEQ ID Nos: 3-6, above are inhibitors of NUFIP2, whereas siRNAs corresponding to SEQ ID Nos: 7-10 are G3BP1 Inhibitors. These siRNAs may be used alone, as a mixture of siRNAs, or in combination with lysosomotropic agents for the treatment of cancer. In embodiments, the cancer is a tumor. In embodiments, the cancer is pancreatic cancer, a glioma, a glioblastoma or a neuroblastoma. Other cancers as described herein are also treating using siRNAs alone or in combination with a lysosomotropic agent.

In embodiments, the siRNA(s) are used naked or presented in lipid nanoparticles as described by Kanasty, et al., Nat. Mat., 12, 967-977 (2013), relevant portions of which are incorporated by reference herein. In other embodiments, the siRNA(s) are presented as conjugates such as dynamic polyconjugates (DPC), antibody conjugates (antibody-SiRNA conjugate) or GalNAc-SiRNA conjugates.

In embodiments, the siRNA(s) is combined with at least one lysosomotropic agent. In embodiments the lysosomotropic agent is a lysosomotropic detergent. In embodiments, the lysosomotropic agent is a lysosomotropic amine containing a moderately basic amine of pKa 5-9. In embodiments, the lysosomotropic amine is sphingosine, O-methyl-serine dodecylamide hydrochloride (MSDH), N-dodecylimidazole or a mixture of these lysosomotropic agents thereof. In embodiments, the the lysomotropic agent is chloroquine, chlorpromazine, thioridazine, aripiprazole, clomipramine, imipramine, desipramine, seramasine, or a mixture thereof. In embodiments, the lysosomotropic agent is glycyl-L-phenylalanine-2-naphthyl amide (GPN), Leu-Leu-OMe (LLOMe) or a mixture thereof.

In an embodiment, it has been discovered that when Integrated Stress Response Inhibitor (ISRIB, N,N′-((1s,4s)-cyclohexane-1,4-diyl)bis(2-(4-chlorophenoxy)acetamide), or an isomer thereof, preferably as the trans isomer depicted below)

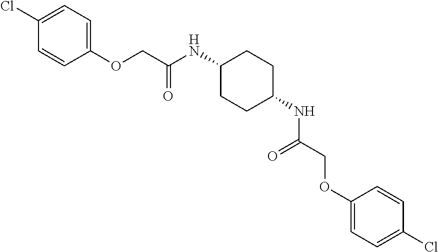 [SMILES: O=C(COc1ccc(Cl)cc1)N[C@H]1CC[C@@H](NC(=O)COc2ccc(Cl)cc2)CC1]

is combined with a lysosomotropic agent as described hereinabove, the combination of agents provides a particularly effective treatment for cancer, especially a tumor. In embodiments, the cancer is pancreatic cancer, a glioma, a glioblastoma or a neuroblastoma. In an embodiment, the method further combines at least one siRNA of SEQ ID Nos 3-10 in combination with ISRIB or an siRNA in combination with ISRIB and a lysosomotropic agent in providing an effective treatment of cancer, including a tumor, and especially pancreatic cancer, a glioma, glioblastoma or a neuroblastoma, among others described herein.

In an embodiment, the present invention is directed to methods of treating autophagy mediated disease states and/or conditions, especially cancer using a modulator, especially an inhibitor of NUFIP2 or G3BP1 (such as one or more siRNAs as described above) which downregulates NUFIP2 or G3BP1). It has been discovered that NUFIP2 or an agonist thereof inactivates or inhibits mTOR and that an antagonist of NUFIP2 upregulates mTOR. The implications of this interaction are that an agonist or upregulator of NUFIP2 will enhance autophagy and an antagonist of NUFIP2 will result in a decrease in autophagy.

In an embodiment, the invention is directed to compositions which comprise an effective (generally, a therapeutically effective) amount of at least one siRNA according to SEQ ID Nos 3-10, formulated in lipid nanoparticles or as conjugates and combined with a lysosomotropic agent. In embodiments, compositions comprise an effective amount of SJ-19-0043 and/or ISIRB, which may be combined with siRNA(s) and optionally lysosomotropic agents, as described herein.

### BRIEF DESCRIPTION OF THE FIGURES

FIGS. 1A-1J show that lysosomal damage induces stress granule formation.

FIG. 1A shows (i) encyclopeDIA/scaffoldDIA analysis of lysosomes purified by anti-HA immunoprecipitation (LysoIP; TMEM192-3×HA) from HEK293T cells treated with or without 1 mM LLOMe for 30 min. Three groups of proteins are labeled (ESCRT components in green, autophagy factors in blue and stress granule components in purple). Scatter (volcano) plot shows log 2 fold change and −Log 10 p-value for the proteins identified and quantified (Liquid Chromatography with tandem mass spectrometry, LC/MS/MS) in three independent experiments. The dotted orange line indicates the significance cut-off (P<0.05). (ii) shows enrichment analysis for biological processes in LysoIP LC/MS/MS. The significant increased proteins in LysoIP LC/MS/MS were analyzed by STRING functional protein association networks. The sub-selection biological processes are displayed. Strength, Log 10(observed/expected) describes how large the enrichment effect is. FIG. 1B shows the analysis for the indicated proteins in whole cell lysates or lysosomes purified by anti-HA immunoprecipitation (LysoIP; TMEM192-3×HA) from HEK293T cells treated with 2 mM LLOMe or 100 μM NaAsO₂ for 30 min. TMEM192-2×FLAG, control. FIG. 1C shows the quantification by high-content microscopy (HCM) of G3BP1 puncta. U20S cells Were treated with Earle's Balanced Salt Solution (EBSS), 4 mM LOMe, 2 mM LLOMe, 200 μM GPN, or 400 μg/mL silica for 30 min. White masks, algorithm-defined cell boundaries (primary objects); red masks, computer-identified G3BP1 puncta (target objects). FIG. 1D shows the mmunofluorescence confocal microscopy analysis of G3BP1. U2OS cells Were treated with 2 mM LLOMe for 30 min and stained for endogenous G3BP1. Scale bar, 5 μm. FIG. 1E shows the Quantification by HCM of G3BP1 puncta in BMM (bone-marrow-derived macrophages) cells treated with 2 mM LLOMe or 100 μM NaAsO₂ for 2 h. Green masks, computer-identified G3BP1 puncta. FIG. 1F shows the Quantification by HCM of Galectin-3 puncta in BMM cells treated with 2 mM LLOMe or 100 μM NaAsO₂ for 2 h. Red masks, computer-identified Galectin-3 puncta. FIG. 1G Immunoblot analysis of eIF2α (S51) phosphorylation in U2OS cells treated with 2 mM LLOMe for 30 min and followed by 1 h washout. FIG. 1H shows an immunoblot analysis of eIF2α (S51) phosphorylation in BMM cells treated with 2 mM LLOMe or 100 μM NaAsO₂ for 2 h. The level of phosphorylation of eIF2α (S51) is quantified based on three independent experiments. FIG. 1I shows the confocal microscopy analysis of G3BP1 (Alexa Fluor 488) and polyA RNA (Cy3-oligo[dT]) by fluorescence in situ hybridization (FISH) in U20S cells treated with 2 mM LLOMe for 30 min. Scale bar, 5/10 μm. FIG. 1J shows the immunoblot analysis of ATF4 and phosphorylation of eIF2α (S51) and 4EBP1 (S65) in U2OS cells treated with 2 mM LLOMe for the indicated time points. The level of ATF4 and phosphorylation of eIF2α (S51) and 4EBP1 (S65) are quantified based on three independent experiments. Ctrl, control (untreated cells). Data, means±SEM; HCM: n≥3 (each experiment: 500 valid primary objects/cells per well, ≥5 wells/sample). †p≥0.05 (not significant), *p<0.05, **p<0.01, ANOVA. See also FIG. 1S.

FIGS. 2A-2E show that PKR transmits lysosomal damage signals leading to stress granule formation. FIG. 2A shows unique peptides and exclusive intensity of PKR identified from LysoIP EncyclopeDIA/scaffoldDIA analysis in three independent experiments, Mann-Whitney U test p-value=0.0495 (LLOMe treatment relative to Ctrl). FIG. 2B shows immunoblot analysis of the phosphorylation of eIF2α (S51) in Huh7 cells transfected with scrambled siRNA as control (SCR) and HRI, PKR, PERK and GCN2 siRNA treated with 2 mM LLOMe for 30 min. The level of phosphorylation of eIF2α (S51) is quantified based on three independent experiments. FIG. 2C shows immunoblot analysis of PKR (T446) and eIF2α (S51) phosphorylation in U2OS cells treated with or without PKR inhibitor 2-AP for 1 h followed by 2 mM LLOMe treatment for 30 min as indicated. FIG. 2D shows quantification by HCM of G3BP1 puncta in Huh7 cells transfected with scrambled siRNA as control (SCR) and HRI, PKR, PERK and GCN2 siRNA treated with 2 mM LLOMe for 30 min. Red masks, computer-identified G3BP1 puncta. FIG. 2E shows quantification by HCM of G3BP1 puncta in U20S cells treated with or without PKR inhibitor 2-AP for 1 h followed by 2 mM LLOMe treatment for 30 min as indicated. Ctrl, control (untreated cells). Data, means±SEM; HCM: n≥3 (each experiment: 500 valid primary objects/cells per Well, ≥5 Well s/sample). *p<0.05, **p<0.01, ANOVA. See also FIG. 2S.

FIGS. 3A-3E show that NUFIP2 is required for both mTOR inactivation and stress granule formation during lysosomal damage. FIG. 3A shows an analysis for the FLAG-NUFIP2 or NUFIP2ΔNLS in whole cell lysates or lysosomes purified by anti-HA immunoprecipitation (LysoIP; TMEM192-3×HA) from Huh7 cells transfected with FLAG-NUFIP2 or NUFIP2ΔNLS after the treatment with 2 mM LLOMe for 30 min. TMEM192-2×FLAG, control. FIG. 3B shows the quantification by HCM of overlaps between mTOR and LAMP2 in U2OS transfected with scrambled siRNA as control (SCR) and NUFIP2 siRNA (NUFIP2^{KD}) treated with 2 mM LLOMe for 30 min. White masks, algorithm-defined cell boundaries. Yellow masks, computer-identified overlap of mTOR and LAMP2. FIG. 3C shows the immunoblot analysis of the phosphorylation of ULK1 (S757) in U2OS cells transfected with scrambled siRNA as control (SCR) and NUFIP2 siRNA (NUFIP2^{KD}) treated with 2 mM LLOMe for 30 min. The level of phosphorylation of ULK1 (S757) is quantified based on three independent experiments. FIG. 3D shows the quantification by HCM of overlaps between mTOR and LAMP2 in parental Huh7 (WT) and NUFIP2-knockout Huh7 cells (Huh7^{NUFIP2-KO)} treated with 2 mM LLOMe for 30 min. Yellow masks, computer-identified overlap of mTOR and LAMP2. FIG. 3E shows an immunoblot analysis of the phosphorylation of ULK1 (S757) and S6K1 (T389) in parental Huh7 (WT) and NUFIP2-knockout Huh7 cells (Huh7^{NUFIP2-KO)} treated with 2 mM LLOMe for 30 min. The level of phosphorylation of ULK1 (S757) and S6K1 (T389) is quantified based on three independent experiments. Ctrl, control (untreated cells). Data, means±SEM; HCM: n≥3 (each experiment: 500 valid primary objects/cells per Well, ≥5 Well s/sample). †p≥0.05 (not significant), *p<0.05, **p<0.01, ANOVA. See also FIG. 4S.

FIGS. 4A-4F show that ragulator abundance and activity on damaged lysosomes is controlled by NUFIP2. FIG. 4A shows a summary of the relevant proteins of mTORC1 signaling in EncyclopeDIA/scaffoldDIA LysoIP LC/MS/MS analysis. FIG. 4B shows an analysis for the indicated proteins in cell lysates or lysosomes purified by anti-HA immunoprecipitation (LysoIP; TMEM192-3×HA) from HEK293T cells treated with 2 mM LLOMe for 30 min. TMEM192-2×FLAG, control. FIG. 4C shows an analysis for the indicated proteins in whole cell lysates or lysosomes purified by anti-HA immunoprecipitation (LysoIP; TMEM192-3×HA) from parental Huh7 (WT) and NUFIP2-knockout Huh7 cells (Huh7^{NUFIP2-KO}) with 2 mM LLOMe for 30 min. TMEM192-2×FLAG, control. The quantification is based on three independent experiments. FIG. 4 D Co-immunoprecipitation analysis of changes in interactions between Ragulator (LAMTOR2) and Rag GTPases (RagA) following treatment with LLOMe in cells transfected with scrambled siRNA as control (SCR) and NUFIP2 siRNA (NUFIP2^{KD}). HEK293T cells stably expressing FLAG-Metap2 (control) or FLAG-LAMTOR2 transfected with scrambled siRNA as control (SCR) and NUFIP2 siRNA (NUFIP2^{KD}) Were treated with 2 mM LLOMe for 30 min. Cell lysates Were immunoprecipitated (IP) with anti-FLAG antibody and immunoblotted for endogenous RagA. The level of interaction between RagA and LAMTOR2 is quantified based on three independent experiments. FIG. 4E shows the co-immunoprecipitation analysis of changes in interactions between Ragulator (LAMTOR2) and Rag GTPases (RagA) following treatment with LLOMe in cells overexpressing GFP (control) or GFP-NUFIP2. HEK293T cells stably expressing FLAG-Metap2 (control) or FLAG-LAMTOR2 transfected with GFP (control) or GFP-NUFIP2 Were treated with 2 mM LLOMe for 30 min. Cell lysates Were immunoprecipitated (IP) with anti-FLAG antibody and immunoblotted for endogenous RagA. The level of interaction between RagA and LAMTOR2 is quantified based on three independent experiments. FIG. 4F Co-immunoprecipitation (CoIP) analysis of interactions between LAMTOR1 and NUFIP2 during lysosomal damage. HEK293T cells stably expressing FLAG (control) or FLAG-NUFIP1 Were treated with 2 mM LLOMe for 30 min. Cell lysates Were immunoprecipitated (IP) with anti-FLAG antibody and immunoblotted for endogenous LAMTOR1. Data, means±SEM; *p<0.05, **p<0.01, ANOVA. See also FIG. 4S.

FIGS. 5A-5M show that Mammalian Atg8s participate in recruitment of NUFIP2 to damaged lysosomes. FIG. 5A shows a summary of the relevant proteins of autophagy factors in EncyclopeDIA/scaffoldDIA LysoIP LC/MS/MS analysis. FIG. 5B shows an analysis for the indicated proteins in cell lysates or lysosomes purified by anti-HA immunoprecipitation (LysoIP; TMEM192-3×HA) from parental HeLa (WT), LC3^{TKO}, GBRP^{TKO} and hexa^{KO} treated with 4 mM LLOMe for 30 min. TMEM192-2×FLAG, control. FIG. 5C shows a GST pull-down assay of in vitro translated and radiolabeled Myc-tagged NUFIP2 with GST or GST-tagged mAtg8. FIG. 5D shows quantification of C. Data (% binding) represents the percentage of the corresponding protein relative to its input. FIG. 5E shows a GST pull-down assay of in vitro translated and radiolabeled Myc-tagged G3BP1 with GST or GST-tagged mAtg8. FIG. 5F shows that quantification of E. Data (% binding) represents the percentage of the corresponding protein relative to its input. FIG. 5G shows the quantification of GST pull-down assay of in vitro translated and radiolabeled Myc-tagged NUFIP2 with GST or GST-tagged GABARAP deletions, FIG. 5SA. Data (% binding) represents the percentage of the corresponding protein relative to its input. FIG. 5 H shows quantification of GST pull-down assay of in vitro translated and radiolabeled Myc-tagged G3BP1 with GST or GST-tagged GABARAP deletions, FIG. 5SC. Data (% binding) represents the percentage of the corresponding protein relative to its input. FIG. 5I shows an immunoblot analysis of the phosphorylation S6K1 (T389) and eIF2α (S51) in parental HeLa (WT), LC3^{TKO} GBRP^{TKO} and hexa^{KO} treated with 4 mM LLOMe for 30 min. FIG. 5J shows the level of phosphorylation S6K1 (T389) is quantified based on three independent experiments. FIG. 5K shows the level of phosphorylation eIF2α (S51) is quantified based on three independent experiments. FIG. 5L shows the quantification by HCM of overlaps between mTOR and LAMP2 in parental HeLa (WT), LC3^{TKO}, GBRP^{TKO} and hexa^{KO} treated with 4 mM LLOMe for 30 min. White masks, algorithm-defined cell boundaries. Yellow masks, computer-identified overlap of mTOR and LAMP2. FIG. 5M shows the Quantification by HCM of G3BP1 puncta. Parental HeLa (WT), LC3^{TKO}, GBRP^{TKO} and hexa^{KO} Were treated with treated with 4 mM LLOMe for 30 min. Red masks, computer-identified G3BP1 puncta. Data, means±SEM; HCM: n≥3 (each experiment: 500 valid primary objects/cells per Well, ≥5 Well s/sample). †p≥0.05 (not significant), **p<0.01, ANOVA. See also FIGS. 5S and 6S.

FIGS. 6A-6H show that Atg8ylation plays a role in mTOR inhibition and competes with stress granule formation during lysosomal damage. FIG. 6A shows an immunoblot analysis of GABARAP lipidation in U2OS cells treated with 2 mM LLOMe for the indicated time points. FIG. 6B shows the quantification by HCM of overlaps between mTOR and LAMP2 in parental Huh7(WT), ATG9^{KO} and ATG3^{KO} treated with 2 mM LLOMe for 30 min. White masks, algorithm-defined cell boundaries. Yellow masks, computer-identified overlap of mTOR and LAMP2. FIG. 6C shows the quantification by HCM of G3BP1 puncta. Parental Huh7(WT), ATG9^{KO} and ATG3^{KO} Were treated with 2 mM LLOMe for 30 min. Red masks, computer-identified G3BP1 puncta. FIG. 6D shows the quantification by HCM of overlaps between mTOR and LAMP2 in parental Huh7(WT), FIP200^{KO} and ATG16L1^{KO} treated with 2 mM LLOMe for 30 min. Yellow masks, computer-identified overlap of mTOR and LAMP2. FIG. 6E shows the quantification by HCM of G3BP1 puncta. Parental Huh7(WT), FIP200^{KO} and ATG16L1^{KO} Were treated with 2 mM LLOMe for 30 min. Red masks, computer-identified G3BP1 puncta. FIG. 6F shows the quantification by HCM of overlaps between mTOR and LAMP2 in parental HeLa (WT), ATG13^{KO} and ATG3^{KO} treated with 4 mM LLOMe for 30 min. Yellow masks, computer-identified overlap of mTOR and LAMP2. FIG. 6G Quantification by HCM of G3BP1 puncta. Parental HeLa (WT), ATG13^{KO} and ATG3^{KO} Were treated with 4 mM LLOMe for 30 min. Red masks, computer-identified G3BP1 puncta. FIG. 6H Schematic summary of the findings in this study. Ctrl, control (untreated cells). Data, means±SEM; HCM: n≥3 (each experiment: 500 valid primary objects/cells per Well, ≥5 Well s/sample). †p≥0.05 (not significant), **p<0.01, ANOVA. See also FIG. 6S.

FIGS. 7A-7D show that diverse pathological agents induce lysosomal damage and stress granule formation response. FIG. 7A Quantification by HCM of G3BP1 and ubiquitin (Ubi) puncta in BMM cells infected with M. tuberculosis strain Erdman or its ESX-1 mutant at MOI=10 for 20 h. White masks, algorithm-defined cell boundaries. Red masks, computer-identified G3BP1 puncta. Green masks, computer-identified ubiquitin puncta. FIG. 7B shows the quantification by HCM of G3BP1 and ubiquitin (Ubi) puncta in U2OS cells treated with FuGENE HD-coated latex beads (Polybead Amino Microsphere) for 16 h. White masks, algorithm-defined cell boundaries. Green masks, computer-identified G3BP1 puncta. Red masks, computer-identified ubiquitin puncta. FIG. 7C shows the quantification by HCM of G3BP1 and galectin-3 (Gal3) puncta in U2OS cells treated with 1 or 10 μg/mL Tau oligomer overnight. Red masks, computer-identified G3BP1 puncta. Green masks, computer-identified Gal3 puncta. FIG. 7D shows the quantification by HCM of G3BP1 and ubiquitin (Ubi) puncta in the constructed HeLa Flp-In^{TetON} GFP-SARS-CoV-2^{ORF3a} cells induced by 1 μg/mL tetracycline (Tet). Red masks, computer-identified G3BP1 puncta. Pink masks, computer-identified ubiquitin puncta. Stress granule (SG). Data, means±SEM; HCM: n≥3 (each experiment: 500 valid primary objects/cells per Well, ≥5 Well s/sample). †p≥0.05 (not significant), *p<0.05, **p<0.01, ANOVA. See also FIG. 6S.

FIGS. 8A-8H show that stress granule formation promotes cell survival in response to lysosomal damage. FIG. 8A) shows quantification by high-content microscopy (HCM) of cell death by a propidium iodide (PI) uptake assay in U2OS wildtype (WT) and G3BP1&2 double knockout (ΔΔG3BP1/2) cells. Cells Were treated with 2 mM LLOMe for 30 min, and then stained with propidium iodide (PI) (dead cells) and Hoechst-33342 (total cells). White masks, algorithm-defined cell boundaries (primary objects); red masks, computer-identified PI⁺ nuclei (target objects). FIG. 8B shows cell death analysis of supernatants of U2OS WT and ΔΔG3BP1/2 cells by a LDH release assay. Cells Were treated with 2 mM LLOMe for 30 min. FIG. 8C shows the quantification by HCM of cell death by a PI uptake assay in human peripheral blood monocyte-derived macrophages (hMDM). Cells Were treated with 2 mM in the presence or absence of 10 μg/ml cycloheximide (CHX) for 30 min, and then stained with PI (dead cells) and Hoechst-33342 (total cells). FIG. 8D shows the confocal microscopy analysis of G3BP1 (Alexa Fluor 488) in hMDM treated with 2 mM LLOMe with or without CHX for 30 min. Scale bar, 10 μm. FIG. 8E shows the quantification using AMNIS of cell death by Live/Dead™ stain kit in hMDM. Cells Were treated with 2 mM LLOMe with or without CHX for 30 min, and then stained using Live/Dead™ stain kit (ThermoFisher). FIG. 8F shows the quantification by HCM of cell death by a PI uptake assay and SG formation by eIF4G in hMDM transfected with scrambled siRNA as control (SCR) or G3BP1 and G3BP2 siRNA for double knockdown (DKD). Cells Were treated with 2 mM LLOMe for 30 min, and then stained with PI (dead cells), Hoechst-33342 (total cells) or eIF4G. (i) HCM images: white masks, algorithm-defined cell boundaries; green masks, computer-identified eIF4G puncta; red masks, computer-identified PI⁺ nuclei (target objects); (ii and iii) corresponding HCM quantification. FIG. 8G shows the cell death analysis of supernatants of hMDM transfected with either scrambled siRNA as control (SCR) or G3BP1 and G3BP2 siRNA for double knockdown (DKD) using a LDH release assay. Cells Were treated with 2 mM LLOMe for 30 min. FIG. 8H shows the schematic summary of the findings in FIGS. 8 and 7S. CTR, control; NT, untreated cells. Data, means±SEM (n=3); HCM: n≥3 (each experiment: 500 valid primary objects/cells per Well, ≥5 Well s/sample). †p≥0.05 (not significant), *p<0.05, **p<0.01, ANOVA. See also FIG. 7S.

FIGS. 9A-9F show that stress granule formation is controlled by eIF2a pathway but not mTORC1 pathway during lysosomal damage. FIG. 9A shows the quantification by HCM of G3BP1 puncta in U2OS cells transfected with either scrambled siRNA as control (SCR) or eIF2α siRNA for knockdown (eIF2α^{KD}). Cells Were treated with 2 mM LLOMe for 30 min. White masks, algorithm-defined cell boundaries; red masks, computer-identified G3BP1 puncta. FIG. 9B shows an immunoblot analysis of mTORC1 activity by phosphorylation of 4EBP1 (S65) in U2OS cells transfected with either scrambled siRNA as control (SCR) or eIF2α siRNA for knockdown (eIF2α^{KD}). Cells Were treated with 2 mM LLOMe for 30 min. FIG. 9C shows an immunoblot analysis of phosphorylation of eIF2α (S51) in U2OS cells overexpressing wildtype RagB (RagB^{WT}) or constitutively active RagB mutant (RagB^{Q99L}) treated with 2 mM LLOMe for 30 min. FIG. 9D shows the quantification by HCM of G3BP1 puncta in U2OS cells overexpressing wildtype RagB (RagB^{WT}) or constitutively active RagB mutant (RagB^{Q99L}). Cells Were treated with 2 mM LLOMe for 30 min. White masks, algorithm-defined cell boundaries; green masks, computer-identified G3BP1 puncta. FIG. 9E show the quantification by HCM of G3BP1 puncta in eIF2α knockdown (eIF2α^{KD}) U2OS cells transfected with FLAG, FLAG-eIF2α^{WT} or FLAG-eIF2α^{S51A}. Cells Were treated with 2 Mm LLOMe for 30 min. White masks, algorithm-defined cell boundaries; red masks, computer-identified G3BP1 puncta. FIG. 9F shows aschematic summary of the findings in FIGS. 2 and S2. NT, untreated cells. Data, means±SEM (n=3); HCM: n≥3 (each experiment: 500 valid primary objects/cells per Well, ≥5 Well s/sample). †p≥0.05 (not significant), **p<0.01, ANOVA. See also FIG. 8S.

FIGS. 10A-10G shows that PKR and its activator PACT regulate eIF2α phosphorylation on damaged lysosomes. FIG. 10A Quantitative liquid chromatography-tandem mass spectrometry (LC/MS/MS) using the data-independent acquisition (DIA) technique to identify eIF2α binding partners that Were proximity-biotinylated by APEX2-eIF2α during lysosomal damage (1 mM LLOMe for 1 h). Scatter (volcano) plot shows log 2 fold change (LLOMe/CTR; spectral counts) and −log 10 p value for the proteins identified and quantified in three independent experiments. Green dots indicate increase in proximity to eIF2α (log 2 fold change ≥1), and red dots indicate decrease in proximity to eIF2α (log 2 fold change ≤−1) during LLOMe treatment. Orange dots indicate values below the statistical significance cut-off (P≥0.05). Bubble size represents a normalized value for the total amount of spectral counts for the protein indicated. PACT, PKR and ALIX proteins are highlighted as purple circles. FIG. 10B shows a Co-IP analysis of interactions between eIF2α and PKR/PACT during lysosomal damage. HEK293T cells expressing FLAG (control) or FLAG-eIF2α Were treated with 1 mM LLOMe for 30 min. Cell lysates Were immunoprecipitated with anti-FLAG antibody and immunoblotted for indicated proteins. FIG. 10C shows Quantification by HCM of G3BP1-GFP puncta in wildtype (WT) or PKR knockout (PKR^{KO}) U2OS G3BP1-GFP cells. Cells Were treated with 2 mM LLOMe for 30 min. White masks, algorithm-defined cell boundaries; green masks, computer-identified G3BP1 puncta. FIG. 10D shows an immunoblot analysis of phosphorylation of eIF2α (S51) and PKR (T446) in WT or PKR^{KO} U2OS G3BP1-GFP cells, as Well as in cells overexpressing FLAG-PKR in PKR^{KO} U2OS G3BP1-GFP cells. Cells Were treated with 2 mM LLOMe for 30 min. FIG. 10E shows (i) the quantification by HCM of G3BP1 puncta in U2OS cells transfected with either scrambled siRNA as control (SCR) or PACT siRNA for knockdown (PACT^{KD}). Cells Were treated with 2 mM LLOMe for 30 min. White masks, algorithm-defined cell boundaries; red masks, computer-identified G3BP1 puncta; and (ii) an immunoblot analysis of phosphorylation of eIF2α (S51) and PKR (T446) in SCR or PACT^{KD} cells; 2 mM LLOMe for 30 min. FIG. 10F shows an analysis of proteins associated with purified lysosomes (LysoIP; TMEM192-3×HA) from HEK293T cells treated with 1 mM LLOMe in the presence or absence of 210 nM imidazolo-oxindole C16 for 1 h. TMEM192-2×FLAG, control. FIG. 10G shows the schematic summary of the findings in FIGS. 10 and 9S. NT, untreated cells. Data, means±SEM (n=3); HCM: n≥3 (each experiment: 500 valid primary objects/cells per Well, ≥5 Well s/sample). †p≥0.05 (not significant), **p<0.01, ANOVA. See also FIG. 9S.

FIGS. 11A-11G show that ALIX and ALG2 are required for stress granule formation by sensing calcium release from damaged lysosomes. FIG. 11A shows quantification by HCM of G3BP1 puncta in U2OS cells transfected with either scrambled siRNA as control (SCR) or ALIX siRNA for knockdown (ALIX^{KD}). Cells Were treated with 2 mM LLOMe for 30 min. White masks, algorithm-defined cell boundaries; green masks, computer-identified G3BP1 puncta. FIG. 11B shows immunoblot analysis of phosphorylation of eIF2α (S51) and PKR (T446) in U2OS cells transfected with either scrambled siRNA as control (SCR) or ALIX siRNA for knockdown (ALIX^{KD}). Cells Were treated with 2 mM LLOMe for 30 min. FIG. 11C shows quantification by HCM of G3BP1 puncta in U2OS cells transfected with scrambled siRNA as control (SCR), ALIX siRNA for knockdown (ALIX^{KD}) or TSG101 siRNA for knockdown (TSG101^{KD}). Cells Were treated with 2 mM LLOMe for 30 min. White masks, algorithm-defined cell boundaries; red masks, computer-identified G3BP1 puncta. FIG. 11D shows an immunoblot analysis of phosphorylation of eIF2α (S51) in U2OS cells transfected with scrambled siRNA as control (SCR), ALIX siRNA for knockdown (ALIX^{KD}) or TSG101 siRNA for knockdown (TSG101^{KD}). Cells Were treated with 2 mM LLOMe for 30 min. FIG. 11E (i) shows quantification by HCM of G3BP1 puncta in U2OS cells pre-treated with 15 μM BAPTA-AM for 1 h, subjected to 2 mM LLOMe treatment for 30 min. White masks, algorithm-defined cell boundaries; red masks, computer-identified G3BP1 puncta. (ii) Immunoblot analysis of phosphorylation of eIF2α (S51) in U2OS cells as described in (i). FIG. 11F (i) Quantification by HCM of G3BP1 puncta in U2OS cells transfected with scrambled siRNA as control (SCR), or ALG2 siRNA for knockdown (ALG2^{KD}). Cells Were treated with 2 mM LLOMe for 30 min. White masks, algorithm-defined cell boundaries; red masks, computer-identified G3BP1 puncta. (ii) Immunoblot analysis of phosphorylation of eIF2α (S51) in U2OS cells as described in (i). FIG. 11G shows the schematic summary of the findings in FIGS. 11 and 10S. NT, untreated cells. CTR, control. Data, means±SEM (n=3); HCM: n≥3 (each experiment: 500 valid primary objects/cells per Well, ≥5 Well s/sample). †p≥0.05 (not significant), **p<0.01, ANOVA. See also FIG. 10S.

FIGS. 12A-12G shows that ALIX promotes the association between PKR and its activator PACT on damaged lysosomes. FIG. 12A shows Co-IP analysis of interactions among ALIX, PKR and PACT during lysosomal damage. HEK293T cells expressing FLAG (control) or FLAG-ALIX Were treated with 1 mM LLOMe for 30 min. Cell lysates Were immunoprecipitated with anti-FLAG antibody and immunoblotted for indicated proteins. FIG. 12B (i) shows a schematic diagram of ALIX mutants used in this study. FL (full length); Bro1 (Bro1 domain); V domain; PRD (proline-rich domain). Numbers, residue positions. (ii) Schematic illustration of the Ca²⁺/ALG-2-induced open conformation of ALIX. FIG. 12C shows Co-IP analysis of interactions among ALIX mutants, PKR and PACT during lysosomal damage. HEK293T cells expressing FLAG tagged ALIX mutants and Myc-PKR Were treated with 1 mM LLOMe for 30 min. Cell lysates Were immunoprecipitated with anti-FLAG antibody and immunoblotted for indicated proteins. FIG. 12D shows Co-IP analysis of interactions between FLAG-PKR and PACT in HEK293T cells transfected with scrambled siRNA as control (SCR), or ALIX siRNA for knockdown (ALIX^{KD}) during lysosomal damage. Cells Were treated with 1 mM LLOMe for 30 min. Cell lysates Were immunoprecipitated with anti-FLAG antibody and immunoblotted for indicated proteins. FIG. 12E shows Co-IP analysis of interactions between PKR and GFP-PACT in HEK293T cells transfected with FLAG, or FLAG-ALIX during lysosomal damage. Cells Were treated with 1 mM LLOMe for 30 min. Cell lysates Were immunoprecipitated with anti-GFP antibody and immunoblotted for indicated proteins. FIG. 12F Analysis of proteins associated with purified lysosomes (LysoIP; TMEM192-3×HA) from HEK293T cells transfected with scrambled siRNA as control (SCR), or ALIX siRNA for knockdown (ALIX^{KD}). Cells Were treated with 1 mM LLOMe for 30 min. FIG. 12G Schematic summary of the findings in FIGS. 5 and S5. One of three independent western Blot experiments shown. See also FIG. 1S1.

FIGS. 13A-13G shows that Galectin-3 inhibits stress granule formation by reducing the association between PKR and PACT during lysosomal damage. FIG. 13A show the quantification by HCM of G3BP1 puncta in U2OS cells transfected with scrambled siRNA as control (SCR), or galectin-3 (Gal3) siRNA for knockdown (Gal3^{KD}). Cells Were treated with 2 mM LLOMe for 30 min. White masks, algorithm-defined cell boundaries; green masks, computer-identified G3BP1 puncta. FIG. 13B shows an immunoblot analysis of phosphorylation of eIF2α (S51) and PKR (T446) in U2OS cells transfected with scrambled siRNA as control (SCR), or galectin-3 (Gal3) siRNA for knockdown (Gal3^{KD}), subjected to 2 mM LLOMe treatment for 30 min. FIG. 13C shows a Co-IP analysis of interactions among FLAG-Gal3, ALIX, PKR and PACT in HEK293T cells during lysosomal damage. Cells Were treated with 1 mM LLOMe for 30 min. Cell lysates Were immunoprecipitated with anti-FLAG antibody and immunoblotted for indicated proteins. FIG. 13D shows a Co-IP analysis of interactions between FLAG-PKR and PACT in HEK293T cells transfected with scrambled siRNA as control (SCR), or Gal3 siRNA for knockdown (Gal3^{KD}) during lysosomal damage. Cells Were treated with 1 mM LLOMe for 30 min. Cell lysates Were immunoprecipitated with anti-FLAG antibody and immunoblotted for indicated proteins. FIG. 13E shows a Co-IP analysis of interactions between Myc-PACT and PKR in HEK293T cells transfected with FLAG, or FLAG-Gal3 during lysosomal damage. Cells Were treated with 1 mM LLOMe for 30 min. Cell lysates Were immunoprecipitated with anti-Myc antibody and immunoblotted for indicated proteins. FIG. 13F shows a Co-IP analysis of interactions among FLAG-ALIX, PKR and PACT in HEK293T cells transfected with GFP, GFP-Gal3 or GFP-Gal3R186S during lysosomal damage. Cells Were treated with 1 mM LLOMe for 30 min. Cell lysates Were immunoprecipitated with anti-FLAG antibody and immunoblotted for indicated proteins. FIG. 13G shows a schematic summary of the findings in FIG. 13. NT, untreated cells. Data, means±SEM (n=3); HCM: n≥3 (each experiment: 500 valid primary objects/cells per Well, ≥5 Well s/sample). **p<0.01, ANOVA. One of three independent Western Blot experiments shown.

FIGS. 14A-14G shows that stress granule formation promotes cell survival in response to lysosomal damage during disease states. FIG. 14A shows quantification by HCM of G3BP1 puncta in U2OS cells infected with wildtype human adenovirus C2 (HAdV-C2^{WT}) or C2 TS1 mutant (HAdV-C2^{TS1}) at MOI=10 for 1 h. White masks, algorithm-defined cell boundaries; red masks, computer-identified G3BP1 puncta. FIG. 14B shows an Immunoblot analysis of phosphorylation of eIF2α (S51) in U2OS cells infected with wildtype human adenovirus C2 (HAdV-C2^{WT}) or C2 TS1 mutant (HAdV-C2^{TS1}) at MOI=10 for 1 h. FIG. 14C shows the quantification by HCM of cell death by a propidium iodide (PI) uptake assay in U2OS wildtype (WT) and G3BP1&2 double knockout (ΔΔG3BP1/2) cells during adenovirus infection. Cells Were infected with wildtype human adenovirus C2 (HAdV-C2^{WT}) at MOI=10 for 1 h, and then stained with propidium iodide PI (dead cells) and Hoechst-33342 (total cells). White masks, algorithm-defined cell boundaries; red masks, computer-identified PI⁺ nuclei. FIG. 14D shows the cell death analysis of supernatants of U2OS WT and ΔΔG3BP1/2 cells by a LDH release assay during SARS-Cov-2^{ORF3a} expression. Cells Were transfected with the GFP-SARS-Cov-2^{ORF3a} construct overnight. FIG. 14E shows the cell death analysis of supernatants of human peripheral blood monocyte-derived macrophages (hMDM) by a LDH release assay during hemozoin exposure. Cells Were treated with 10 μg/ml hemozoin for 4 h in the presence or absence of 1 μg/ml cycloheximide (CHX). (F) Quantification using AMNIS of cell death by Live/Dead™ stain kit in hMDM during silica treatment. Cells Were treated with 200 μg/mL silica for 4 h in the presence or absence of 1 μg/ml cycloheximide (CHX), and then stained using Live/Dead™ stain kit (ThermoFisher). FIG. 14G shows the quantification using AMNIS of cell death by Live/Dead™ stain kit in hMDM during the treatment of tau oligomer. Cells were treated with 10 μg/mL tau oligomer for 4 h in the presence or absence of 1 μg/ml cycloheximide (CHX), and then stained using Live/Dead™ stain kit (ThermoFisher). CTR, control. Data, means±SEM (n=3); HCM: n≥3 (each experiment: 500 valid primary objects/cells per Well, ≥5 Well s/sample). *p<0.05, **p<0.01, ANOVA. See also FIG. 12S.

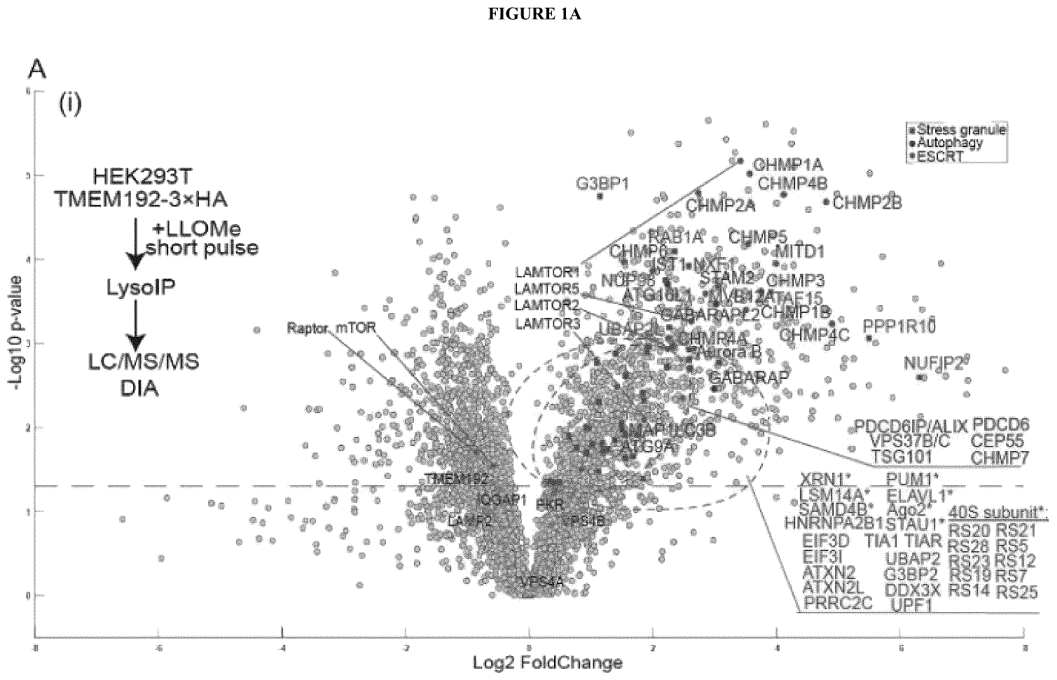

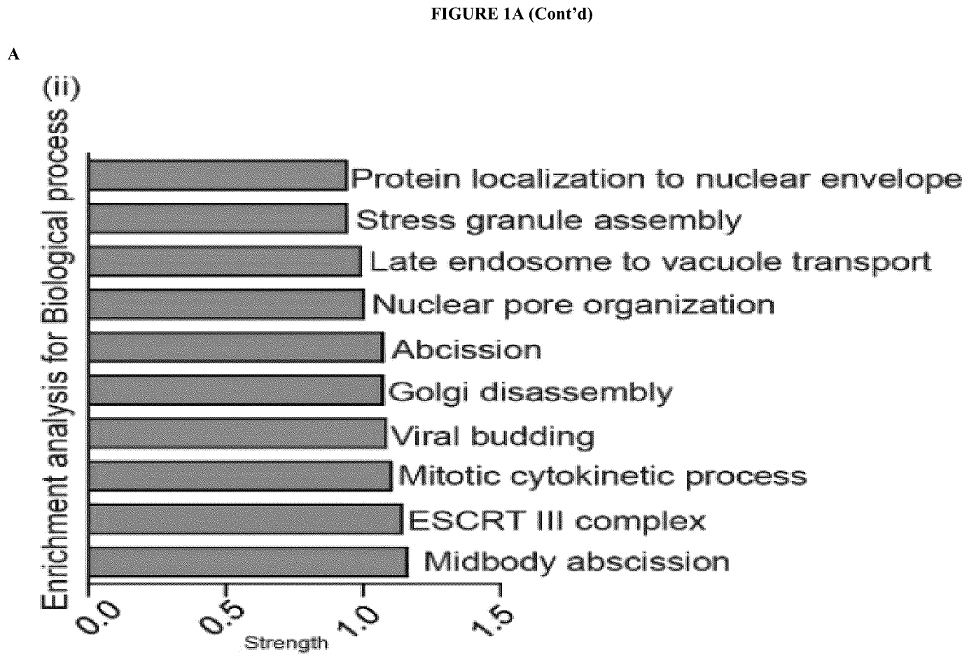

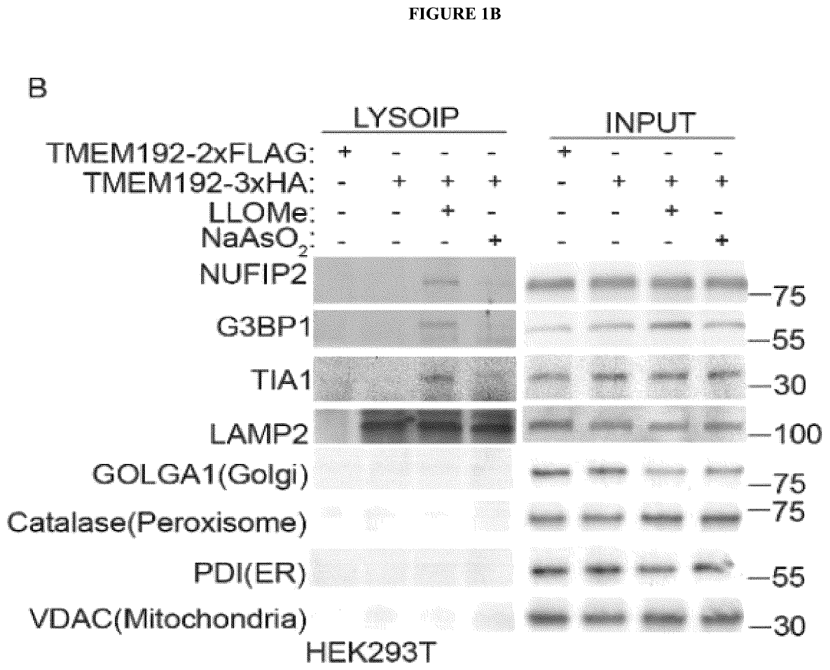

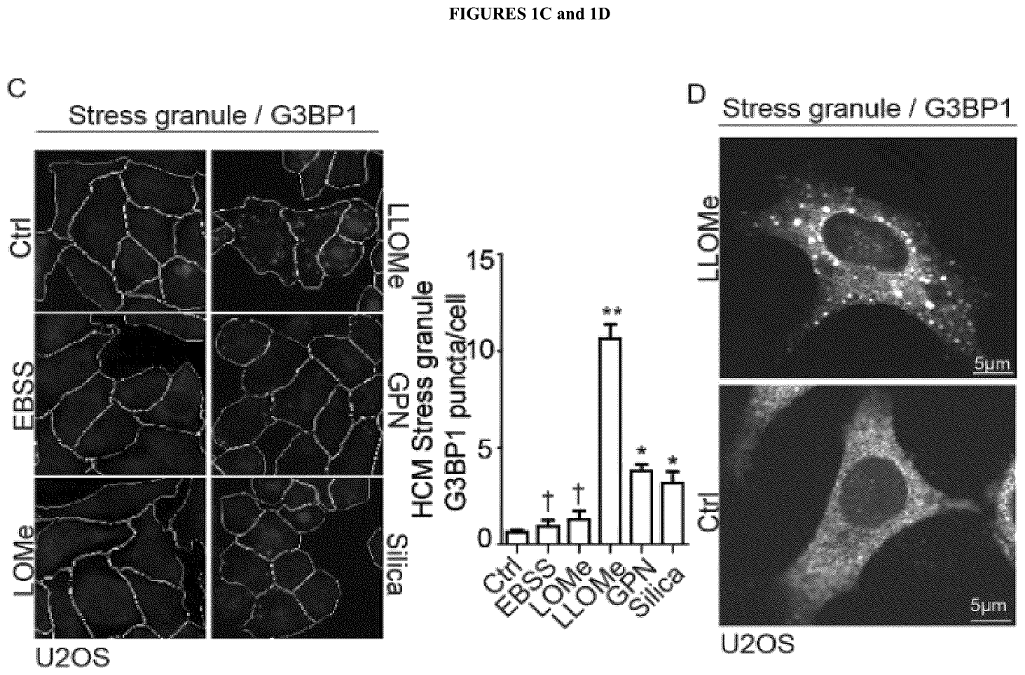

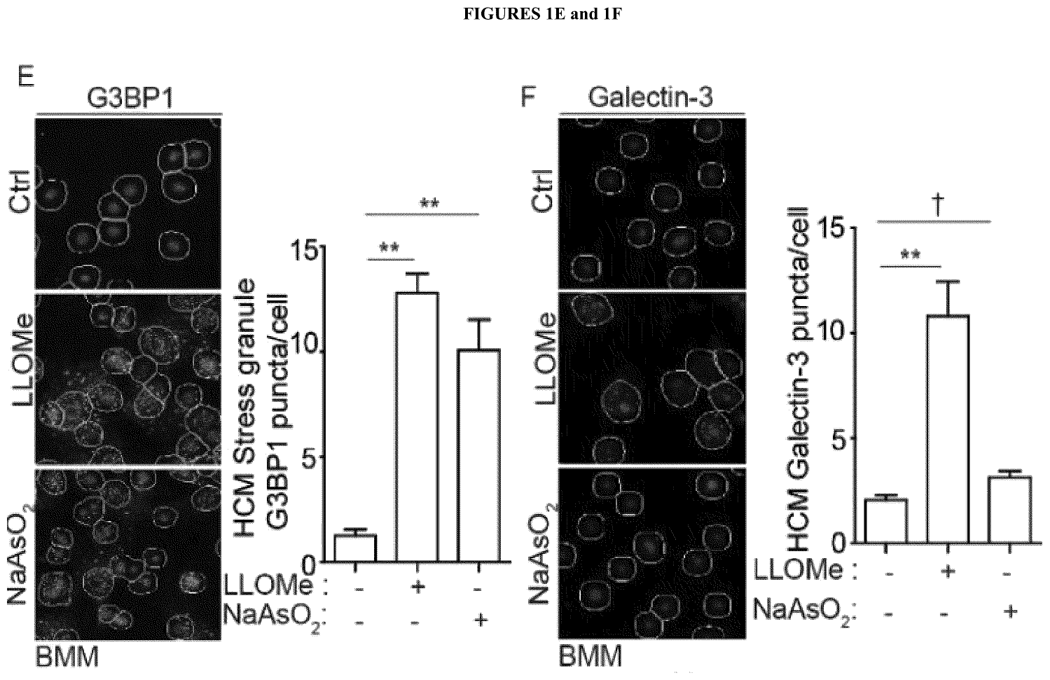

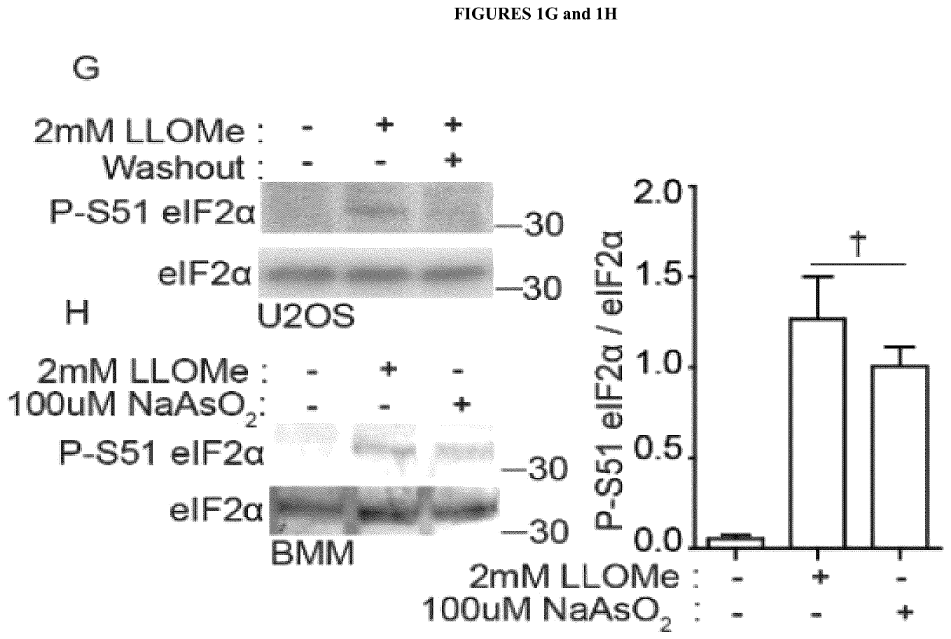

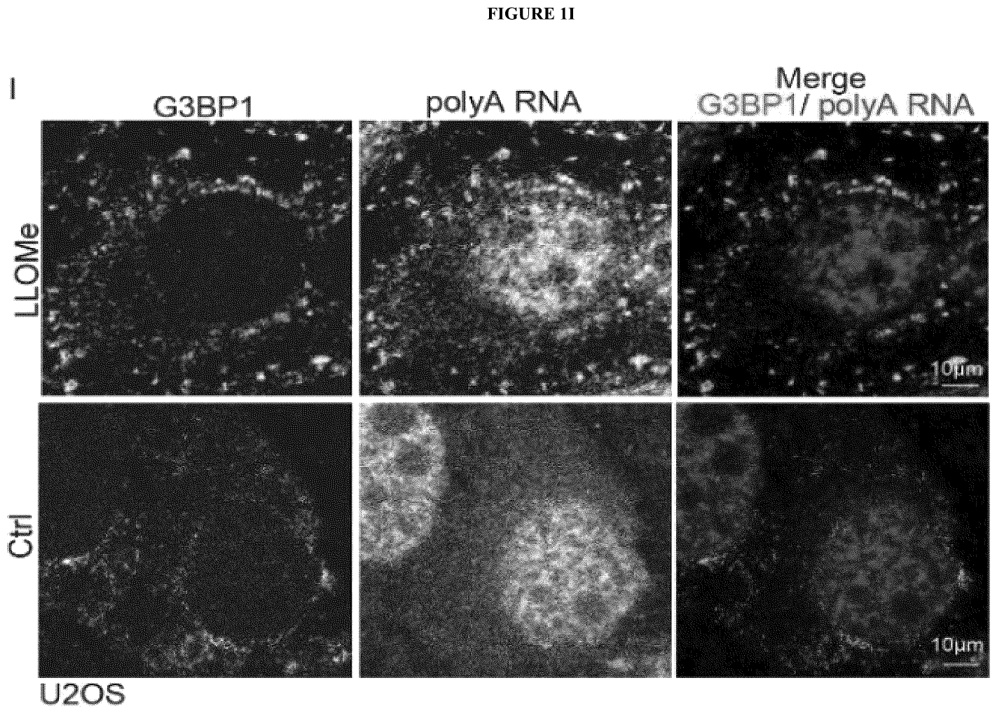

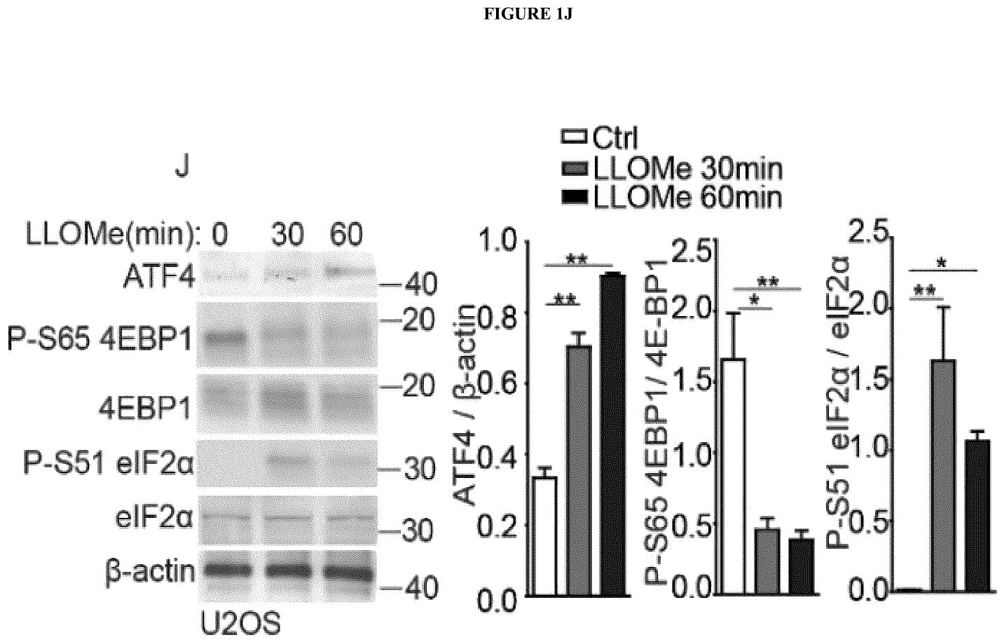

FIGS. 1SA-1SH shows that Lysosomal proteome changes and stress granule formation during lysosomal damage. FIG. 1SA (i) shows EncyclopeDIA/scaffoldDIA analysis of lysosomes purified by anti-HA immunoprecipitation (LYSOIP; TMEM192-3×HA) from HEK293T cells treated with or without 1 mM LLOMe for 30 min. Scatter (volcano) plot shows log 2 fold change and −Log 10 p-value for the proteins identified and quantified (LC/MS/MS) in three independent experiments. The dotted line indicates the significance cut-off (P<0.05). (ii) Enrichment analysis for biological processes in LysoIP LC/MS/MS. The significant increased proteins in LysoIP LC/MS/MS Were analyzed by STRING functional enrichment analysis. The sub-selection biological processes are displayed. FDR, false discovery rate. FIG. 1SB shows the quantification by HCM of DCP1a and G3BP1 puncta in U2OS cells treated with 2 mM LLOMe for 30 min. FIG. 1SC shows the quantification by HCM of G3BP1 puncta in Huh7 cells treated with 2 mM LLOMe for 30 min. White masks, algorithm-defined cell boundaries (primary objects); Green masks, computer-identified G3BP1 puncta (target objects). FIG. 1SD shows the quantification by HCM of TIA1 puncta in U2OS cells treated with 2 mM LLOMe for 30 min. Red masks, computer-identified TIA1 puncta. FIG. 1SE shows the quantification by HCM of TIA1 puncta in HeLa cells treated with 4 mM LLOMe for 30 min. Red masks, computer-identified TIA1 puncta. FIG. 1SF shows the quantification by HCM of G3BP1 puncta in U2OS cells treated with 2 mM LLOMe for 30 min and followed by 1 h washout. Red masks, computer-identified G3BP1 puncta. FIG. 1SG shows an immunoblot analysis of eIF2α (S51) phosphorylation in HEK293T cells treated with 2 mM LLOMe or 100 μM NaAsO₂ for 30 min. (H) Schematic summary of the findings in FIG. 1. Ctrl, control (untreated cells). Data, means±SEM; HCM: n≥3 (each experiment: 500 valid primary objects/cells per Well, ≥5 Well s/sample). *p<0.05, **p<0.01, ANOVA. See also FIG. 1.

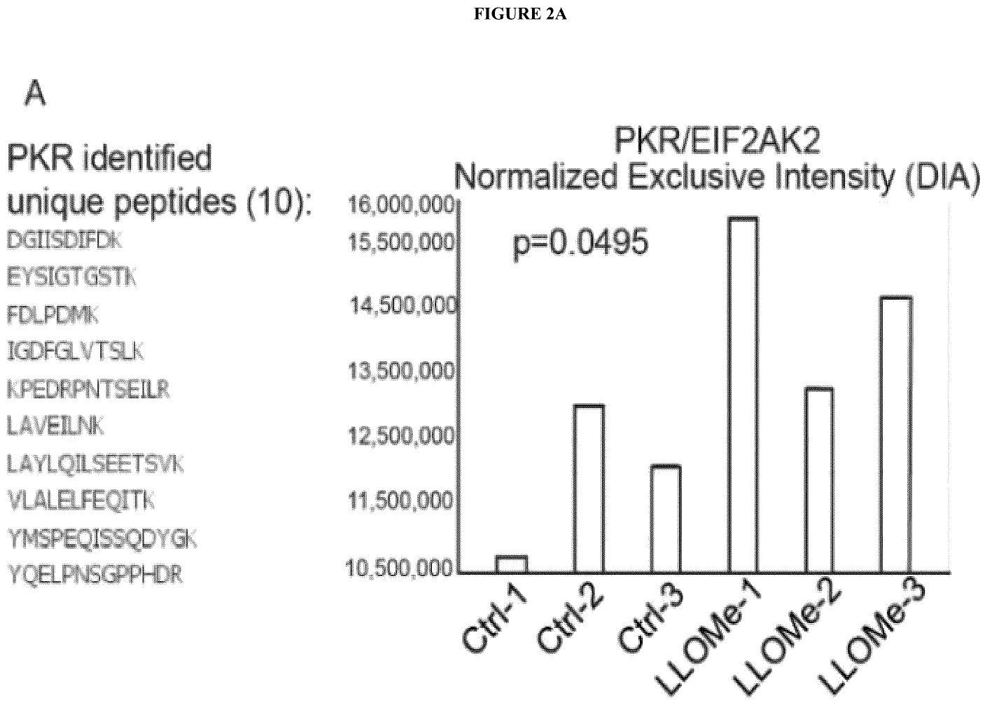

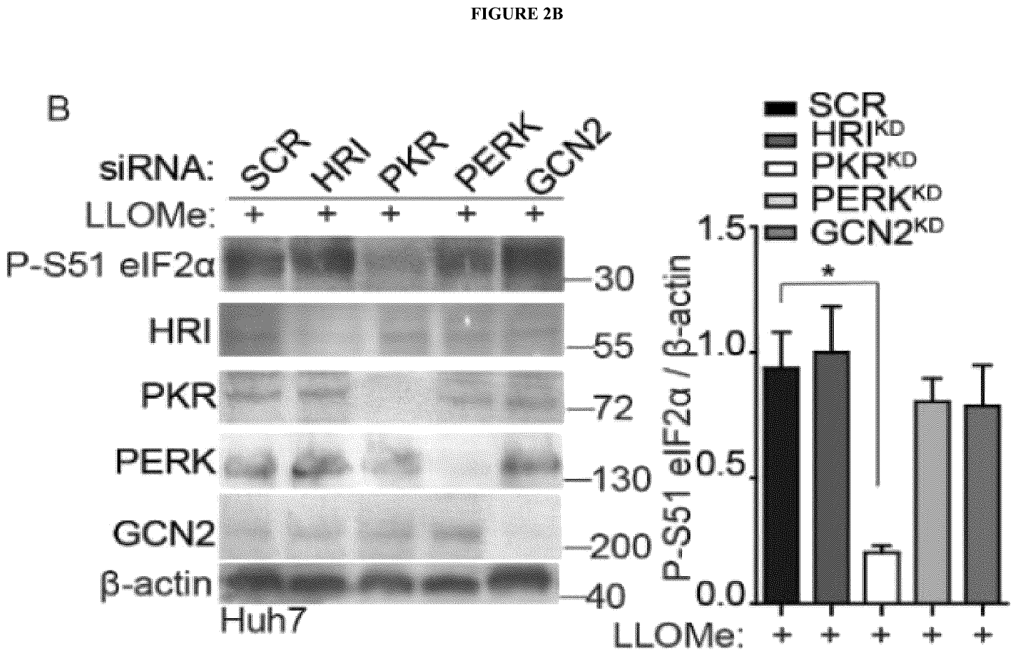

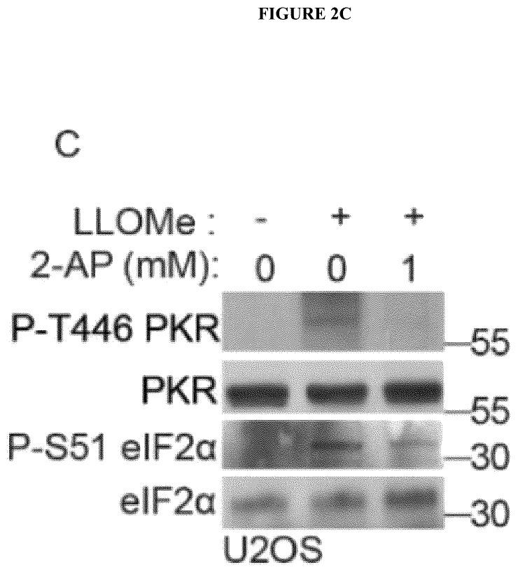

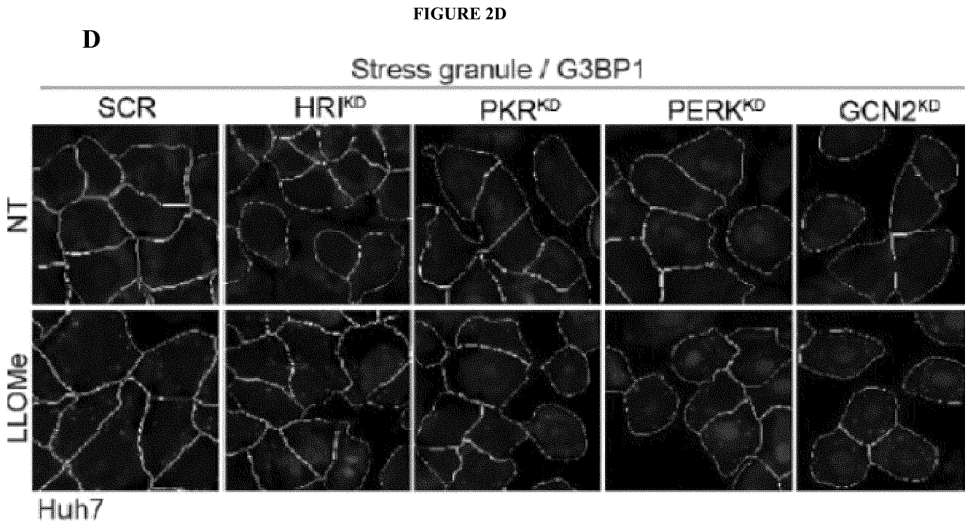

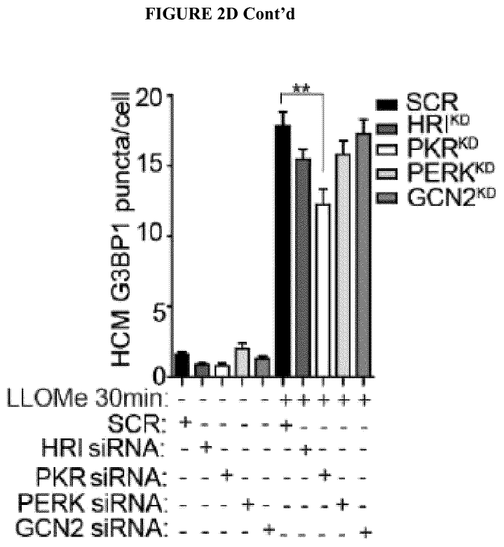

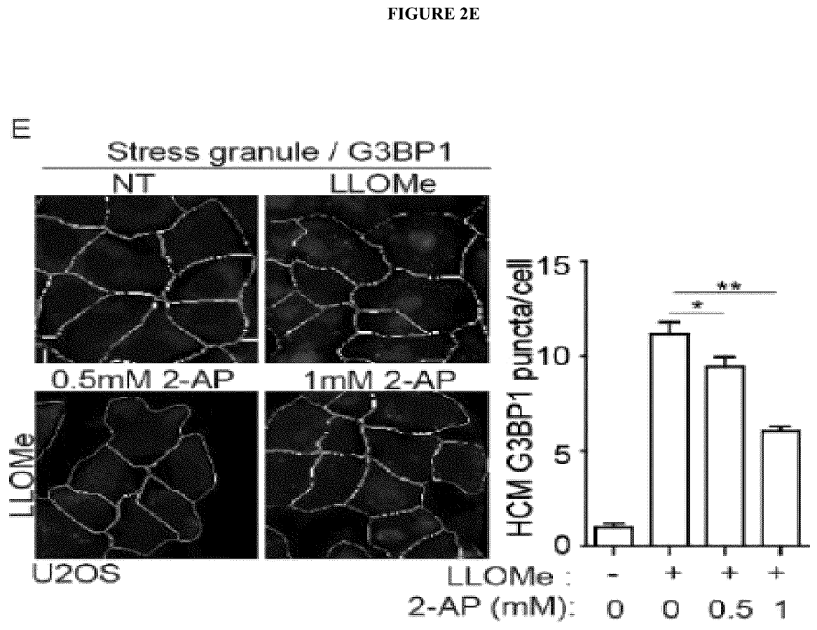

FIGS. 2SA-2SG show the cellular transcriptional response during lysosomal damage. FIG. 2SA RNAseq analysis of the change on gene expression in HEK293T cells in response to 1 mM LLOMe treatment for 30 min. Scatter (volcano) plot shows log 2 fold change and −Log 10 p-value for the genes identified in three independent experiments. Red dots indicate the genes downregulated; green dots indicate the genes upregulated. The dotted line indicates the significance cut-off (P<0.05). FIG. 2SB shows an immunoblot analysis of DUSP1 expression level and ERK2 (T185/187) phosphorylation in HEK293T cells treated with 1 mM LLOMe for 30 min. FIG. 2SC shows an immunoblot analysis of TFEB (S142) phosphorylation in U2OS cells treated with 1 mM LLOMe for 30 min. Ctrl, control (untreated cells). FIG. 2SD shows an immunoblot analysis of ERK2 (T185/187) and TFEB (S142) phosphorylation in Huh7 cells transfected with scrambled siRNA as control (SCR) and DUSP1 siRNA treated with 1 mM LLOMe for 30 min. FIG. 2SE shows the quantification by HCM of TFEB nuclear translocation in Huh7 cells treated with or without 530 nM ERK2 inhibitor AZD6244 for 2 h followed by 1 mM LLOMe for 30 min. Blue: nuclei, Hoechst 33342. Red: anti-TFEB antibody, Alexa Fluor 568. White masks, computer-algorithm-defined cell boundaries. Pink masks, computer-identified nuclear TFEB based on the average intensity of Alexa Fluor 568 fluorescence. FIG. 2SF shows an immunoblot analysis of ERK2 (T185/187) and TFEB (S142) phosphorylation in Huh7 cells treated with or without 530 nM ERK2 inhibitor AZD6244 for 2 h followed by 1 mM LLOMe for 30 min. FIG. 2SG shows the quantification by HCM of G3BP1 puncta in U2OS cells treated with or without 210 nM imidazolo-oxindole C16 for 2 h followed by 2 mM LLOMe for 30 min. Red masks, computer-identified G3BP1 puncta. FIG. 2SH shows the quantification by HCM of G3BP1 puncta in Huh7 cells transfected with scrambled siRNA as control (SCR) and RNASET2 siRNA treated with 2 mM LLOMe for 30 min. Red masks, computer-identified G3BP1 puncta. Ctrl, control (untreated cells). Data, means±SEM; HCM: n≥3 (each experiment: 500 valid primary objects/cells per Well, ≥5 Well s/sample). **p<0.01, ANOVA.

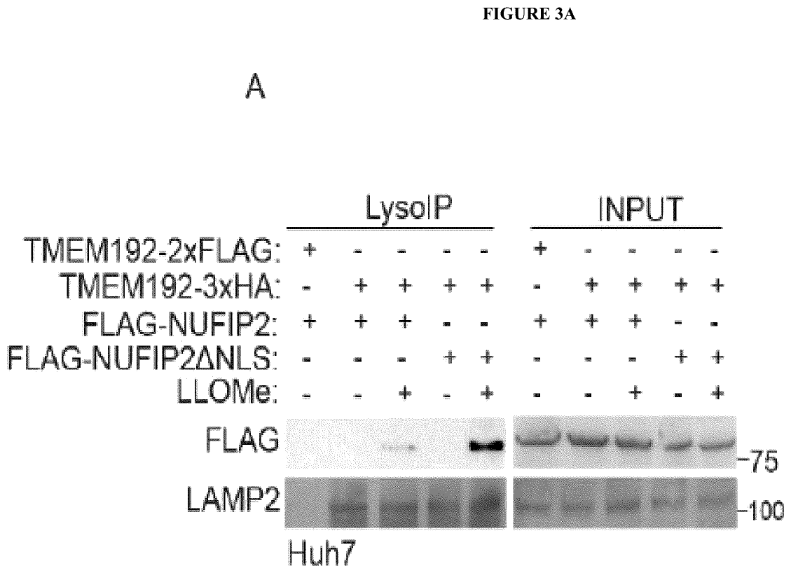

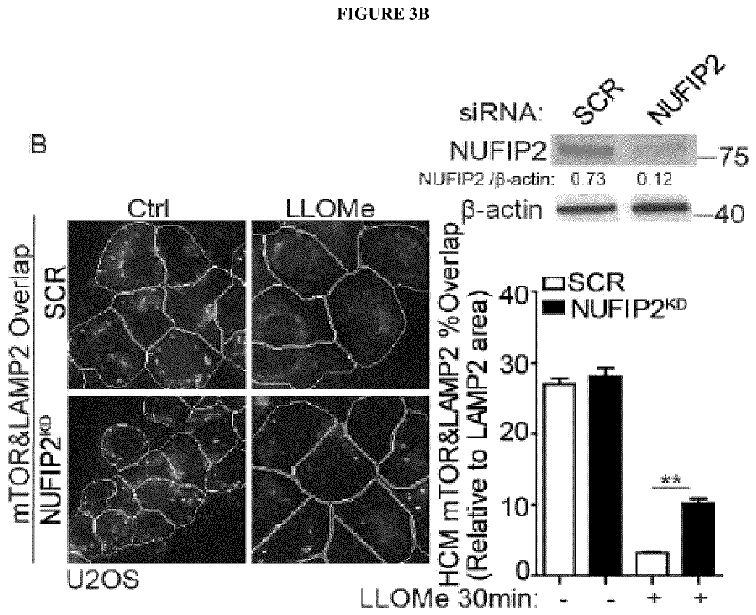

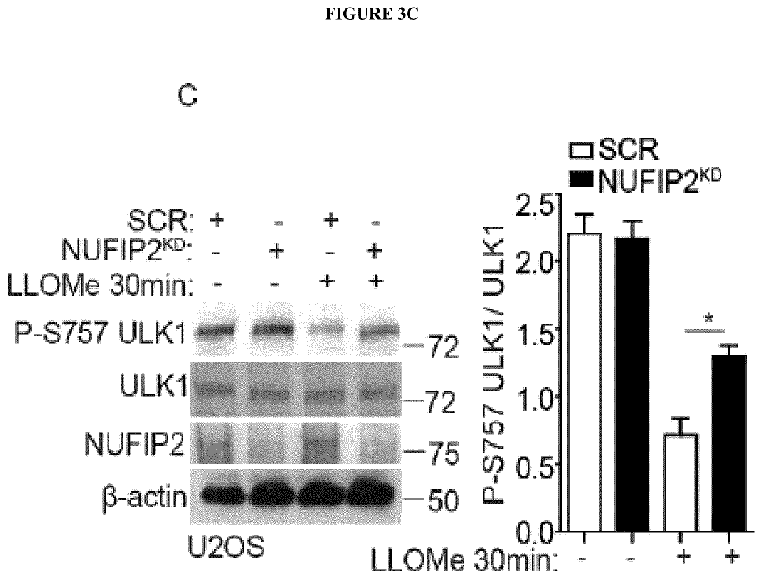

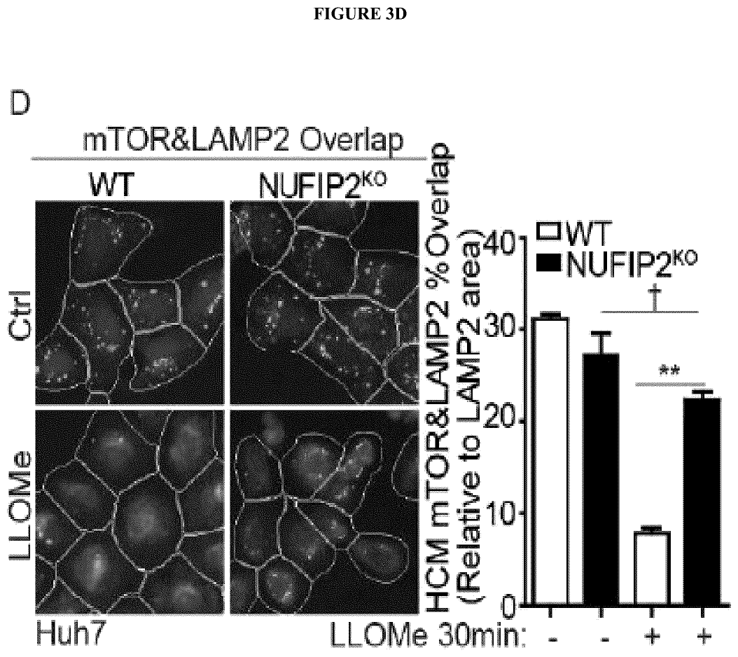

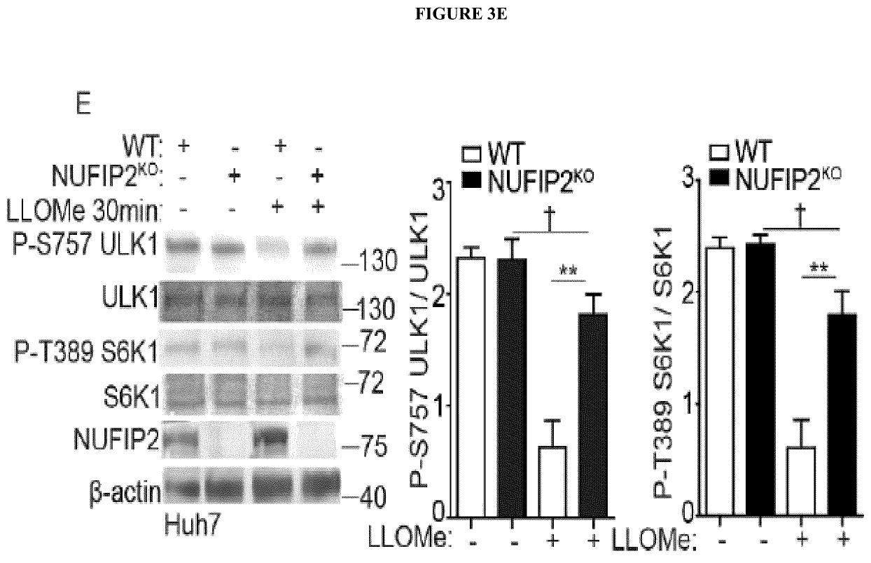

FIGS. 3SA-3SC show that stress granules induced by lysosomal damage show dynamic interactions with lysosomes. FIG. 3SA shows an immunofluorescence confocal microscopy analysis of G3BP1 and LAMP2. U2OS cells Were treated with 2 mM LLOMe for 30 min and stained for endogenous G3BP1 and LAMP2. Scale bar, 5 μm. FIG. 3SB shows live-cell fluorescence imaging analysis of mCherry-G3BP1 and GFP-LAMP1. U2OS cells expressing mCherry-G3BP1 and GFP-LAMP1 Were incubated with 2 mM LLOMe during live-cell fluorescence imaging. Arrows, the representative regions at the indicated timepoint. FIG. 3SC shows the zoom view of the representative regions in panel C. Ctrl, control (untreated cells).

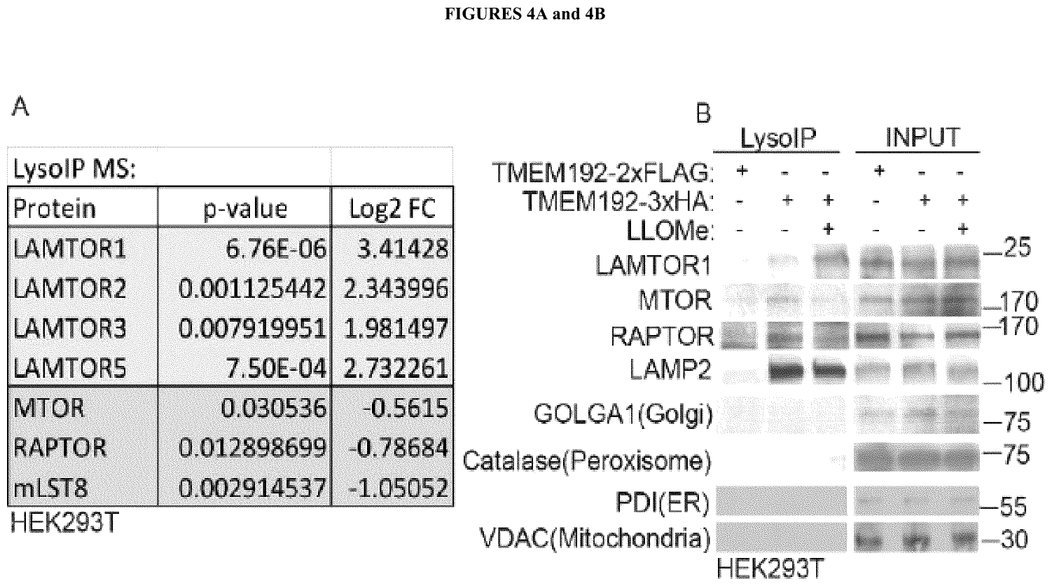

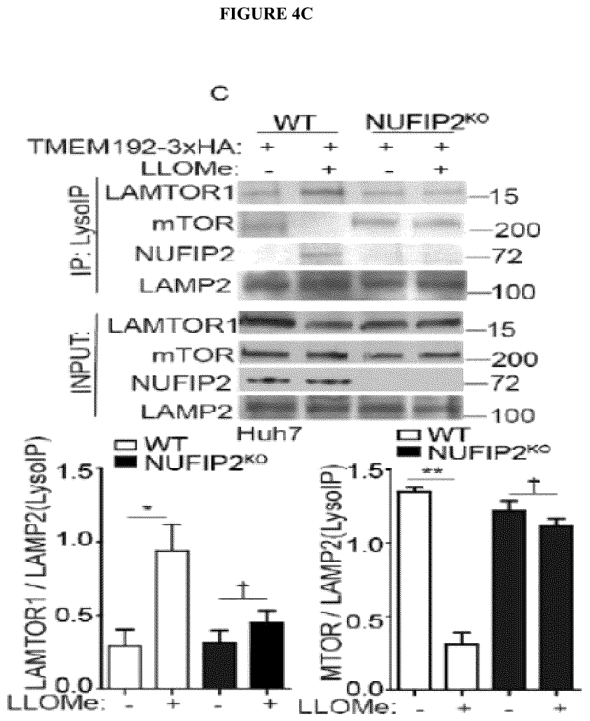

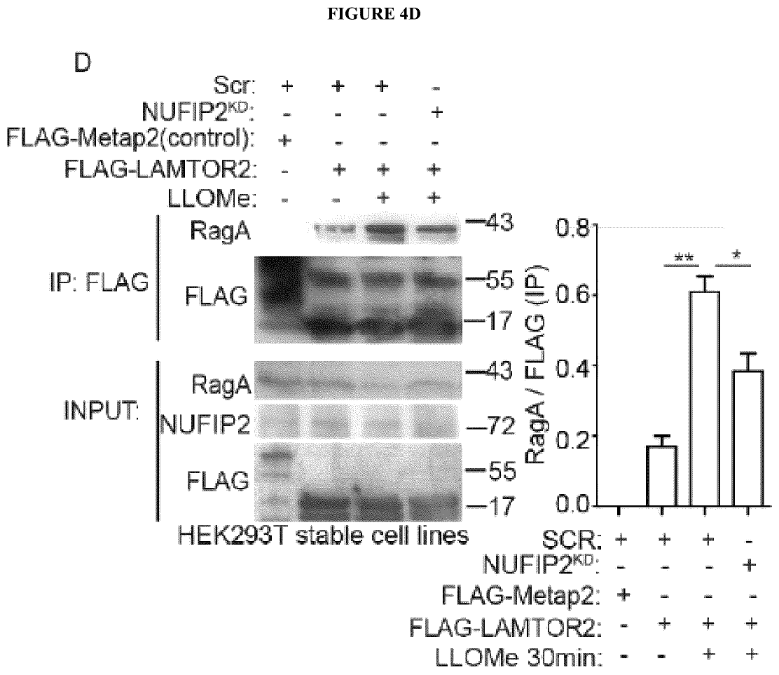

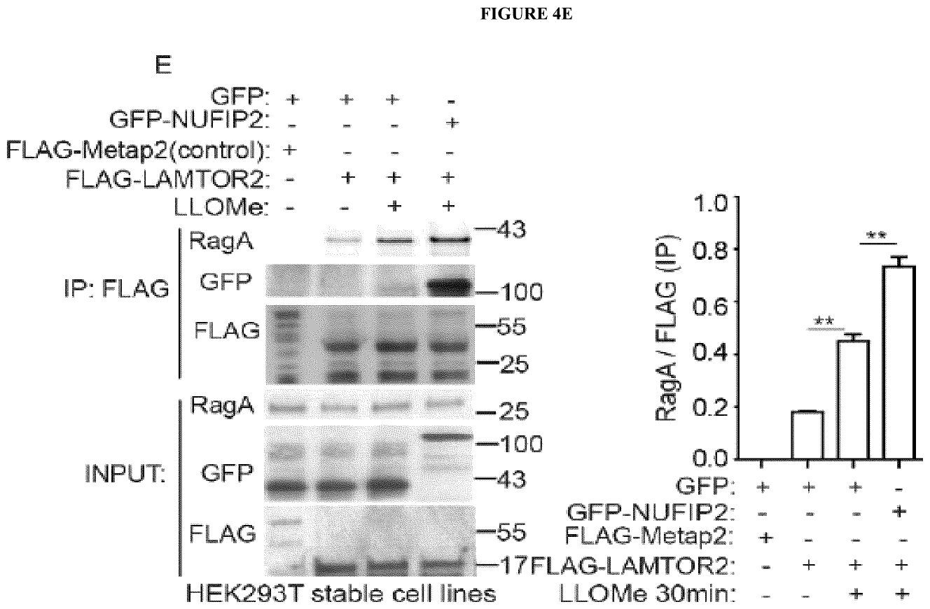

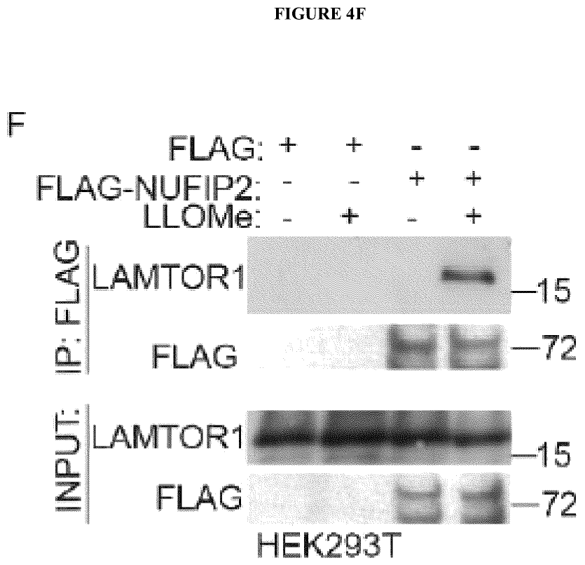

FIGS. 4SA-4SH show that NUFIP2 exits nucleus and localizes to lysosomes upon damage. FIG. 4SA shows immunofluorescence confocal microscopy analysis of G3BP1 and NUFIP2. Huh7 cells Were treated with 2 mM LLOMe for 30 min and stained for endogenous G3BP1 and NUFIP2. Scale bar, 5 μm. FIG. 4SB shows an immunoblot analysis of NUFIP2 distribution in nuclear or postnuclear of Huh7 cells transfected with FLAG-NUFIP2 or NUFIP2ΔNLS after the treatment with 2 mM LLOMe for 30 min. FIG. 4SC shows the NLS (nuclear localization signal) analysis of NUFIP2 by cNLS Mapper. The sequence in red (SEQ ID NO: 1), predicted NLS in NUFIP2, was deleted for generating NUFIP2ΔNLS. FIG. 4SD shows an immunoblot validation of NUFIP2-knockout in Huh7 cells. #E7 was used in the following experiments, named as Huh7^{NUFIP2-KO}. FIG. 4SE shows an immunoblot analysis of the phosphorylation of ULK1 (S757) and S6K1 (T389) in parental HeLa (WT) and TSC2-knockout HeLa cells (TSC2^{KO}) treated with 2 mM LLOMe for 30 min. FIG. 4SF shows an immunoblot analysis of the phosphorylation of ULK1 (S757) and S6K1 (T389) in HEK293T cells or HEK293T cells stably expressing constitutively active RagB GTPase (RagB^{Q99L}) treated with 2 mM LLOMe for 30 min. FIG. 4SG show the quantification by HCM of overlaps between mTOR and LAMP2 in Gal8WT^{HeLa} or Gal8KO^{HeLa} cells treated with 2 mM LLOMe for 30 min. White masks, algorithm-defined cell boundaries. Yellow masks, computer-identified overlap of mTOR and LAMP2. FIG. 4SH shows the quantification by HCM of G3BP1 puncta in Gal8WT^{HeLa} or Gal8KO^{HeLa} cells treated with 2 mM LLOMe for 30 min. Red masks, computer-identified G3BP1 puncta. Ctrl, control (untreated cells). Data, means±SEM; HCM: n≥3 (each experiment: 500 valid primary objects/cells per Well, ≥5 Well s/sample). †p≥0.05 (not significant), **p<0.01, ANOVA. See also FIG. 3 and FIG. 4.

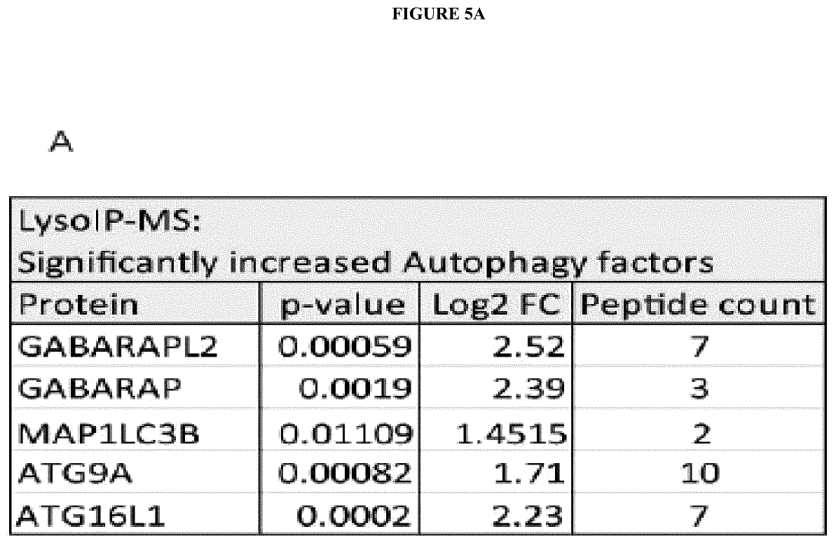

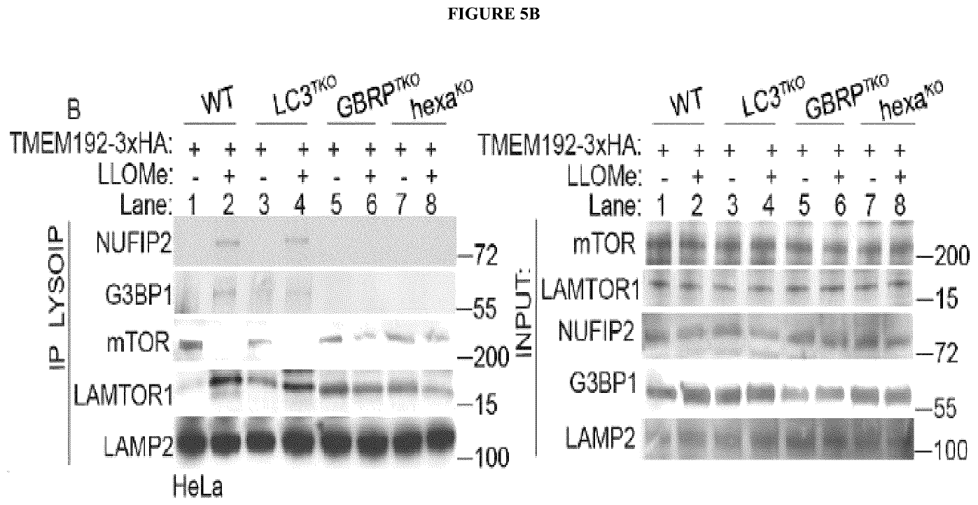

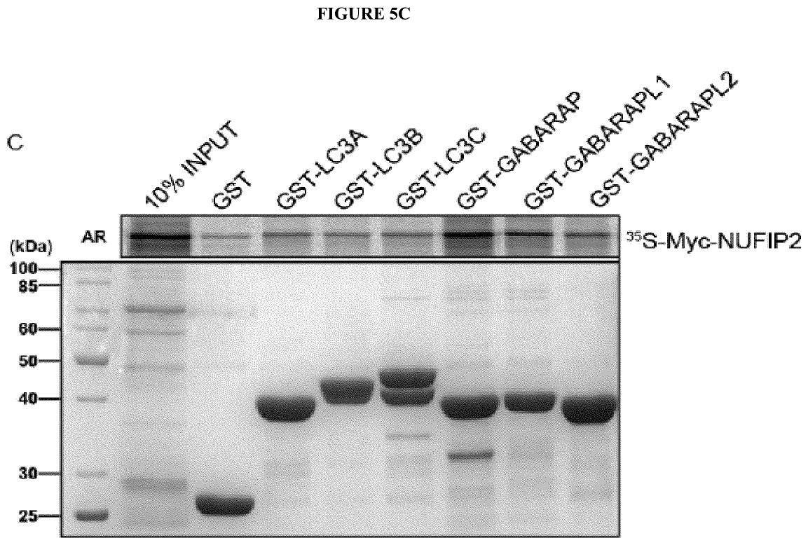

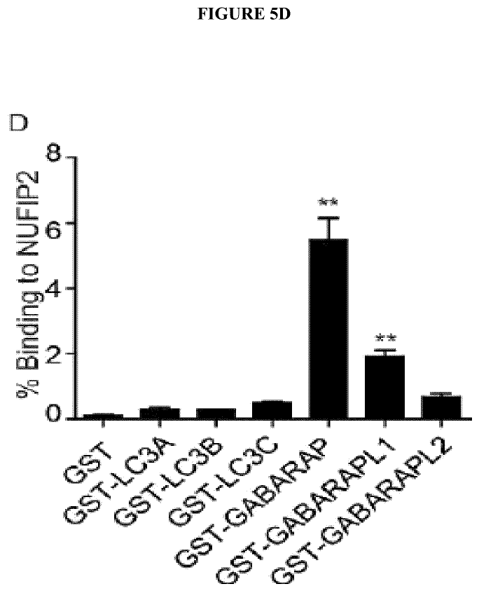

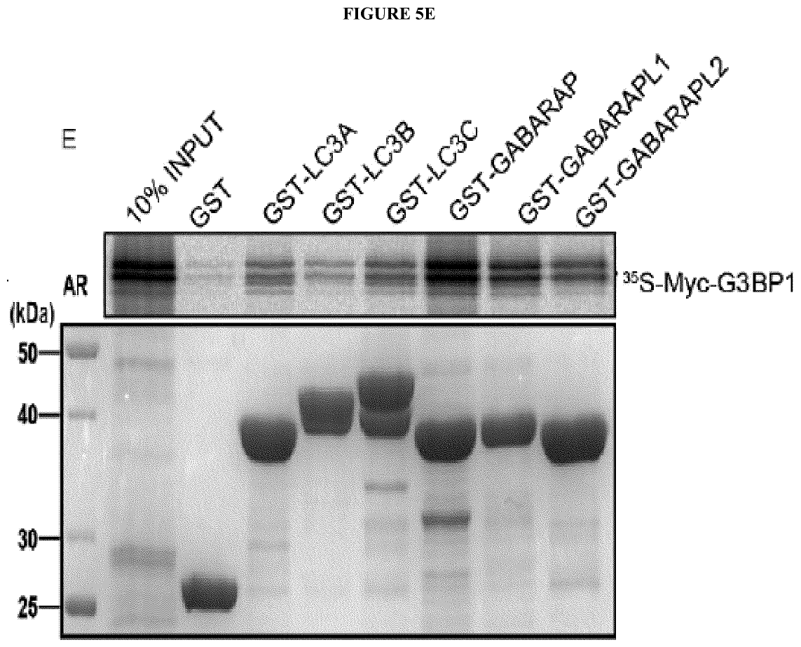

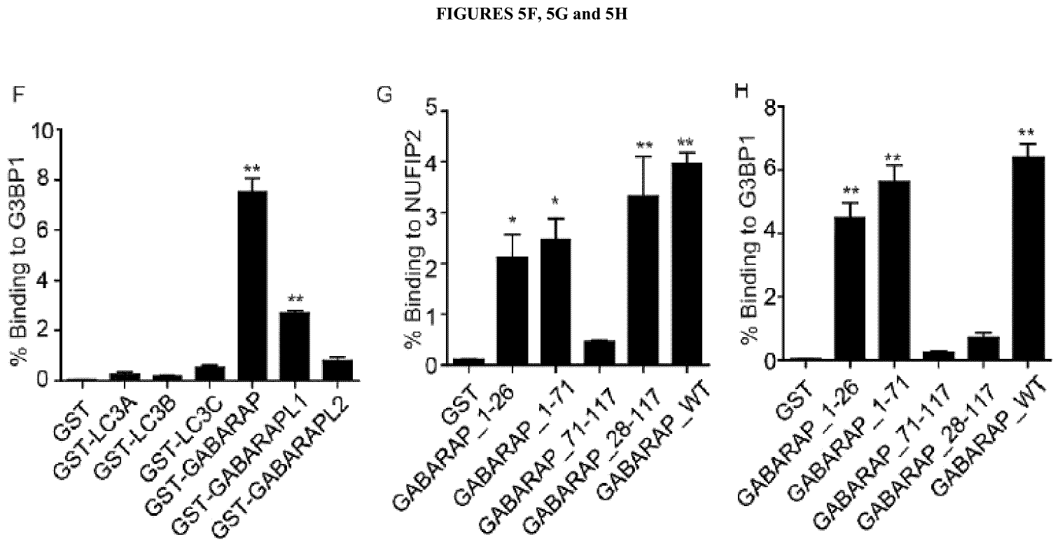

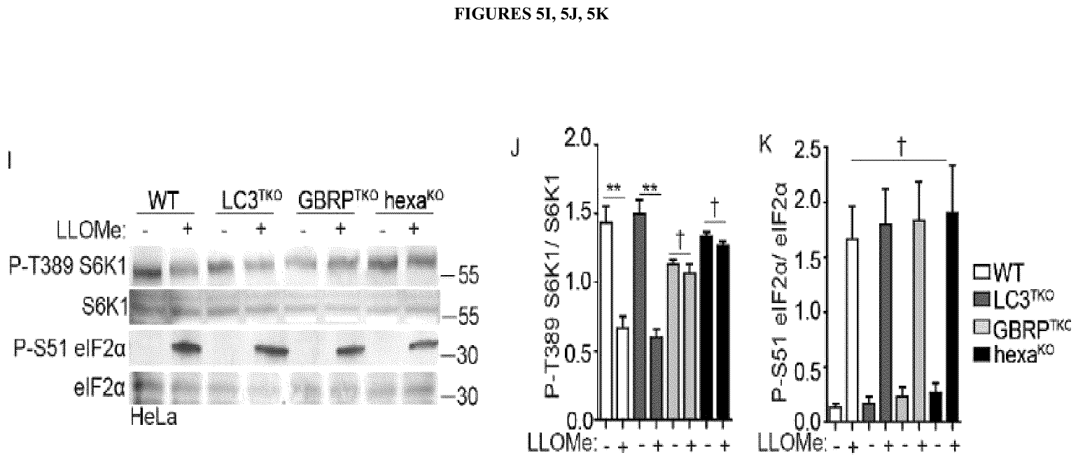

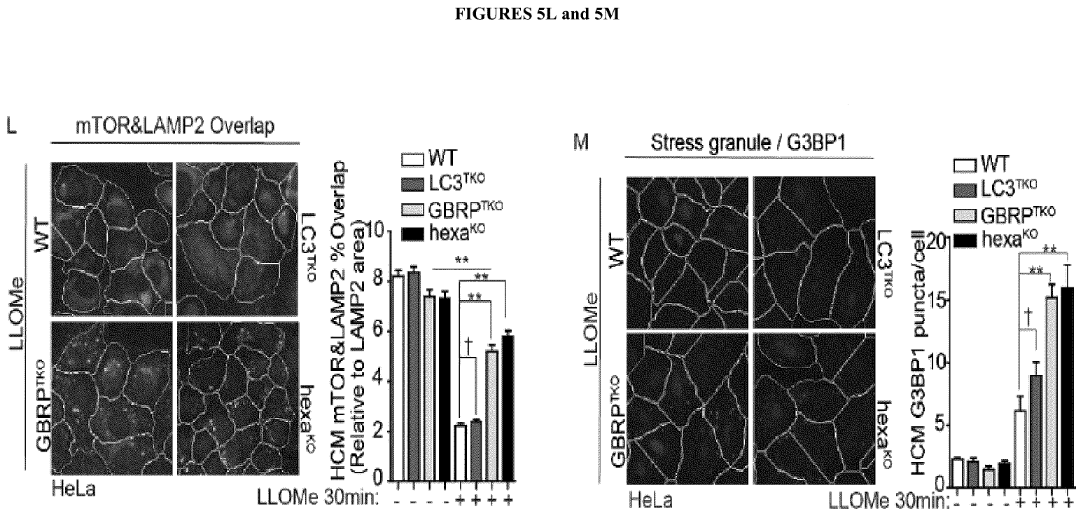

FIGS. 5SA-5SJ show that GABARAPs interact directly with NUFIP2 and G3BP1. FIG. 5SA GST pull-down assay of in vitro translated and radiolabeled Myc-tagged NUFIP2 with GST or GST-tagged GABARAP deletions. FIG. 5SB shows a GST pull-down assay of in vitro translated and radiolabeled Myc-tagged NUFIP2/G3BP1 with GST or GST-tagged GABARAP mutants. FIG. 5SC GST pull-down assay of in vitro translated and radiolabeled Myc-tagged G3BP1 with GST or GST-tagged GABARAP deletions. FIG. 5SD shows a summary of interactions between GABARAP and G3BP1. FIG. 5SE shows a GST pull-down assay of in vitro translated and radiolabeled Myc-tagged G3BP1 with GST or GST-tagged NUFIP2. FIG. 5SF shows that the quantification of E. Data (% binding) represents the percentage of the corresponding protein relative to its input. FIG. 5SG shows a GST pull-down assay of in vitro translated and radiolabeled Myc-tagged NUFIP2 with GST or GST-G3BP1. FIG. 5SH shows an immunoblot analysis of interaction between NUFIP2 and G3BP1 in HEK293T cells transfected with FLAG-NUFIP2 with 2 mM LLOMe for 30 min. FIG. 5SI shows HCM images of untreated cells in FIG. 5SJ. FIG. 5SJ shows HCM images of untreated cells in FIG. 5K. Ctrl, control (untreated cells). Data, means±SEM; HCM: n≥3 (each experiment: 500 valid primary objects/cells per Well, ≥5 Well s/sample). **p<0.01, ANOVA. See also FIG. 5.

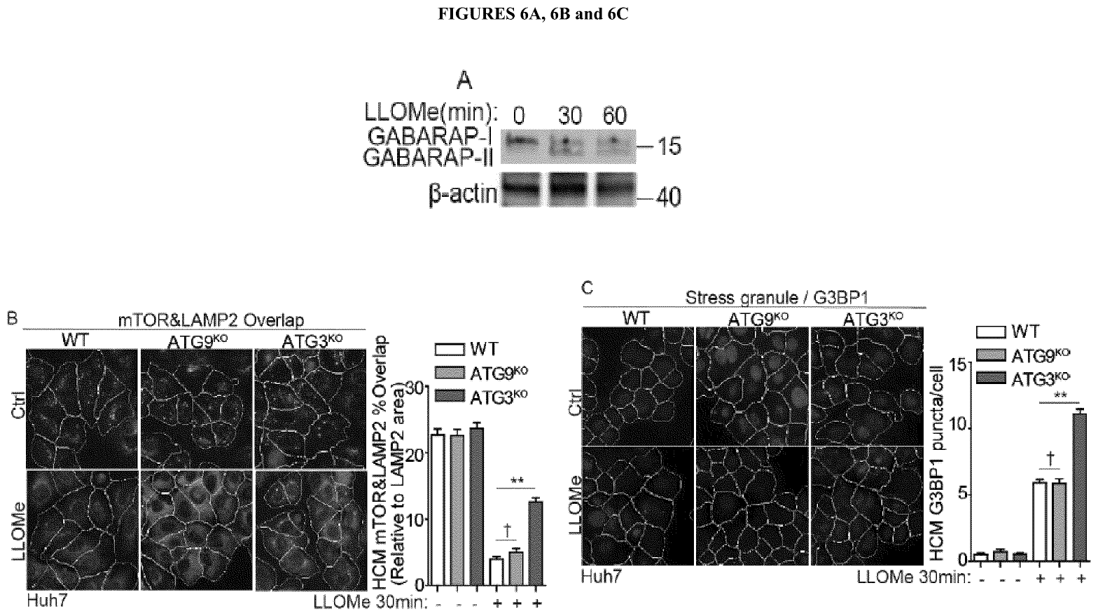

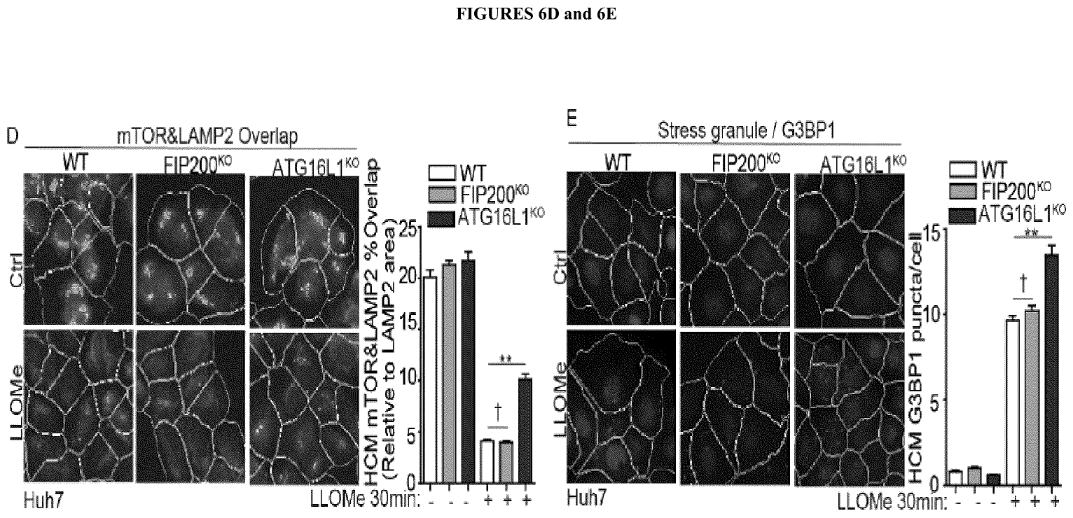

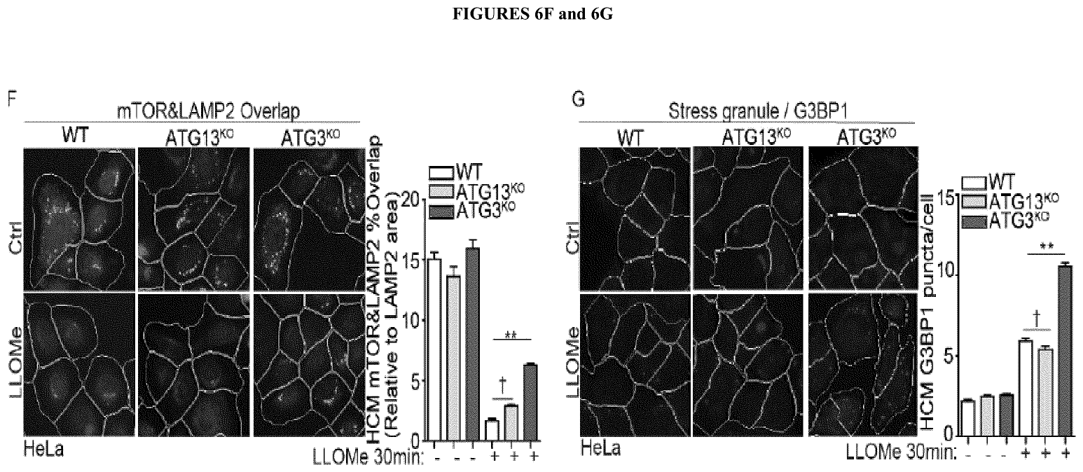

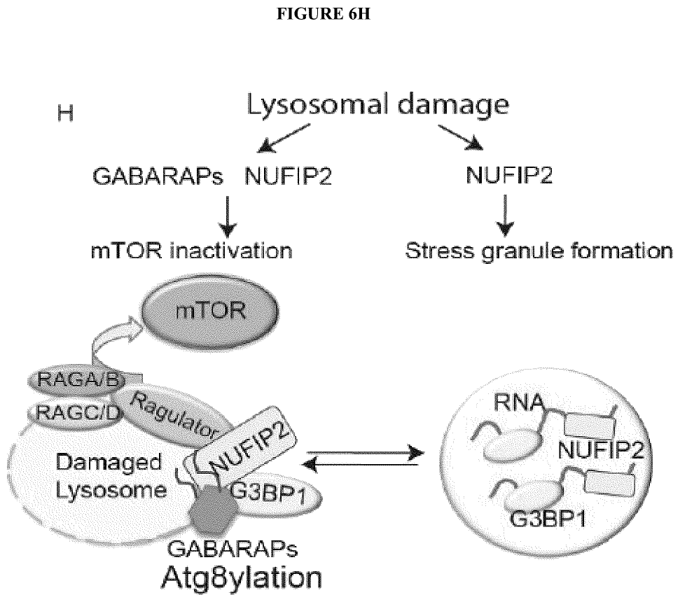

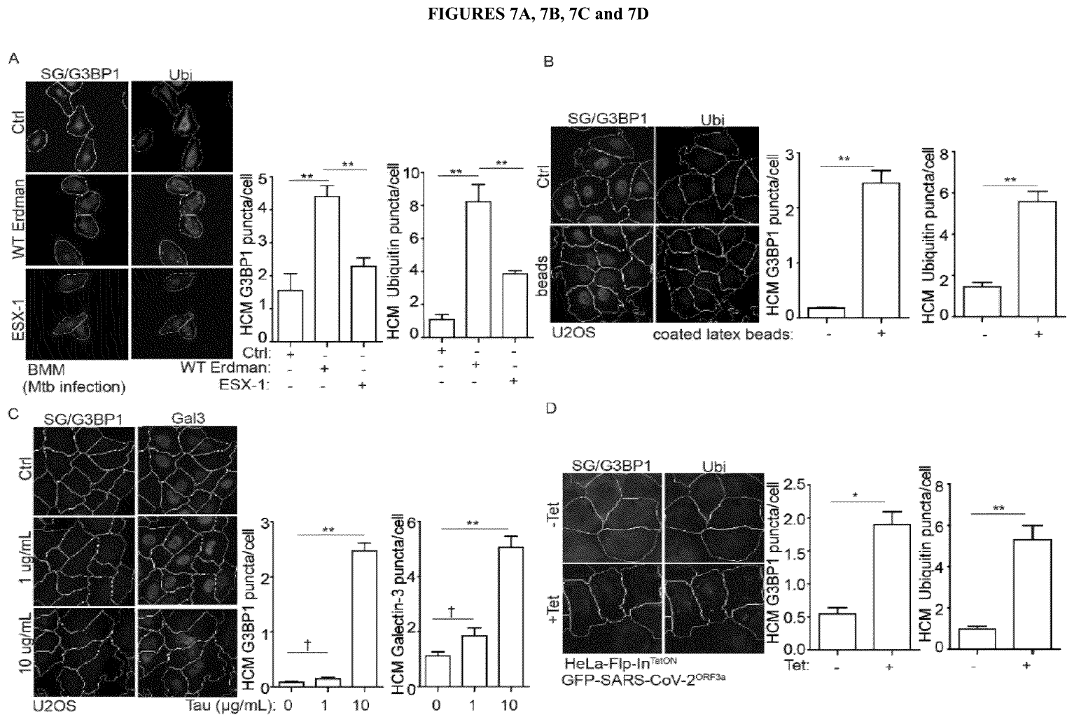

FIGS. 6SA-6SI show that GABARAPs participate in mTOR inactivation in response to lysosomal damage. FIG. 6SA shows the quantification by HCM of overlaps between mTOR and LAMP2 in HeLa (WT), GBRP^{TKO} and GBRP^{TKO} transfected with GFP-GABARAP/GABARAPL1/GABARAPL. Cells treated with 4 mM LLOMe for 30 min. White masks, algorithm-defined cell boundaries. Yellow masks, computer-identified overlap of mTOR and LAMP2. FIG. 6SB shows the quantification by HCM of G3BP1 puncta in HeLa (WT), GBRP^{TKO} and GBRP^{TKO} transfected with GFP-GABARAP/GABARAPL1/GABARAPL. Cells Were treated with 4 mM LLOMe for 30 min. Red masks, computer-identified G3BP1 puncta. FIG. 6SC shows a schematic summary of the findings in this study. FIG. 6SD shows a WB analysis of ATG9, ATG3 and β-actin in Huh7 cells. FIG. 6SE shows a WB analysis of FIP200, ATG16L1 and β-actin in Huh7 cells. FIG. 6SF shows a WB analysis of ATG3, ATG13 and β-actin in Hela cells. FIG. 6SG shows a WB analysis of the expression of GFP-SARS-CoV-2^{ORF3a} in HeLa Flp-In^{TetON} GFP-SARS-CoV-2^{ORF3a} cells induced by tetracycline (Tet) for 16 h. FIG. 6SH shows an immunoblot analysis of interaction between GCN1 and GFP-ORF3a in HEK293T Flp-InTetON GFP-SARS-CoV-2^{ORF3a} cells induced by 1 μg/mL tetracycline (Tet) for 16 h. Cell lysates Were immunoprecipitated (IP) with anti-GFP antibody and immunoblotted for endogenous GCN1. FIG. 6SI shows an immunoblot analysis of interaction between GCN1 and ORF3a in HEK293T cells transfected with GFP or GFP-ORF3a. Cell lysates Were immunoprecipitated (IP) with anti-GFP antibody and immunoblotted for endogenous GCN1. Ctrl, control (untreated cells). Data, means±SEM; HCM: n≥3 (each experiment: 500 valid primary objects/cells per Well, ≥5 Well s/sample). †p≥0.05 (not significant), **p<0.01, ANOVA. See also FIG. 5, FIG. 6 and FIG. 7.

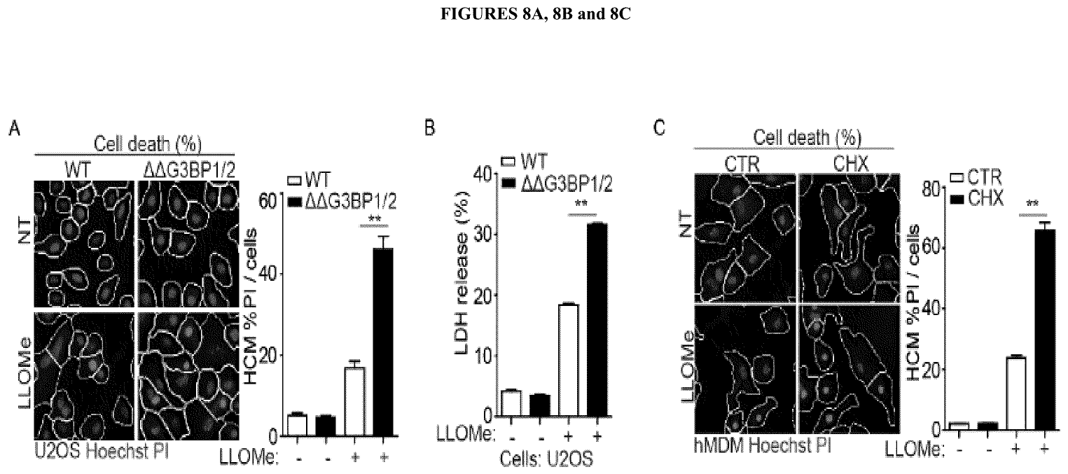

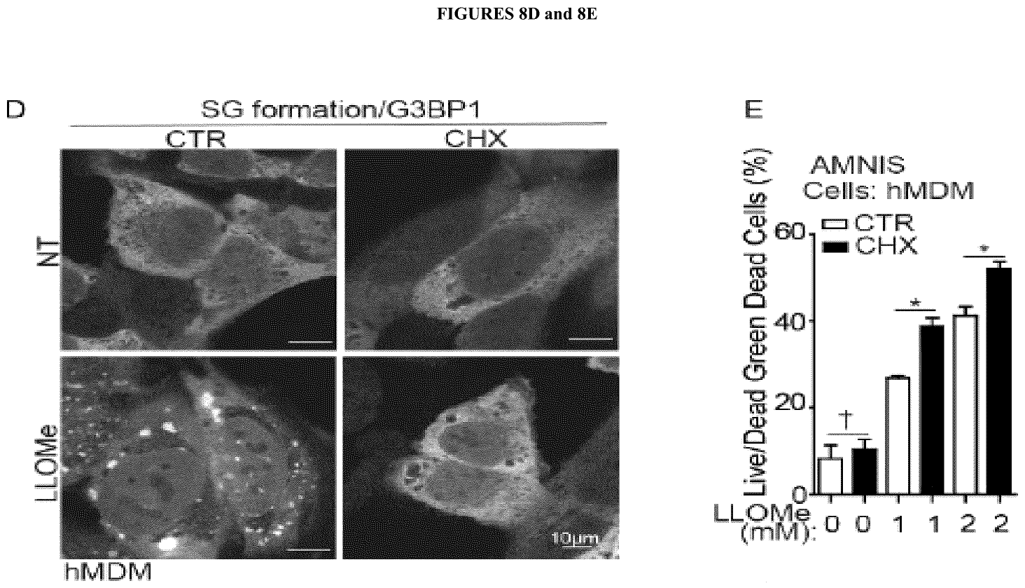

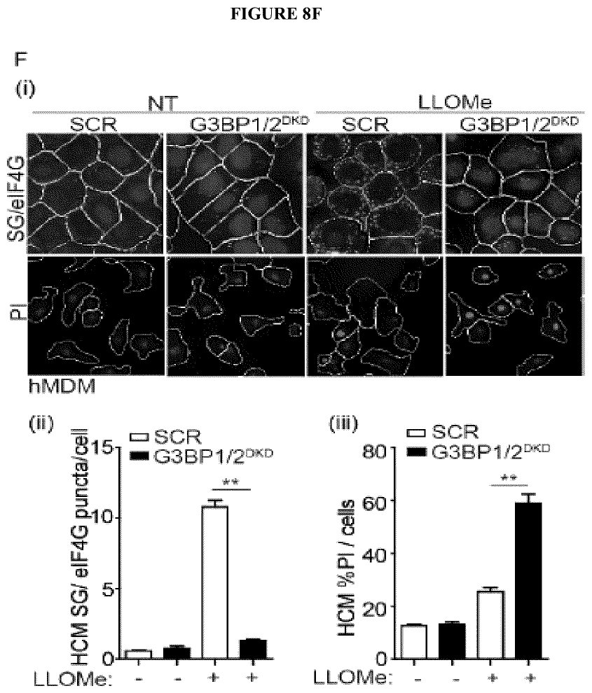

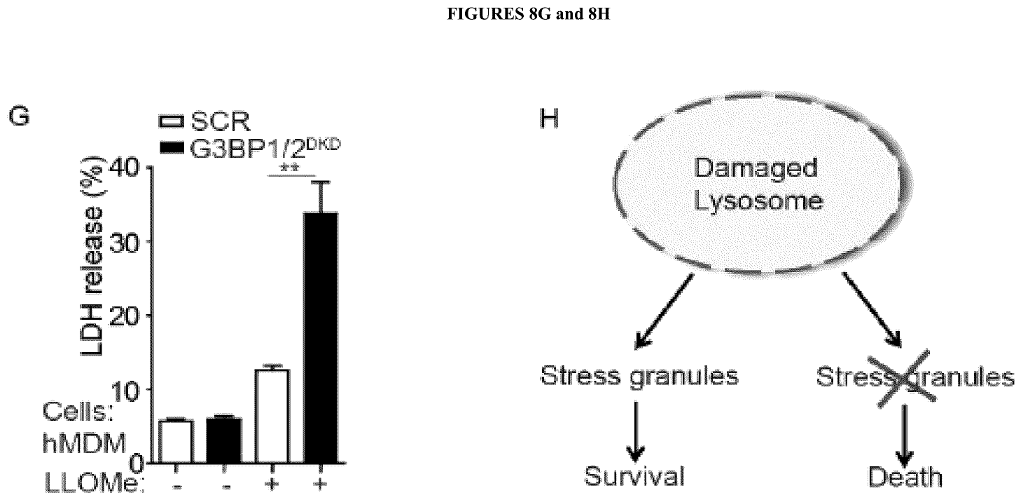

FIGS. 7SA-7SG shows that lysosomal proteome changes and stress granule formation during lysosomal damage. FIG. 7SA (i) EncyclopeDIA/scaffoldDIA analysis of lysosomes purified by anti-HA immunoprecipitation (LYSOIP; TMEM192-3×HA) from HEK293T cells treated with or without 1 mM LLOMe for 30 min. Scatter (volcano) plot shows log 2 fold change and −Log 10 p-value for the proteins identified and quantified (LC/MS/MS) in three independent experiments. The dotted line indicates the significance cut-off (P<0.05). (ii) Enrichment analysis for biological processes in LysoIP LC/MS/MS. The significant increased proteins in LysoIP LC/MS/MS Were analyzed by STRING functional enrichment analysis. The sub-selection biological processes are displayed. FDR, false discovery rate. FIG. 7SB show the quantification by HCM of DCP1a and G3BP1 puncta in U2OS cells treated with 2 mM LLOMe for 30 min. FIG. 7SC shows the quantification by HCM of G3BP1 puncta in Huh7 cells treated with 2 mM LLOMe for 30 min. White masks, algorithm-defined cell boundaries (primary objects); Green masks, computer-identified G3BP1 puncta (target objects). FIG. 7SD shows the quantification by HCM of TIA1 puncta in U2OS cells treated with 2 mM LLOMe for 30 min. Red masks, computer-identified TIA1 puncta. FIG. 7SE shows the quantification by HCM of TIA1 puncta in Hela cells treated with 4 mM LLOMe for 30 min. Red masks, computer-identified TIA1 puncta. FIG. 7SF shows the quantification by HCM of G3BP1 puncta in U2OS cells treated with 2 mM LLOMe for 30 min and followed by 1 h washout. Red masks, computer-identified G3BP1 puncta. FIG. 7SG shows an immunoblot analysis of eIF2α (S51) phosphorylation in HEK293T cells treated with 2 mM LLOMe or 100 μM NaAsO₂ for 30 min. FIG. 7SH shows a schematic summary of the findings in FIG. 8. Ctrl, control (untreated cells). Data, means±SEM; HCM: n≥3 (each experiment: 500 valid primary objects/cells per Well, ≥5 Well s/sample). *p<0.05, **p<0.01, ANOVA. See also FIG. 8.

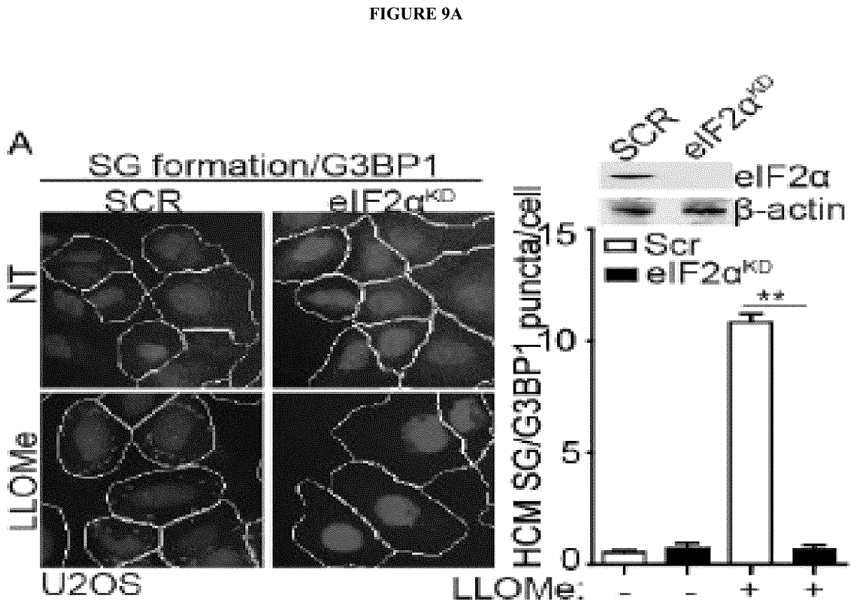

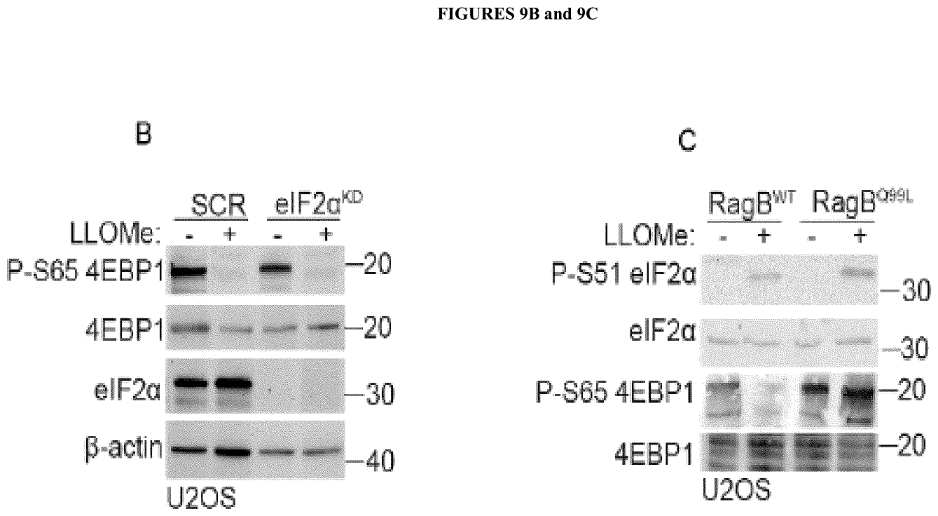

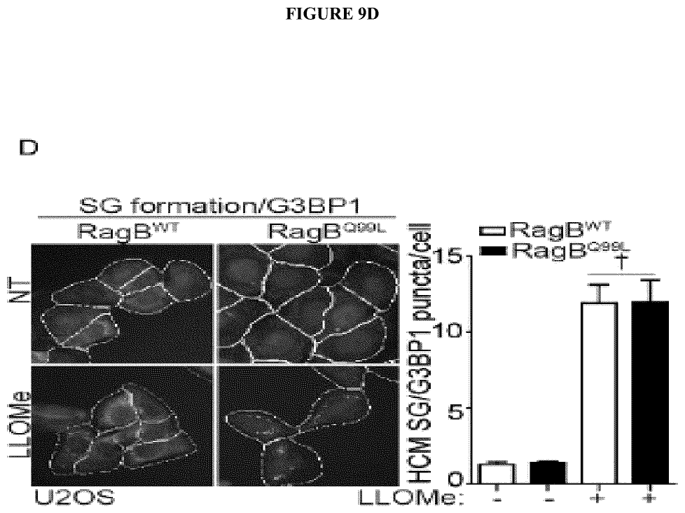

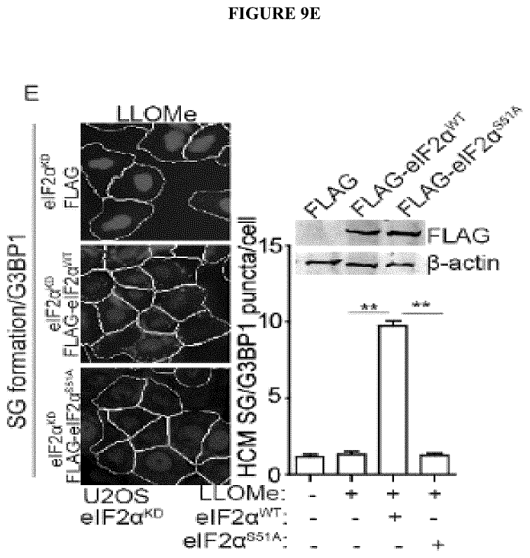

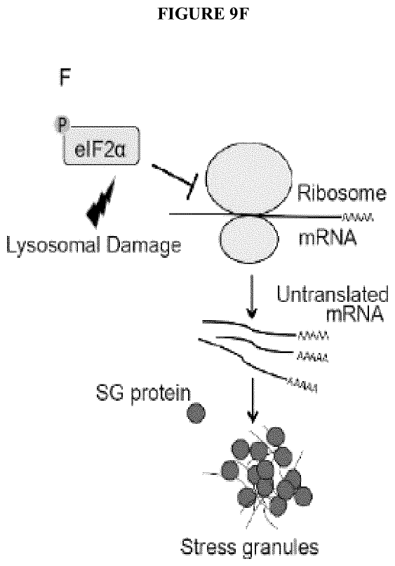

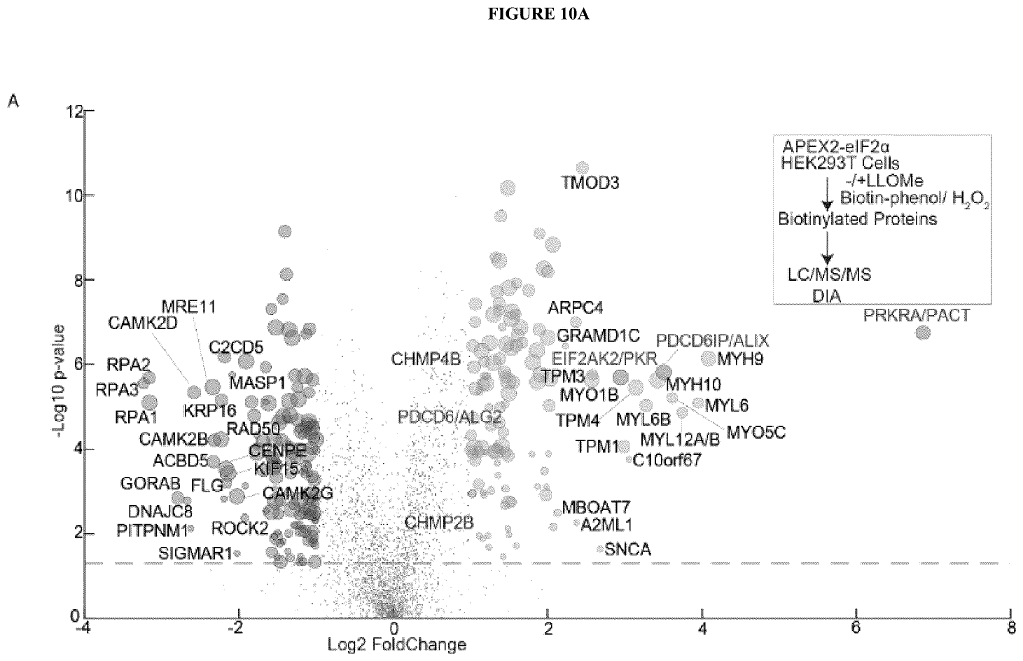

FIGS. 8SA-8SC show that the eIF2α and mTORC1 signaling pathways are uncoupled in response to lysosomal damage. FIG. 8SA Immunoblot analysis of phosphorylation of eIF2α (S51) and 4EBP1 (S65) in U2OS cells treated with the indicated dose of LLOMe for 30 min. FIG. 8SB shows the quantification by HCM of overlaps between mTOR and LAMP2 or G3BP1 puncta in U2OS cells. Cells Were treated with EBSS, 2 mM LLOMe or 100 UM NaAsO₂ for 30 min. White masks, algorithm-defined cell boundaries; green masks, computer-identified overlap between mTOR and LAMP2; red masks, computer-identified G3BP1 puncta. FIG. 8SC shows an immunoblot analysis of phosphorylation of eIF2α (S51) and S6K1 (T389) in U2OS cells treated as in FIG. 8SB. CTR, control. Data, means±SEM (n=3); HCM: n≥3 (each experiment: 500 valid primary objects/cells per Well, ≥5 Well s/sample). †p≥0.05 (not significant), **p<0.01, ANOVA. See also FIG. 9.

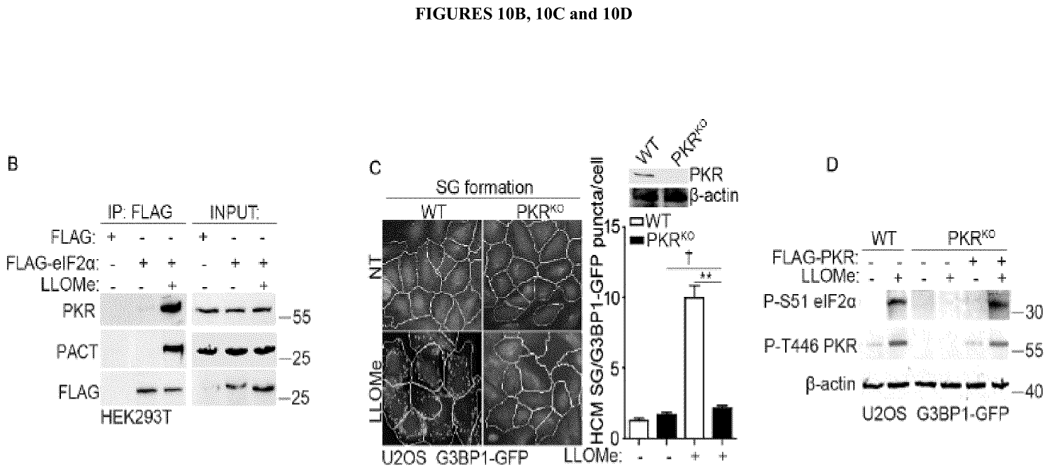

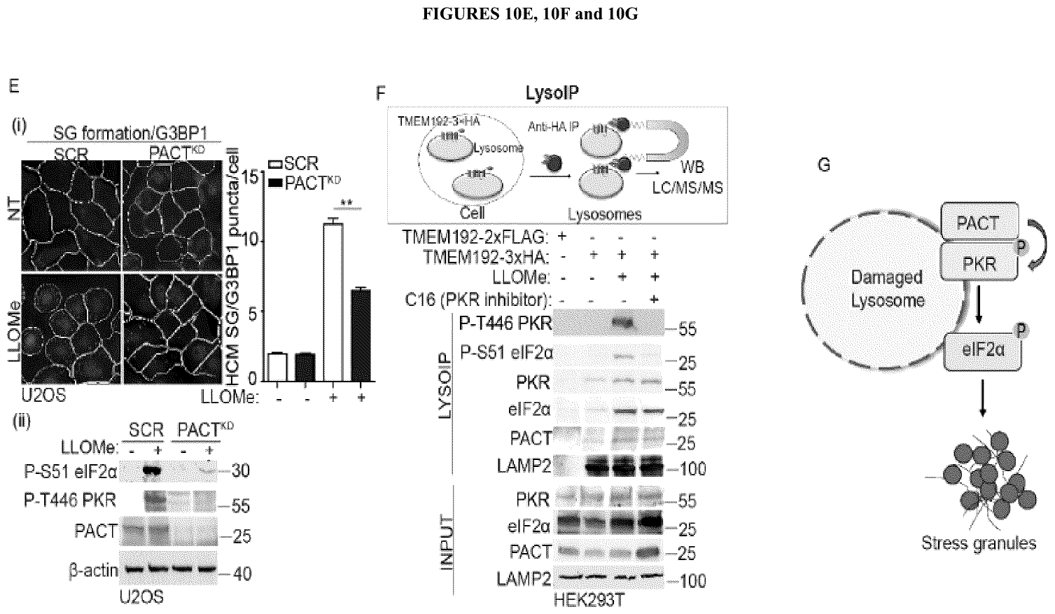

FIGS. 9SA-9SE show that PKR, PACT and eIF2α are associated with damaged lysosomes. FIG. 3SA shows an immunoblot analysis of phosphorylation of eIF2α (S51) in U2OS cells transfected with either scrambled siRNA as control (SCR) or MARK2 siRNA for knockdown (MARK2^{KD}). Cells Were treated with 2 mM LLOMe for 30 min. FIG. 9SB shows the summary of the literature on the detected peptide count of PKR, PACT and eIF2α in the proteomic analysis of lysosomes based on LysoIP LC/MS/MS analysis. FIG. 9SC shows the confocal microscopy imaging of GFP-PKR and LAMP2 in U2OS cells treated with 2 mM LLOMe for 30 min. Scale bar, 5 μm. FIG. 9SD shows the confocal microscopy imaging of GFP-PACT and LAMP2 in U2OS cells treated with 2 mM LLOMe for 30 min. Scale bar, 5 μm. FIG. 9SE shows the confocal microscopy imaging of GFP-eIF2α and LAMP2 in U2OS cells treated with 2 mM LLOMe for 30 min. Scale bar, 5 μm. See also FIG. 10.

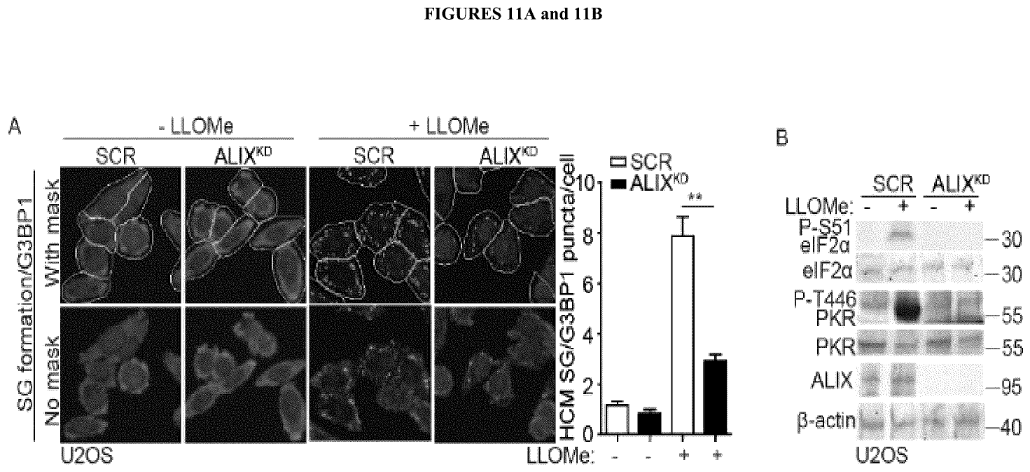

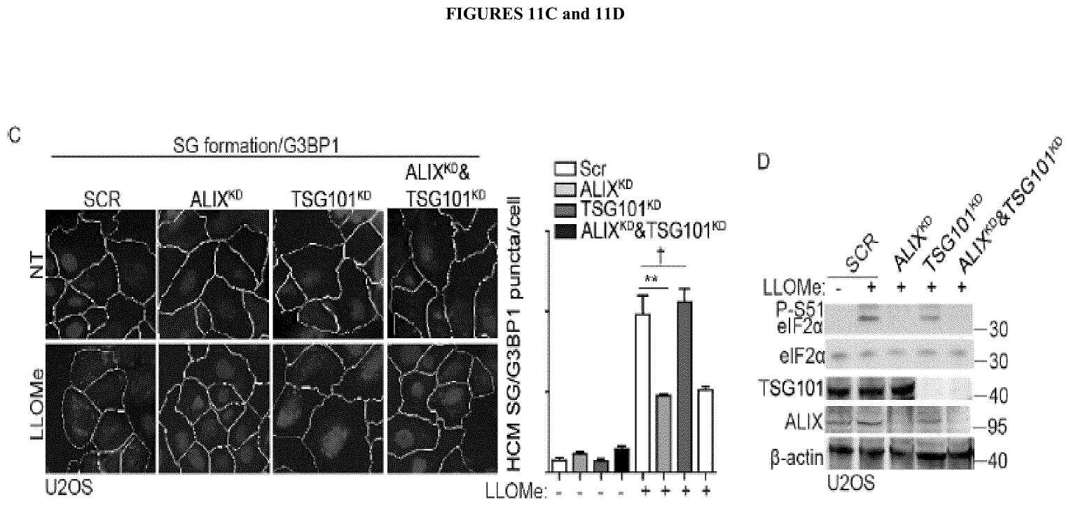

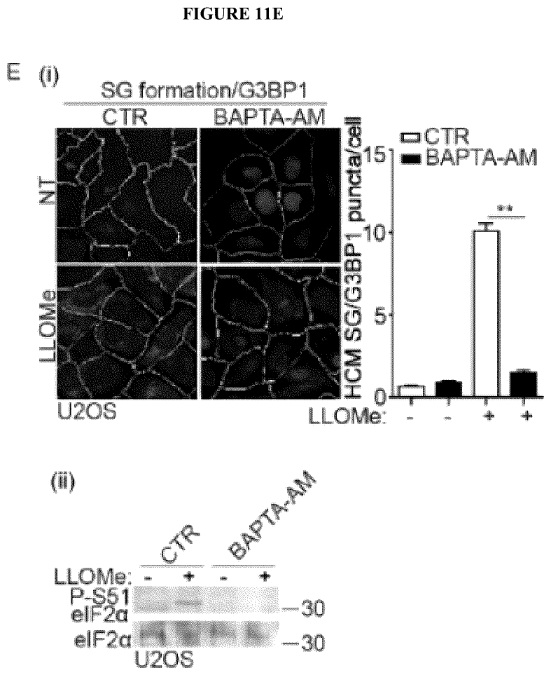

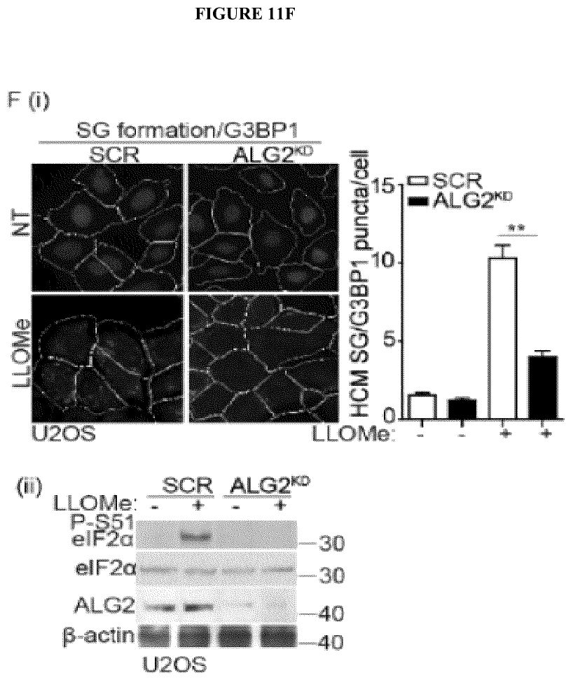

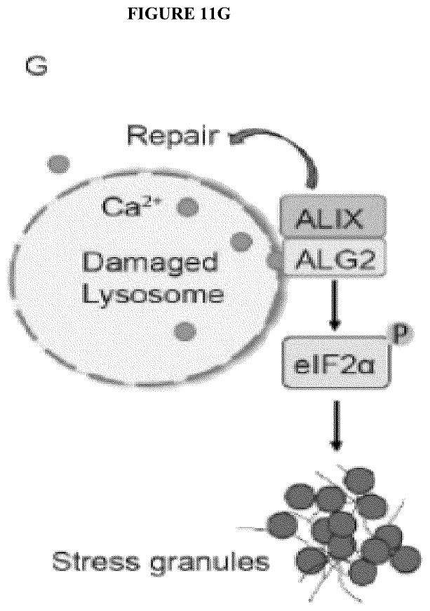

FIGS. 10SA-10SC shows that ALIX regulates stress granule formation during lysosomal damage. FIG. 10SA) shows the quantification by HCM of G3BP1 puncta in U2OS cells transfected with scrambled siRNA as control (SCR), CHMP2B siRNA for knockdown (CHMP2B^{KD}) or CHMP4B siRNA for knockdown (CHMP4B^{KD}). Cells Were treated with 2 mM LLOMe for 30 min. White masks, algorithm-defined cell boundaries; red masks, computer-identified G3BP1 puncta. FIG. 10SB show an immunoblot analysis of phosphorylation of eIF2α (S51) in U2OS transfected with scrambled siRNA as control (SCR), CHMP2B siRNA for knockdown (CHMP2B^{KD}) or CHMP4B siRNA for knockdown (CHMP4B^{KD}), subjected to 2 mM LLOMe treatment for 30 min. FIG. 10SC shows the quantification by HCM of ALIX puncta in U2OS cells transfected with scrambled siRNA as control (SCR), or ALG2 siRNA for knockdown (ALG2^{KD}), or pre-treated with 15 μM BAPTA-AM for 1 h. Cells Were treated with 2 mM LLOMe for 30 min. White masks, algorithm-defined cell boundaries; green masks, computer-identified ALIX puncta. NT, untreated cells. Data, means±SEM (n=3); HCM: n≥3 (each experiment: 500 valid primary objects/cells per Well, ≥5 Well s/sample). †p≥0.05 (not significant), **p<0.01, ANOVA. See also FIG. 11.

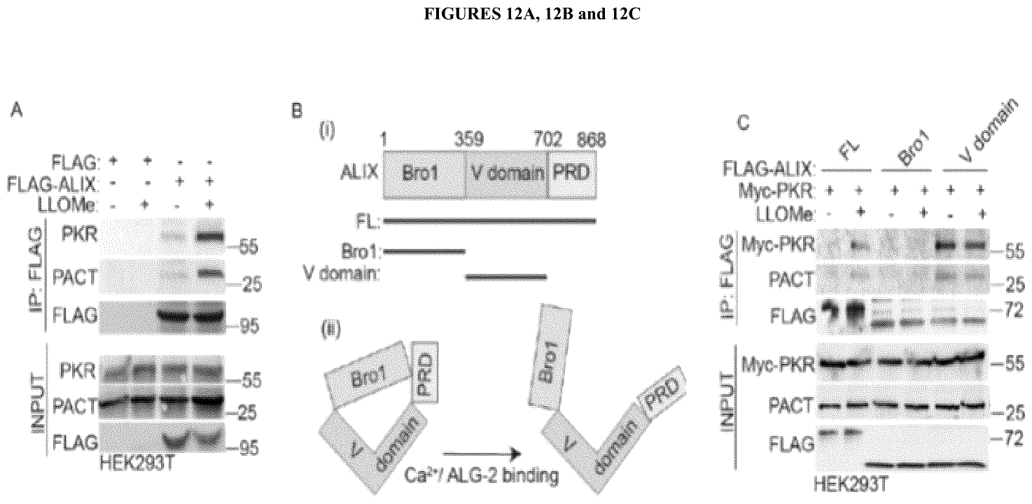

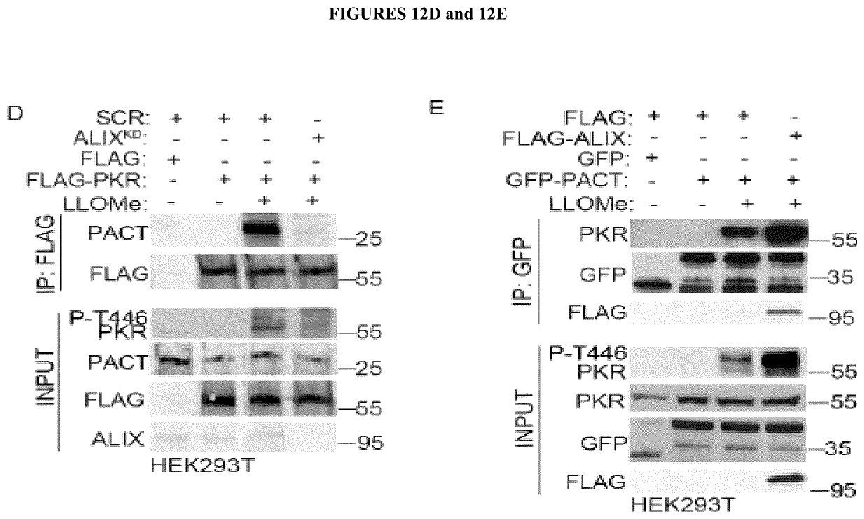

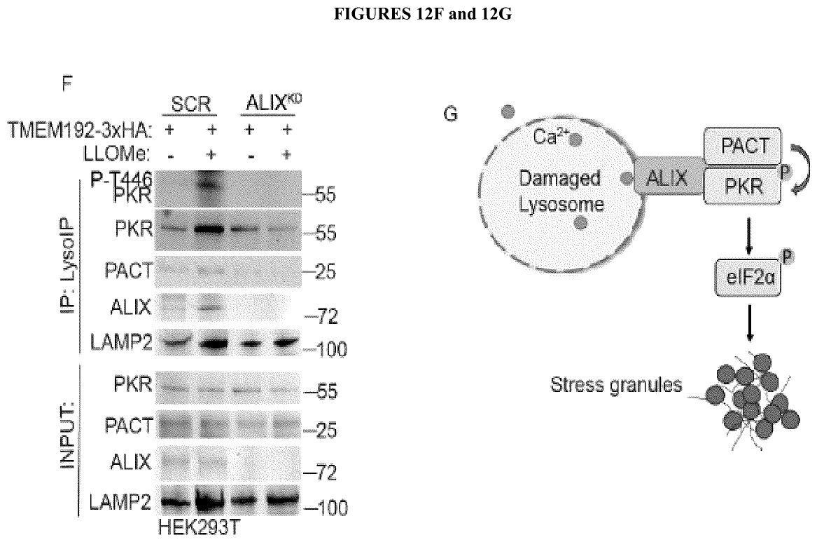

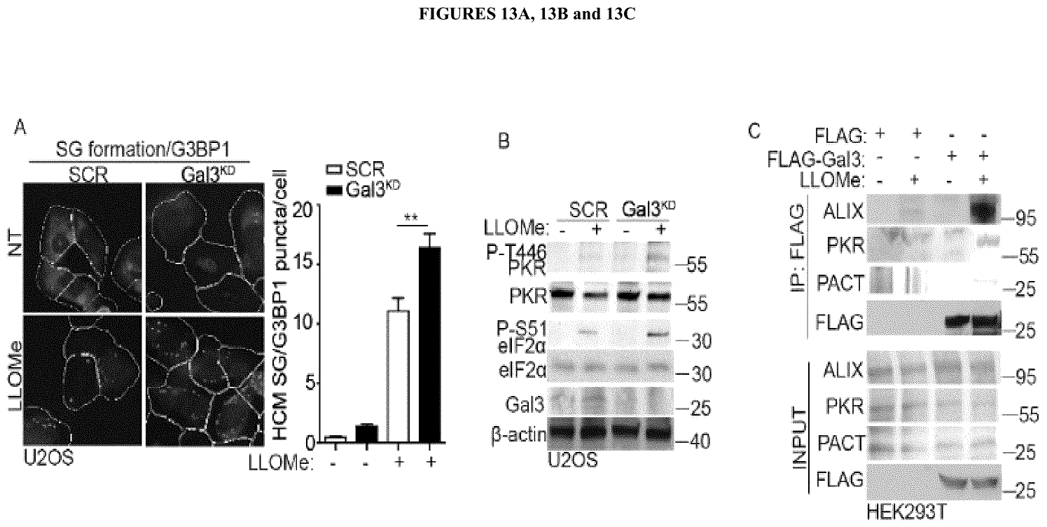

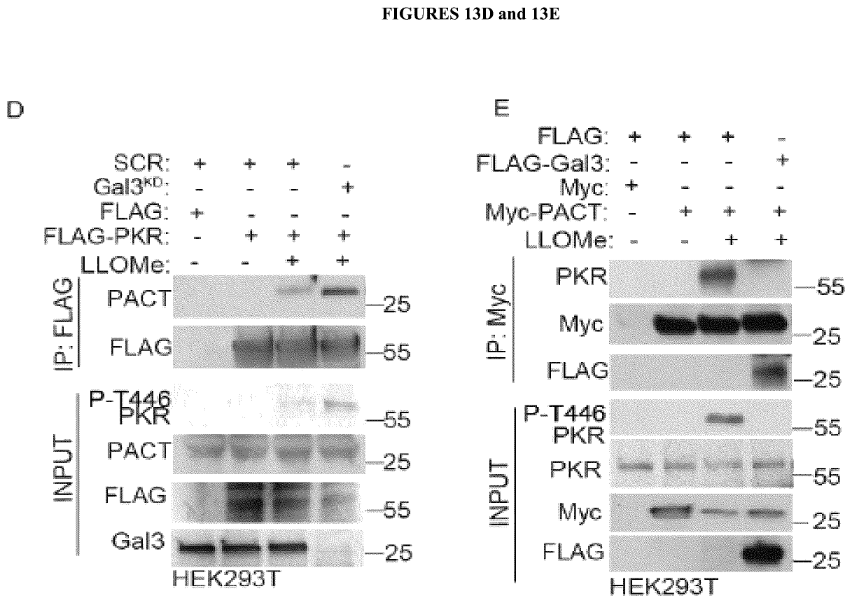

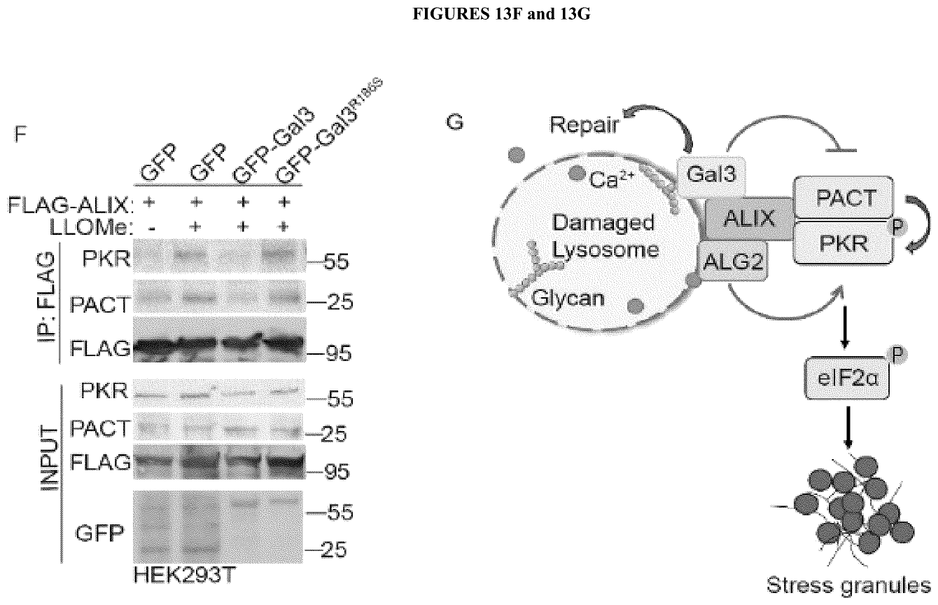

FIGS. 11SA-11SD show that PKR and PACT associate with ALIX during lysosomal damage. FIG. 11SA shows the HDOCK-predicted interaction between ALIX and PKR. FIG. 11SB shows the HDOCK-predicted interaction between ALIX and PACT. FIG. 11SC shows the confocal microscopy imaging of GFP-PKR/PACT and ALIX in U2OS cells treated with 2 mM LLOMe for 30 min. Scale bar, 5 μm. FIG. 11SD shows the quantification by HCM of ALIX puncta in U2OS cells transfected with scrambled siRNA as control (SCR), PKR siRNA for knockdown (PKR^{KD}), or PACT siRNA for knockdown (PACT^{KD}). Cells Were treated with 2 mM LLOMe for 30 min. White masks, algorithm-defined cell boundaries; green masks, computer-identified ALIX puncta. NT, untreated cells. Data, means±SEM (n=3); HCM: n≥3 (each experiment: 500 valid primary objects/cells per Well, ≥5 Well s/sample). †p≥0.05 (not significant), ANOVA. See also FIG. 12.

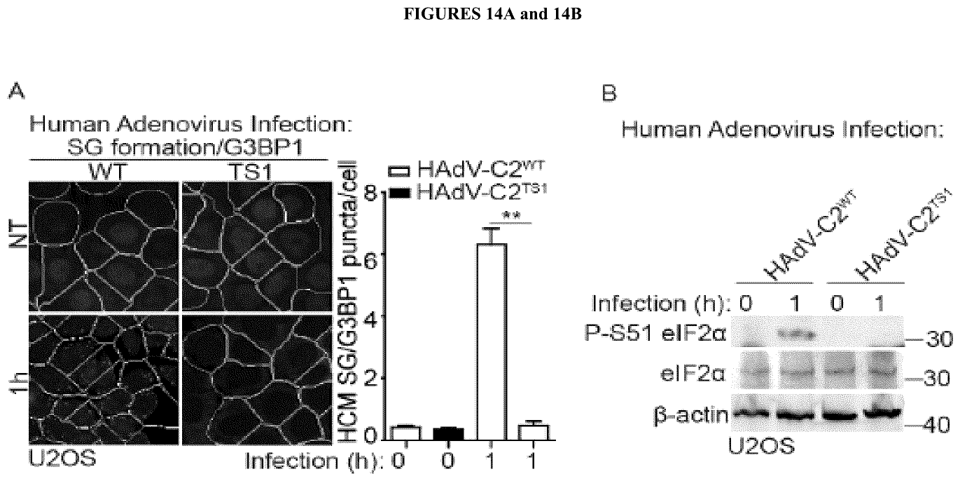

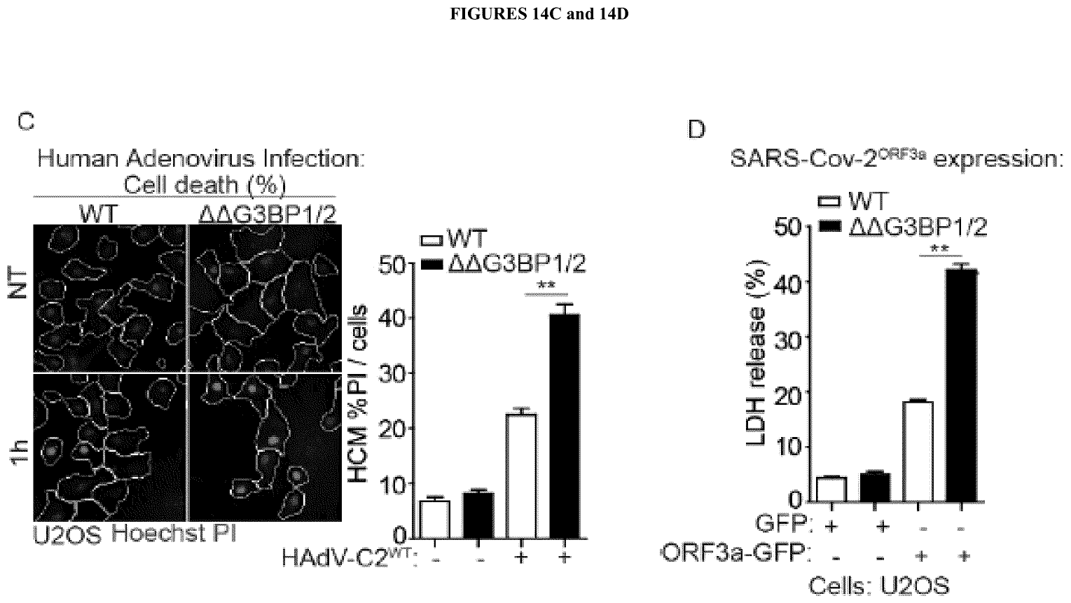

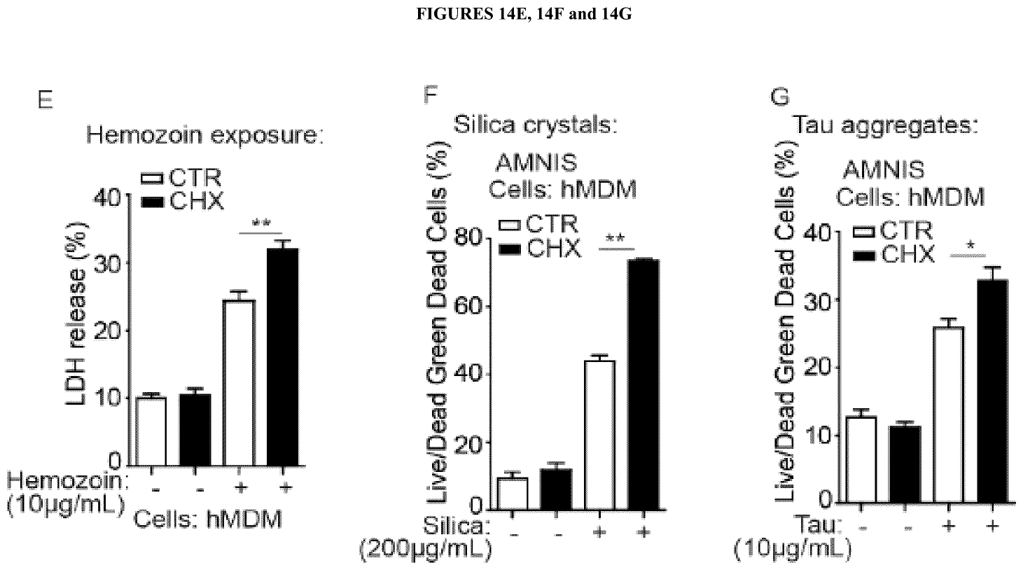

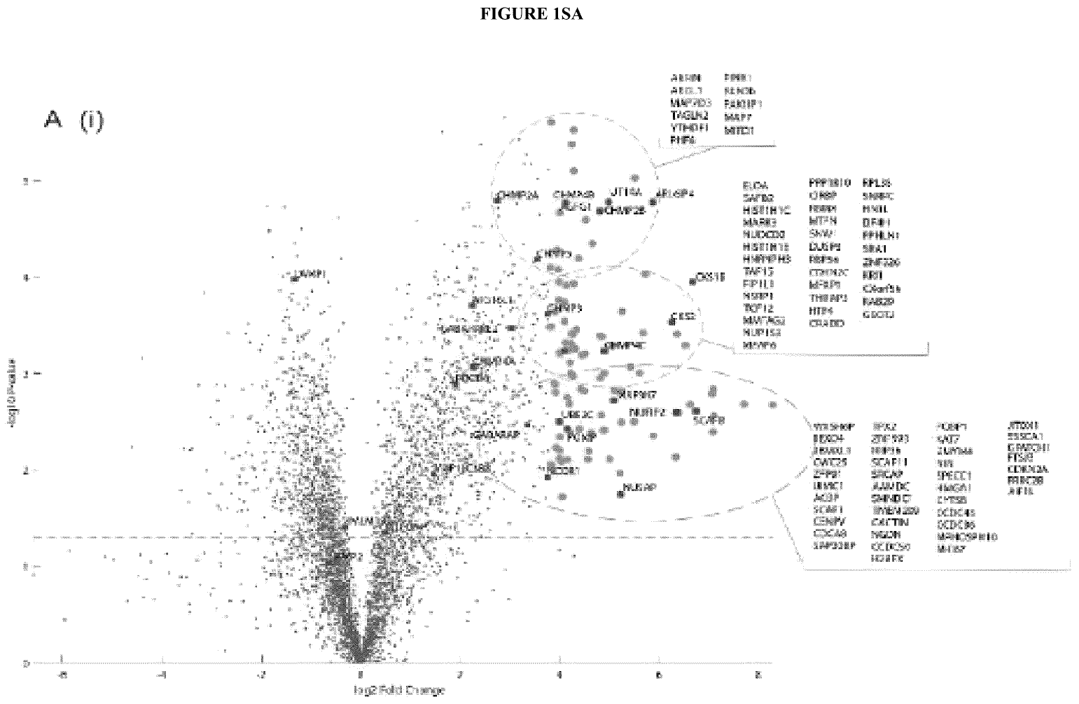

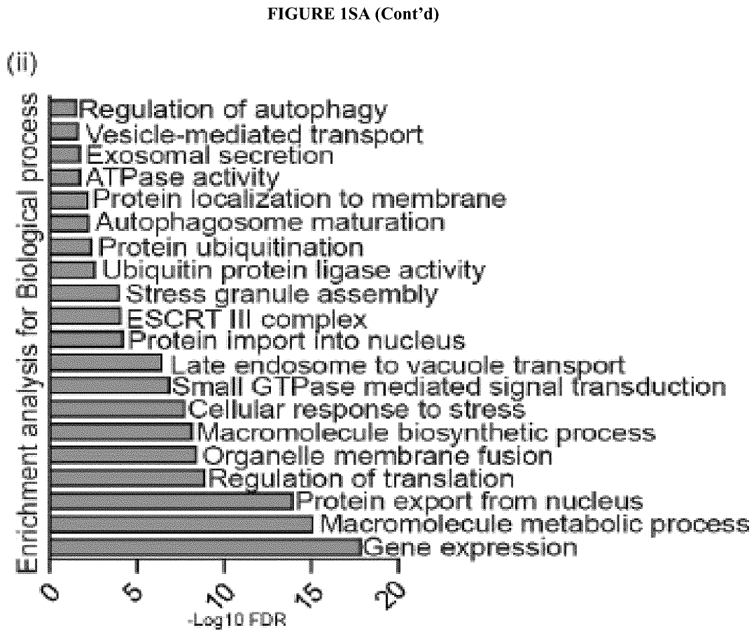

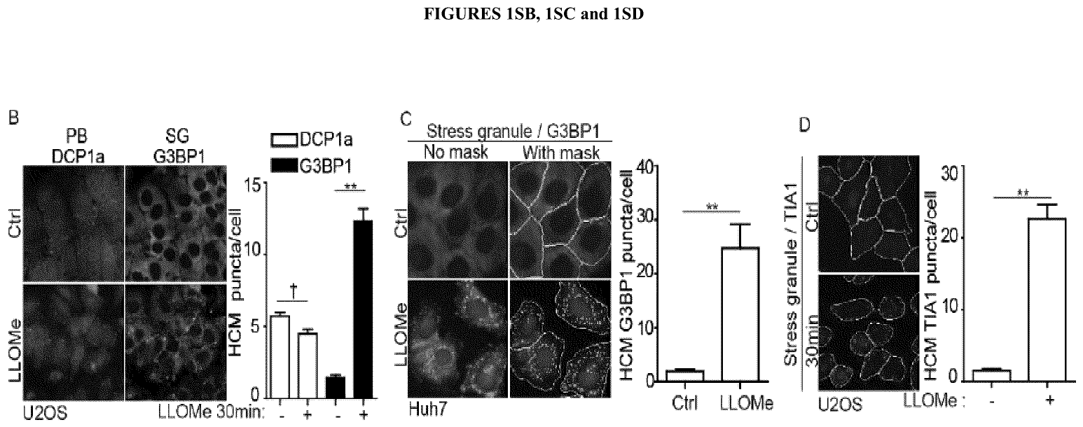

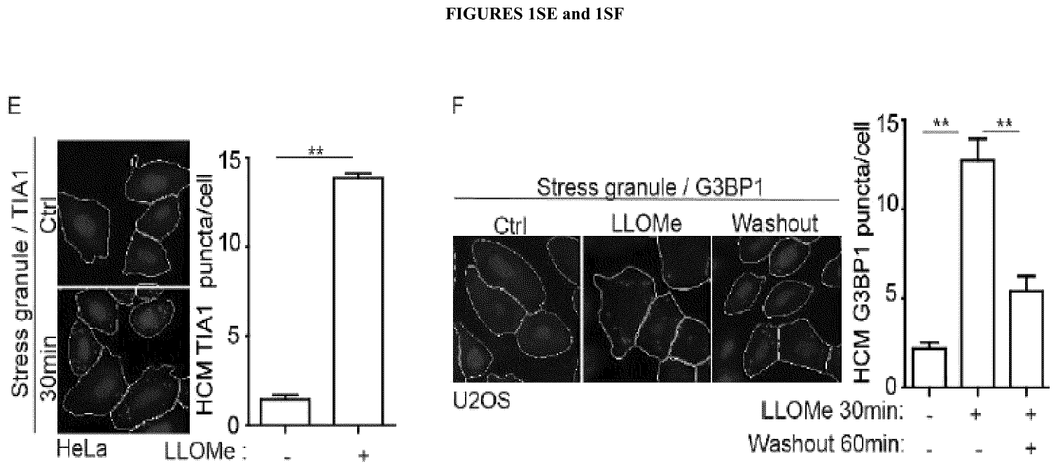

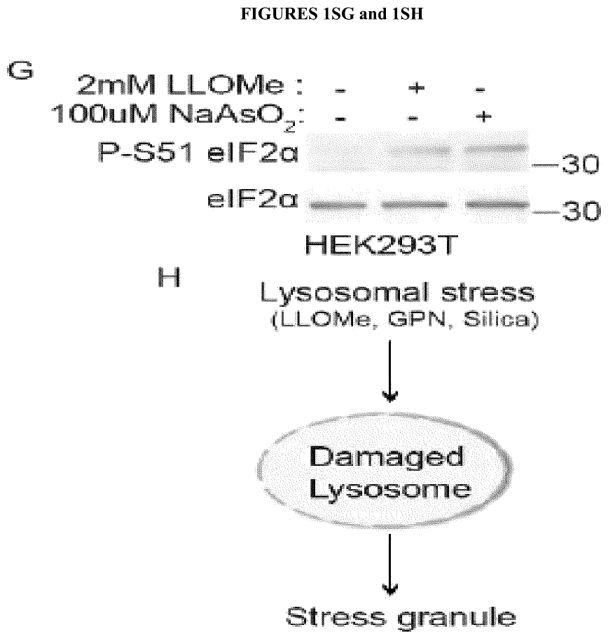

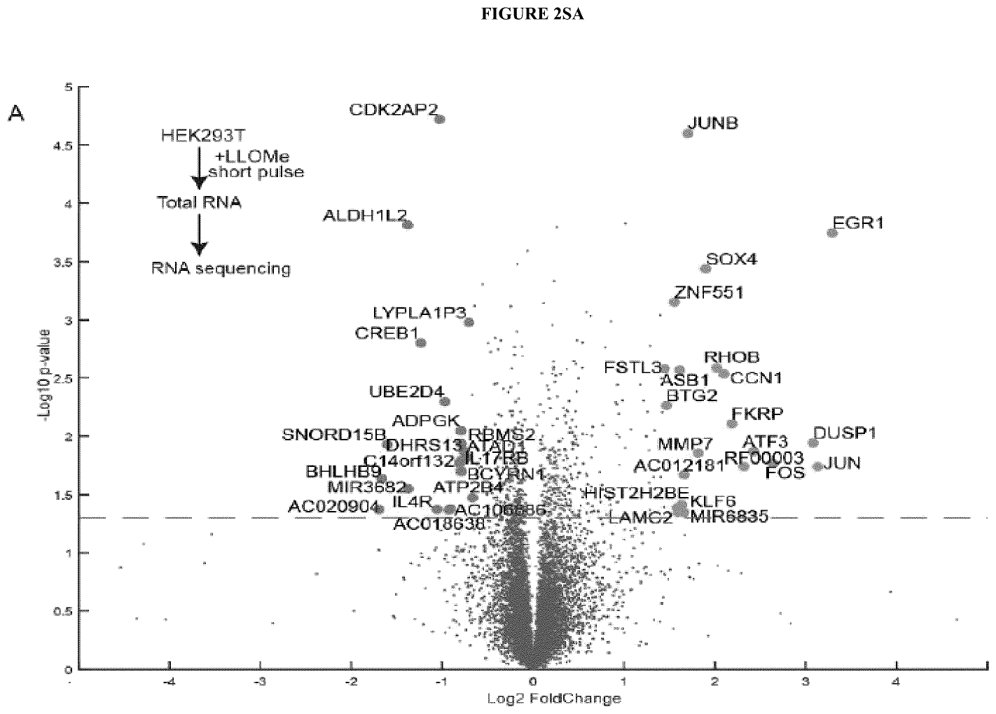

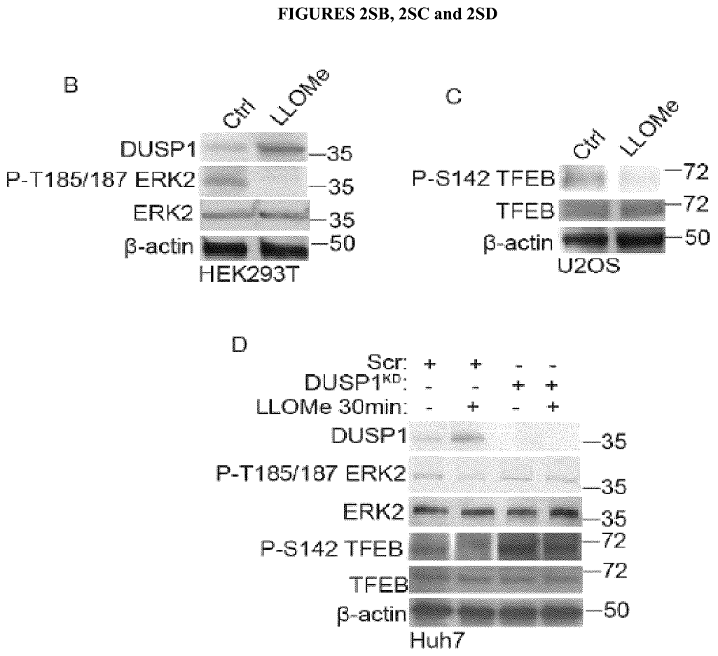

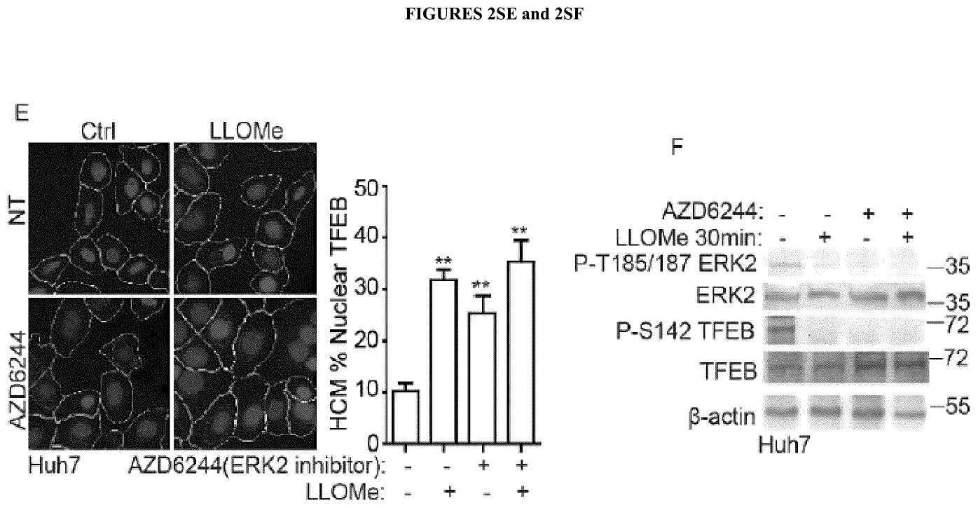

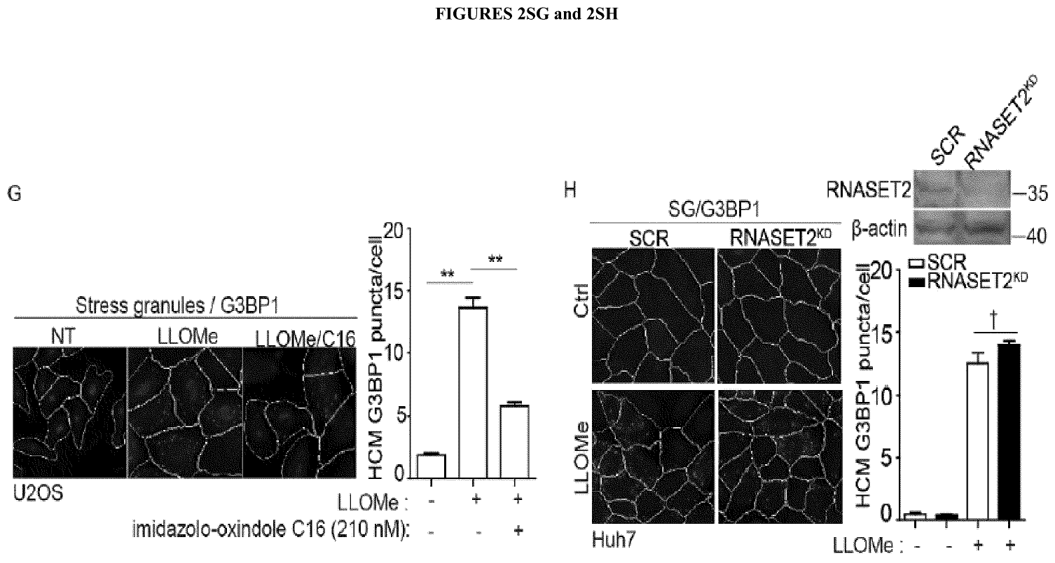

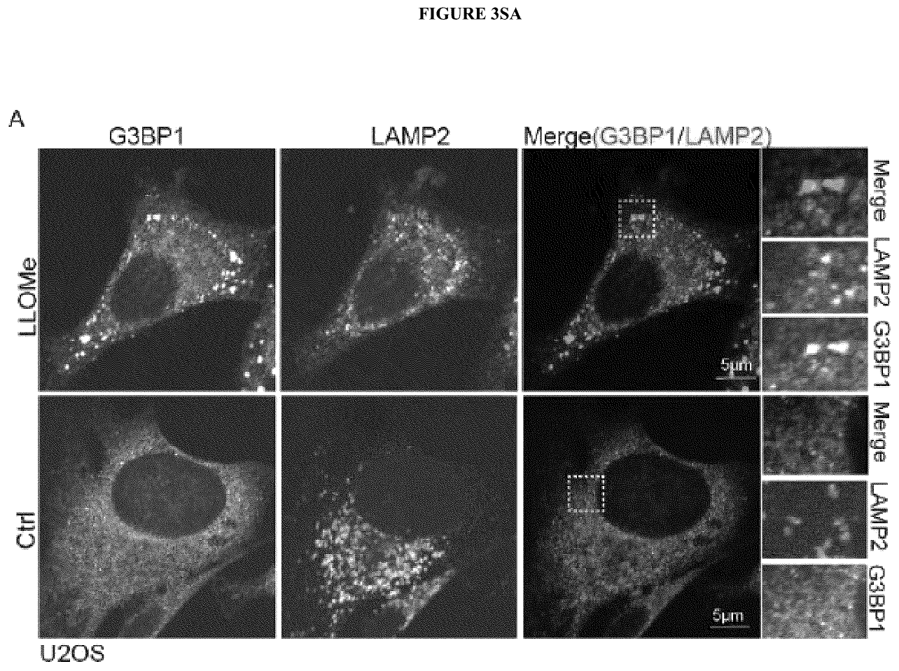

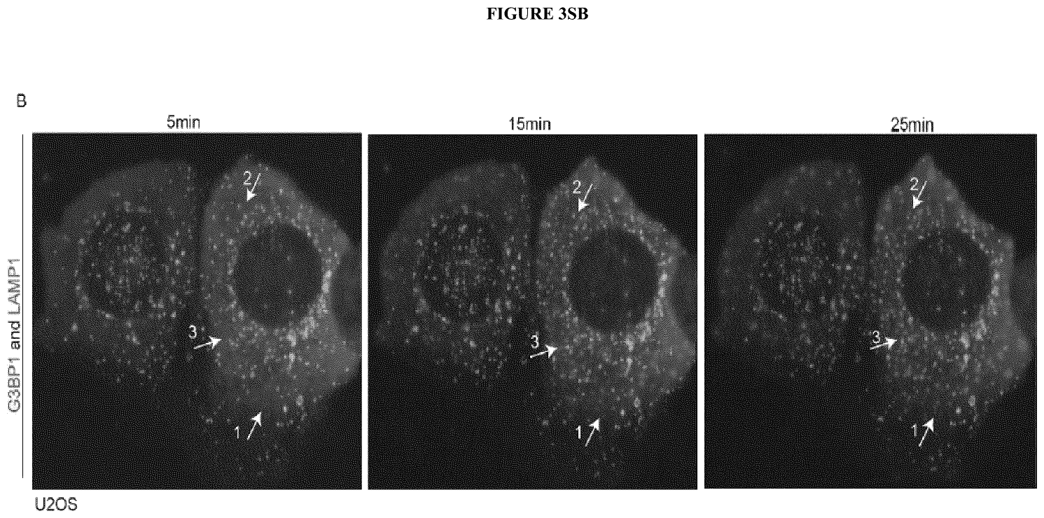

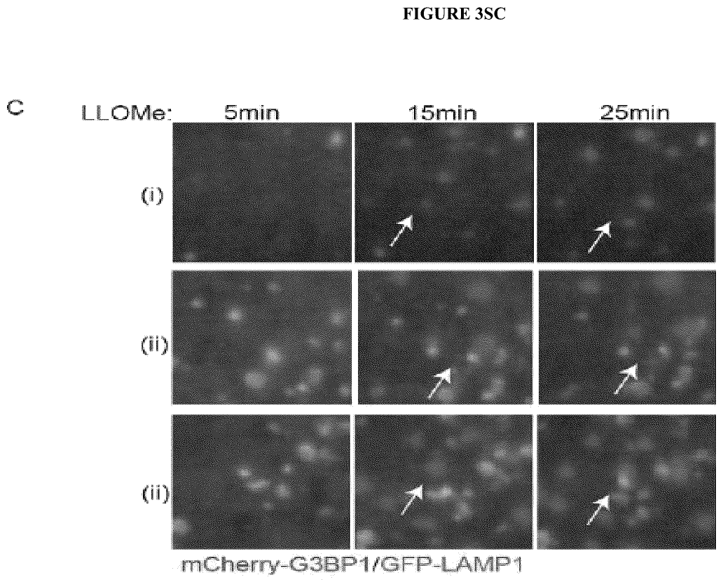

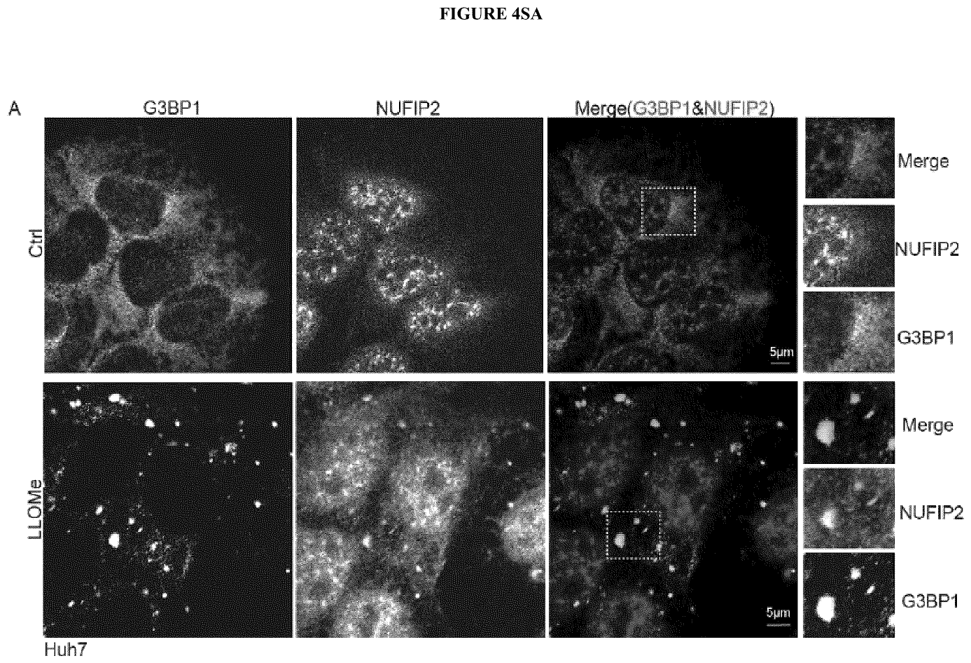

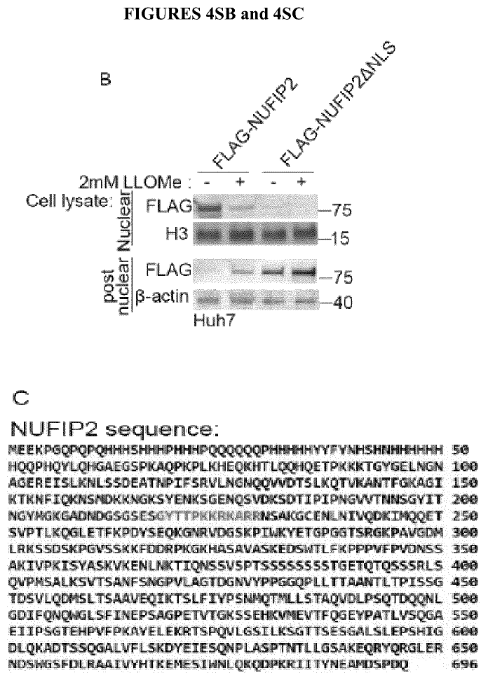

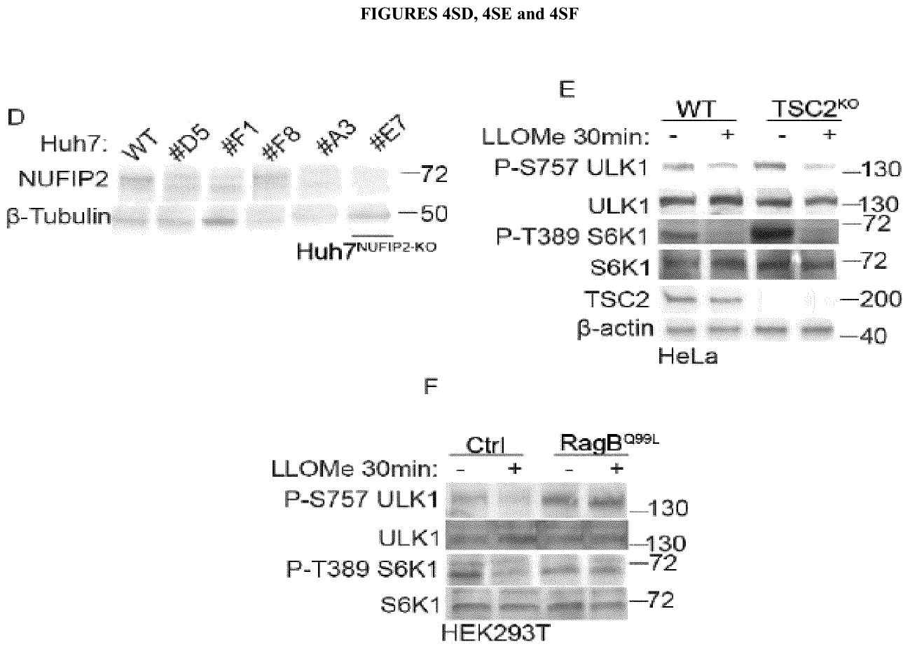

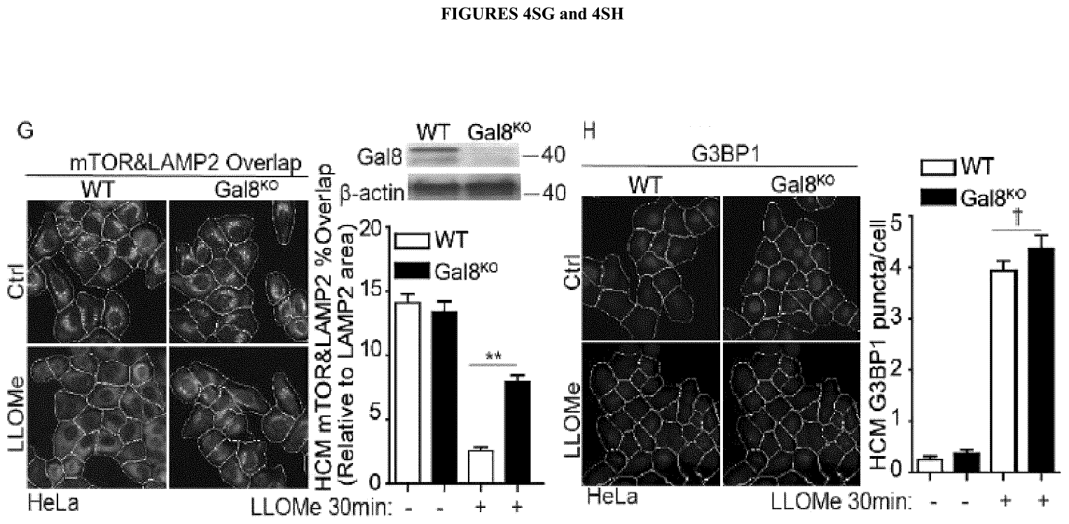

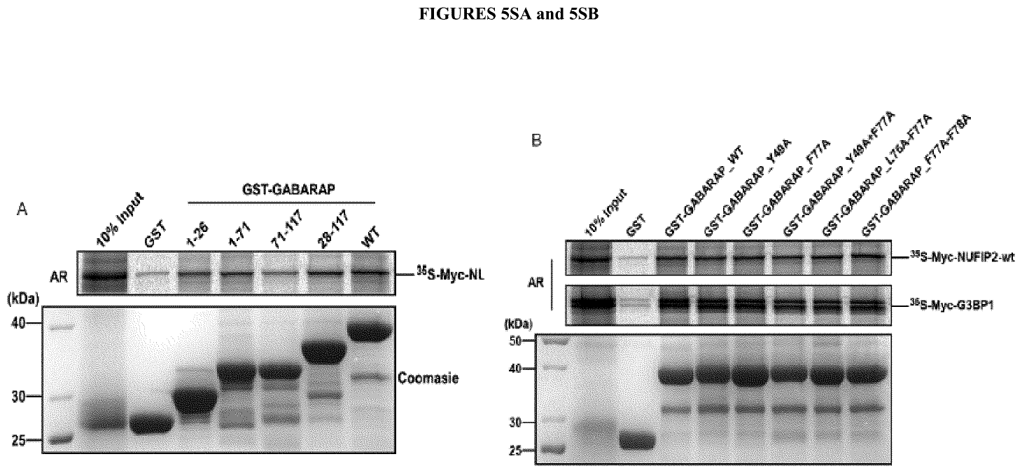

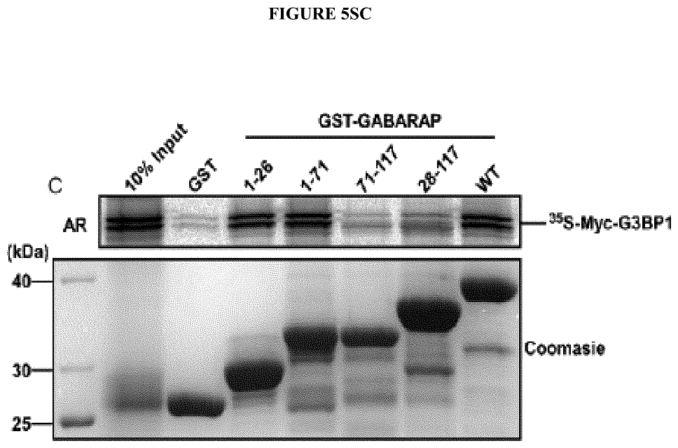

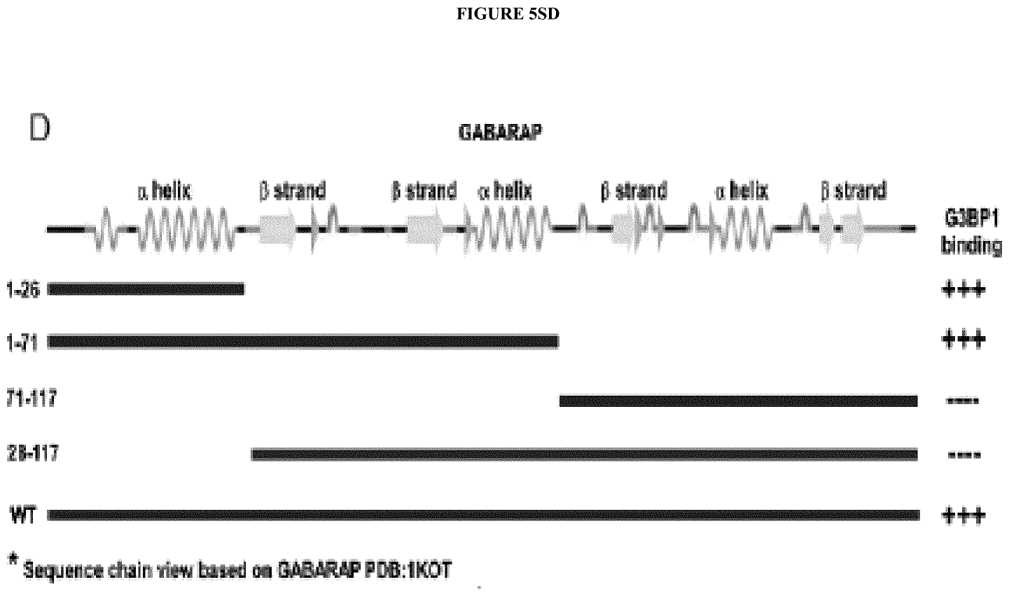

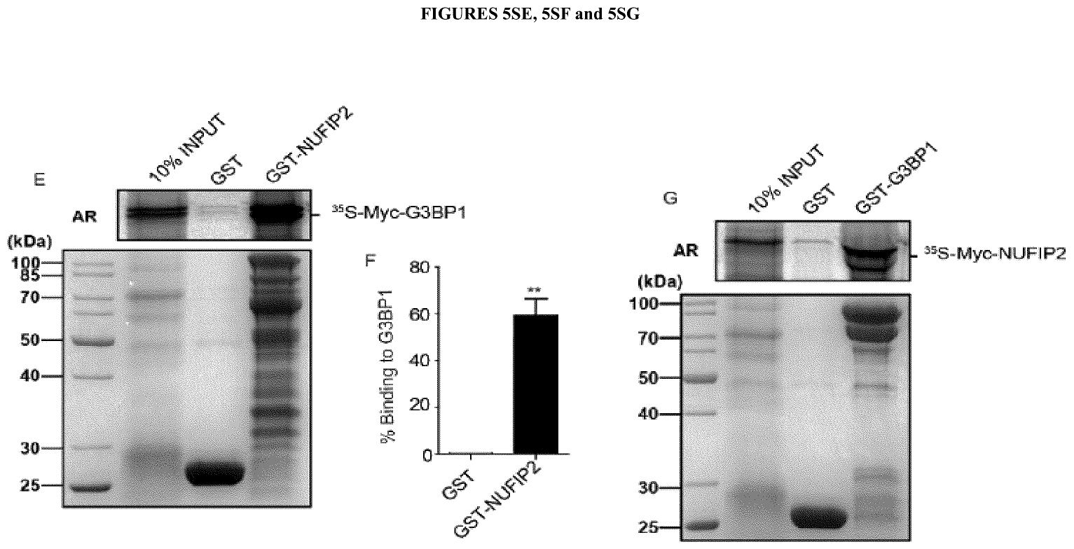

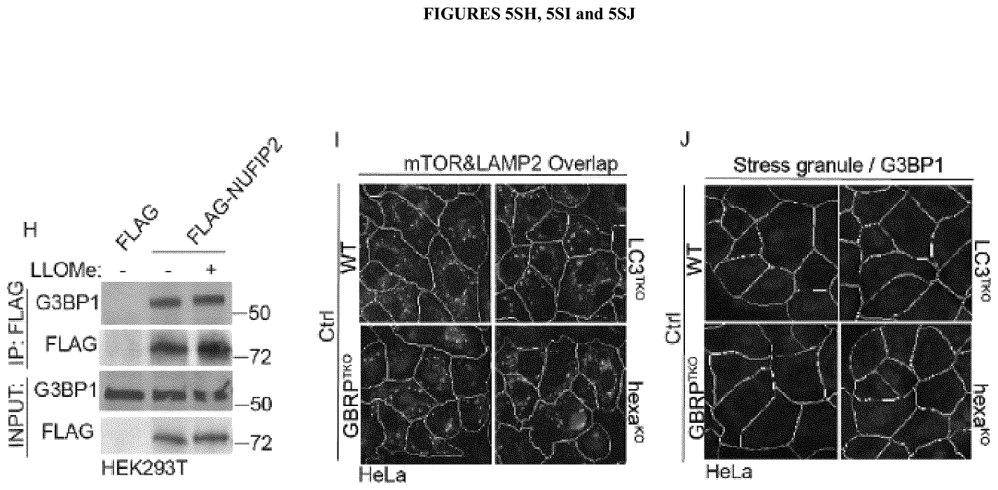

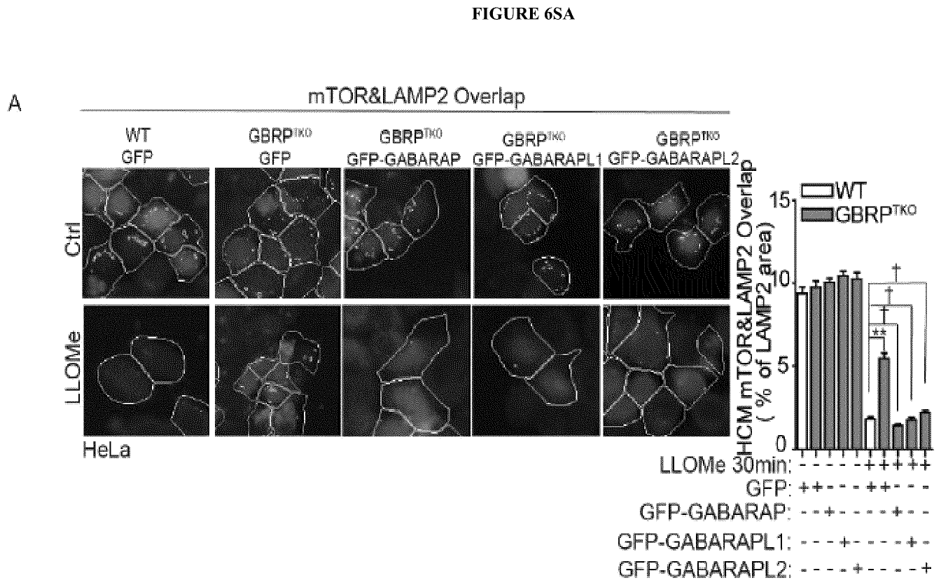

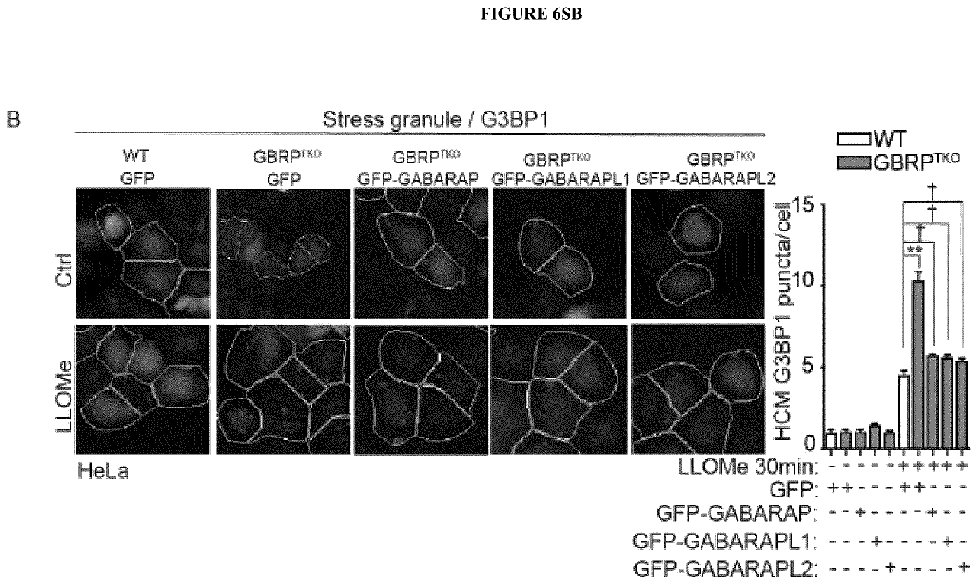

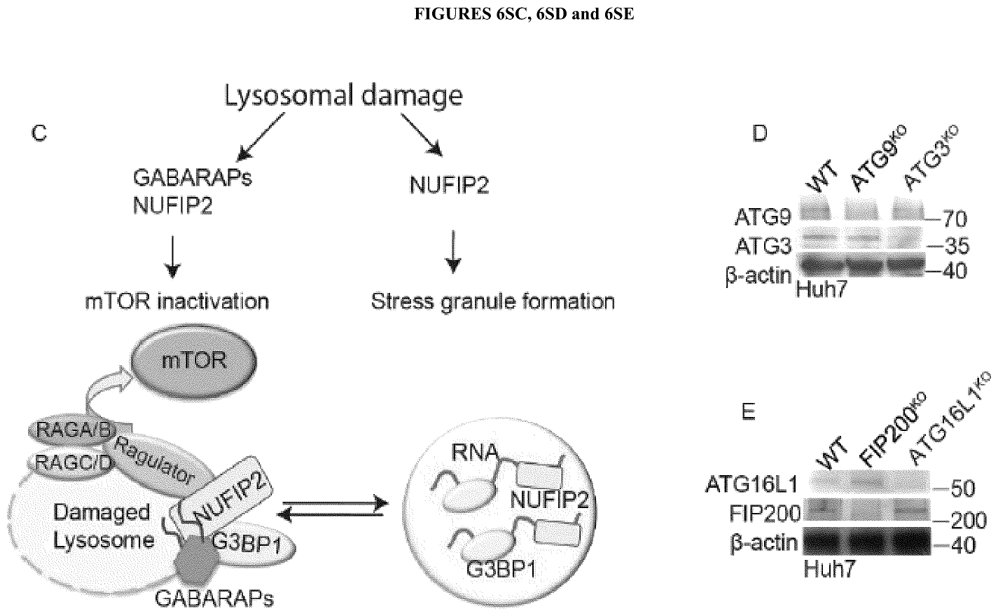

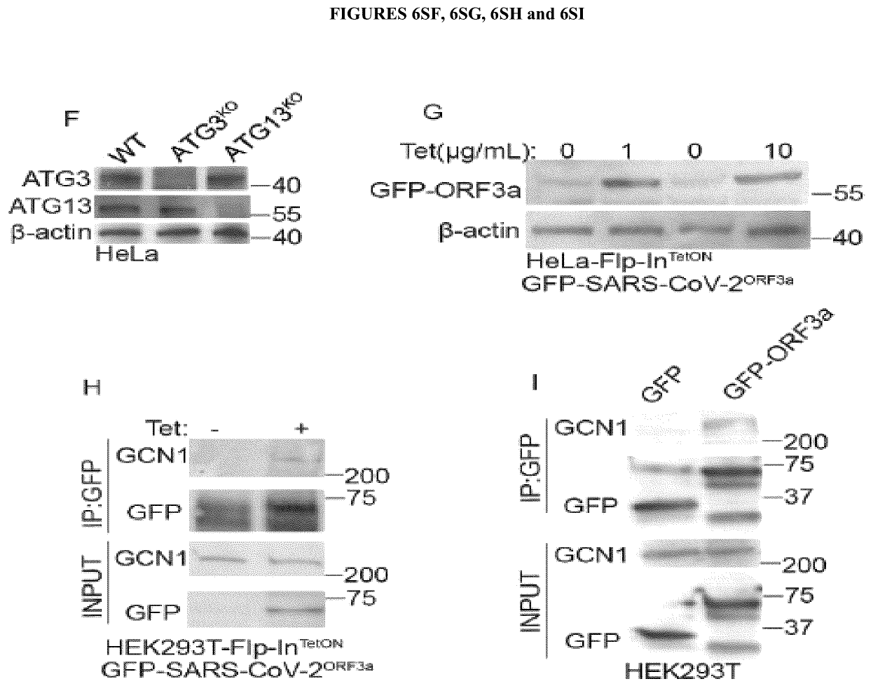

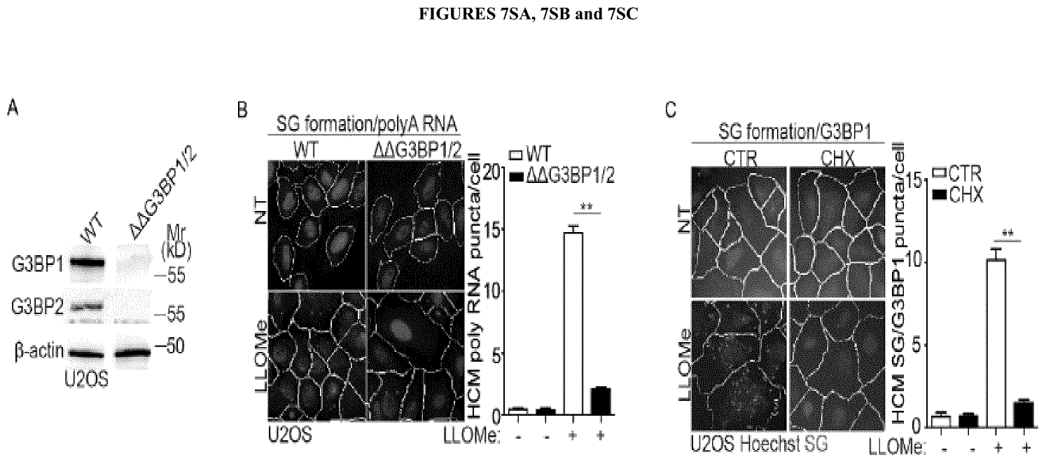

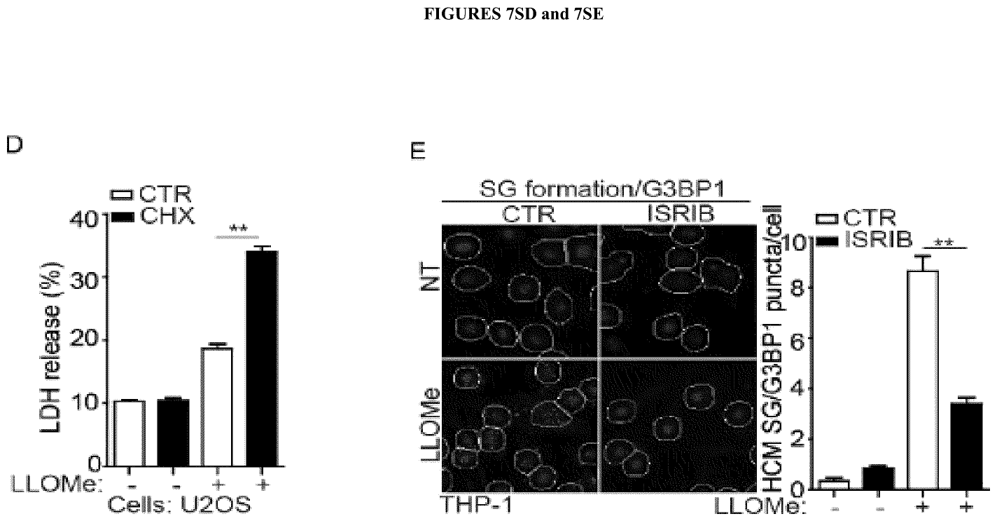

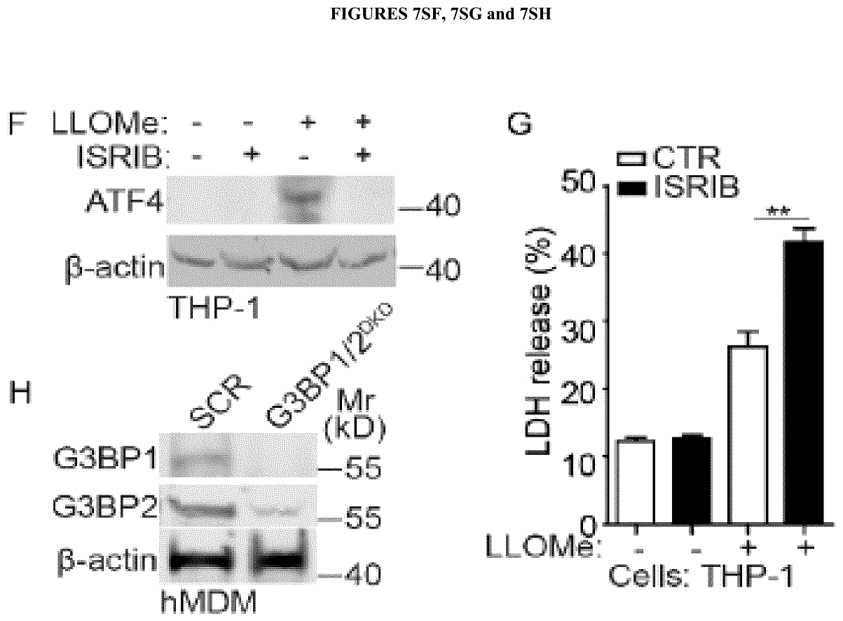

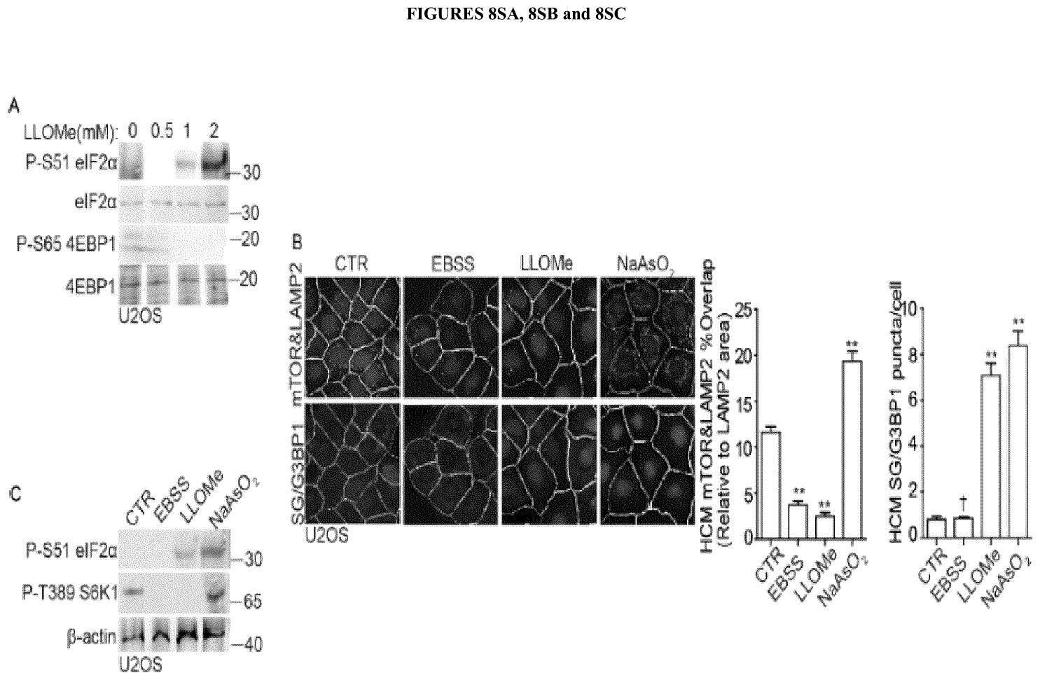

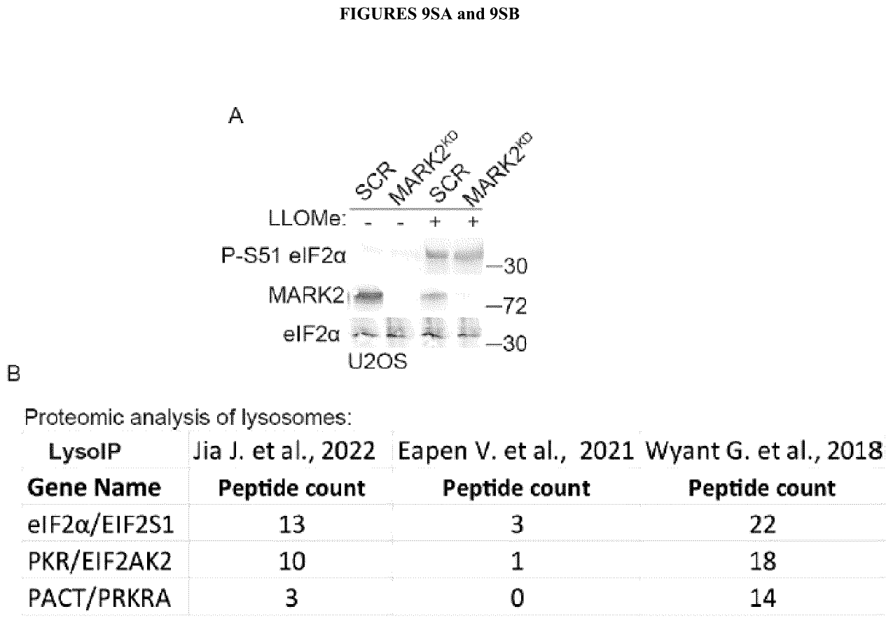

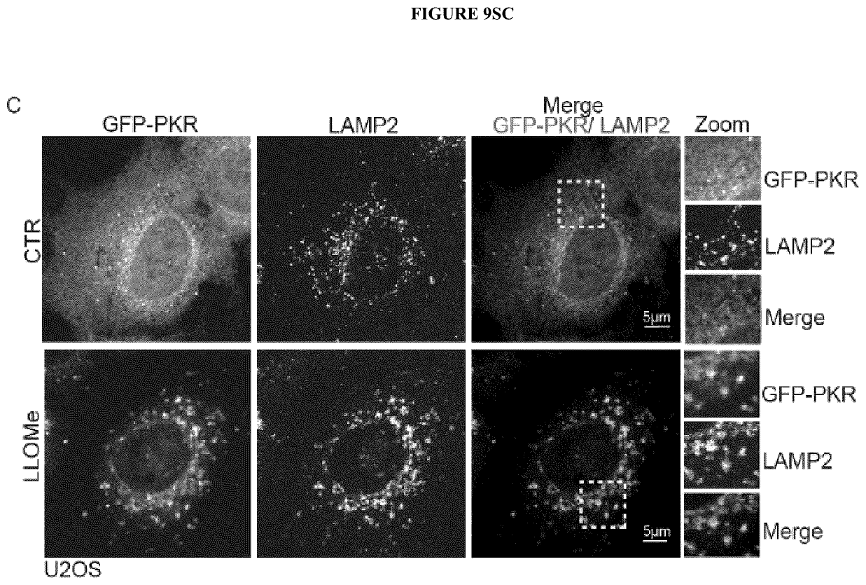

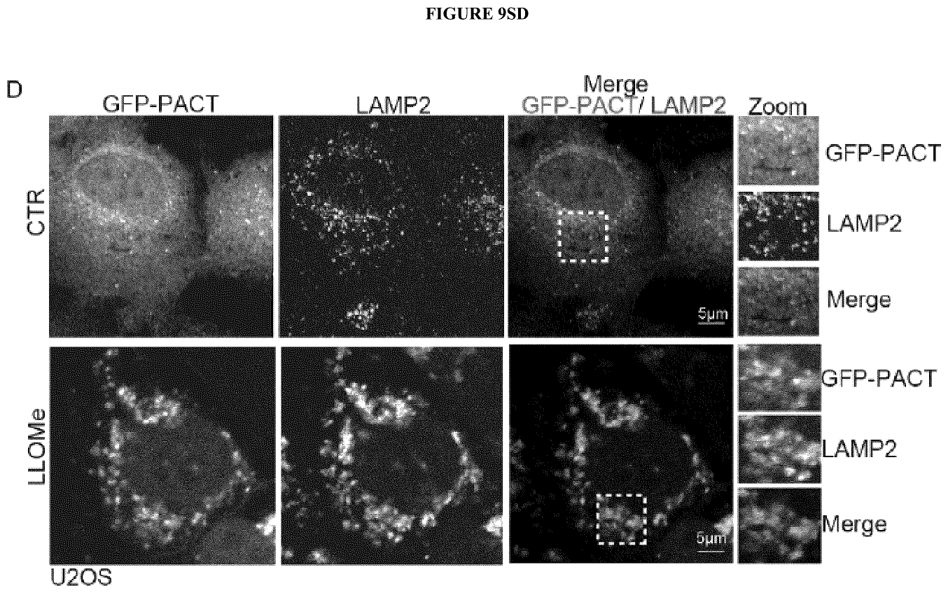

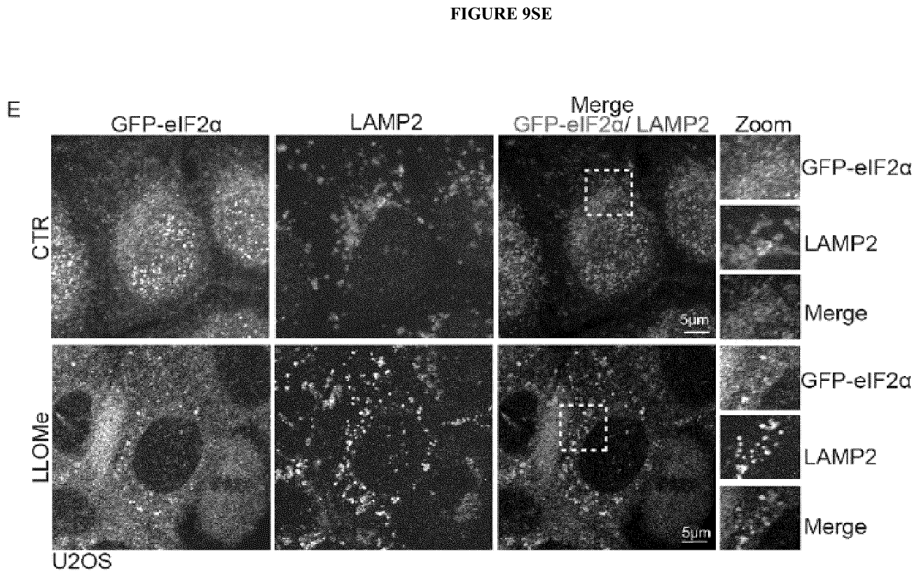

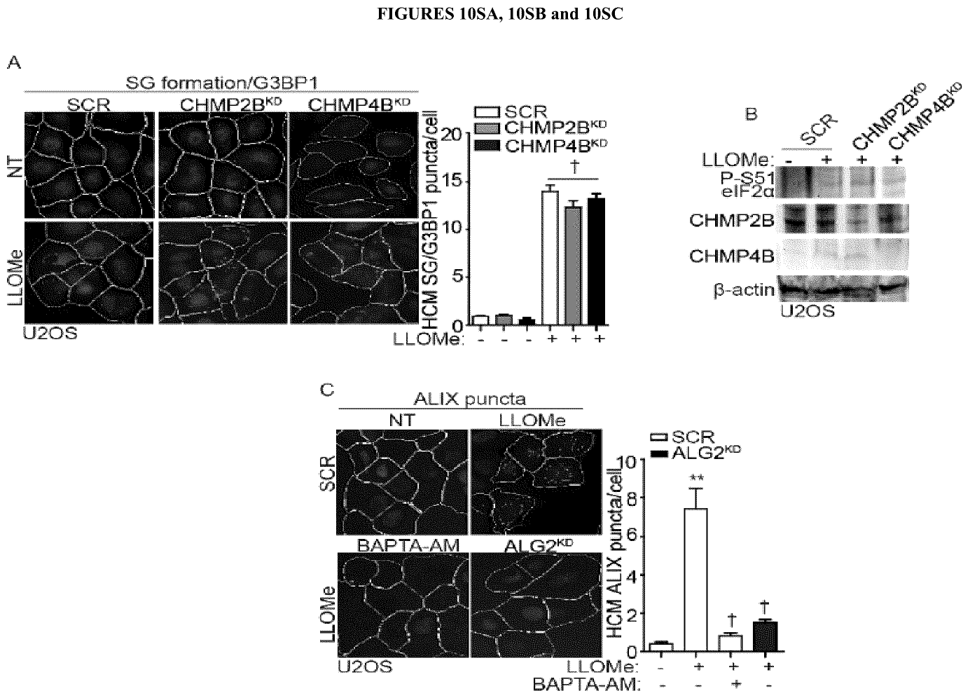

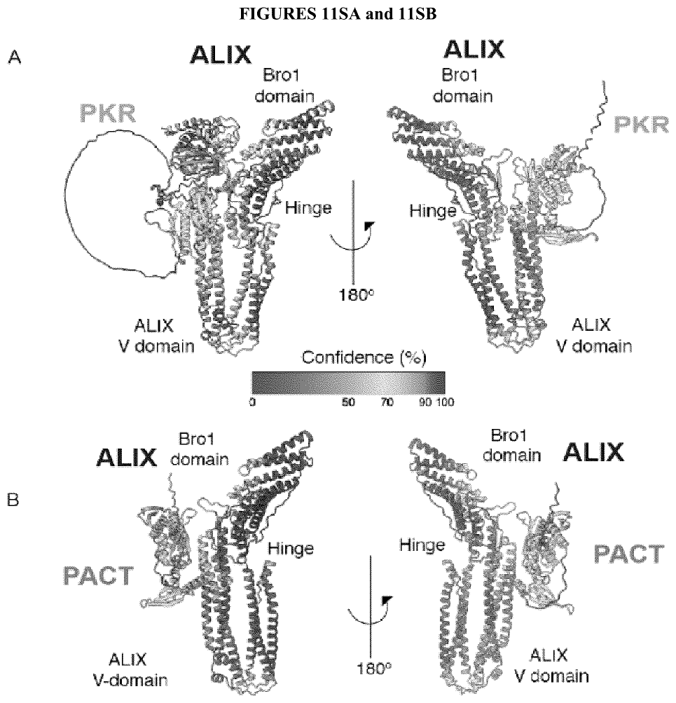

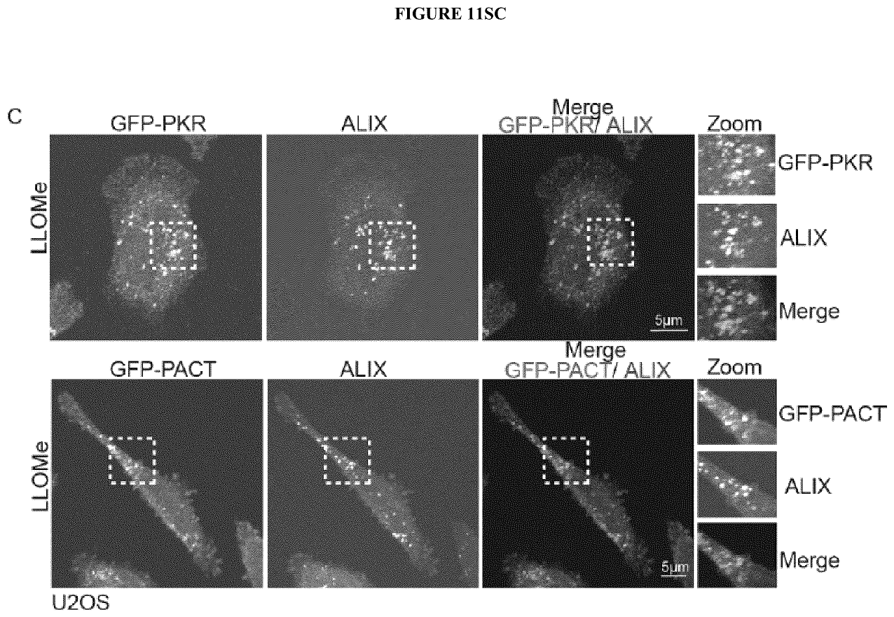

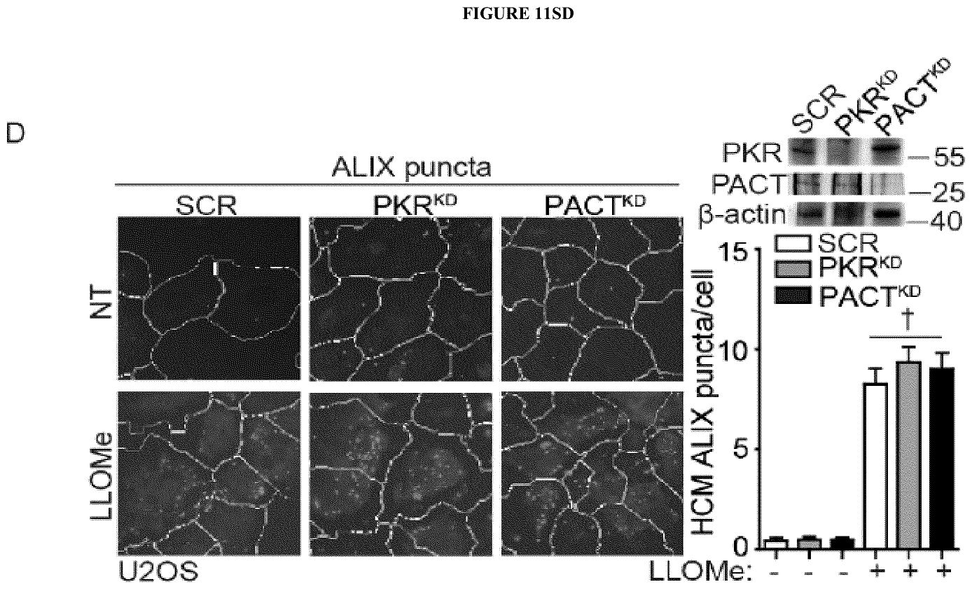

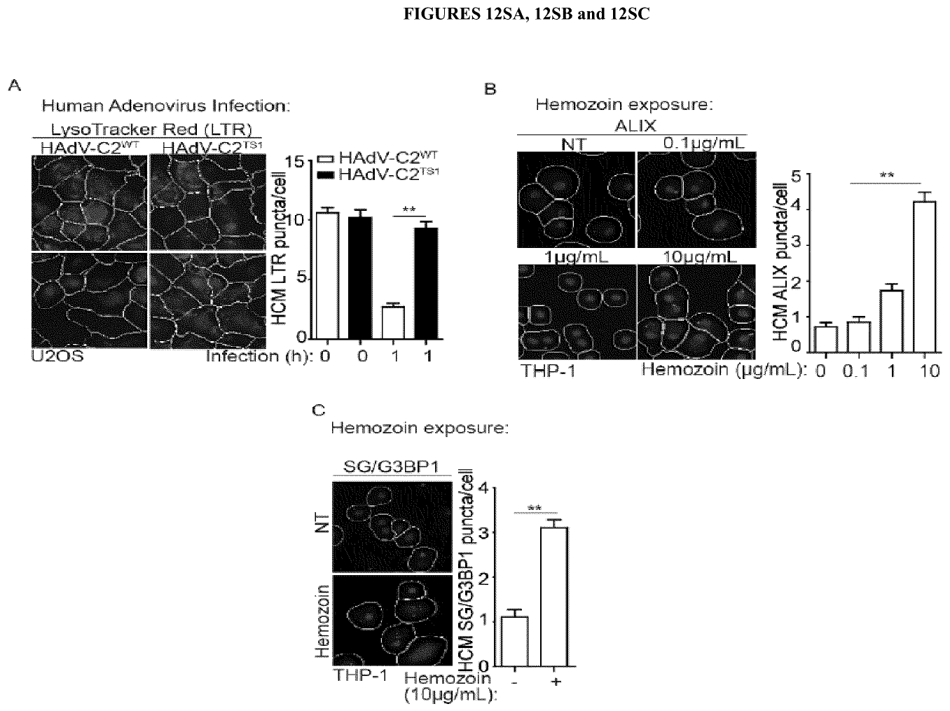

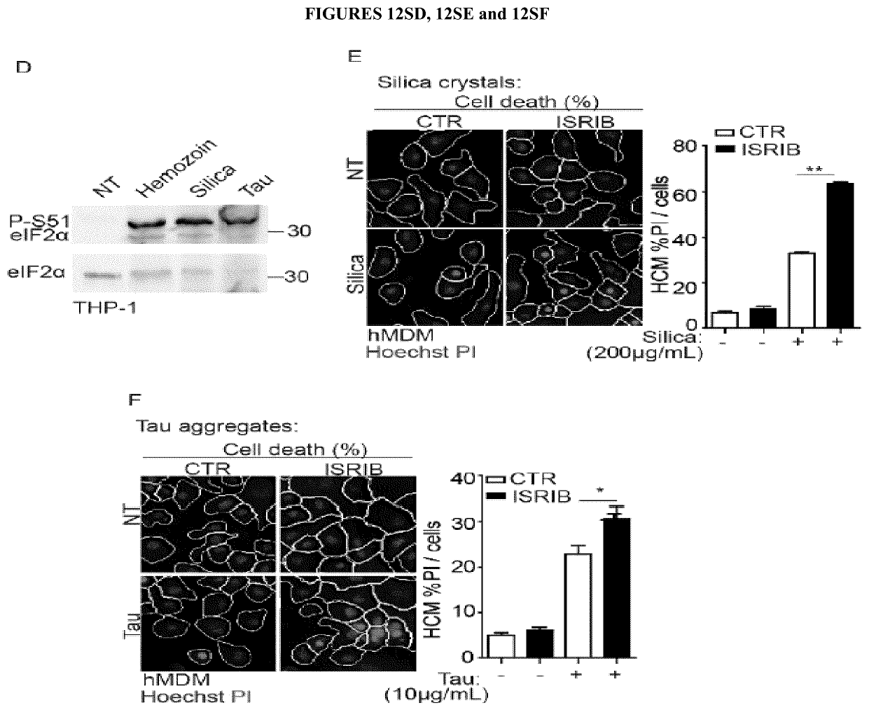

FIGS. 12SA-12SF. Stress granules are important for cell survival in response to lysosomal damage in disease states. FIG. 12SA shows the quantification by HCM of status of acidified organelles assessed by LysoTracker Red (LTR) in U2OS cells infected with wildtype human adenovirus C2 (HAdV-C2^{WT}) or C2 TS1 mutant (HAdV-C2^{TS1}) at MOI=10 for 1 h. White masks, algorithm-defined cell boundaries; red masks, computer-identified LTR puncta. FIG. 12SB shows the quantification by HCM of ALIX puncta in THP-1 cells treated with hemozoin for 4 h at the indicated dose. White masks, algorithm-defined cell boundaries; green masks, computer-identified ALIX puncta. FIG. 12SC shows the quantification by HCM of G3BP1 puncta in THP-1 cells treated with 10 μg/ml hemozoin for 4 h. White masks, algorithm-defined cell boundaries; red masks, computer-identified G3BP1 puncta. FIG. 12SD shows an immunoblot analysis of phosphorylation of eIF2α (S51) in THP-1 cells treated with 10 g/ml hemozoin, 200 μg/mL silica or 10 μg/mL tau oligomer for 4 h. FIG. 12SE shows the quantification by HCM of cell death by a propidium iodide (PI) uptake assay in human peripheral blood monocyte-derived macrophages (hMDM) during silica treatment. Cells Were treated with 200 μg/mL silica for 4 h in the presence or absence of 100 nM ISRIB, and then stained with propidium iodide PI (dead cells) and Hoechst-33342 (total cells). White masks, algorithm-defined cell boundaries; red masks, computer-identified PI⁺ nuclei. FIG. 12SF shows the quantification by HCM of cell death by a propidium iodide (PI) uptake assay in human peripheral blood monocyte-derived macrophages (hMDM) during the treatment of tau oligomer. Cells Were treated with 10 μg/mL tau oligomer for 4 h in the presence or absence of 100 nM ISRIB, and then stained with propidium iodide PI (dead cells) and Hoechst-33342 (total cells). White masks, algorithm-defined cell boundaries; red masks, computer-identified PI+ nuclei. CTR, control; NT, untreated cells. Data, means±SEM (n=3); HCM: n≥3 (each experiment: 500 valid primary objects/cells per Well, ≥5 Well s/sample). *p<0.05, **p<0.01, ANOVA. See also FIG. 14.

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound or composition (such as a modulator of NUFIP2 or G3BP1 as an agonist, often an antagonist, upregulator or down-regulator of the expression of NUFIP2 or G3BP1 and optionally, an autophagy modulator agent) disclosed herein and includes tautomers, regioisomers, geometric isomers as applicable, and also where applicable, stereoisomers, including diastereomers, optical isomers (e.g. enantiomers) thereof, as well as pharmaceutically acceptable salts or alternative salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers, including enantiomer and/or diastereomers and epimers, where applicable in context. The term also refers, in context to prodrug forms of compounds, such as siRNAs which have been modified as conjugates such as dynamic polyconjugates (DPC) as taught for example by Rozema, et al. PNAS, Vol. 104, no. 32, pp 12982-12987, Aug. 7, 2007, GalNAc-SiRNA conjugates (e.g., 2′-ribose positions conjugated with N-acetylgalactoseamine), antibody-SiRNA conjugates (as taught by Cao, et al, Medicine in Drug Discovery, Vol. 15, September 2022, 100128, among others) or siRNA incorporated into lipid nanoparticles to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the modulation of autophagy within the context of a particular treatment or alternatively, the effect of a bioactive agent which is coadministered with the autophagy modulator or lysosomotropic agent in the treatment of disease.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibiting its progression, or ameliorating, lessening or suppressing one or more symptom of an autophagy mediated disease state or condition, especially including excessive inflammation caused by the disease state and/or condition. Treatment, as used herein, encompasses therapeutic treatment and in certain instances, prophylactic treatment (i.e., reducing the likelihood of a disease or condition occurring), depending on the context of the administration of the composition and the disease state, disorder and/or condition to be treated.

As used herein, the term “autophagy mediated disease state or condition” refers to a disease state or condition that results from disruption in autophagy or cellular self-digestion. In particular embodiments, the disease is cancer, especially a cancerous tumor, especially pancreatic cancer, a glioma, glioblastoma or a neuroblastoma. Other cancers which can be treated using the present invention include those which are described herein. Autophagy is a cellular pathway involved in protein and organelle degradation, and has a large number of connections to human disease. Autophagic dysfunction which causes disease is associated with metabolic disorders, neurodegeneration, autoimmune diseases, microbial infections cancer, aging, cardiovascular diseases and metabolic diseases, among numerous other disease states and/or conditions. In embodiments, the autophagic dysfunction is often associated with cancer, especially a tumorous cancer, especially pancreatic cancer, a glioma, glioblastoma or a neuroblastoma. Although autophagy plays a principal role as a protective process for the cell, it also plays a role in cell death.

The term “lysosomotropic agent” is used to describe an agent which is combined with a modulator/inhibitor of NUFIP2 or G3BP1, such as an siRNA according to SEQ ID Nos 3-10 or SJ-19-0043 inhibitor to provide compositions according to the present invention which are particularly effective in the treatment of autophagy-related disease states or conditions as otherwise described herein, especially cancer, including pancreatic cancer, glioma, glioblastoma or neuroblastoma. Lysosomotropic agents include, for example, lipophilic or amphipathic compounds which contain a basic moiety which becomes protonated and trapped in a lysosome. Lysosomotropic agents for use in the present inventon include, for example, lysosomotropic detergents such as a lysosomotropic amine containing a moderately basic amine of pKa 5-9. Examples of such lysosomotropic detergents include sphingosine, O-methyl-serine dodecylamine hydrochloride (MSDH) and N-dodecylimidazole, among others, as well as numerous drugs including chloroquine, chlorpromazine, thioridazine, aripiprazole, clomipramine, imipramine, desipramine and seramasine, among others. Additional lysosomotropic agents include glycyl-L-phenylalanine-2-naphthyl amide (GPN) and Leu-Leu-OMe (LLOMe).

The term “autophagy modulator agent” or “additional autophagy modulator” is used to describe an optional agent which is used in the compositions and/or methods according to the present invention in order to enhance or inhibit an autophagy response in an autophagy mediated disease state which is otherwise treated, ameliorated, inhibited and/or resolved by another agent as set forth herein (e.g. a modulator of NUFIP2 or G3BP1 or a modulator of the expression of NUFIP2 or G3BP1, especially an inhibitor such as an siRNA of SEQ ID Nos 3-10, or optionally in combination with a lysosomotropic agent). Additional autophagy modulators include, but are not limited to, autophagy agonists (such as flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, ambroxol, norcyclobenzaprine, diperodon, nortriptyline or a mixture thereof or their pharmaceutically acceptable salts). Additional autophagy modulators which may be used in the present invention to inhibit, prevent and/or treat an autophagy mediated disease state and/or condition include one or more of benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin, Dyclonine, Salbutamol, Flavoxate, Amoxapine, Fenofibrate, Pimethixene and mixtures thereof. The autophagy modulator may be included as optional agents in compositions according to the present invention or used in conjugation with therapies as otherwise described herein to treat an autophagy mediated disease state or condition.

The term “co-administration” or “combination therapy” is used to describe a therapy in which at least two active compounds in effective amounts are used to treat an autophagy mediated disease state or condition as otherwise described herein, either at the same time or within dosing or administration schedules defined further herein or ascertainable by those of ordinary skill in the art. Although the term co-administration preferably includes the administration of two active compounds to the patient at the same time, it is not necessary that the compounds be administered to the patient at the same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which one active agent is administered at approximately the same time (contemporaneously), or from about one to several minutes to about 24 hours or more after or before the other active agent is administered.

In yet additional embodiments, additional bioactive agents may be further included in compositions according to the present invention in combination with agents which control mTor response to endomembrane damage (e.g. an antagonist of NUFIP2 or G3BP1 or a down regulator of the expression of NUFIP2 or G3BP1 or a mixture thereof, which may optionally be combined with a lysosomotropic agent and/or an autophagy modulator) or other inhibitor such as SJ-19-0043 or ISRIB.

The terms “cancer” and “neoplasia” are used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the terms malignant neoplasia and cancer are used synonymously to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, glioma, glioblastoma, neuroblastoma, hairy cell leukemia, mouth/pharynx, oesophagus, larynx, kidney cancer and lymphoma, among others, which may be treated by one or more compounds according to the present invention. In certain aspects, the cancer which is treated is pancreatic cancer, glioma, glioblastoma ore neuroblastoma, among others.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, stomach and thyroid; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, glioblastomas, neuroblastomas, astrocytomas, oligodendrogliomas, ependymomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17th ed. (Whitehouse Station, N.J.: Merck Research Laboratories, 1999) 973-74, 976, 986, 988, 991). All of these neoplasms may be treated using compounds and compositions according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, glioma, glioblastoma, neuroblastoma, hairy cell leukemia, mouth/pharynx, oesophagus, larynx, kidney cancer and lymphoma, among others, which may be treated by one or more compounds according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer, including for example, a drug resistant pancreatic cancer, a glioma, glioblastoma and/or neuroblastoma, among others. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a beneficial, often synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, agent(s) which may be co-administered with compounds according to the present invention in the treatment of cancer. These agents include chemotherapeutic agents and include one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bel-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser (But) 6, Azgly 10] (pyro-Glu-His-Trp-Ser-Tyr-D-Ser (Bu t)-Leu-Arg-Pro-Azgly-NH 2 acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilimumab, nivolomuab, pembrolizumab, dabrafenib, trametinib and vemurafenib among others.

Co-administration of one of the formulations of the invention with another anticancer agent will often result in a synergistic enhancement of the anticancer activity of the other anticancer agent, an unexpected result. One or more of the present formulations comprising a siRNA according to SEQ ID Nos 3-10 and optionally a lysosomotropic agent as described herein may optionally be further combined with an additional anticancer agent as described herein or another bioactive agent (e.g., antiviral agent, antihyperproliferative disease agent, agents which treat chronic inflammatory disease, among others as otherwise described herein).

According to various embodiments, the combination of compositions and/or compounds according to the present invention may be used for treatment or prevention purposes in the form of a pharmaceutical composition. This pharmaceutical composition may comprise one or more of an active ingredient as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intrathecal, intestinal, parenteral, oral, buccal, nasal, intramuscular, transcutaneous, transdermal, intranasal, intraperitoneal, and topical. The pharmaceutical compositions may be immediate release, sustained/controlled release, or a combination of immediate release and sustained/controlled release depending upon the compound(s) to be delivered, the compound(s), if any, to be coadministered, as well as the disease state and/or condition to be treated with the pharmaceutical composition. A pharmaceutical composition may be formulated with differing compartments or layers in order to facilitate effective administration of any variety consistent with good pharmaceutical practice.

The subject or patient may be chosen from, for example, a human, a mammal such as domesticated animal, or other animal. The subject may have one or more of the disease states, conditions or symptoms associated with autophagy as otherwise described herein.

The compounds according to the present invention may be administered in an effective amount to treat or reduce the likelihood of an autophagy-mediated disease and/or condition, especially cancer, as well as one or more symptoms associated with the disease state or condition. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of an autophagy mediated disease state, condition and/or symptom for a human patient is that which is an effective amount and may range from as little as 100 μg or even less to at least about 500 mg to several grams or more, which may be administered in a manner consistent with the delivery of the drug and the disease state or condition to be treated, often cancer, including the stage of the disease state. In the case of oral administration, active is generally administered from one to four times or more daily. Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient is an effective amount and preferably ranges from about 0.05 mg/kg to about 20 mg/kg, about 0.1 mg/kg to about 7.5 mg/kg, about 0.25 mg/kg to about 6 mg/kg., about 1.25 to about 5.7 mg/kg.

The dose of a compound according to the present invention may be administered at the first signs of the onset of cancer. The dose of active ingredient may be administered at the first sign of relevant symptoms prior to diagnosis, but in anticipation of the disease or disorder or in anticipation of decreased bodily function or any one or more of the other symptoms or secondary disease states or conditions associated with an autophagy mediated disorder to condition.

The present invention thus relates to the following embodiments, among others.

A method of treating an autophagy mediated disease, especially cancer, in a patient in need comprising administering to the patient an effective amount of a NUFIP2 or G3BP1 inhibitor or an inhibitor of the expression of NUFIP2 or G3BP1 or a mixture thereof, optionally in combination with a lysosomotropic agent.

The method wherein the composition includes a lysosomotropic agent.

The method wherein the lysosomotropic agent is a lipophilic or amphipathic compound which contains a basic moiety which becomes protonated and trapped in a lysosome.

The method the lysosomotropic agent is a lysosomotropic detergent.

The method wherein the lysosomotropic detergent is a lysosomotropic amine containing a moderately basic amine of pKa 5-9.

The method wherein the lysosomotropic amine is sphingosine, O-methyl-serine dodecylamine hydrochloride (MSDH), N-dodecylimidazole, or a mixture thereof.

The method wherein the lysomotropic agent is chloroquine, chlorpromazine, thioridazine, aripiprazole, clomipramine, imipramine, desipramine, seramasine, or a mixture thereof.

The method wherein the lysosomotropic agent is glycyl-L-phenylalanine-2-naphthyl amide (GPN), Leu-Leu-OMe (LLOMe) or a mixture thereof.

The method wherein the autophagy mediated disease state is cancer.

The method wherein the autophagy mediated disease state is cancer, especially pancreatic cancer, a glioma, glioblastoma or a neuroblastoma.

The method further including an additional cancer agent to treat the cancer.

The method wherein the autophagy mediated disease state is a metabolic syndrome disease or cancer.

Other embodiments of the present invention relate to pharmaceutical compositions including:

A pharmaceutical composition comprising an effective amount of an inhibitor of NUFIP2 or G3B1P or am inhibitor of the expression of NUFIP2 or G3B1P, especially an inhibitor of NUFIP2 or G3B1P or a mixture of inhibitors thereof, especially at least one siRNA according to SEQ ID Nos: 3-10 hereof, optionally in combination with a lysosomotropic agent.

The composition which includes a lysosomotropic agent.

The composition wherein the lysosomotropic agent is a lipophilic or amphipathic compound which contains a basic moiety which becomes protonated and trapped in a lysosome.

The composition wherein the lysosomotropic agent is a lysosomotropic detergent.

The composition wherein the lysosomotropic detergent is a lysosomotropic amine containing a moderately basic amine of pKa 5-9.

The composition wherein the lysosomotropic amine is sphingosine, O-methyl-serine dodecylamine hydrochloride (MSDH), N-dodecylimidazole or a mixture thereof.

The composition wherein the lysomotropic agent is chloroquine, chlorpromazine, thioridazine, aripiprazole, clomipramine, imipramine, desipramine, seramasine, or a mixture thereof.

The composition wherein the lysosomotropic agent is glycyl-L-phenylalanine-2-naphthyl amide (GPN), Leu-Leu-OMe (LLOMe) or a mixture thereof.

These and other aspects and embodiments of the invention described above, are described further in the following illustrative examples which are provided for illustration of the present invention and are not to be taken to limit the present invention in any way.

### Examples (First Set)

### Summary of Experiments

Pursuant to the following described experiments, the inventors report that lysosomal damage is a hitherto unknown inducer of stress granule (SG) formation and that the process termed membrane Atg8ylation coordinates SG formation with mTOR inactivation during lysosomal stress. SGs Were induced by lysosome-damaging agents including SARS-CoV-2^{ORF3a}, Mycobacterium tuberculosis, and proteopathic tau. During damage, mammalian Atg8s directly interacted with the core SG proteins NUFIP2 and G3BP1 whereas Atg8ylation was needed for their recruitment to damaged lysosomes whereupon NUFIP2 contributed to mTOR inactivation via the Ragulator-RagA/B complex. Thus, cells employ membrane Atg8ylation to control and coordinate SG and mTOR responses to lysosomal damage.

### Lysosomal Proteome Changes During Lysosomal Damage

To complement studies conducted by the inventors of cellular homeostatic responses to lysosomal damage carried out by proximity biotinylation proteomics (Jia et al., 2018; Jia et al., 2020a; Jia et al., 2020b), here the inventors carried out whole organelle proteomic analyses of normative and damaged lysosomes purified by LysoIP (Abu-Remaileh et al., 2017; Jia et al., 2020a; Jia et al., 2020b) (FIGS. 1A and S1A). The inventors chose short pulse for lysosomal damage to capture early events minimizing more advanced degradative processes such as autophagy and other late stage components of the MERIT response (Jia et al., 2020b, c). Cells Were treated for 30 min with Leu-Leu-O-Me (LLOMe), which is a substrate by reverse peptidase reaction of cathepsin C resulting in growing polymers in the lysosomal lumen causing membrane damage and permeabilization (Aits et al., 2015; Jia et al., 2018; Jia et al., 2020a; Jia et al., 2020b; Papadopoulos et al., 2017; Thiele and Lipsky, 1990; Yoshida et al., 2017) Quantitative DIA proteomic analyses was carried out (FIGS. 1A and S1A) of damaged (early damage) vs. undamaged lysosomes in HEK293T cells stably expressing TMEM192-3×HA following a Well-established procedure for lysosomal purification (Abu-Remaileh et al., 2017; Jia et al., 2020a; Jia et al., 2020b) (FIGS. 1A and S1A). The mass spectrometry data confirmed our prior observations (Jia et al., 2018) that mTOR and Raptor dissociate from lysosomes upon damage (FIG. 1A(i)) whereas STRING protein interaction network functional analysis (Szklarczyk et al., 2021) revealed enrichment of components of a number of biological processes including several previously not associated with lysosomal damage (FIGS. 1A(ii) and S1A(ii)).

As an independent control and measure of early cellular response to lysosomal injury, RNAseq analysis was carried out (FIG. 2S A). RNAseq data revealed that during early lysosomal damage, several genes Were induced including DUSP1 (FIG. 2SA). DUSP1 is a phosphatase inhibiting ERK2 activation (Kirk et al., 2020; Sun et al., 1993), whereas ERK2 is an upstream kinase for TFEB (Napolitano and Ballabio, 2016; Puertollano et al., 2018; Settembre et al., 2011). The inventors tested DUSP1 protein levels and the status of ERK2 and TFEB and found: (i) that DUSP1 was increased (FIG. 2SB); (ii) that ERK2 was dephosphorylated (FIG. 2SB); (iii) that TFEB was dephosphorylated at its Ser142 residue, a known site for phosphorylation by ERK2 (Napolitano and Ballabio, 2016; Puertollano et al., 2018; Settembre et al., 2011) (FIG. 2SC); and (iv) that this depended on DUSP1 (FIG. 2SD). Finally, nuclear translocation of TFEB and dephosphorylation at Ser142 in response to lysosomal damage (Chauhan et al., 2016; Settembre et al., 2012) was observed at early time point and was equal in magnitude to the one caused by ERK2 inhibitor AZD6244 (FIGS. S2E, F). The inventors conclude, that early lysosomal damage and the chosen time point elicits a relevant cellular response consistent with prior observations (Chauhan et al., 2016) (Jia et al., 2020b; Nakamura et al., 2020).

An abundance of ESCRT proteins was detected including ALIX (PDCD6IP) and TSG101, shown to contribute to lysosomal damage repair (Jia et al., 2020b; Radulovic et al., 2018; Skowyra et al., 2018) and all ESCRT-III components (CHMP1A, CHMP1B, CHMP2A, CHMP2B, CHMP3, CHMP4A,B,C, CHMP5, CHMP6, CHMP7 and IST1) (FIG. 1A, green).

Another category of proteins detected in DIA proteomic analysis of damaged lysosomes were autophagy-associated components (FIG. 1A, blue), prominently GABARAP (3 unique peptides), GABARAPL2 (7 unique peptides), and ATG16L1 (7 unique peptides). These proteins were enriched upon lysosomal damage and represent components of the mammalian Atg8s (mAtg8s) lipidation process primarily known in the context of autophagy (Mizushima, 2020), but also found on non-autophagic membranes where they perform noncanonical functions (Galluzzi and Green, 2019; Guo et al., 2017b; Heckmann and Green, 2019; Lee et al., 2020b; Leidal et al., 2020b). With the short damage pulse, the inventors observed 2 peptides for MAP1LC3B (LC3B). Another ATG protein implicated in membrane repair (Claude-Taupin et al., 2021), ATG9A (10 unique peptides), showed mild enrichment on damaged lysosomes (FIG. 1A). ATG9A's partner IQGAP1, which interacts directly with CHMP2A (Claude-Taupin et al., 2021) and functions in membrane damage repair, was also prominently present (43 unique peptides identified) albeit it showed no dynamic change during the short damage pulse. Thus, the global proteomic analysis was consistent with the ESCRT components being dynamically recruited and participating in repair of damaged lysosomes (Jia et al., 2020b; Radulovic et al., 2018; Skowyra et al., 2018). The evidence of autophagy factors gathering at the damaged lysosomes was consistent with prior studies (Maejima et al., 2013) (Jia et al., 2018) (Eapen et al., 2021; Jia et al., 2020b)

### Proteomics of Damaged Lysosomes Reveals Connections to Stress Granule Components

Our proteomic analyses of purified damaged lysosomes revealed abundance of proteins best known for their presence in stress granules (SGs) (FIG. 1A, purple). SGs are canonically induced in response to stressors such as heat shock (Nover et al., 1983), oxidative stress (Kedersha et al., 1999) and viral infection (Srivastava et al., 1998; Williams, 2001), however lysosomal damage has hitherto not been reported as an inducer of SGs. SG composition is complex and, depending upon conditions and complementary genomic vs. proteomic approaches approach, can include 274 to 411 proteins (Jain et al., 2016; Yang et al., 2020). These sets of proteins include the proposed core of 36 SG proteins (Yang et al., 2020). Our LysoIP proteomic analysis includes 32 out of the 36 core proteins. Of these, 20 showed statistically significant increase by quantitative DIA analysis (FIG. 1A). Comparing our LysoIP proteomic data with other summaries of proteins associated with SGs (Ivanov et al., 2019), the inventors detected 13 additional exclusive SG proteins and 10 shared between SGs and P-bodies. Of these, 15 showed statistically significant increase by quantitative proteomics of damaged lysosomes (FIG. 1A), for a total of 55 SG proteins in LysoIP MS with 27 of those showing increased association with damaged lysosomes. SGs include stalled preinitiation complexes with 40S ribosomal subunit (Ivanov et al., 2019; Kedersha and Anderson, 2002; Kedersha et al., 2005; Riggs et al., 2020). The inventors detected 30 out of 33 human 40S proteins (Nakao et al., 2004) in our proteomic dataset, with 10 of those showing statistically significant increase in association with damaged lysosomes (FIG. 1A). Thus, our quantitative proteomics analysis detected increased association of SG proteins with damaged lysosomes, including the conventional marker proteins for SGs, G3BP1 and TIA1 (FIG. 1A) (Gilks et al., 2004; Kedersha et al., 2005). Another more recently widely accepted marker of SGs (Markmiller et al., 2018; Yang et al., 2020; Youn et al., 2018), NUFIP2 was prominent in our LysoIP MS, and showed one of the highest enrichments upon lysosomal damage (FIG. 1A). By LysoIP immunoblotting, the inventors confirmed that NUFIP2, G3BP1, and TIA1 are enriched on damaged lysosomes but not on lysosomes purified from cells treated with arsenite, a conventional inducer of SG formation response (Jain et al., 2016; Kedersha et al., 1999) (FIG. 1B). Using previously characterized G3BP1-GFP U2OS cells (Mackenzie et al., 2017) the inventors further confirmed that G3BP1, a principal component of SGs, is recruited to lysosomes upon damage but not under arsenite-treatment conditions in our experimental conditions. This is evidenced by G3BP1's interactions with LAMP1 and LAMP2 almost exclusively under lysosomal damage conditions. Thus The inventors conclude that proteins that are primarily known for being components of SGs are recruited to lysosomal membranes upon damage.

### Lysosomal Damage Induces Stress Granule Formation

The inventors tested whether lysosomal damage induces SG formation using the conventional marker of SGs G3BP1 (Jain et al., 2016; Kedersha et al., 2005; Yang et al., 2020). For this, the inventors employed a panel of cell lines (U2OS; FIGS. 1C and 1SB) and Huh7 and HeLa cells (FIGS. 1SC and 1SE) and in primary cells (BMMs, murine bone marrow derived macrophages; FIG. 1E) to establish whether the process of SG formation in response to lysosomal damage is universal or occurring only in a limited number of cell types. For unbiased quantification, the inventors employed high content microscopy (HCM), which depends on epifluorescence mode to capture all cellular profiles in a volume. HCM allows statistically highly powered (each experiment with machine-identified 500 valid primary objects/cells per Well, ≥5 Well s/sample) with operator-independent image acquisition and data analysis (Claude-Taupin et al., 2021; Jia et al., 2018; Jia et al., 2020a; Jia et al., 2020b; Kumar et al., 2021a). The inventors did not use HEK 293T cells for HCM due to their propensity not to form monolayers compatible with HCM quantifications. To supplement HCM epiflorescence imaging (FIG. 1C), the inventors ran in parallel confocal microscopy images providing better resolution of profiles' morphology (FIG. 1D). In all cell lines and primary cells tested, SG formation was observed upon lysosomal damage and quantified by HCM.

In U2OS cells, the human osteosarcoma epithelial cell line that allows morphological analyses and is suitable for high content microscopy (HCM), 30 min of LLOMe treatment caused morphologically detectable SGs (FIG. 1D). This was quantified by HCM, indicating a robust SG formation response in cells subjected to lysosomal damage by LLOMe (FIG. 1C). Unlike SG formation response, LLOMe treatment did not induce P-body (Ivanov et al., 2019; Kedersha et al., 2005) formation in U2OS cells, as assessed by the DCP1a marker exclusive to P-bodies (Ivanov et al., 2019; Kedersha et al., 2005) (FIG. 1SB). A strong SG formation response was observed with GPN, another biochemical agent causing lysosomal damage (Berg et al., 1994; Jia et al., 2018; Jia et al., 2020b), and in cells treated with agents such as silica crystals that physically damage lysosomal membranes (Hornung et al., 2008; Jia et al., 2018; Maejima et al., 2013) (FIG. 1C). In contrast, starvation in EBSS, a common method of inducing autophagy or inhibiting mTOR (Deretic and Kroemer, 2021), did not cause SG response (FIG. 1C). As another control for LLOMe, the inventors used LOMe, a methoxy esterified leucine (instead of esterified Leu dipeptide) (Zoncu et al., 2011), and it did not induce SG formation (FIG. 1C).

SG response was detected in other cells, including Huh7 cells, the human hepatocyte-derived carcinoma cell line (FIG. 1SC). SGs Were detected in murine primary bone marrow derived macrophages (BMM) subjected to LLOMe treatment (FIG. 1E). This response was as robust as a response to canonical SG inducer arsenite (FIG. 1E). Arsenite however did not induce lysosomal damage, monitored by Galectin-3 response, a conventional marker for lysosomal damage (Aits et al., 2015; Maejima et al., 2013) (Jia et al., 2020b) (FIG. 1F). The inventors finally confirmed SG response to lysosomal damage using another key immunofluorescence marker for SGs, TIA1 (FIGS. 1SC, E).

Activation of specific protein kinases has been established as a part of SG response, including eIF2α (Ivanov et al., 2019; Kedersha et al., 1999). The inventors tested whether lysosomal damage with LLOMe induces phosphorylation of eIF2α. LLOMe treatment of U2OS cells for 30 min induced phosphorylation of eIF2α on Ser51, whereas a recovery from lysosomal damage by LLOMe washout (Maejima et al., 2013) (Jia et al., 2018; Jia et al., 2020b) subsided eIF2α pS51 response (FIG. 1G). This correlated with a reduction in the number of SGs upon LLOMe washout (FIG. 1S F). In BMM cells subjected to lysosomal damage, the levels of eIF2α pS51, corresponding to GDP-locked state of this kinase that normally cycles through its GTP-bound state to initiate new rounds of translation (McCormick and Khaperskyy, 2017), Were similar to those in cells treated with arsenite (FIG. 1H). A similar increase in eIF2α pS51 in response to LLOMe or arsenite was observed in HEK293T cells used in our proteomic studies (FIG. 1SG). In summary, based on the observed hallmarks of conventional SG response, the inventors conclude that lysosomal damage is a newly identified noncanonical stimulus for induction of canonical SGs (FIG. 1SH).

The SGs monitored by G3BP1 puncta were authentic SGs as they completely overlapped with polyA RNA probe (Cy3-oligo-dT) detected by fluorescence in situ hybridization (FISH) (FIG. 1I), since functional SGs sequester translationally arrested mRNAs (Anderson and Kedersha, 2006; Ivanov et al., 2019; Nover et al., 1989). SGs contribute to stress-induced translation arrest which suppresses bulk cap-dependent protein synthesis, but enhances selective translation of ATF4 (Vattem and the inventors k, 2004), which is a part of integrated stress response (ISR) (Lu et al., 2004) (Costa-Mattioli and Walter, 2020). When The inventors tested whether LLOMe-induced lysosomal damage affected ATF4 expression, The inventors found that LLOMe treatment increased ATF4 expression over time (FIG. 1J). Paralleling the treatment was inhibition of mTOR as measured by 4EBP1 phosphorylation (FIG. 1J). Increased ATF4 expression is associated with the dephosphorylation of eIF2α (Novoa et al., 2003), reflected in reduced levels of phosphorylated eIF2α at later times during LLOMe treatment (FIG. 1J). Thus, lysosomal damage induces SGs and aspects of selective translation.

### PKR Transmits Lysosomal Damage Signals Leading to Stress Granule Formation

How might lysosomal damage be perceived and relayed to the systems that regulate SG formation? Mammalian eIF2α can be phosphorylated by four kinases relaying distinct stressors: (i) HRI (EIF2AK1) is activated by oxidative stress or heat shock (Lu et al., 2001; McEwen et al., 2005). (ii) PKR (EIF2AK2), a double-stranded RNA-dependent kinase, recognizes dsRNA during viral infection (Srivastava et al., 1998; Williams, 2001); (iii) PERK (EIF2AK3), a resident ER protein, is activated by ER stress (Patil and Walter, 2001); and (iv) GCN2 (EIF2AK4) is activated by amino acid deprivation (Kimball, 2001). In our proteomic analyses of purified damaged lysosomes, only PKR (10 unique peptides) was detected (FIG. 2A). A trend in PKR increase upon damage was observed by quantitative DIA proteomic analysis of damaged vs. undamaged lysosomes (FIG. 2A). Thus, the inventors tested whether PKR and other eIF2α kinases (HRI, PERK and GCN2) Were required to transmit lysosomal damage and cause eIF2α phosphorylation. Of the four tested, only a knockdown of PKR abrogated eIF2α phosphorylation in response to lysosomal damage by LLOMe (FIG. 2B). PKR was activated, as assessed by its phosphorylation at Thr446, in cells subjected to lysosomal damage (FIG. 2C). An inhibitor of PKR, 2-aminopurine (2-AP) (Lu et al., 2012; Thomis and Samuel, 1993) inhibited eIF2α phosphorylation in cells treated with LLOMe (FIG. 2C). Thus, PKR is responsible for eIF2α phosphorylation in response to lysosomal damage.

When the inventors tested the effects of knockdowns of all four eIF2α on SG formation in response to lysosomal damage, only a knockdown of PKR showed statistically significant reduction in SG formation induced by LLOMe treatment (FIG. 2D). Furthermore, 2-AP inhibited, in a dose-response fashion, SG formation in response to LLOMe treatment (FIG. 2E). A more specific inhibitor of PKR, imidazolo-oxindole C16, also reduced SG formation during lysosomal damage (FIG. 2SG). PKR recognizes dsRNA during viral infections (Srivastava et al., 1998; Williams, 2001). The inventors thus tested the possibility that RNA potentially released from damaged lysosomes could activate PKR. The inventors knocked down lysosomal RNase RNASET2 (Haud et al., 2011) but did not detect a reduction in SG formation in response to LLOMe (FIG. 2SH). Although this does not rule out that substances released from damaged lysosomes activate PKR, the inventors note that PKR can be activated in dsRNA-independent manner via interactors such as PACT (PRKRA) (Li et al., 2006), which is found in our LysoIP proteomic analyses. Whereas the signaling details activating PKR during lysosomal damage remain to be defined, the inventors nevertheless conclude that PKR, an upstream kinase regulating eIF2α and SG formation, associates with lysosomes and that it is important in sensing lysosomal damage and transmitting damage-associated signals to the SG formation systems.

Stress Granules Induced by Lysosomal Damage Show Dynamic Interactions with Lysosomes

Do SGs induced by lysosomal damage associate with lysosomes? The inventors addressed this by imaging analysis. By confocal fluorescence microscopy, the majority of G3BP1-positive SGs formed during lysosomal damage were either independent of lysosomes or juxtaposed to lysosomes (FIG. 3SA). By HCM quantification, only 10% of G3BP1⁺ SGs appeared associated with lysosomes (LAMP2) 30 min after exposure to lysosomal damaging agent LLOMe. The inventors then asked the question whether SGs formed in response to lysosomal damage initiate as a nidus on the lysosomal surface. Using live microscopy, the inventors observed that the majority of SGs Were forming in locations independent of lysosomes (FIG. 3SB). Whereas overall, the lysosomes and SGs appeared relatively static, there Were nevertheless three types of dynamic events suggesting changing relationships vis-à-vis each other Supplementary Movies 2-4): (i) lysosomes and SGs remained independent of each other (FIG. 3SC subpanel (i)); (ii) SGs appeared to be associated with lysosomes initially but then separated (FIG. 3SC subpanel (ii)); and (iii) lysosomes and SGs started separately but then associated (FIG. 3SC subpanel (iii)). Thus, albeit some dynamic bi-directional interactions between the profiles corresponding to lysosomes and SGs Were detected, the majority of SGs as morphologically discernible profiles Were separate from lysosomes. However, this does not exclude the possibility that individual protein components (e.g. NUFIP2) normally abundant in SGs are present on lysosomes, as has been shown by PCR-based fluorescence imaging assays for G3BP1 under specific conditions of recovery from starvation (Prentzell et al., 2021).

### NUFIP2 Exits Nucleus and Localizes to Lysosomes Upon Damage

Despite the separation between lysosomes and SGs as morphologically visualized profiles, our MS data with LysoIP indicated that certain protein components of SGs are enriched on damaged lysosomes. A top hit for this was NUFIP2 (FIG. 1A), a widely appreciated component of SGs (Markmiller et al., 2018; Yang et al., 2020; Youn et al., 2018). The inventors observed using confocal microscopy that NUFIP2 before LLOMe treatment was mostly in the nucleus of Huh7 cells, separated from the cytosolic G3BP1 (FIG. 4SA). Upon lysosomal damage, NUFIP2 translocated from the nucleus into the cytosol (FIG. S4A), which was also observed by biochemically analyzing distribution in nuclear vs postnuclear cell lysate preparations (FIG. 4SB). A bioinformatics analysis of NUFIP2's primary structure, using consensus/algorithm (Kosugi et al., 2009) revealed a presence of a candidate nuclear localization signal (NLS) in NUFIP2 (FIG. 4SC). When the inventors deleted the putative NUFIP2 NLS, NUFIP2 appeared absent in the nuclear fraction, i.e. was retained in the cytoplasm (FIG. 4SB). Since NUFIP2 WT was found on purified lysosomes only after lysosomal damage (FIG. 1B), the inventors wondered whether NUFIP2ΔNLS would be by default on lysosomes. However, LysoIP analysis showed that NUFIP2ΔNLS did not partition to lysosomes by default but also required additional signals generated during lysosomal damage to translocate to the lysosomes (FIG. 3A). Thus, NUFIP2 translocates to lysosomes upon damage.

NUFIP2 Contributes to mTOR Inactivation During Lysosomal Damage

Recent studies have indicated that components of SGs, such as G3BP1 associated in earlier proteomic studies with NUFIP2 (Sowa et al., 2009), can reside on lysosomes, and have additional, noncanonical functions outside of the scope of SG formation, including effects on mTOR activity (Prentzell et al., 2021). The inventors tested whether NUFIP2, the top hit in terms of SG components' enrichment on damaged lysosomes (FIG. 1A), played a role in mTOR inactivation during lysosomal damage (Chauhan et al., 2016; Jia et al., 2018). A knockdown of NUFIP2 reduced mTOR desorption from the lysosomes (FIG. 3B), which serves as a visual proxy for mTOR inactivation in response to various inputs including lysosomal damage (Castellano et al., 2017; Jia et al., 2018; Sancak et al., 2010). This was confirmed by testing ULK1 phosphorylation by mTOR at Ser757, which was diminished in cells treated with LLOMe, but less so in cells knocked down for NUFIP2 (FIG. 3C). The inventors generated a CRISPR knockout of NUFIP2 in Huh7 cells (Huh7^{NUFIP2-KO}) (FIG. 4SD). mTOR in Huh7^{NUFIP2-KO} cells resisted inactivation in response to lysosomal damage, quantified by HCM of its desorption from lysosomes (FIG. 3D). This was also reflected in levels of S6K (Thr389) and ULK1 (Ser757) phosphorylation, which resisted reduction in Huh7^{NUFIP2-KO} cells, normally seen upon lysosomal damage (Jia et al., 2018) (FIG. 3E).

### Ragulator Abundance and Activity on Damaged Lysosomes is Controlled by NUFIP2

The inventors have previously shown that mTOR is inactivated during lysosomal damage via Ragulator-RagA/B system by inactivating Ragulator's GEF activity toward RagA/B, which in turn normally keep mTOR active (Jia et al., 2018). Our MS data of purified lysosomes after the damage revealed that four Ragulator components (LAMTOR1, 2, 3 and 5) Were elevated (FIGS. 1A and 4A), which was confirmed for LAMTOR1 by LysoIP Western blot analysis (FIG. 4B). This increase in LAMTOR1 presence on damaged lysosomes depended on NUFIP2 (FIG. 4C). Prior studies (Thedieck et al., 2013; Wippich et al., 2013) have shown that mTORC1 component Raptor is sequestered to SGs during canonical stress conditions (arsenite) to regulate mTOR signaling. Our proteomic data (FIG. 1A) with changes in signaling components regulating mTORC1 on lysosomes upon damage indicate reduced Raptor on lysosomes, which is compatible with its reported sequestration in SGs (Thedieck et al., 2013; Wippich et al., 2013). In contrast, the components of the Ragulator complex increased on damaged lysosomes (FIG. 4A).

Activation state of the Ragulator can be assessed by increased interactions between LAMTOR2 (p14) and RagA when RagA is in its inactive, GDP-bound form (Bar-Peled et al., 2012; Castellano et al., 2017; Jia et al., 2018). Using this established approach, the inventors quantified complexes between RagA and LAMTOR2, and found them to be increased (reflecting inactive RagA state) during lysosomal damage, in keeping with our prior studies (Jia et al., 2018), but this was reduced in HEK293T cells stably expressing FLAG-LAMTOR2 knocked down for NUFIP2 (FIG. 4D), indicating that NUFIP2 is required for inactivation of the Ragulator complex. Conversely, overexpression of NUFIP2 further increased the elevated association between FLAG-LAMTOR2 and endogenous RagA during lysosomal damage (FIG. 4E). Thus, NUFIP2 is required for RagA inactivation based on an established assay (Bar-Peled et al., 2012; Castellano et al., 2017; Jia et al., 2018). Furthermore, NUFIP2 was in complexes with LAMTOR1, but only under lysosomal damaging conditions (FIG. 4F). The inventors interpret the observed increase in components of the Ragulator complex on damaged lysosomes in the face of diminished levels of the mTORC1 complex, mTOR, Raptor, and mLST8, as contributing to or compensating for mTOR inactivation, with the latter possibly preconditioning the cells to return to normal mTOR activation state. The inventors conclude that NUFIP2, a functional component of SGs (Markmiller et al., 2018; Yang et al., 2020; Youn et al., 2018) (FIG. 3C) is also an important regulator of mTOR via Ragulator during lysosomal damage.

The observation that NUFIP2 is a new regulator of mTOR prompted us to test the previously reported specific regulators of mTOR inactivation during lysosomal damage (Jia et al., 2018). As previously shown, TSC2 was not necessary to inhibit mTOR, judged by similar inhibition of S6K (Thr389) and ULK1 (Ser757) phosphorylation (FIG. 4SE), whereas RagB was, since cells stably expressing constitutively active RagB^{Q99L} (Castellano et al., 2017; Jia et al., 2018; Sancak et al., 2008) resisted loss of mTOR activity induced by LLOMe (FIG. 4SF), as previously reported for GPN damage (Jia et al., 2018). The inventors next tested LGALS8 (Galectin-8; Gal8), the principal sensor transducing lysosomal damage to inhibit the Ragulator-RagA/B system (Jia et al., 2018). Whereas Gal8 was, as expected, needed to fully inhibit mTOR based on its retention on lysosomes in Gal8KO^{HeLa} cells exposed to LLOMe (FIG. 4SG). Gal8 had no effect on SG formation. G3BP1 puncta formed as robustly in Gal8KO^{HeLa} cells as in parental WT cells treated with LLOMe (FIG. 4SH). Thus, whereas both Gal8 and NUFIP2 contribute to mTOR inactivation following lysosomal damage, only NUFIP2 participates in SG formation. These observations reveal convergence of NUFIP2 and Gal8 upon mTOR inactivation, and divergence in their roles (NUFIP2) or absence of roles (Gal8) in SG formation in response to lysosomal damage.

### Mammalian Atg8s Participate in Recruitment of NUFIP2 to Damaged Lysosomes

In a previous report (Markmiller et al., 2018) with arsenite induced SGs, proximity labeling of mAtg8 proteins was reported when using G3BP1-APEX2. Thus, The inventors considered the possibility that mAtg8s, usually considered to function primarily in the process of clearance of damaged lysosomes in a process termed lysophagy (Maejima et al., 2013), could play an additional role in recruitment of NUFIP2 to damaged lysosomes. Our MS analysis revealed enrichment of GABARAP, GABARAPL2 and LC3B on damaged lysosomes (FIG. 5A). Among other autophagy factors increased on purified lysosomes after a short pulse (30 min) of LLOMe-induced damage Were ATG16L1 and ATG9A (FIG. 5A), whereas other canonical autophagy factors Were not enriched/responsive to lysosomal damage. The inventors next tested whether mAtg8s played a role in NUFIP2 recruitment to damaged lysosomes. For this, the inventors used the previously characterized (Gu et al., 2019b; Kumar et al., 2020; Nguyen et al., 2016) Hexa^{KO} HeLa cells with inactivated six mAtg8s (LC3A, B, C, and GABARAP, L1 and L2), LC3TKO HeLa cells with inactivated three LC3s (LC3A, B and C), and GBRP^{TKO} HeLa cells with inactivated all three GABARAPs (GABARAP, L1 and L2). Hexa^{KO} HeLa cells lost the ability to recruit NUFIP2 to damaged lysosomes, as determined by Western blot analysis of lysosomes purified by LysoIP (FIG. 5B). The GABARAP subset of mAtg8s was responsible for the recruitment of NUFIP2, since LC3TKO HeLa retained the ability to recruit NUFIP2 whereas GBRP^{TKO} HeLa cells did not (FIG. 5B). GABARAPs Were also key for departure of mTOR from damaged lysosomes (FIG. 5B; lanes 5 and 6). LAMTOR1 (p18) inversely mirrored mTOR by being enriched on damaged lysosomes (FIG. 5B; lanes 1 and 2). LAMTOR1 enrichment on damaged lysosomes was lost and GBRP^{TKO} HeLa (FIG. 5B; lanes 5 and 6) but not in LC3^{TKO} HeLa (FIG. 5B; lanes 3 and 4). Thus, mAtg8s, specifically GABARAPs, do not only function in autophagy, but have noncanonical roles in recruitment of NUFIP2 to the lysosome upon damage.

GABARAPs Interact Directly with NUFIP2 and G3BP1

Because GABARAPs were required for NUFIP2, and furthermore for G3BP1 association with damaged lysosomes (FIG. 5B), the inventors tested the possibility that they interact. In GST pulldowns between NUFIP2 and a full panel of mAtg8s and in parallel with G3BP1, GABARAP showed strong association with either of the proteins (FIGS. 5C-F). Some appreciable association was also observed with GABARAPL1 (FIGS. 5C-F). Deletion mapping of GABARAP required for interactions with NUFIP2 indicated that two epitopes, one N-terminally located and another one more centrally located (FIGS. 5G and S5A), suggesting that the binding site is not a single linear epitope, such as the previously reported LIR docking site (LDS) or UIM-docking site (UDS) sites (Behrends et al., 2010; Johansen and Lamark, 2020; Marshall et al., 2019). Nevertheless, the inventors tested single LDS (Y49A), UDS (F77A) and double LDS/UDS mutant GABARAP for binding to NUFIP2, and none of the mutations in these key residues defining LDS or UDS affected association with NUFIP2 in GST pulldowns (FIG. 5SB).

G3BP1 has recently been reported to associate with lysosomes (Prentzell et al., 2021) and as seen in our LysoIP preparations (FIG. 5B). G3BP1 association with damaged lysosomes also depended on GABARAPs (FIG. 5B). The inventors thus tested whether G3BP1 can associate with mAtg8s and found in GST pulldown assays that it interacted directly with GABARAP (FIGS. 5E, F). Furthermore, deletion mapping confirmed that the N-terminal region of GABARAP interacts with G3BP1 (FIGS. 5H, S5C and S5D). Individual or combined LDS and UDS mutants of GABARAP still bound G3BP1 (FIG. 5SB). Finally, NUFIP2 and G3BP1 directly (FIG. 5SE) and very strongly (with 60% of the input [³⁵S] Myc-G3BP1 being bound to its NUFIP2 partner (FIG. 5SF)) interacted with each other in GST pulldown experiments. This interaction was also observed in reverse pulldown experiments (FIG. 5SG). G3BP1 and NUFIP2 constitutively (independently of lysosomal damage) interacted in co-IP experiments (FIG. 5SH).

GABARAPs Participate in mTOR Inactivation but not in eIF2α Phosphorylation in Response to Lysosomal Damage

Based on strong associations in functional and binding experiments between GABARAPs and NUFIP2 and the requirement for NUFIP2 in mTOR inactivation during lysosomal damage, the inventors tested whether NUFIP2's interactors GABARAPs also played a role in mTOR inactivation. Using S6K phosphorylation as a conventional measure of mTOR activity, the inventors detected an expected drop in pT389 S6K levels upon lysosomal damage with LLOMe, a relationship that was preserved in LC3^{TKO} HeLa cells (FIGS. 5I, J). In contrast mTOR inactivation was not observed in Hexa^{KO} and GBRP^{TKO} HeLa cells (FIGS. 5I, J). The inventors next tested by HCM whether mTOR desorption from damaged lysosomes was affected by mAtg8s. As with reduction in S6K phosphorylation, mTOR association with lysosomes diminished upon lysosomal damage in WT and LC3^{TKO} He La cells but not as readily in Hexa^{KO} and GBRP^{TKO} HeLa cells (FIGS. 5L and 5SI).

The inventors then wondered whether eIF2α phosphorylation, which is a marker of canonical SG formation (Ivanov et al., 2019; Riggs et al., 2020) and is strongly induced by lysosomal damage (FIGS. 1G, H), might also be affected by GABARAPs. However, contrary to our expectations, the mAtg8s subgroup or mAtg8s as a whole Were not affecting eIF2α response to lysosomal damage (FIGS. 5I, K). This suggests a separation of functions of GABARAPs in control of mTOR inactivation vs. SG formation response. In keeping with this interpretation, SG formation in response to lysosomal damage was only increased in Hexa^{KO} and GBRP^{TKO} cells (FIGS. 5M and S5J). The inventors interpret these findings as evidence of separation between the function of mAtg8s (specifically GABARAPs) in mTOR inactivation and SG levels, and that these are most likely two competing processes.

The inventors tested which of the GABARAPs Were responsible for both mTOR inactivation and competition with SG formation. For this, the inventors introduced into GBRP^{TKO} HeLa cells individual GFP fusions with GABARAP, GABARAPL1 or GABARAPL2. All three GABARAPs individually Were capable of complementing the mTOR inactivation phenotype, quantified by HCM of mTOR dissociation from the lysosomes upon damage (FIG. 6SA). All three GABARAPs individually Were also able to suppress increased SG formation in GBRP^{TKO} HeLa cells subjected to lysosomal damage (FIG. 6SB). In conclusion, GABARAPs control mTOR inactivation and independently affect SG levels by redistributing NUFIP2 to act upon Ragulator and mTOR on damaged lysosomes (FIG. 6SC).

Atg8ylation Plays a Role in mTOR Inhibition and Competes with Stress Granule Formation During Lysosomal Damage

Recently, the concept of Atg8ylation has been introduced as a general membrane stress and remodeling response and a unifying mechanism for various roles of mAtg8 lipidation in diverse processes beyond their conventional association with canonical autophagy (Kumar et al., 2021b). Thus, the inventors tested whether Atg8ylation plays a role in mTOR inactivation by mAtg8s, specifically GABARAPs in conjunction with their binding partner NUFIP2. GABARAP lipidation has been reported in lysosomal preparations (LysoIP) upon damage (Eapen et al., 2021). GABARAP was lipidated during LLOMe treatment (FIG. 6A). The inventors next quantified by HCM the effects of various ATG mutants that participate either in mAtg8 lipidation, which affects many noncanonical processes as Well as canonical autophagy (Galluzzi and Green, 2019; Kumar et al., 2021b), or in ATG factors specializing in canonical autophagy (Galluzzi and Green, 2019; Levine and Kroemer, 2019; Morishita and Mizushima, 2019). In pairwise comparisons with the Atg8ylation (mAtg8 lipidation) mutant ATG3^{KO} in Huh7 cells, ATG9A (Claude-Taupin et al., 2021) a canonical autophagy gene did not affect either mTOR inactivation or SG formation in response to lysosomal damage, whereas ATG3^{KO} (FIG. 6SD) countered mTOR inactivation (quantified by HCM of mTOR desorption form lysosomes) and enhanced SG formation (FIGS. 6B, C). In another pairwise comparison between the Atg8ylation mutant ATG16L1^{KO} and a canonical autophagy factor FIP200^{KO} (FIG. 6SE), ATG16L1^{KO} reduced mTOR inactivation and enhanced SG formation, whereas FIP200^{KO} did not (FIGS. 6D, E). In the last comparison employed between ATG3^{KO} (Atg8ylation mutant) (Kumar et al., 2020) and ATG13^{KO} (canonical autophagy mutant) in HeLa cells (FIG. 6SF), the above relationships held up, i.e. ATG3^{KO} decreased mTOR inactivation and enhanced SG formation in response to lysosomal damage, whereas ATG13^{KO} did not (FIGS. 6F, G). In conclusion, Atg8ylation is important for mTOR inactivation during lysosomal damage and it antagonizes SG formation in response to the same stimulus, and competes for factors such as NUFIP2 (FIG. 6H). The competition model is consistent with the absence of mAtg8s' effects on eIF2α phosphorylation during lysosomal damage.

### Diverse Pathological Agents Induce Lysosomal Damage and Stress Granule Formation Response

The inventors tested whether the above molecular and cell biological processes associated with lysosomal damage are observed in cell affected by agents causing or modeling pathology and disease. There are reports of bacteria or their products, such as invasive Shigella flexneri or Escherichia coli subtilase cytotoxin as inhibiting or inducing SG formation (Tsutsuki et al., 2016; Vonaesch et al., 2016; Vonaesch et al., 2017). Mycobacterium tuberculosis can permeabilize intracellular vacuoles in which it resides and affect the endolysosomal system in infected macrophages (Manzanillo et al., 2012; Sturgill-Koszycki et al., 1996) and cause lysosomal damage (Chauhan et al., 2016). Hence, the inventors wondered whether virulent M. tuberculosis Erdman with its membrane penetrating (Manzanillo et al., 2012) and lysosomal damage (Chauhan et al., 2016) capabilities can induce SGs upon infection of host cells. Murine bone marrow derived macrophages (BMMs) were infected with M. tuberculosis Erdman wild type and SG formation was quantified after 20 h of infection (FIG. 7A). As a positive control for endomembrane damage the inventors monitored the ubiquitin response (Chauhan et al., 2016; Jia et al., 2020a; Yoshida et al., 2017), which paralleled that of SG formation (FIG. 7A). In contrast, when BMMs Were infected with M. tuberculosis Erdman mutant in ESX-1, a factor required for permeabilization of endomembranes by M. tuberculosis (Manzanillo et al., 2012), both SG formation and ubiquitin puncta formation response where diminished (FIG. 7A). The inventors further modeled events associated with phagocytosis of membrane permeabilizing bacteria, such as M. tuberculosis, using FuGENE HD-coated latex beads (with FuGENE HD used as a membrane damaging agent), and observed similar SG formation and ubiquitin puncta responses in U2OS cells (FIG. 7B).

The inventors and others have reported that protopathic tau induces lysosomal damage (Jia et al., 2020b; Papadopoulos et al., 2017). Treatment of U2OS cells with protopathic tau (Jia et al., 2020b) induced both SG formation response and galectin 3 puncta formation (FIG. 7C), the latter being used as a lysosome damage marker (Aits et al., 2015; Maejima et al., 2013) (Jia et al., 2020b).

Lastly, The inventors tested a factor encoded by SARS-CoV-2, ORF3a. ORF3a from SARS-COV is known to cause lysosomal damage (Yue et al., 2018) and evidence for reduced acidification of lysosomes with SARS-CoV-2^{ORF3a} has been presented (Ghosh et al., 2020). The inventors generated a stable HeLa cell line with tetracycline controllable with FLIP-IN GFP-SARSC-CoV-2^{ORF3a} (FIG. 6SG). Upon induction of SARSC-CoV-2^{ORF3a} expression with tetracycline the inventors observed SG formation and ubiquitin puncta response, a well-established marker of lysosomal damage (Jia et al., 2020a; Koerver et al., 2019; Maejima et al., 2013; Papadopoulos et al., 2017; Yoshida et al., 2017), consistent with ORF3a causing lysosomal damage and that this in turn induces SG formation (FIG. 7D). Thus, the relationships presented in this work are of relevance for multiple pathogenic insults of significance for major human diseases.

The inventors carried out ORF3a interactome analysis by constructing HEK293T Flp-In^{TetON} GFP-SARS-CoV-2^{ORF3a} cells and carrying out DIA proteomic analysis with immunopurified GFP-ORF3a. The HOPS component VPS39 was observed as one of the enriched ORF3a interactors in our proteomic study. VPS39 has been reported in global SARS-CoV-2 interactome studies by others (Gordon et al., 2020; Stukalov et al., 2021). HOPS control endosomal organelle maturation and trafficking en route to lysosomes, and thus may potentially effect lysosomal function, compartment integrity and its susceptibility to stress (Pols et al., 2013; Solinger and Spang, 2013). The effects of ORF3a on HOPS have also been validated in the context of lysosomal function within the autophagy pathway (Miao et al., 2021). STRING functional association protein networks analyses indicated 64 and 85 entries assigned to lysosomal membranes and lysosomes. Among most abundant proteins found in our DIA proteomic analysis was GCN1, an upstream regulator of GCN2-eIF2α-ATF4 axis during repression of global protein synthesis (Pochopien et al., 2021). The inventors validated SARS-CoV2^{ORF3a} interaction with GCN1 in Co-IPs (FIGS. S6H and S6I). These analyses indicate that ORF3a imposes a concerted role on lysosomal function and host cell translational apparatus, which is reflected in the known effects of coronaviruses (Nakagawa et al., 2018; Raaben et al., 2007) and SARS-CoV-2 (Gordon et al., 2020; Perdikari et al., 2020) on stress granule formation in host cells.

### Discussion Examples (First Set)

In this study the inventors report that lysosomal damage induces SG formation as a part of cellular homeostatic responses to stressors that require adjustments in cellular biosynthetic, anabolic and catabolic processes until organellar functionality is restored. SG formation complements the reported mTOR inactivation during lysosomal damage (Eapen et al., 2021; Goodwin et al., 2021c; Jia et al., 2018; Koerver et al., 2019). Together, SG formation and mTOR inhibition cover two key aspects of global adjustments to protein translation during stress (Costa-Mattioli and Walter, 2020; Lu et al., 2004). An unanticipated role for mAtg8s, specifically the GABARAP subset, includes their dual action in balancing SG formation and mTOR inactivation, which at first may appear as a competition between the two processes but instead likely provides proper tuning of the two systems participating in protein translation arrest. This may intersect with selective translation of stress protective elements during integrated stress response (ISR) (Costa-Mattioli and Walter, 2020; Lu et al., 2004).

The inventors found that mTOR inactivation and SG formation are coupled via GABARAPs. This coupling depends on Atg8 lipidation machinery and is a manifestation of the process referred to as Atg8ylation elicited by lysosomal membrane stress (Kumar et al., 2021b). This is a new phenomenon, increasing the breath of diverse processes affected by Atg8ylation (Kumar et al., 2021b), which include control of TFEB via phosphatases (Kumar et al., 2020; Nakamura et al., 2020), selective microautophagy (Lee et al., 2020a), micro-ER-phagy (Loi et al., 2019), effects on exosomes and extracellular vesicles (Guo et al., 2017a; Leidal et al., 2020a), viral budding (Beale et al., 2014), plasma membrane protection via blebbing (Tan et al., 2018), LC3-associated micropinocytosis (Sønder et al., 2021), and other processes collectively referred to as noncanonical autophagy: LAP (Galluzzi and Green, 2019; Sanjuan et al., 2007), LANDO (Heckmann et al., 2019), CASM/SMAC (Goodwin et al., 2021a; Goodwin et al., 2021b). Of note, Atg8ylation has also been used as a term for the recently described protein Atg8ylation (Agrotis et al., 2019; Carosi et al., 2021; Nguyen et al., 2021), expanding the spectrum of potential targets beyond direct membrane Atg8ylation.

The key elements in coupling SG formation and mTOR inactivation via Atg8ylation are NUFIP2 and G3BP1, which interact with GABARAPs as shown here. Both NUFIP2, tested here, and G3BP1 reported elsewhere play a role in inhibiting mTOR under different conditions and act through complementary mechanisms: NUFIP2 acts through Ragulator-Rag complex as shown here, to inactivate mTOR in response to lysosomal damage, whereas G3BP1 works via TSC2-Rheb to modulate mTOR reactivation when cells are re-fed with amino acids and insulin signaling is restored (Prentzell et al., 2021). In our study, the conventional starvation conditions known to inhibit mTOR did not induce SG in keeping with a previous report (Prentzell et al., 2021). Several studies have shown that mTORC1 components are also sequestered to SGs during canonical SG-inducing conditions (heat shock or oxidative stress) (Takahara and Maeda, 2012; Thedieck et al., 2013; Wippich et al., 2013) or noncanonical SG-inducing condition (osmotic stress) (Aulas et al., 2017; van Leeuwen and Rabouille, 2019) (Wippich et al., 2013). In each of these cases, as in our study with lysosomal damage, there is suppression of mTORC1 activity under a variety of conditions. There are also reports of activation of the mTORC1 effector S6K1 upon mild arsenite treatment and that this in turn phosphorylates eIF2α and promotes SG formation (Sfakianos et al., 2018). The effects of Atg8ylation and GABARAPs transmitted to mTOR on lysosomes via NUFIP2 may complement additional mechanisms (Takahara and Maeda, 2012; Thedieck et al., 2013; Wippich et al., 2013) of mTOR inhibition by SGs, and that some of NUFIP2 effects could be explained by indirect effects on SG formation. However, NUFIP2 is not essential for SG assembly (Yang et al., 2020).

G3BP1 is an essential component acting redundantly with G3BP2 in SG formation, whereas NUFIP2 has been reported as non-essential for SG formation in response to the classical inducer arsenite (Yang et al., 2020). Of significance, G3BP1 and NUFIP2 directly interact as inferred by APEX2-G3BP1 proximity biotinylation proteomic studies (Markmiller et al., 2018) and established here in GST-pulldowns. Both proteins bind to GABARAPs, which may be essential for their function in mTOR inactivation but based on increase in the SG cellular content in GBRP^{TKO}, GABARAPs probably do not contribute to SG formation albeit all three GABARAPs have been observed in the proteomic analyses of SGs (Markmiller et al., 2018).

The positive role of GABARAPs and Atg8ylation in mTOR inactivation and their negative role in SG formation seem to represent a competition between these two processes for a limited supply of GABARAPs as a shared component. However, it is likely that this ensures coupling of the two processes contributing to selective shutdown of translation (McCormick and Khaperskyy, 2017). This may reflect the physiological need to balance the two key processes, one working though mTOR-4EBP and the other through PKR-eIF2α, both contributing to the common purpose of shutting down the cap-dependent translation while favoring selective translation of stress response systems. Considering the canonical (McCormick and Khaperskyy, 2017) and noncanonical (Emara et al., 2012; Fujimura et al., 2012) types of SG responses, they may fit in the continuum of how cells balance mTOR inactivation with the eIF2α phosphorylation. Of note, osmotic stress induces noncanonical SG formation without eIF2α phosphorylation (Aulas et al., 2017; van Leeuwen and Rabouille, 2019), and thus differs from the SG-induction by lysosomal damage.

PKR activation is classically associated with viral infections (McCormick and Khaperskyy, 2017; Srivastava et al., 1998) although other stress inputs have been linked to PKR (Gal-Ben-Ari et al., 2019; Nakamura et al., 2010; Williams, 1999). PKR has recently been reported to positively affect lysosomal function by an unknown mechanism (Pataer et al., 2020), which is in keeping with our finding that at least a fraction of PKR is located at the lysosome. In our study, PKR was the key upstream kinase affecting eIF2α phosphorylation in response to lysosomal damage.

The lysosomal damage as a signal for SG formation may be of relevance for multiple disease states. SGs play roles in cancer supporting cancer cell fitness (Choi et al., 2019; Grabocka and Bar-Sagi, 2016; Somasekharan et al., 2015). The tumor microenvironment challenges cancer cells by hypoxia, nutrient starvation, ER stress, osmotic stress and chemotherapy drugs, which promotes SGs formation in cancer cells (Anderson et al., 2015), whereas lysosomal damaging agents have been considered as cancer therapeutics (Bonam et al., 2019; Fehrenbacher and Jäättelä, 2005; Qiu and Xia, 2020). The use of lysosomal damaging drugs, as modeled by LLOMe treatment here, may have to be balanced against cytoprotective responses afforded to cancer cells by SGs.

SG responses have been linked to neurodegenerative diseases (Ash et al., 2014; Liu-Yesucevitz et al., 2010; Taylor et al., 2016; Vanderweyde et al., 2012). Pathological protein aggregates in Alzheimer's disease contain core SG proteins, such as TIA1/R and G3BP1 (Ash et al., 2014). The inventors and others have reported that protopathic tau causes lysosomal damage (Jia et al., 2020b; Papadopoulos et al., 2017). Independent studies have reported that tau can induce SGs. However, the mechanism of how tau promotes SG formation has remained unclear until now. The observation reported here that lysosomal damage caused by tau is accompanied by SG formation provides the missing link in these phenomena.

As shown in our study, SARS-CoV-2^{ORF3a} induces SGs along with the lysosomal damage. SGs are of significance in viral infections (Lindquist et al., 2010; Panas et al., 2012) acting as cellular defense against viruses (McCormick and Khaperskyy, 2017). Viral infections can activate PKR which phosphorylates eIF2a (Srivastava et al., 1998) and trigger SG formation to inhibit viral translation (Balachandran et al., 2000; Williams, 2001). Many viruses suppress SG formation by various defense mechanisms, such as cleavage of G3BP1, the key SG protein, through a viral proteinase or inactivation of PKR (Khaperskyy et al., 2012; White et al., 2007). SARS-CoV-2 Nucleocapsid protein prevents SGs formation and interferes with host gene expression to promote viral production (Luo et al., 2021; Nabeel-Shah et al., 2020; Savastano et al., 2020). These powerful viral mechanisms may counter the effects of ORF3a when expressed in isolation.

Bacterial infections are less frequently linked to SGs, and studies have reported opposing effects. Shigella flexneri infection prevents SGs assembly in response to exogenous stresses (Vonaesch et al., 2016; Vonaesch et al., 2017). Subtilase cytotoxin produced by STEC Shiga toxigenic Escherichia coli strains can induce SG formation in various cells through ER stress and a PERK-eIF2α dependent pathway (Tsutsuki et al., 2016) whereas enterotoxigenic ETEC E. coli inhibit SG formation tested with arsenite as an inducer (Velasquez et al., 2020). SGs are nevertheless a component of a broader integrated stress response ISR (Costa-Mattioli and Walter, 2020; Lu et al., 2004). Specifically, the ISR component PKR-dependent eIF2α phosphorylation (Lu et al., 2004) has been recently identified as an important aspect (Bhattacharya et al., 2021) of Mycobacterium tuberculosis pathology modeled by necrotic granulomas in C3HeB/FeJ mice (Bhattacharya et al., 2021). C3HeB/FeJ, or derivatives of B6 mice carrying the sst1-suscepible (sst1S) genotype (B6.Sst1S mice) possess the C3HeB/FeJ-derived sst1 locus on a B6 background and develop Well-organized necrotic TB granulomas in the lungs. Thus, our finding that virulent M. tuberculosis induces SG response in infected macrophages is of high relevance in pathogenesis of tuberculosis.

How PKR senses lysosomal perturbations remains to be determined. Among other limitations of this work, which remain to be addressed, is the mechanism of recruitment of GABARAPs to damaged lysosomes. GABARAP is one of mAtg8s undergoing lipidation in response to lysosomal damage as shown here. This may be related to the increased presence observed in our MS of ATG16L1 on damaged lysosomes. ATG16L1 is a key component of the E3 ligase driving mAtg8s' lipidation during membrane Atg8ylation (Kumar et al., 2021b) as Well as the newly described process of protein Atg8ylation (Agrotis et al., 2019; Carosi et al., 2021; Nguyen et al., 2021). ATG16L1 is known to interact with V-ATPase (Xu et al., 2019), however our proteomic data indicate synchronous reduction of V-ATPase subunits on lysosomes during early damage. Alternatively, ATG16L1 can bind to ubiquitin (Fujita et al., 2013) and there is a strong ubiquitylation response associated with lysosomal damage (Jia et al., 2020a; Papadopoulos et al., 2017). Additional work is needed to address the exact mechanism of ATG16L1 increase on damaged lysosomes. Another area for future study is the differentiation between the newly described process of protein Atg8ylation (Agrotis et al., 2019; Carosi et al., 2021; Nguyen et al., 2021) vis-a-vis the membrane Atg8ylation (Kumar et al., 2021b), however that applies broadly to the majority of canonical and noncanonical autophagy-related processes.

In summary, GABARAPs and Atg8ylation balance two important aspects of translational suppression via mTOR and SGs. Atg8ylation and mAtg8s play a hitherto unrecognized function in the fine tuning of translational arrest at the interface with the ISR in cells exposed to sources of lysosomal stress in various disease and physiological conditions.

### Examples (Second Set)

### Summary of Experiments

Lysosomal damage poses a significant threat to cell survival. The inventors' previous work demonstrates that lysosomal damage induces stress granule (SG) formation. However, the importance of SG formation in determining cell fate and the precise mechanisms through which lysosomal damage triggers SG formation remains unclear. The below described experiments show that SG formation is initiated via a novel calcium-dependent pathway and plays a protective role in promoting cell survival in response to lysosomal damage. Mechanistically, the inventors demonstrate that during lysosomal damage, ALIX, a calcium-activated protein, transduces lysosomal damage signals by sensing calcium leakage to induce SG formation by controlling the phosphorylation of eIF2α. ALIX modulates eIF2α phosphorylation by regulating the association between PKR and its activator PACT, with galectin-3 exerting a negative effect on this process. Results also show that this regulatory event for SG formation occurs on damaged lysosomes. Additional experiments also reveal that SG formation plays a crucial role in enhancing cell survival when lysosomal damage is caused by various factors such as SARS-CoV-2 ORF3a, adenovirus infection, malarial pigment (hemozoin), proteopathic tau, and environmental hazards. Collectively, these investigations reveal novel insights into the molecular regulation of SG formation triggered by lysosomal damage and shed light on their implications in various diseases associated with damaged lysosomes and SGs.

### Stress Granule Formation Promotes Cell Survival in Response to Lysosomal Damage.

How does SG formation affect cell fate during lysosomal damage? The inventors utilized SG deficient U2OS cells (human osteosarcoma epithelial cell line) genetically lacking both G3BP1 and G3BP2 (ΔΔG3BP1/2) (Nancy Kedersha et al., 2016), which are essential factors for SG formation (Guillén-Boixet et al., 2020; Nancy Kedersha et al., 2016; P. Yang et al., 2020) (FIG. 7SA). The inventors quantified the number of SGs using the canonical SG marker polyA RNA (Ivanov et al., 2019) via high-content microscopy (HCM) (FIG. S1B) and verified the depletion of SG formation in ΔΔG3BP1/2 cells when exposed to the lysosome-specific damaging agent L-leucyl-L-leucine methyl ester (LLOMe) (Jia et al., 2022; Tan et al., 2022; Thiele et al., 1990) (FIG. 7SB). A propidium iodide (PI) uptake assay measuring plasma membrane integrity (Crowley et al., 2016; Liu et al., 2023) was adapted to quantify cell survival during lysosomal damage using HCM. The inventors found significant cell death upon LLOMe treatment in ΔΔG3BP1/2 cells compared to wildtype (WT) U2OS cells (FIG. 1A). This was additionally confirmed by using a lactate dehydrogenase (LDH) release assay measuring non-specific leak from cells (Chan et al., 2013; Kumar et al., 2018) (FIG. 1B). Further, the inventors pharmacologically blocked SG assembly through the use of cycloheximide which freezes ribosomes on translating mRNAs and reduces the accumulation of free untranslated mRNA (Freibaum et al., 2021; N. Kedersha et al., 2000). Consistent with previous reports (Bussi et al., 2023; Jia et al., 2022), cycloheximide treatment inhibited SG formation in U2OS cells, as evidenced by the absence of G3BP1 puncta following LLOMe treatment (FIG. 7SC). This suppression of SG formation led to reduced cell survival, as indicated by increased LDH release in the face of lysosomal damage (FIG. 7SD). Previously The inventors reported that LLOMe treatment induced phosphorylation of eIF2α (Jia et al., 2022), a critical signal for SG formation (Ivanov et al., 2019; N. Kedersha et al., 2000). The small molecule ISRIB (integrated stress response inhibitor) can also act as an SG inhibitor, effectively counteracting the downstream effects of eIF2α phosphorylation, such as ATF4 (Activating transcription factor 4) expression (Rabouw et al., 2019; Sidrauski et al., 2015). The inventors prevented SG formation using ISRIB upon lysosomal damage (FIG. 7SE) and observed a corresponding reduction in ATF4 expression levels in THP-1 cells (the human monocytic cell line) (FIG. 7SF). The prevention of SG formation by ISRIB also caused a decrease in cell survival in THP-1 cells (FIG. 7SG). The protective effects of SG formation in response to lysosomal damage Were also observed in primary cells using human peripheral blood monocyte-derived macrophages (hMDM). This includes that the significant increase in cell death during LLOMe treatment, as quantified by the PI uptake assay when SG formation was inhibited by cycloheximide in hMDM (FIGS. 1C, D). This was further confirmed by measuring the viability of live hMDM (without the fixation) using an AMNIS imaging flow cytometer (FIG. 1E). Knockdown of both G3BP1 and G3BP2 in hMDM (G3BP1/2DKD) resulted in a reduction of SG formation as evaluated by a key SG marker, eIF4G puncta, during LLOMe treatment (FIGS. 1F (i, ii) and S1H). Elevated cell death, as quantified by PI uptake assay (FIG. 1F (i, iii)) and the LDH release assay (FIG. 1G), was detected in G3BP1/2^{DKD} in response to LLOMe treatment. In summary, SG formation is a cytoprotective response to lysosomal damage (FIG. 1H).

Stress Granule Formation is Controlled by eIF2α Pathway but not mTORC1 Pathway During Lysosomal Damage.

Considering the significance of SG formation during lysosomal damage, what mechanisms regulate SG formation in response to such damage? SG formation occurs as a consequence of protein translation arrest during cellular stress (Riggs et al., 2020; Youn et al., 2019). eIF2α phosphorylation and mTORC1 inactivation are two key upstream events that lead to protein translation arrest and subsequently trigger SG formation (Cotto et al., 1999; Emara et al., 2012; McCormick et al., 2017). Consistent with our earlier studies (Jia et al., 2018; Jia et al., 2022), the inventors confirmed that LLOMe treatment induced eIF2α phosphorylation and mTORC1 inactivation in a dose-dependent manner in U2OS cells (FIG. 8SA). To investigate the role of eIF2α and mTORC1 pathways in regulating SG formation upon lysosomal damage, the inventors initially knocked down eIF2α in U2OS cells (eIF2α^{KD}) (FIG. 2A). This revealed that eIF2α is necessary for SG formation upon lysosomal damage, which was reflected by the depletion of SG formation in eIF2α^{KD} cells during LLOMe treatment (FIG. 2A). In addition, mTORC1 activity in eIF2α^{KD} cells was examined by detecting the phosphorylation of its substrate 4EBP1 (Ser65), revealing that mTORC1 inactivation was not affected by eIF2α depletion upon lysosomal damage (FIG. 2B). This indicates that eIF2α phosphorylation and mTORC1 inactivation are two uncoupled events during lysosomal damage. This was further confirmed by the lack of change in eIF2α phosphorylation upon lysosomal damage in cells expressing constitutively active RagB^{Q99L}, which keeps mTORC1 in an active state (Abu-Remaileh et al., 2017; Sancak et al., 2010) (FIG. 2C). Additionally, SG formation was not affected in cells expressing RagB^{Q99L} in response to lysosomal damage (FIG. 2D). This uncoupled relationship between eIF2α phosphorylation and mTORC1 inactivation in SG formation is also reflected in various cellular stress conditions, including amino acid starvation and arsenic stress. The inventors found that amino acid starvation resulted in mTORC1 inactivation (assessed by mTOR dissociation from the lysosomes (Abu-Remaileh et al., 2017; Jia et al., 2022) but not eIF2α phosphorylation or SG formation as in previous reports (Prentzell et al., 2021; X. Wang et al., 2008) (FIGS. S2B, C). In contrast, arsenic stress led to eIF2α phosphorylation and SG formation while activating mTORC1 activity, consistent with earlier studies (Q. Y. Chen et al., 2018; Prentzell et al., 2021; Thedieck et al., 2013) (FIGS. S2B, C). The key role of eIF2α phosphorylation in SG formation during lysosomal damage was further demonstrated by the ability to complement eIF2α WT but not its phosphorylation site mutant (eIF2α S51A) (N. L. Kedersha et al., 1999) in eIF2φ^{KD} cells to restore SG formation (FIG. 2E). In summary, eIF2α phosphorylation is a major upstream event for SG formation in response to lysosomal damage (FIG. 2F).

Proteomics Proximity Analysis of eIF2α Upon Lysosomal Damage Reveals that its Phosphorylation is Driven by PKR and PACT.

To further investigate the mechanisms that trigger eIF2α phosphorylation in response to lysosomal damage, a dynamic proteomic analysis using proximity biotinylation was conducted. The inventors identified and compared the interacting partners of eIF2α through LC/MS/MS, utilizing APEX2-eIF2α fusion, under both control and lysosomal damage (LLOMe) conditions (for a total of three independent experiments). The volcano plot of this proteomic analysis showed dynamic changes in the proximity of cellular proteins to APEX2-eIF2α during lysosomal damage (FIG. 3A). Within the top twenty candidates showing increased association with eIF2α in response to lysosomal damage, the inventors found the expected candidate PKR (EIF2AK2), which was previously reported by our group as a potential upstream kinase responsible for eIF2α phosphorylation during lysosomal damage (Jia et al., 2022) (FIG. 3A). Interestingly, the activator of PKR, PACT (PRKRA) (Patel et al., 1998), prominently emerged with the most significant fold increase following lysosomal damage (FIG. 3A). PACT is known to facilitate the stress-induced phosphorylation and activation of PKR through direct interaction (Patel et al., 1998; Singh et al., 2012). This interaction disrupts PKR's self-inhibition, leading to PKR autophosphorylation including at Thr446, which converts it into its fully active form capable of phosphorylating protein substrates, such as eIF2α (Chukwurah et al., 2021; Sadler et al., 2007). The inventors confirmed increased interactions of PKR and PACT with eIF2α upon lysosomal damage by co-immunoprecipitation (co-IP) between FLAG-eIF2α and endogenous PKR and PACT (FIG. 3B). Next, the inventors examined whether PKR and PACT are functionally necessary for eIF2α phosphorylation triggered by lysosomal damage. Previously, the inventors knocked down four widely recognized upstream kinases of eIF2α (HRI, PKR, PERK, and GCN2) (Pakos-Zebrucka et al., 2016), and found that only the knockdown of PKR resulted in the inhibition of eIF2α phosphorylation and SG formation (Jia et al., 2022). To confirm these findings, the inventors generated a CRISPR knockout of PKR (PKR^{KO}) in SG reporter cells (U2OS G3BP1-GFP). In these PKR^{KO} cells, the formation of SG induced by lysosomal damage was completely inhibited, as quantified by the puncta of G3BP1-GFP using HCM (FIG. 3C). In line with this, the phosphorylation of eIF2α and PKR was also abolished (FIG. 3D). Conversely, the overexpression of PKR in PKR^{KO} cells led to a restoration of phosphorylation of eIF2α and PKR during lysosomal damage (FIG. 3D). Recently, MARK2 was identified as the fifth kinase responsible for eIF2α phosphorylation in response to proteotoxic stress (Lu et al., 2021). However, the inventors found that MARK2 did not regulate eIF2α phosphorylation during lysosomal damage (FIG. 9SA). Next, PKR's activator, PACT, was examined by knocking it down in U2OS cells (PACT^{KD}). The inventors observed a decrease in PKR activation, eIF2a phosphorylation, and SG formation observed in PACT^{KD} cells during lysosomal damage (FIG. 3E). This finding aligns with the role of PKR in controlling eIF2α phosphorylation and SG formation. Thus, both PKR and its activator PACT regulate eIF2α phosphorylation for SG formation during lysosomal damage.

PKR and PACT Control eIF2α Phosphorylation on Damaged Lysosomes.

The inventors previously performed proteomic analyses of lysosomes that Were purified using LysoIP (Jia et al., 2022), a Well-established approach to isolate lysosomes by the lysosomal membrane protein TMEM192 (Abu-Remaileh et al., 2017; Jia et al., 2020c). These analyses indicate the presence of PKR, PACT, and eIF2α on lysosomes (FIG. 9SB). This finding is further supported by similar results from LysoIP proteomic analysis conducted by other research groups (Eapen et al., 2021; Wyant et al., 2018) (FIG. 9SB). Using LysoIP immunoblotting, the inventors confirmed the presence of PKR, PACT and eIF2α on lysosomes and found an elevation in their association with damaged lysosomes (FIG. 3F). The inventors also observed that the phosphorylation of both PKR and eIF2α occurred on damaged lysosomes. Notably, this effect was effectively blocked by a specific PKR's inhibitor, imidazolo-oxindole C16, known for its ability to inhibit PKR's autophosphorylation by binding to PKR's ATP-binding pocket (Gal-Ben-Ari et al., 2019; Jammi et al., 2003; Tronel et al., 2014) (FIG. 3F). Moreover, through confocal fluorescence microscopy, an increased association of PKR, PACT, and eIF2α was detected with damaged lysosomes (FIGS. S3C-E). In summary, the inventors conclude that PKR and its activator, PACT, regulate eIF2α phosphorylation on damaged lysosomes (FIG. 3G).

ALIX and ALG2 are Required for Stress Granule Formation by Sensing Calcium Release from Damaged Lysosomes.

In our proteomic analysis of eIF2α binding partners (FIG. 3A), the inventors observed an increased association between eIF2α and ESCRT components such as ALIX, CHMP2B, and CHMP4B following lysosomal damage. Specifically, ALIX showed a greater than 10-fold increase (FIG. 3A). The inventors next determined whether these ESCRT components Were involved in eIF2α phosphorylation and SG formation triggered by lysosomal damage. Upon lysosomal damage, the inventors observed a significant reduction in SG formation upon knockdown of ALIX in U2OS cells (ALIX^{KD}), as quantified by G3BP1 puncta using HCM (FIGS. 4A, C). This was also reflected in the decreased phosphorylation of eIF2α and PKR in ALIX^{KD} cells during LLOMe treatment (FIGS. 4B, D), indicating an impact of ALIX on the upstream signaling of SG formation. However, the knockdown of CHM2B or CHMP4B had no discernible effect on SG formation and its upstream events (FIGS. S4A, B). Previous studies showed that the depletion of both ALIX and TSG101 effectively impedes lysosomal repair by eliminating ESCRT recruitment (Niekamp et al., 2022; Radulovic et al., 2018; Skowyra et al., 2018). The inventors found that TSG101 has no effect on the regulation of SG formation upon lysosomal damage. This is supported by the absence of any significant changes in SG formation and eIF2α phosphorylation in TSG101 knockdown U2OS cells (TSG101^{KD}) (FIGS. 4C, D). ALIX has been reported to sense lysosomal damage through the detection of calcium leakage, which is facilitated by its calcium binding partner, ALG2 (W. Chen et al., 2024; Jia et al., 2020a; Niekamp et al., 2022; Skowyra et al., 2018). Notably, ALG2 exhibited increased proximity to eIF2α upon lysosomal damage (FIG. 3A). To further determine the regulatory role of ALIX in SG formation upon lysosomal damage, the inventors utilized BAPTA-AM, the calcium chelator and ALG2 knockdown U2OS cells (ALG2^{KD}) to prevent the recruitment of ALIX to damaged lysosomes as previously reported (Jia et al., 2020a; Skowyra et al., 2018). This was confirmed by the observed decrease in ALIX puncta formation upon lysosomal damage in cells treated with BAPTA-AM or in ALG2^{KD} cells (FIG. 10SC). Importantly, The inventors also observed a significant reduction in SG formation and eIF2α phosphorylation in cells treated with BAPTA-AM, or in ALG2^{KD} cells during lysosomal damage (FIGS. 4E, F). Thus, the inventors conclude that ALIX and its partner, ALG2, modulate eIF2α phosphorylation by sensing calcium leakage as lysosomal damage signal, thereby initiating SG formation (FIG. 4G).

ALIX Associates with PKR and PACT in Response to Lysosomal Damage.

Given that eIF2α phosphorylation is initiated by its upstream kinase PKR, and its activator PACT (FIG. 3), our subsequent investigation delved into exploring the relationship among ALIX, PKR and PACT. Using a co-IP assay, the inventors tested the interaction between FLAG-ALIX and endogenous PKR and PACT. Their interactions Were notably enhanced following treatment with LLOMe (FIG. 5A). ALIX is composed of three distinct domains: Bro1 domain, V domain, and proline-rich domain (PRD) (FIG. 5B). These domains have the potential to remain inactive due to intramolecular interactions but can be activated through interaction with ALG2 in a calcium-dependent manner (Maki et al., 2016; Scheffer et al., 2014; Sun et al., 2015; Vietri et al., 2020) (FIG. 5B). Next, the inventors generated the domain deletions of ALIX (FIG. 5B (i)). The mapping analysis of ALIX domains necessary for binding to PKR and PACT revealed the indispensable role of the V domain in their interaction (FIG. 5C). Additionally, increased associations among full-length ALIX, PKR and PACT were observed upon LLOMe treatment (FIG. 5C), suggesting that lysosomal damage activates ALIX by releasing its V domain for association with PKR and PACT. This is corroborated by the interaction of the V domain of ALIX with PKR and PACT, even in cells that Were not subjected to lysosome damage induced by LLOMe (FIG. 5C). The interactions between ALIX and PKR, as well as ALIX and PACT, Were also predicted using the protein-protein docking server HDOCK, resulting in scores of 0.8398 for ALIX-PKR and 0.8325 for ALIX-PACT, reflecting a high level of confidence (Yan et al., 2020) (FIGS. S5A, B). Furthermore, through confocal fluorescence microscopy, the inventors observed the association among ALIX, PKR, and PACT during lysosomal damage (FIG. 11SC). Thus, ALIX interacts with PKR and PACT in response to lysosomal damage.

### ALIX Promotes the Association Between PKR and its Activator PACT on Damaged Lysosomes.

Next, the inventors quantified by HCM the ALIX puncta response to lysosomal damage in cells where PKR or PACT had been knocked down. The inventors observed that the presence or absence of PKR and PACT did not affect ALIX response to lysosomal damage (FIG. 11SD). This suggests that ALIX may potentially precede PKR and PACT for eIF2α phosphorylation upon lysosomal damage. Considering the decrease in the phosphorylation of PKR in ALIX^{KD} cells and the increased association among ALIX, PKR, and PACT following lysosomal damage (FIGS. 4B, D, 5A), the inventors hypothesize that ALIX regulates PKR phosphorylation by modulating the association between PKR and its activator, PACT, during lysosomal damage. Using co-IP assays, the inventors confirmed the formation of complexes between FLAG-PKR and endogenous PACT during lysosomal damage (FIG. 5D). However, this interaction was reduced in ALIX^{KD} HEK293T cells (FIG. 5D), resulting in decreased PKR phosphorylation during LLOMe treatment. Conversely, the overexpression of ALIX led to a further enhancement in the increased association between GFP-PACT and endogenous PKR, and this was accompanied by an increase in PKR phosphorylation during lysosomal damage (FIG. 5E). These data indicates that ALIX is essential for PKR phosphorylation by controlling the interaction between PKR and PACT during lysosomal damage. Next, the inventors examined whether this regulatory event occurred on damaged lysosomes by conducting LysoIP immunoblotting in ALIX^{KD} HEK293T cells. In this assay, the inventors observed that ALIX^{KD} HEK293T cells no longer displayed PKR phosphorylation on damaged lysosomes, accompanied by a reduced recruitment of PKR and PACT to lysosomes, as determined by Western blot analysis of lysosomes isolated using LysoIP (FIG. 5F). This suggests that ALIX is responsible for the recruitment and regulation of PKR and PACT on damaged lysosomes. In summary, the inventors conclude that ALIX recruits PKR and its activator, PACT, to damaged lysosomes and regulates the activation of PKR by enhancing its association with PACT, consequently leading to eIF2α phosphorylation and SG formation (FIG. 5G).

### Galectin-3 Inhibits Stress Granule Formation by Reducing the Association Between PKR and PACT During Lysosomal Damage.

Previously, the inventors reported that galectin-3 (Gal3), a β-galactoside-binding protein that recognizes damage-exposed glycan, can recruit ALIX to damaged lysosomes and promote ESCRT function for lysosomal repair and restoration (Jia et al., 2020a). The inventors examined whether Gal3 is involved in the regulatory process of SG formation during lysosomal damage. In U2OS cells subjected to Gal3 knockdown (Gal3^{KD}), the inventors observed an elevated level of SG formation, quantified by the formation of G3BP1 puncta using HCM (FIG. 6A). This result was consistent with our earlier report showing an increase in SGs in Gal3 knockout HeLa cells (Jia et al., 2022). Here, the inventors further detected the upstream signaling events leading to SG formation in Gal3^{KD} U2OS cells and observed an increase in the phosphorylation of PKR and eIF2α in the absence of Gal3 following LLOMe treatment (FIG. 6B). These data indicate that Gal3 has a negative effect on the activation of PKR and eIF2α, thereby affecting SG formation during lysosomal damage. Next, the relationship among Gal3, PKR, and PACT was tested. The co-IP results showed that Gal3 can be in protein complexes with ALIX, PKR, and PACT upon lysosomal damage (FIG. 6C). When determining if Gal3 can control the association between PKR and PACT, the inventors found an increase in their association in the absence of Gal3 (FIG. 6D). This was further confirmed by the increased PKR phosphorylation under the same conditions. On the contrary, when Gal3 was overexpressed, it led to a reduction in the interaction between PKR and PACT, consequently resulting in reduced PKR phosphorylation upon LLOMe treatment (FIG. 6E). The inventors interpret the inhibitory role of Gal3 in the association between PKR and PACT as a result of their competition for ALIX. Consistent with this interpretation, the inventors observed a reduced interaction among ALIX, PACT, and PKR in Gal3-overexpressing cells during LLOMe treatment (FIG. 6F). However, when the inventors overexpressed the Gal3R186S mutant, which has been previously shown to lose the ability to recognize damaged lysosomes (Aits et al., 2015; Jia et al., 2020a), it failed to regulate the protein complex of ALIX, PACT, and PKR upon lysosomal damage (FIG. 6F). Moreover, given our previous finding that Gal3 facilitates ESCRT-mediated lysosomal repair via ALIX (Jia et al., 2020a), these observations provide evidence of Gal3's role in balancing ALIX-mediated lysosomal repair and ALIX-mediated SG formation (FIG. 6G). Thus, the inventors conclude that the recruitment of Gal3 to damaged lysosomes plays an inhibitory effect on the regulation of the upstream processes of SG formation by decreasing the association between PKR and PACT (FIG. 6G).

### Stress Granule Formation Promotes Cell Survival in Response to Lysosomal Damage in the Context of Disease States.

Lysosomal damage serves as both a cause and consequence of many disease conditions, including infectious and neurodegenerative diseases (Amaral et al., 2023; Ballabio et al., 2020; Bonam et al., 2019; Fehrenbacher et al., 2005). The inventors tested whether the above molecular and cellular processes that transduce lysosomal damage signals to induce SG formation are important for cell survival in disease contexts. Lysosomal damage can occur from viral infections including those caused by non-enveloped adenovirus and enveloped SARS-CoV-2 infections (Aits et al., 2013; Barlan et al., 2011; Daussy et al., 2020; Thurston et al., 2012; Fengjuan Wang et al., 2018). Adenovirus enters cells through endocytosis and damages lysosomes by releasing its protease, which allows access to the cytosol and subsequently the nucleus for replication (A. Barlan et al., 2011; Greber et al., 1996; Pied et al., 2022; Wiethoff et al., 2015). The inventors employed the wildtype human adenovirus species C2 (HAdV-C2^{WT}) and its protease-deficient mutant TS1 (HAdV-C2^{TS1}), the latter lacking the ability to damage lysosomes (Gallardo et al., 2021; Greber et al., 1996; Martinez et al., 2015). U2OS cells Were infected with either HAdV-C2^{WT} or HAdV-C2^{TS1} and the lysosomal damage marker LysoTracker Red (LTR), which measures lysosomal acidification (Chazotte, 2011; Jia et al., 2020a; Pierzyńska-Mach et al., 2014), was quantified by HCM in infected cells. Consistent with earlier findings (Luisoni et al., 2015; Martinez et al., 2015; Pied et al., 2022), HAdV-C2^{WT} led to a reduction in LTR⁺ profiles, whereas HAdV-C2^{TS1} did not show such an effect (FIG. 12SA). Additionally, SG formation and eIF2α phosphorylation Were detected in cells infected with HAdV-C2^{WT} but not in those infected with HAdV-C2^{TS1} (FIGS. 7A, B). These results imply that lysosomal damage triggered by HAdV-C2 infection can activate the eIF2α pathway, resulting in SG formation. The inventors then tested whether SG formation is important for cell survival during HAdV-C2 infection. In SG-deficient U2OS (ΔΔG3BP1/2) cells, compared to wildtype U2OS cells, the inventors observed an elevated level of cell death, using a PI uptake assay, during HAdV-C2^{WT} infection (FIG. 7C). In addition, the inventors expanded on our previous investigations showing that lysosomal damage induced by the expression of SARS-CoV-2 ORF3a protein can also trigger SG formation (Jia et al., 2022). Following the overexpression of SARS-CoV-2 ORF3a in U2OS cells, a notable rise in cell death was observed through an LDH release assay in ΔΔG3BP1/2 cells compared to control cells (FIG. 7D). Collectively, SG formation triggered by lysosomal damage emerges as a crucial process for cell survival during for the viral infections examined.

Additionally, other disease-associated agents in the context of human parasitic infections were examined that have the potential to damage lysosomes, such as malarial pigment (hemozoin). This parasitic agent is a crystalline and insoluble byproduct of hemoglobin digestion by Plasmodial species that is phagocytosed by circulating monocytes and neutrophils, and tissue macrophages, thus promoting immunopathological effects in human malaria (Anyona et al., 2022; Coronado et al., 2014; Guerra et al., 2019; Moore et al., 2004; Schwarzer et al., 1992; Weissbuch et al., 2008). Treatment of human monocytic THP-1 with physiological concentrations of hemozoin (0.1, 1.0, and 10.0 mg/mL) for 4 h, dose-dependently induced lysosomal damage, monitored by ALIX puncta formation serving as a lysosomal repair marker (Jia et al., 2020c; Radulovic et al., 2018; Skowyra et al., 2018) (FIG. 12SB). While a previous report showed that hemozoin is rapidly ingested by human monocytes and exclusively localized in normally acidified phagolysosomes (Schwarzer et al., 2001), our findings suggest that hemozoin can perturb lysosomal membranes. Differences in the studies may be due to cell types, dosage, and treatment duration. In addition, stimulation with hemozoin (10.0 mg/mL) for 4 h, resulted in both SG formation and eIF2α phosphorylation (FIGS. S6C, D). Blocking SG formation with cycloheximide in hMDM cells, showed an increased cell death as measured by LDH release assay in response to hemozoin treatment (10.0 mg/mL) (FIG. 7E). Moreover, the inventors examined other lysosomal damaging agents, such as silica crystals associated with silicosis (Hornung et al., 2008; Mossman et al., 1998; J. Wang et al., 2017) and tau aggregates implicated in Alzheimer's disease (Flavin et al., 2017; C. Papadopoulos et al., 2017). The inventors have previously reported that both silica crystals and tau aggregates induce lysosomal damage, leading to SG formation (Jia et al., 2022). This effect was further confirmed by detecting eIF2α phosphorylation in hMDM cells in response to the treatment of silica crystals or tau aggregates (FIG. 12SD). The prevention of SG formation with cycloheximide during the treatment of silica crystals or tau aggregates led to augmented cell death, as assessed using an AMNIS imaging flow cytometer in hMDM cells (FIGS. 7F, G). Similarly, the application of another SG inhibitor, ISRIB to inhibit SG formation triggered by silica crystals or tau aggregate, produced a comparable effect on cell death of hMDM cells, measured by PI uptake assay (FIGS. S6E, F). In summary, our findings suggest that SG formation induced by lysosomal damage is important for cell survival against diverse pathogenic challenges associated with major human diseases.

### Discussion

In this study, the inventors uncovered the regulation and significance of SG formation in response to lysosomal damage, providing insights into the interaction between membrane-bound organelles and membrane-less condensates. Through unbiased approaches, including proteomic analysis and high content microscopy, the inventors defined a novel signaling pathway that transmits calcium leakage from damaged lysosomes to induce eIF2α phosphorylation, ultimately leading to SG formation, thus promoting cell survival. This study aligns with recent research indicating the role of SGs in plugging damaged membranes and aiding in lysosomal repair (Bussi et al., 2023), underscoring SG formation as a vital cellular protective mechanism against lysosomal damage, essential for survival.

How does the cell detect lysosomal damage to initiate SGs? Our study revealed the significant involvement of a calcium signal in this process. Lysosomes function as key intracellular calcium reservoirs for various cellular activities (Lloyd-Evans et al., 2020; Xu et al., 2015). The inventors found that ALIX and ALG2 sense calcium leakage from damaged lysosomes, leading to the activation of ALIX's role in regulating PKR's activity. This involves control of the association between PKR and its activator, PACT, resulting in the phosphorylation of eIF2α. Notably, the inventors found that the role of ALIX and ALG2 in controlling eIF2α phosphorylation is distinct from their established function in ESCRT-mediated lysosomal repair. This suggests the multifaceted roles of ALIX and ALG2 as calcium sensors in coordinating cellular responses to lysosomal damage. Furthermore, our findings also indicate the intricate and adaptable nature of calcium signaling pathways in coordinating various cellular defense mechanisms against lysosomal damage. This extends beyond their involvement in TFEB nuclear translocation and phosphoinositide-mediated rapid lysosomal repair (Medina et al., 2015; Nakamura et al., 2020; Tan et al., 2022).

SGs consist of RNA-binding proteins and untranslated mRNA, both playing a crucial role in the process of phase separation (Millar et al., 2023). In addition to the calcium signal the inventors reported here as a trigger for SG formation during lysosomal damage, a recent study suggests that a decrease in pH can also induce SG formation on damaged lysosomes (Bussi et al., 2023). This is in line with the reported role of pH in G3BP1-driven SG condensation (Guillen-Boixet et al., 2020). However, the latter report indicates that pH may not directly regulate the RNA-binding affinity of G3BP1 but instead influences protein-protein interactions. It is worth noting that these experiments Were conducted in an in vitro system and the presence of mRNA. Therefore, it raises the possibility that multiple mechanisms may collaborate to trigger SG formation by controlling protein-protein interaction or the accumulation of untranslated mRNA in response to lysosomal damage. To understand the signaling mechanism responsible for the accumulation of untranslated mRNA, our study suggests a calcium-dependent pathway that induces untranslated mRNA for SG formation by controlling eIF2α phosphorylation. Thus, both pH and calcium-dependent pathways can collaboratively contribute to SG formation during lysosomal damage. Moreover, considering the central role of lysosomes as the main degradation center for diverse cellular components, such as RNA (Lawrence et al., 2019), and the recognition of lysosomal damage that can be sensed by various cellular mechanisms (Aits et al., 2015; Jia et al., 2022; Napolitano et al., 2016; Chrisovalantis Papadopoulos et al., 2017), the leakage of certain lysosomal contents or the activation of other lysosomal damage sensors may also contribute to the activation of PKR, eIF2α phosphorylation, or the regulation of SG formation.

Phosphorylation of eIF2α is a key event in SG formation as it causes the shutdown in global translation and the accumulation of untranslated mRNA, which triggers the phase separation, ultimately leading to SG formation (Ivanov et al., 2019; Riggs et al., 2020). However, there are instances of SG formation that occur independently of eIF2α phosphorylation, potentially regulated by translational shutdown through the mTORC1 pathway (Emara et al., 2012; Fujimura et al., 2012). Nevertheless, this does not appear to be the case for SG formation in response to lysosomal damage. Our data indicate that upon lysosomal damage, eIF2α phosphorylation is the primary driver for SG formation, though the impact of mTORC1 inactivation on translation shutdown and SG formation cannot be entirely ruled out. Importantly, the uncoupled relationship between mTORC1 inactivation and eIF2α phosphorylation in SG formation may be attributed to their differential impacts on protein translation events and mRNA entry into SGs. For example, mTORC1 inactivation primarily inhibits the translation pre-initiation, while eIF2α phosphorylation can impede the recruitment of the large ribosomal subunit to mRNA (Holz et al., 2005; Jackson et al., 2010). Recent research suggests that having just one large ribosomal subunit on mRNA is enough to prevent the recruitment of mRNA into SGs, while extended ribosome-free regions on mRNA are insufficient for SG formation (Fedorovskiy et al., 2023). Thus, mTORC1 inactivation may result in ribosome-free regions on mRNA, but alone, it is insufficient to prompt mRNA entry into SGs. The prevention of large ribosomal subunits on mRNA through eIF2α phosphorylation appears to be a crucial factor triggering this process and contributing to SG formation in the context of lysosomal damage. In addition, through the examination of SG formation in galectin knockout cells, the inventors recently showed (Jia et al., 2022) that galectin-8 does not influence SG formation. This finding supports the premise that eIF2α phosphorylation and mTORC1 inactivation are dissociated events during lysosomal damage, as the inventors have previously reported that galectin-8 can modulate mTORC1 activity under similar conditions (Jia et al., 2018). Furthermore, recent research has highlighted lysosomes as pivotal hubs in metabolic signaling, involving mTORC1 and AMPK pathways (Carroll et al., 2017; Jia et al., 2018; Jia et al., 2020b; Zoncu et al., 2011). Our findings regarding the regulation of eIF2α phosphorylation on damaged lysosomes, along with our earlier findings showing mTORC1 inactivation on damaged lysosomes (Jia et al., 2018), propose an innovative role for lysosomes as central command centers in orchestrating protein translation signaling during stress conditions.

Understanding the function of SGs during stress, especially in the context of lysosomal damage, remains limited. A recent report highlights the reparative role of SGs through their association with damaged lysosomes (Bussi et al., 2023). This finding aligns with our prior research; however, in our study, the inventors observed SGs at a distance from damaged lysosomes (Jia et al., 2022). This observation challenges the notion of SGs primarily serving as plugs and suggests a broader spectrum of roles for SGs in response to lysosomal damage. Given the significance of SG formation in supporting cell survival during lysosomal damage, as reported here, it is highly unlikely that SGs can undertake multiple tasks in restoring cellular homeostasis for survival. For example, considering SGs sequester non-translating mRNA (Khong et al., 2017), they may play roles in protecting mRNA and controlling mRNA fate of the transcriptome during lysosomal damage. Moreover, SG formation intersects with the integrated stress response (ISR), which can optimize the cell response by reprogramming gene expression to promote cellular recovery (Pakos-Zebrucka et al., 2016). The impact of SG formation on ISR may also enhance cellular fitness. Additionally, the involvement of SGs in various cellular processes, e.g., intracellular transport dynamics, ribosome biogenesis, and cell signaling (Gorsheneva et al., 2024; Ripin et al., 2023; M. L. Zhang et al., 2024), may further contribute to cellular survival upon lysosomal damage.

Recognizing lysosomal damage as a critical internal physiological trigger for SGs highlights the importance of enhancing our understanding of SG formation in disease contexts. The inventors detected the role of SG formation in cell survival within disease-specific contexts using a series of pathological reagents to induce lysosomal damage. Given the strong association of these reagents with both lysosomal damage and SG formation, delving into the molecular mechanisms governing the interaction between lysosomal damage and SGs provide valuable insights for therapeutic efforts.

### Discussion Examples (Second Set)

These experiments uncovered the regulation and significance of SG formation in response to lysosomal damage, providing insights into the interaction between membrane-bound organelles and membrane-less condensates. Through unbiased approaches, including proteomic analysis and high content microscopy, the inventors defined a novel signaling pathway that transmits calcium leakage from damaged lysosomes to induce eIF2α phosphorylation, ultimately leading to SG formation, thus promoting cell survival. This study aligns with recent research indicating the role of SGs in plugging damaged membranes and aiding in lysosomal repair (Bussi et al., 2023), underscoring SG formation as a vital cellular protective mechanism against lysosomal damage, essential for survival.

How does the cell detect lysosomal damage to initiate SGs? Our study revealed the significant involvement of a calcium signal in this process. Lysosomes function as key intracellular calcium reservoirs for various cellular activities (Lloyd-Evans et al., 2020; Xu et al., 2015). The inventors found that ALIX and ALG2 sense calcium leakage from damaged lysosomes, leading to the activation of ALIX's role in regulating PKR's activity. This involves control of the association between PKR and its activator, PACT, resulting in the phosphorylation of eIF2α. Notably, the inventors found that the role of ALIX and ALG2 in controlling eIF2α phosphorylation is distinct from their established function in ESCRT-mediated lysosomal repair. This suggests the multifaceted roles of ALIX and ALG2 as calcium sensors in coordinating cellular responses to lysosomal damage. Furthermore, our findings also indicate the intricate and adaptable nature of calcium signaling pathways in coordinating various cellular defense mechanisms against lysosomal damage. This extends beyond their involvement in TFEB nuclear translocation and phosphoinositide-mediated rapid lysosomal repair (Medina et al., 2015; Nakamura et al., 2020; Tan et al., 2022).

SGs consist of RNA-binding proteins and untranslated mRNA, both playing a crucial role in the process of phase separation (Millar et al., 2023). In addition to the calcium signal the inventors reported here as a trigger for SG formation during lysosomal damage, a recent study suggests that a decrease in pH can also induce SG formation on damaged lysosomes (Bussi et al., 2023). This is in line with the reported role of pH in G3BP1-driven SG condensation (Guillén-Boixet et al., 2020). However, the latter report indicates that pH may not directly regulate the RNA-binding affinity of G3BP1 but instead influences protein-protein interactions. It is worth noting that these experiments Were conducted in an in vitro system and the presence of mRNA. Therefore, it raises the possibility that multiple mechanisms may collaborate to trigger SG formation by controlling protein-protein interaction or the accumulation of untranslated mRNA in response to lysosomal damage. To understand the signaling mechanism responsible for the accumulation of untranslated mRNA, our study suggests a calcium-dependent pathway that induces untranslated mRNA for SG formation by controlling eIF2α phosphorylation. Thus, both pH and calcium-dependent pathways can collaboratively contribute to SG formation during lysosomal damage. Moreover, considering the central role of lysosomes as the main degradation center for diverse cellular components, such as RNA (Lawrence et al., 2019), and the recognition of lysosomal damage that can be sensed by various cellular mechanisms (Aits et al., 2015; Jia et al., 2022; Napolitano et al., 2016; Chrisovalantis Papadopoulos et al., 2017), the leakage of certain lysosomal contents or the activation of other lysosomal damage sensors may also contribute to the activation of PKR, eIF2α phosphorylation, or the regulation of SG formation.

Phosphorylation of eIF2α is a key event in SG formation as it causes the shutdown in global translation and the accumulation of untranslated mRNA, which triggers the phase separation, ultimately leading to SG formation (Ivanov et al., 2019; Riggs et al., 2020). However, there are instances of SG formation that occur independently of eIF2α phosphorylation, potentially regulated by translational shutdown through the mTORC1 pathway (Emara et al., 2012; Fujimura et al., 2012). Nevertheless, this does not appear to be the case for SG formation in response to lysosomal damage. Our data indicate that upon lysosomal damage, eIF2α phosphorylation is the primary driver for SG formation, though the impact of mTORC1 inactivation on translation shutdown and SG formation cannot be entirely ruled out. Importantly, the uncoupled relationship between mTORC1 inactivation and eIF2α phosphorylation in SG formation may be attributed to their differential impacts on protein translation events and mRNA entry into SGs. For example, mTORC1 inactivation primarily inhibits the translation pre-initiation, while eIF2α phosphorylation can impede the recruitment of the large ribosomal subunit to mRNA (Holz et al., 2005; Jackson et al., 2010). Recent research suggests that having just one large ribosomal subunit on mRNA is enough to prevent the recruitment of mRNA into SGs, while extended ribosome-free regions on mRNA are insufficient for SG formation (Fedorovskiy et al., 2023). Thus, mTORC1 inactivation may result in ribosome-free regions on mRNA, but alone, it is insufficient to prompt mRNA entry into SGs. The prevention of large ribosomal subunits on mRNA through eIF2α phosphorylation appears to be a crucial factor triggering this process and contributing to SG formation in the context of lysosomal damage. In addition, through the examination of SG formation in galectin knockout cells, the inventors recently showed (Jia et al., 2022) that galectin-8 does not influence SG formation. This finding supports the premise that eIF2α phosphorylation and mTORC1 inactivation are dissociated events during lysosomal damage, as the inventors have previously reported that galectin-8 can modulate mTORC1 activity under similar conditions (Jia et al., 2018). Furthermore, recent research has highlighted lysosomes as pivotal hubs in metabolic signaling, involving mTORC1 and AMPK pathways (Carroll et al., 2017; Jia et al., 2018; Jia et al., 2020b; Zoncu et al., 2011). Our findings regarding the regulation of eIF2α phosphorylation on damaged lysosomes, along with our earlier findings showing mTORC1 inactivation on damaged lysosomes (Jia et al., 2018), propose an innovative role for lysosomes as central command centers in orchestrating protein translation signaling during stress conditions.

Understanding the function of SGs during stress, especially in the context of lysosomal damage, remains limited. A recent report highlights the reparative role of SGs through their association with damaged lysosomes (Bussi et al., 2023). This finding aligns with our prior research; however, in our study, the inventors observed SGs at a distance from damaged lysosomes (Jia et al., 2022). This observation challenges the notion of SGs primarily serving as plugs and suggests a broader spectrum of roles for SGs in response to lysosomal damage. Given the significance of SG formation in supporting cell survival during lysosomal damage, as reported here, it is highly unlikely that SGs can undertake multiple tasks in restoring cellular homeostasis for survival. For example, considering SGs sequester non-translating mRNA (Khong et al., 2017), they may play roles in protecting mRNA and controlling mRNA fate of the transcriptome during lysosomal damage. Moreover, SG formation intersects with the integrated stress response (ISR), which can optimize the cell response by reprogramming gene expression to promote cellular recovery (Pakos-Zebrucka et al., 2016). The impact of SG formation on ISR may also enhance cellular fitness. Additionally, the involvement of SGs in various cellular processes, e.g., intracellular transport dynamics, ribosome biogenesis, and cell signaling (Gorsheneva et al., 2024; Ripin et al., 2023; M. L. Zhang et al., 2024), may further contribute to cellular survival upon lysosomal damage.

Recognizing lysosomal damage as a critical internal physiological trigger for SGs highlights the importance of enhancing our understanding of SG formation in disease contexts. The inventors detected the role of SG formation in cell survival within disease-specific contexts using a series of pathological reagents to induce lysosomal damage. Given the strong association of these reagents with both lysosomal damage and SG formation, delving into the molecular mechanisms governing the interaction between lysosomal damage and SGs may provide valuable insights for future therapeutic efforts.

All references cited herein, where relevant, are incorporated by reference and made a part of this application. The present invention is further described as set forth in one or more of the claims which follow.

### REFERENCES (FOR FIRST SET OF EXPERIMENTS)

Abu-Remaileh, M., Wyant, G. A., Kim, C., Laqtom, N. N., Abbasi, M., Chan, S. H., Freinkman, E., and Sabatini, D. M. (2017). Lysosomal metabolomics reveals V-ATPase- and mTOR-dependent regulation of amino acid efflux from lysosomes. Science 358, 807-813.
Agrotis, A., von Chamier, L., Oliver, H., Kiso, K., Singh, T., and Ketteler, R. (2019). Human ATG4 autophagy proteases counteract attachment of ubiquitin-like LC3/GABARAP proteins to other cellular proteins. Journal of Biological Chemistry 294, 12610-12621.
Aits, S., Kricker, J., Liu, B., Ellegaard, A. M., Hamalisto, S., Tvingsholm, S., Corcelle-Termeau, E., Hogh, S., Farkas, T., Holm Jonassen, A., et al. (2015). Sensitive detection of lysosomal membrane permeabilization by lysosomal galectin puncta assay. Autophagy 11, 1408-1424.
Alberti, S., Gladfelter, A., and Mittag, T. (2019). Considerations and challenges in studying liquid-liquid phase separation and biomolecular condensates. Cell 176, 419-434.
Anderson, P., and Kedersha, N. (2002). Stressful initiations. Journal of cell science 115, 3227-3234.
Anderson, P., and Kedersha, N. (2006). RNA granules. Journal of Cell Biology 172, 803-808.
Anderson, P., Kedersha, N., and Ivanov, P. (2015). Stress granules, P-bodies and cancer. Biochimica et biophysica acta 1849, 861-870.
Ash, P. E., Vanderweyde, T. E., Youmans, K. L., Apicco, D. J., and Wolozin, B. (2014). Pathological stress granules in Alzheimer's disease. Brain Res 1584, 52-58.
Aulas, A., Fay, M. M., Lyons, S. M., Achorn, C. A., Kedersha, N., Anderson, P., and Ivanov, P. (2017). Stress-specific differences in assembly and composition of stress granules and related foci. Journal of cell science 130, 927-937.
Balachandran, S., Roberts, P. C., Brown, L. E., Truong, H., Pattnaik, A. K., Archer, D. R., and Barber, G. N. (2000). Essential role for the dsRNA-dependent protein kinase PKR in innate immunity to viral infection. Immunity 13, 129-141.
Bar-Peled, L., Schweitzer, L. D., Zoncu, R., and Sabatini, D. M. (2012). Ragulator Is a GEF for the Rag GTPases that Signal Amino Acid Levels to mTORC1. Cell 150, 1196-1208.
Beale, R., Wise, H., Stuart, A., Ravenhill, B. J., Digard, P., and Randow, F. (2014). A LC3-interacting motif in the influenza A virus M2 protein is required to subvert autophagy and maintain virion stability. Cell host & microbe 15, 239-247.
Behrends, C., Sowa, M. E., Gygi, S. P., and Harper, J. W. (2010). Network organization of the human autophagy system. Nature 466, 68-76.
Berg, T. O., Stromhaug, E., Lovdal, T., Seglen, O., and Berg, T. (1994). Use of glycyl-L-phenylalanine 2-naphthylamide, a lysosome-disrupting cathepsin C substrate, to distinguish between lysosomes and prelysosomal endocytic vacuoles. The Biochemical journal 300 (Pt 1), 229-236.
Betz, C., and Hall, M. N. (2013). Where is mTOR and what is it doing there? Journal of Cell Biology 203, 563-574.
Bhattacharya, B., Xiao, S., Chatterjee, S., Urbanowski, M., Ordonez, A., Ihms, E. A., Agrahari, G., Lun, S., Berland, R., Pichugin, A., et al. (2021). The integrated stress response mediates necrosis in murine Mycobacterium tuberculosis granulomas. The Journal of clinical investigation 131.
Bonam, S. R., Wang, F., and Muller, S. (2019). Lysosomes as a therapeutic target. Nature Reviews Drug Discovery 18, 923-948.
Buchan, J. R., Kolaitis, R. M., Taylor, J. P., and Parker, R. (2013). Eukaryotic stress granules are cleared by autophagy and Cdc48/VCP function. Cell 153, 1461-1474.
Carosi, J. M., Nguyen, T. N., Lazarou, M., Kumar, S., and Sargeant, T. J. (2021). ATG8ylation of proteins: A way to cope with cell stress? The Journal of cell biology 220.
Castellano, B. M., Thelen, A. M., Moldavski, O., Feltes, M., van der Welle, R. E., Mydock-McGrane, L., Jiang, X., van Eijkeren, R. J., Davis, O. B., Louie, S. M., et al. (2017). Lysosomal cholesterol activates mTORC1 via an SLC38A9-Niemann-Pick C1 signaling complex. Science 355, 1306-1311.
Chauhan, S., Kumar, S., Jain, A., Ponpuak, M., Mudd, M. H., Kimura, T., Choi, S. W., Peters, R., Mandell, M., Bruun, J. A., et al. (2016). TRIMs and Galectins Globally Cooperate and TRIM16 and Galectin-3 Co-direct Autophagy in Endomembrane Damage Homeostasis. Developmental cell 39, 13-27.
Chitiprolu, M., Jagow, C., Tremblay, V., Bondy-Chorney, E., Paris, G., Savard, A., Palidwor, G., Barry, F. A., Zinman, L., Keith, J., et al. (2018). A complex of C9ORF72 and p62 uses arginine methylation to eliminate stress granules by autophagy. Nature communications 9, 2794.
Choi, S., Sa, M., Cho, N., Kim, K. K., and Park, S. H. (2019). Rbfox2 dissociation from stress granules suppresses cancer progression. Exp Mol Med 51, 1-12.
Claude-Taupin, A., Jia, J., Bhujabal, Z., Garfa-Traore, M., Kumar, S., da Silva, G. P. D., Javed, R., Gu, Y., Allers, L., Peters, R., et al. (2021). ATG9A protects the plasma membrane from programmed and incidental permeabilization. Nature cell biology.
Collier, N. C., Heuser, J., Levy, M. A., and Schlesinger, M. J. (1988). Ultrastructural and biochemical analysis of the stress granule in chicken embryo fibroblasts. The Journal of cell biology 106, 1131-1139.
Collier, N. C., and Schlesinger, M. J. (1986). The dynamic state of heat shock proteins in chicken embryo fibroblasts. The Journal of cell biology 103, 1495-1507.
Costa-Mattioli, M., and Walter, P. (2020). The integrated stress response: From mechanism to disease. Science 368.
Cotto, J. J., and Morimoto, R. I. (1999). Stress-induced activation of the heat-shock response: cell and molecular biology of heat-shock factors. Biochem Soc Symp 64, 105-118.
Deretic, V. (2021). Autophagy in inflammation, infection, and immunometabolism. Immunity 54, 437-453.
Deretic, V., and Kroemer, G. (2021). Autophagy in metabolism and quality control: opposing, complementary or interlinked functions? Autophagy, 1-10.
Durgan, J., Lystad, A. H., Sloan, K., Carlsson, S. R., Wilson, M. I., Marcassa, E., Ulferts, R., Webster, J., Lopez-Clavijo, A. F., and Wakelam, M. J. (2021). Non-canonical autophagy drives alternative ATG8 conjugation to phosphatidylserine. Molecular cell 81, 2031-2040. e2038.
Eapen, V. V., Swarup, S., Hoyer, M. J., Paulo, J. A., and Harper, W. (2021). Quantitative proteomics reveals the selectivity of ubiquitin-binding autophagy receptors in the turnover of damaged lysosomes by lysophagy. eLife 10, e72328.
Ellison, C. J., Kukulski, W., Boyle, K. B., Munro, S., and Randow, F. (2020). Transbilayer movement of sphingomyelin precedes catastrophic breakage of enterobacteria-containing vacuoles. Current Biology 30, 2974-2983. e2976.
Emara, M. M., Fujimura, K., Sciaranghella, D., Ivanova, V., Ivanov, P., and Anderson, P. (2012). Hydrogen peroxide induces stress granule formation independent of eIF2alpha phosphorylation. Biochemical and biophysical research communications 423, 763-769.
Fehrenbacher, N., and Jäättelä, M. (2005). Lysosomes as targets for cancer therapy. Cancer research 65, 2993-2995.
Fujimura, K., Sasaki, A. T., and Anderson, P. (2012). Selenite targets eIF4E-binding protein-1 to inhibit translation initiation and induce the assembly of non-canonical stress granules. Nucleic acids research 40, 8099-8110.
Fujita, N., Morita, E., Itoh, T., Tanaka, A., Nakaoka, M., Osada, Y., Umemoto, T., Saitoh, T., Nakatogawa, H., Kobayashi, S., et al. (2013). Recruitment of the autophagic machinery to endosomes during infection is mediated by ubiquitin. The Journal of cell biology 203, 115-128.
Gal-Ben-Ari, S., Barrera, I., Ehrlich, M., and Rosenblum, K. (2019). PKR: A kinase to remember. Frontiers in molecular neuroscience 11, 480.
Gallouzi, I. E., Brennan, C. M., Stenberg, M. G., Swanson, M. S., Eversole, A., Maizels, N., and Steitz, J. A. (2000). HuR binding to cytoplasmic mRNA is perturbed by heat shock. Proceedings of the National Academy of Sciences of the United States of America 97, 3073-3078.
Galluzzi, L., and Green, D. R. (2019). Autophagy-Independent Functions of the Autophagy Machinery. Cell 177, 1682-1699.
Ghosh, S., Dellibovi-Ragheb, T. A., Kerviel, A., Pak, E., Qiu, Q., Fisher, M., Takvorian, P. M., Bleck, C., Hsu, V. W., Fehr, A. R., et al. (2020). beta-Coronaviruses Use Lysosomes for Egress Instead of the Biosynthetic Secretory Pathway. Cell 183, 1520-1535 e1514.
Gilks, N., Kedersha, N., Ayodele, M., Shen, L., Stoecklin, G., Dember, L. M., and Anderson, P. (2004). Stress granule assembly is mediated by prion-like aggregation of TIA-1. Mol Biol Cell 15, 5383-5398.
Goodwin, J., Walkup, W. G., Hooper, K., Li, T., Kishi-Itakura, C., Ng, A., Lehmberg, T., Jha, A., Kommineni, S., and Fletcher, K. (2021a). GABARAP membrane conjugation sequesters the FLCN-FNIP tumor suppressor complex to activate TFEB and lysosomal biogenesis. bioRxiv.
Goodwin, J. M., Walkup I V, W. G., Hooper, K., Li, T., Kishi-Itakura, C., Ng, A., Lehmberg, T., Jha, A., Kommineni, S., and Fletcher, K. (2021b). GABARAP sequesters the FLCN-FNIP tumor suppressor complex to couple autophagy with lysosomal biogenesis. Science Advances 7, eabj2485.
Goodwin, J. M., Walkup, W. G. t., Hooper, K., Li, T., Kishi-Itakura, C., Ng, A., Lehmberg, T., Jha, A., Kommineni, S., Fletcher, K., et al. (2021c). GABARAP sequesters the FLCN-FNIP tumor suppressor complex to couple autophagy with lysosomal biogenesis. Sci Adv 7, eabj2485.
Gordon, D. E., Jang, G. M., Bouhaddou, M., Xu, J., Obernier, K., White, K. M., O'Meara, M. J., Rezelj, V. V., Guo, J. Z., and Swaney, D. L. (2020). A SARS-CoV-2 protein interaction map reveals targets for drug repurposing. Nature 583, 459-468.
Grabocka, E., and Bar-Sagi, D. (2016). Mutant KRAS Enhances Tumor Cell Fitness by Upregulating Stress Granules. Cell 167, 1803-1813 e1812.
Gu, Y., Princely Abudu, Y., Kumar, S., Bissa, B., Choi, S. W., Jia, J., Lazarou, M., Eskelinen, E. L., Johansen, T., and Deretic, V. (2019a). Mammalian Atg8 proteins regulate lysosome and autolysosome biogenesis through SNARE s. The EMBO journal 38, e101994.
Gu, Y., Princely Abudu, Y., Kumar, S., Bissa, B., Choi, S. W., Jia, J., Lazarou, M., Eskelinen, E. L., Johansen, T., and Deretic, V. (2019b). Mammalian Atg8 proteins regulate lysosome and autolysosome biogenesis through SNAREs. The EMBO journal 38, e101994.
Guo, H., Chitiprolu, M., Roncevic, L., Javalet, C., Hemming, F. J., Trung, M. T., Meng, L., Latreille, E., de Souza, C. T., and McCulloch, D. (2017a). Atg5 disassociates the VIVO-ATPase to promote exosome production and tumor metastasis independent of canonical macroautophagy. Developmental cell 43, 716-730. e717.
Guo, H., Chitiprolu, M., Roncevic, L., Javalet, C., Hemming, F. J., Trung, M. T., Meng, L., Latreille, E., Tanese de Souza, C., McCulloch, D., et al. (2017b). Atg5 Disassociates the VIVO-ATPase to Promote Exosome Production and Tumor Metastasis Independent of Canonical Macroautophagy. Developmental cell 43, 716-730 e717.
Han, T. W., Kato, M., Xie, S., Wu, L. C., Mirzaei, H., Pei, J., Chen, M., Xie, Y., Allen, J., and Xiao, G. (2012). Cell-free formation of RNA granules: bound RNAs identify features and components of cellular assemblies. Cell 149, 768-779.
Haud, N., Kara, F., Diekmann, S., Henneke, M., Willer, J. R., Hillwig, M. S., Gregg, R. G., MacIntosh, G. C., Gärtner, J., and Alia, A. (2011). rnaset2 mutant zebrafish model familial cystic leukoencephalopathy and reveal a role for RNase T2 in degrading ribosomal RNA. Proceedings of the National Academy of Sciences 108, 1099-1103.
Heckmann, B. L., and Green, D. R. (2019). LC3-associated phagocytosis at a glance. Journal of cell science 132.
Heckmann, B. L., Teubner, B. J., Tummers, B., Boada-Romero, E., Harris, L., Yang, M., Guy, C. S., Zakharenko, S. S., and Green, D. R. (2019). LC3-associated endocytosis facilitates β-amyloid clearance and mitigates neurodegeneration in murine Alzheimer's disease. Cell 178, 536-551. e514.
Hornung, V., Bauernfeind, F., Halle, A., Samstad, E. O., Kono, H., Rock, K. L., Fitzgerald, K. A., and Latz, E. (2008). Silica crystals and aluminum salts activate the NALP3 inflammasome through phagosomal destabilization. Nature immunology 9, 847-856.
Ivanov, P., Kedersha, N., and Anderson, P. (2019). Stress Granules and Processing Bodies in Translational Control. Cold Spring Harb Perspect Biol 11.
Jain, S., Wheeler, J. R., Walters, R. W., Agrawal, A., Barsic, A., and Parker, R. (2016). ATPase-Modulated Stress Granules Contain a Diverse Proteome and Substructure. Cell 164, 487-498.
Jia, J., Abudu, Y. P., Claude-Taupin, A., Gu, Y., Kumar, S., Choi, S. W., Peters, R., Mudd, M. H., Allers, L., Salemi, M., et al. (2018). Galectins Control mTOR in Response to Endomembrane Damage. Molecular cell 70, 120-135 e128.
Jia, J., Bissa, B., Brecht, L., Allers, L., Choi, S. W., Gu, Y., Zbinden, M., Burge, M. R., Timmins, G., Hallows, K., et al. (2020a). AMPK, a Regulator of Metabolism and Autophagy, Is Activated by Lysosomal Damage via a Novel Galectin-Directed Ubiquitin Signal Transduction System. Molecular cell 77, 951-969 e959.
Jia, J., Claude-Taupin, A., Gu, Y., Choi, S. W., Peters, R., Bissa, B., Mudd, M. H., Allers, L., Pallikkuth, S., Lidke, K. A., et al. (2020b). Galectin-3 Coordinates a Cellular System for Lysosomal Repair and Removal. Developmental cell 52, 69-87 e68.
Jia, J., Claude-Taupin, A., Gu, Y., Choi, S. W., Peters, R., Bissa, B., Mudd, M. H., Allers, L., Pallikkuth, S., Lidke, K. A., et al. (2020c). MERIT, a cellular system coordinating lysosomal repair, removal and replacement. Autophagy 16, 1539-1541.
Johansen, T., and Lamark, T. (2020). Selective Autophagy: ATG8 Family Proteins, LIR Motifs and Cargo Receptors. Journal of molecular biology 432, 80-103.
Kato, M., Han, T. W., Xie, S., Shi, K., Du, X., Wu, L. C., Mirzaei, H., Goldsmith, E. J., Longgood, J., and Pei, J. (2012). Cell-free formation of RNA granules: low complexity sequence domains form dynamic fibers within hydrogels. Cell 149, 753-767.
Kedersha, N., and, and Anderson, P. (2002). Stress granules: sites of mRNA triage that regulate mRNA stability and translatability. Biochemical Society transactions 30, 963-969.
Kedersha, N., and Anderson, P. (2007). Mammalian stress granules and processing bodies. Methods Enzymol 431, 61-81.
Kedersha, N., Chen, S., Gilks, N., Li, W., Miller, I. J., Stahl, J., and Anderson, P. (2002). Evidence that ternary complex (eIF2-GTP-tRNA(i)(Met))-deficient preinitiation complexes are core constituents of mammalian stress granules. Mol Biol Cell 13, 195-210.
Kedersha, N., Stoecklin, G., Ayodele, M., Yacono, P., Lykke-Andersen, J., Fritzler, M. J., Scheuner, D., Kaufman, R. J., Golan, D. E., and Anderson, P. (2005). Stress granules and processing bodies are dynamically linked sites of mRNP remodeling. The Journal of cell biology 169, 871-884.
Kedersha, N. L., Gupta, M., Li, W., Miller, I., and Anderson, P. (1999). RNA-binding proteins TIA-1 and TIAR link the phosphorylation of eIF-2 alpha to the assembly of mammalian stress granules. The Journal of cell biology 147, 1431-1442.
Khaperskyy, D. A., Hatchette, T. F., and McCormick, C. (2012). Influenza A virus inhibits cytoplasmic stress granule formation. FASEB journal: official publication of the Federation of American Societies for Experimental Biology 26, 1629-1639.
Kim, J., Kundu, M., Viollet, B., and Guan, K. L. (2011). AMPK and mTOR regulate autophagy through direct phosphorylation of Ulk1. Nature cell biology 13, 132-141.
Kimball, S. R. (2001). Regulation of translation initiation by amino acids in eukaryotic cells. Prog Mol Subcell Biol 26, 155-184.
Kirk, S. G., Samavati, L., and Liu, Y. (2020). MAP kinase phosphatase-1, a gatekeeper of the acute innate immune response. Life Sci 241, 117157.
Klionsky, D. J., Petroni, G., Amaravadi, R. K., Baehrecke, E. H., Ballabio, A., Boya, P., Bravo-San Pedro, J. M., Cadwell, K., Cecconi, F., and Choi, A. M. (2021). Autophagy in major human diseases. The EMBO journal, e108863.
Koerver, L., Papadopoulos, C., Liu, B., Kravic, B., Rota, G., Brecht, L., Veenendaal, T., Polajnar, M., Bluemke, A., and Ehrmann, M. (2019). The ubiquitin-conjugating enzyme UBE 2 QL 1 coordinates lysophagy in response to endolysosomal damage. EMBO reports 20, e48014.
Kosugi, S., Hasebe, M., Tomita, M., and Yanagawa, H. (2009). Systematic identification of cell cycle-dependent yeast nucleocytoplasmic shuttling proteins by prediction of composite motifs. Proceedings of the National Academy of Sciences 106, 10171-10176.
Kumar, S., Jain, A., Choi, S. W., da Silva, G. P. D., Allers, L., Mudd, M. H., Peters, R. S., Anonsen, J. H., Rusten, T. E., Lazarou, M., et al. (2020). Mammalian Atg8 proteins and the autophagy factor IRGM control mTOR and TFEB at a regulatory node critical for responses to pathogens. Nature cell biology 22, 973-985.
Kumar, S., Jain, A., Farzam, F., Jia, J., Gu, Y., Choi, S. W., Mudd, M. H., Claude-Taupin, A., Wester, M. J., Lidke, K. A., et al. (2018). Mechanism of Stx17 recruitment to autophagosomes via IRGM and mammalian Atg8 proteins. The Journal of cell biology 217, 997-1013.
Kumar, S., Javed, R., Mudd, M., Pallikkuth, S., Lidke, K. A., Jain, A., Tangavelou, K., Gudmundsson, S. R., Ye, C., and Rusten, T. E. (2021a). Mammalian hybrid pre-autophagosomal structure HyPAS generates autophagosomes. Cell 184, 5950-5969. e5922.
Kumar, S., Jia, J., and Deretic, V. (2021b). Atg8ylation as a general membrane stress and remodeling response. Cell Stress 5, 128-142.
Lee, C., Lamech, L., Johns, E., and Overholtzer, M. (2020a). Selective lysosome membrane turnover is induced by nutrient starvation. Developmental cell 55, 289-297. e284.
Lee, C., Lamech, L., Johns, E., and Overholtzer, M. (2020b). Selective Lysosome Membrane Turnover Is Induced by Nutrient Starvation. Developmental cell 55, 289-297 e284.
Leidal, A. M., Huang, H. H., Marsh, T., Solvik, T., Zhang, D., Ye, J., Kai, F., Goldsmith, J., Liu, J. Y., and Huang, Y.-H. (2020a). The LC3-conjugation machinery specifies the loading of RNA-binding proteins into extracellular vesicles. Nature cell biology 22, 187-199.
Leidal, A. M., Huang, H. H., Marsh, T., Solvik, T., Zhang, D., Ye, J., Kai, F., Goldsmith, J., Liu, J. Y., Huang, Y. H., et al. (2020b). The LC3-conjugation machinery specifies the loading of RNA-binding proteins into extracellular vesicles. Nature cell biology 22, 187-199.
Levine, B., and Kroemer, G. (2019). Biological Functions of Autophagy Genes: A Disease Perspective. Cell 176, 11-42.
Li, S., Peters, G. A., Ding, K., Zhang, X., Qin, J., and Sen, G. C. (2006). Molecular basis for PKR activation by PACT or dsRNA. Proceedings of the National Academy of Sciences 103, 10005-10010.
Li, T. Y., Sun, Y., Liang, Y., Liu, Q., Shi, Y., Zhang, C.-S., Zhang, C., Song, L., Zhang, P., and Zhang, X. (2016). ULK1/2 constitute a bifurcate node controlling glucose metabolic fluxes in addition to autophagy. Molecular cell 62, 359-370.
Lindquist, M. E., Lifland, A. W., Utley, T. J., Santangelo, P. J., and Crowe, J. E., Jr. (2010). Respiratory syncytial virus induces host RNA stress granules to facilitate viral replication. Journal of virology 84, 12274-12284.
Liu-Yesucevitz, L., Bilgutay, A., Zhang, Y. J., Vanderweyde, T., Citro, A., Mehta, T., Zaarur, N., McKee, A., Bowser, R., Sherman, M., et al. (2010). Tar DNA binding protein-43 (TDP-43) associates with stress granules: analysis of cultured cells and pathological brain tissue. PloS one 5, e13250.
Loi, M., Raimondi, A., Morone, D., and Molinari, M. (2019). ESCRT-III-driven piecemeal micro-ER-phagy remodels the ER during recovery from ER stress. Nature communications 10, 1-12.
Lu, B., Nakamura, T., Inouye, K., Li, J., Tang, Y., Lundback, P., Valdes-Ferrer, S. I., Olofsson, P. S., Kalb, T., Roth, J., et al. (2012). Novel role of PKR in inflammasome activation and HMGB1 release. Nature 488, 670-674.
Lu, L., Han, A.-P., and Chen, J.-J. (2001). Translation initiation control by heme-regulated eukaryotic initiation factor 2a kinase in erythroid cells under cytoplasmic stresses. Molecular and cellular biology 21, 7971-7980.
Lu, P. D., Jousse, C., Marciniak, S. J., Zhang, Y., Novoa, I., Scheuner, D., Kaufman, R. J., Ron, D., and Harding, H. P. (2004). Cytoprotection by pre-emptive conditional phosphorylation of translation initiation factor 2. The EMBO journal 23, 169-179.
Luo, L., Li, Z., Zhao, T., Ju, X., Ma, P., Jin, B., Zhou, Y., He, S., Huang, J., Xu, X., et al. (2021). SARS-CoV-2 nucleocapsid protein phase separates with G3BPs to disassemble stress granules and facilitate viral production. Sci Bull (Beijing).
Mackenzie, I. R., Nicholson, A. M., Sarkar, M., Messing, J., Purice, M. D., Pottier, C., Annu, K., Baker, M., Perkerson, R. B., and Kurti, A. (2017). TIA1 mutations in amyotrophic lateral sclerosis and frontotemporal dementia promote phase separation and alter stress granule dynamics. Neuron 95, 808-816. e809.
Maejima, I., Takahashi, A., Omori, H., Kimura, T., Takabatake, Y., Saitoh, T., Yamamoto, A., Hamasaki, M., Noda, T., Isaka, Y., et al. (2013). Autophagy sequesters damaged lysosomes to control lysosomal biogenesis and kidney injury. The EMBO journal 32, 2336-2347.
Manzanillo, P. S., Shiloh, M. U., Portnoy, D. A., and Cox, J. S. (2012). Mycobacterium Tuberculosis Activates the DNA-Dependent Cytosolic Surveillance Pathway within Macrophages. Cell host & microbe 11, 469-480.
Markmiller, S., Soltanieh, S., Server, K. L., Mak, R., Jin, W., Fang, M. Y., Luo, E. C., Krach, F., Yang, D., Sen, A., et al. (2018). Context-Dependent and Disease-Specific Diversity in Protein Interactions within Stress Granules. Cell 172, 590-604 e513.
Marshall, R. S., Hua, Z., Mali, S., McLoughlin, F., and Vierstra, R. D. (2019). ATG8-Binding UIM Proteins Define a New Class of Autophagy Adaptors and Receptors. Cell 177, 766-781 e724.
McCormick, C., and Khaperskyy, D. A. (2017). Translation inhibition and stress granules in the antiviral immune response. Nat Rev Immunol 17, 647-660.
McEwen, E., Kedersha, N., Song, B., Scheuner, D., Gilks, N., Han, A., Chen, J. J., Anderson, P., and Kaufman, R. J. (2005). Heme-regulated inhibitor kinase-mediated phosphorylation of eukaryotic translation initiation factor 2 inhibits translation, induces stress granule formation, and mediates survival upon arsenite exposure. The Journal of biological chemistry 280, 16925-16933.
Miao, G., Zhao, H., Li, Y., Ji, M., Chen, Y., Shi, Y., Bi, Y., Wang, P., and Zhang, H. (2021). ORF3a of the COVID-19 virus SARS-CoV-2 blocks HOPS complex-mediated assembly of the SNARE complex required for autolysosome formation. Developmental cell 56, 427-442. e425.
Mizushima, N. (2020). The ATG conjugation systems in autophagy. Current opinion in cell biology 63, 1-10.
Morishita, H., and Mizushima, N. (2019). Diverse Cellular Roles of Autophagy. Annu Rev Cell Dev Biol 35, 453-475.
Nabeel-Shah, S., Lee, H., Ahmed, N., Marcon, E., Farhangmehr, S., Pu, S., Burke, G. L., Ashraf, K., Wei, H., Zhong, G., et al. (2020). SARS-CoV-2 Nucleocapsid protein attenuates stress granule formation and alters gene expression via direct interaction with host mRNAs. bioRxiv, 2020.2010.2023.342113.
Nakagawa, K., Narayanan, K., Wada, M., and Makino, S. (2018). Inhibition of stress granule formation by Middle East respiratory syndrome coronavirus 4a accessory protein facilitates viral translation, leading to efficient virus replication. Journal of virology 92, e00902-00918.
Nakamura, S., Shigeyama, S., Minami, S., Shima, T., Akayama, S., Matsuda, T., Esposito, A., Napolitano, G., Kuma, A., and Namba-Hamano, T. (2020). LC3 lipidation is essential for TFEB activation during the lysosomal damage response to kidney injury. Nature cell biology 22, 1252-1263.
Nakamura, T., Furuhashi, M., Li, P., Cao, H., Tuncman, G., Sonenberg, N., Gorgun, C. Z., and Hotamisligil, G. S. (2010). Double-stranded RNA-dependent protein kinase links pathogen sensing with stress and metabolic homeostasis. Cell 140, 338-348.
Nakao, A., Yoshihama, M., and Kenmochi, N. (2004). RPG: the Ribosomal Protein Gene database. Nucleic acids research 32, D168-170.
Napolitano, G., and Ballabio, A. (2016). TFEB at a glance. Journal of cell science 129, 2475-2481.
Nguyen, T. N., Padman, B. S., Usher, J., Oorschot, V., Ramm, G., and Lazarou, M. (2016). Atg8 family LC3/GABARAP proteins are crucial for autophagosome-lysosome fusion but not autophagosome formation during PINK1/Parkin mitophagy and starvation. The Journal of cell biology 215, 857-874.
Nguyen, T. N., Padman, B. S., Zellner, S., Khuu, G., Uoselis, L., Lam, W. K., Skulsuppaisarn, M., Lindblom, R. S., Watts, E. M., and Behrends, C. (2021). ATG4 family proteins drive phagophore growth independently of the LC3/GABARAP lipidation system. Molecular cell 81, 2013-2030. e2019.
Nover, L., Scharf, K. D., and Neumann, D. (1983). Formation of cytoplasmic heat shock granules in tomato cell cultures and leaves. Molecular and cellular biology 3, 1648-1655.
Nover, L., Scharf, K. D., and Neumann, D. (1989). Cytoplasmic heat shock granules are formed from precursor particles and are associated with a specific set of mRNAs. Molecular and cellular biology 9, 1298-1308.
Novoa, I., Zhang, Y., Zeng, H., Jungreis, R., Harding, H. P., and Ron, D. (2003). Stress-induced gene expression requires programmed recovery from translational repression. The EMBO journal 22, 1180-1187.
Panas, M. D., Varjak, M., Lulla, A., Eng, K. E., Merits, A., Karlsson Hedestam, G. B., and McInerney, G. M. (2012). Sequestration of G3BP coupled with efficient translation inhibits stress granules in Semliki Forest virus infection. Mol Biol Cell 23, 4701-4712.
Papadopoulos, C., Kirchner, P., Bug, M., Grum, D., Koerver, L., Schulze, N., Poehler, R., Dressler, A., Fengler, S., Arhzaouy, K., et al. (2017). VCP/p97 cooperates with YOD1, UBXD1 and PLAA to drive clearance of ruptured lysosomes by autophagy. The EMBO journal 36, 135-150.
Pataer, A., Ozpolat, B., Shao, R., Cashman, N. R., Plotkin, S. S., Samuel, C. E., Lin, S. H., Kabil, N. N., Wang, J., Majidi, M., et al. (2020). Therapeutic targeting of the PI4K2A/PKR lysosome network is critical for misfolded protein clearance and survival in cancer cells. Oncogene 39, 801-813.
Patil, C., and Walter, P. (2001). Intracellular signaling from the endoplasmic reticulum to the nucleus: the unfolded protein response in yeast and mammals. Current opinion in cell biology 13, 349-355.
Perdikari, T. M., Murthy, A. C., Ryan, V. H., Watters, S., Naik, M. T., and Fawzi, N. L. (2020). SARS-CoV-2 nucleocapsid protein phase-separates with RNA and with human hnRNPs. The EMBO journal 39, e106478.
Pochopien, A. A., Beckert, B., Kasvandik, S., Berninghausen, O., Beckmann, R., Tenson, T., and Wilson, D. N. (2021). Structure of Gen1 bound to stalled and colliding 80S ribosomes. Proceedings of the National Academy of Sciences 118.
Pols, M. S., ten Brink, C., Gosavi, P., Oorschot, V., and Klumperman, J. (2013). The HOPS proteins hVps41 and hVps39 are required for homotypic and heterotypic late endosome fusion. Traffic 14, 219-232.
Prentzell, M. T., Rehbein, U., Cadena Sandoval, M., De Meulemeester, A. S., Baumeister, R., Brohee, L., Berdel, B., Bockwoldt, M., Carroll, B., Chowdhury, S. R., et al. (2021). G3BPs tether the TSC complex to lysosomes and suppress mTORC1 signaling. Cell 184, 655-674 e627.
Puertollano, R., Ferguson, S. M., Brugarolas, J., and Ballabio, A. (2018). The complex relationship between TFEB transcription factor phosphorylation and subcellular localization. The EMBO journal 37, e98804.
Qiu, J., and Xia, Y. (2020). Killing cancer cells by rupturing their lysosomes. Nature nanotechnology 15, 252-253.
Raaben, M., Groot Koerkamp, M. J., Rottier, P. J., and De Haan, C. A. (2007). Mouse hepatitis coronavirus replication induces host translational shutoff and mRNA decay, with concomitant formation of stress granules and processing bodies. Cellular microbiology 9, 2218-2229.
Radulovic, M., Schink, K. O., Wenzel, E. M., Nahse, V., Bongiovanni, A., Lafont, F., and Stenmark, H. (2018). ESCRT-mediated lysosome repair precedes lysophagy and promotes cell survival. The EMBO journal 37.
Riggs, C. L., Kedersha, N., Ivanov, P., and Anderson, P. (2020). Mammalian stress granules and P bodies at a glance. Journal of cell science 133.
Sancak, Y., Bar-Peled, L., Zoncu, R., Markhard, A. L., Nada, S., and Sabatini, D. M. (2010). Ragulator-Rag complex targets mTORC1 to the lysosomal surface and is necessary for its activation by amino acids. Cell 141, 290-303.
Sancak, Y., Peterson, T. R., Shaul, Y. D., Lindquist, R. A., Thoreen, C. C., Bar-Peled, L., and Sabatini, D. M. (2008). The Rag GTPases bind raptor and mediate amino acid signaling to mTORC1. Science 320, 1496-1501.
Sanjuan, M. A., Dillon, C. P., Tait, S. W., Moshiach, S., Dorsey, F., Connell, S., Komatsu, M., Tanaka, K., Cleveland, J. L., Withoff, S., et al. (2007). Toll-like receptor signalling in macrophages links the autophagy pathway to phagocytosis. Nature 450, 1253-1257.
Savastano, A., Ibanez de Opakua, A., Rankovic, M., and Zweckstetter, M. (2020). Nucleocapsid protein of SARS-CoV-2 phase separates into RNA-rich polymerase-containing condensates. Nature communications 11, 6041.
Settembre, C., Di Malta, C., Polito, V. A., Garcia Arencibia, M., Vetrini, F., Erdin, S., Erdin, S. U., Huynh, T., Medina, D., Colella, P., et al. (2011). TFEB links autophagy to lysosomal biogenesis. Science 332, 1429-1433.
Settembre, C., Zoncu, R., Medina, D. L., Vetrini, F., Erdin, S., Erdin, S., Huynh, T., Ferron, M., Karsenty, G., and Vellard, M. C. (2012). A lysosome-to-nucleus signalling mechanism senses and regulates the lysosome via mTOR and TFEB. The EMBO journal 31, 1095-1108.
Sfakianos, A. P., Mellor, L. E., Pang, Y. F., Kritsiligkou, P., Needs, H., Abou-Hamdan, H., Desaubry, L., Poulin, G. B., Ashe, M. P., and Whitmarsh, A. J. (2018). The mTOR-S6 kinase pathway promotes stress granule assembly. Cell Death & Differentiation 25, 1766-1780.
Shin, H. R., and Zoncu, R. (2020). The lysosome at the intersection of cellular growth and destruction. Developmental cell.
Skowyra, M. L., Schlesinger, P. H., Naismith, T. V., and Hanson, P. I. (2018). Triggered recruitment of ESCRT machinery promotes endolysosomal repair. Science 360.
Solinger, J. A., and Spang, A. (2013). Tethering complexes in the endocytic pathway: CORVET and HOPS. The FEBS journal 280, 2743-2757.
Somasekharan, S. P., El-Naggar, A., Leprivier, G., Cheng, H., Hajee, S., Grunewald, T. G., Zhang, F., Ng, T., Delattre, O., Evdokimova, V., et al. (2015). YB-1 regulates stress granule formation and tumor progression by translationally activating G3BP1. The Journal of cell biology 208, 913-929.
Sønder, S. L., Häger, S. C., Heitmann, A. S. B., Frankel, L. B., Dias, C., Simonsen, A. C., and Nylandsted, J. (2021). Restructuring of the plasma membrane upon damage by LC3-associated macropinocytosis. Science Advances 7, eabg 1969.
Sowa, M. E., Bennett, E. J., Gygi, S. P., and Harper, J. W. (2009). Defining the human deubiquitinating enzyme interaction landscape. Cell 138, 389-403.
Srivastava, S. P., Kumar, K. U., and Kaufman, R. J. (1998). Phosphorylation of eukaryotic translation initiation factor 2 mediates apoptosis in response to activation of the double-stranded RNA-dependent protein kinase. The Journal of biological chemistry 273, 2416-2423.
Stukalov, A., Girault, V., Grass, V., Karayel, O., Bergant, V., Urban, C., Haas, D. A., Huang, Y., Oubraham, L., Wang, A., et al. (2021). Multilevel proteomics reveals host perturbations by SARS-CoV-2 and SARS-COV. Nature 594, 246-252.
Sturgill-Koszycki, S., Schaible, U. E., and Russell, D. G. (1996). Mycobacterium-containing phagosomes are accessible to early endosomes and reflect a transitional state in normal phagosome biogenesis. The EMBO journal 15, 6960-6968.
Sun, H., Charles, C. H., Lau, L. F., and Tonks, N. K. (1993). MKP-1 (3CH134), an immediate early gene product, is a dual specificity phosphatase that dephosphorylates MAP kinase in vivo. Cell 75, 487-493.
Szklarczyk, D., Gable, A. L., Nastou, K. C., Lyon, D., Kirsch, R., Pyysalo, S., Doncheva, N. T., Legeay, M., Fang, T., and Bork, P. (2021). The STRING database in 2021: customizable protein-protein networks, and functional characterization of user-uploaded gene/measurement sets. Nucleic acids research 49, D605-D612.
Takahara, T., and Maeda, T. (2012). Transient sequestration of TORC1 into stress granules during heat stress. Molecular cell 47, 242-252.
Tan, J. M., Mellouk, N., Osborne, S. E., Ammendolia, D. A., Dyer, D. N., Li, R., Brunen, D., van Rijn, J. M., Huang, J., and Czuczman, M. A. (2018). An ATG16L1-dependent pathway promotes plasma membrane repair and limits Listeria monocytogenes cell-to-cell spread. Nature microbiology 3, 1472-1485.
Taylor, J. P., Brown, R. H., Jr., and Cleveland, D. W. (2016). Decoding ALS: from genes to mechanism. Nature 539, 197-206.
Thedieck, K., Holzwarth, B., Prentzell, M. T., Boehlke, C., Kläsener, K., Ruf, S., Sonntag, A. G., Maerz, L., Grellscheid, S.-N., and Kremmer, E. (2013). Inhibition of mTORC1 by astrin and stress granules prevents apoptosis in cancer cells. Cell 154, 859-874.
Thiele, D. L., and Lipsky, P. E. (1990). Mechanism of L-leucyl-L-leucine methyl ester-mediated killing of cytotoxic lymphocytes: dependence on a lysosomal thiol protease, dipeptidyl peptidase I, that is enriched in these cells. Proceedings of the National Academy of Sciences of the United States of America 87, 83-87.
Thomis, D. C., and Samuel, C. E. (1993). Mechanism of interferon action: evidence for intermolecular autophosphorylation and autoactivation of the interferon-induced, RNA-dependent protein kinase PKR. Journal of virology 67, 7695-7700.
Thurston, T. L., Wandel, M. P., von Muhlinen, N., Foeglein, A., and Randow, F. (2012). Galectin 8 targets damaged vesicles for autophagy to defend cells against bacterial invasion. Nature 482, 414-418.
Tsutsuki, H., Yahiro, K., Ogura, K., Ichimura, K., Iyoda, S., Ohnishi, M., Nagasawa, S., Seto, K., Moss, J., and Noda, M. (2016). Subtilase cytotoxin produced by locus of enterocyte effacement-negative Shiga-toxigenic Escherichia coli induces stress granule formation. Cell Microbiol 18, 1024-1040.
van Leeuwen, W., and Rabouille, C. (2019). Cellular stress leads to the formation of membraneless stress assemblies in eukaryotic cells. Traffic 20, 623-638.
Vanderweyde, T., Yu, H., Varnum, M., Liu-Yesucevitz, L., Citro, A., Ikezu, T., Duff, K., and Wolozin, B. (2012). Contrasting pathology of the stress granule proteins TIA-1 and G3BP in tauopathies. The Journal of neuroscience: the official journal of the Society for Neuroscience 32, 8270-8283.
Vattem, K. M., and Wek, R. C. (2004). Reinitiation involving upstream ORFs regulates ATF4 mRNA translation in mammalian cells. Proceedings of the National Academy of Sciences 101, 11269-11274.
Velasquez, F., Marin-Rojas, J., Soto-Rifo, R., Torres, A., Del Canto, F., and Valiente-Echeverria, F. (2020). Escherichia coli HS and Enterotoxigenic Escherichia coli Hinder Stress Granule Assembly. Microorganisms 9.
Vonaesch, P., Campbell-Valois, F. X., Dufour, A., Sansonetti, P. J., and Schnupf, P. (2016). Shigella flexneri modulates stress granule composition and inhibits stress granule aggregation. Cell Microbiol 18, 982-997.
Vonaesch, P., Sansonetti, P. J., and Schnupf, P. (2017). Immunofluorescence Analysis of Stress Granule Formation After Bacterial Challenge of Mammalian Cells. J Vis Exp.
Wang, B., Maxwell, B. A., Joo, J. H., Gwon, Y., Messing, J., Mishra, A., Shaw, T. I., Ward, A. L., Quan, H., Sakurada, S. M., et al. (2019). ULK1 and ULK2 Regulate Stress Granule Disassembly Through Phosphorylation and Activation of VCP/p97. Molecular cell 74, 742-757 e748.
Weber, S. C., and Brangwynne, C. P. (2012). Getting RNA and protein in phase. Cell 149, 1188-1191.
White, J. P., Cardenas, A. M., Marissen, W. E., and Lloyd, R. E. (2007). Inhibition of cytoplasmic mRNA stress granule formation by a viral proteinase. Cell Host Microbe 2, 295-305.
Williams, B. R. (1999). PKR; a sentinel kinase for cellular stress. Oncogene 18, 6112-6120.
Williams, B. R. (2001). Signal integration via PKR. Sci STKE 2001, re2.
Wippich, F., Bodenmiller, B., Trajkovska, M. G., Wanka, S., Aebersold, R., and Pelkmans, L. (2013). Dual specificity kinase DYRK3 couples stress granule condensation/dissolution to mTORC1 signaling. Cell 152, 791-805.
Xu, Y., Zhou, P., Cheng, S., Lu, Q., Nowak, K., Hopp, A.-K., Li, L., Shi, X., Zhou, Z., and Gao, W. (2019). A bacterial effector reveals the V-ATPase-ATG16L1 axis that initiates xenophagy. Cell 178, 552-566. e520.
Yang, P., Mathieu, C., Kolaitis, R. M., Zhang, P., Messing, J., Yurtsever, U., Yang, Z., Wu, J., Li, Y., Pan, Q., et al. (2020). G3BP1 Is a Tunable Switch that Triggers Phase Separation to Assemble Stress Granules. Cell 181, 325-345 e328.
Yoshida, Y., Yasuda, S., Fujita, T., Hamasaki, M., Murakami, A., Kawawaki, J., Iwai, K., Saeki, Y., Yoshimori, T., Matsuda, N., et al. (2017). Ubiquitination of exposed glycoproteins by SCF(FBXO27) directs damaged lysosomes for autophagy. Proceedings of the National Academy of Sciences of the United States of America 114, 8574-8579.
Youn, J. Y., Dunham, W. H., Hong, S. J., Knight, J. D. R., Bashkurov, M., Chen, G. I., Bagci, H., Rathod, B., MacLeod, G., Eng, S. W. M., et al. (2018). High-Density Proximity Mapping Reveals the Subcellular Organization of mRNA-Associated Granules and Bodies. Molecular cell 69, 517-532 e511.
Yue, Y., Nabar, N. R., Shi, C. S., Kamenyeva, O., Xiao, X., Hwang, I. Y., Wang, M., and Kehrl, J. H. (2018). SARS-Coronavirus Open Reading Frame-3a drives multimodal necrotic cell death. Cell death & disease 9, 904.
Zoncu, R., Bar-Peled, L., Efeyan, A., Wang, S., Sancak, Y., and Sabatini, D. M. (2011). mTORC1 senses lysosomal amino acids through an inside-out mechanism that requires the vacuolar H (+)-ATPase. Science 334, 678-683.

### REFERENCES (FOR SECOND SET of EXPERIMENTS)

Abu-Remaileh, M., Wyant, G. A., Kim, C., Laqtom, N. N., Abbasi, M., Chan, S. H., . . . Sabatini, D. M. (2017). Lysosomal metabolomics reveals V-ATPase- and mTOR-dependent regulation of amino acid efflux from lysosomes. Science, 358(6364), 807-813. doi: 10.1126/science.aan6298
Advani, V. M., & Ivanov, P. (2020). Stress granule subtypes: an emerging link to neurodegeneration. Cell Mol Life Sci, 77(23), 4827-4845. doi: 10.1007/s00018-020-03565-0
Aits, S., & Jäättelä, M. (2013). Lysosomal cell death at a glance. Journal of cell science, 126(9), 1905-1912.
Aits, S., Kricker, J., Liu, B., Ellegaard, A.-M., Hämälisto, S., Tvingsholm, S., . . . Holm Jonassen, A. (2015). Sensitive detection of lysosomal membrane permeabilization by lysosomal galectin puncta assay. Autophagy, 11(8), 1408-1424.
Amaral, O., Martins, M., Oliveira, A. R., Duarte, A. J., Mondragão-Rodrigues, I., & Macedo, M. F. (2023). The Biology of Lysosomes: From Order to Disorder. Biomedicines, 11(1), 213.
Anyona, S. B., Cheng, Q., Raballah, E., Hurwitz, I., Lambert, C. G., McMahon, B. H., . . . Perkins, D. J. (2022). Ingestion of hemozoin by peripheral blood mononuclear cells alters temporal gene expression of ubiquitination processes. Biochemistry and Biophysics Reports, 29, 101207.
Ballabio, A., & Bonifacino, J. S. (2020). Lysosomes as dynamic regulators of cell and organismal homeostasis. Nat Rev Mol Cell Biol, 21(2), 101-118. doi: 10.1038/s41580-019-0185-4
Barlan, Danthi, P., & Wiethoff, C. (2011). Lysosomal localization and mechanism of membrane penetration influence nonenveloped virus activation of the NLRP3 inflammasome. Virology, 412(2), 306-314.
Barlan, A., Griffin, T., McGuire, K., & Wiethoff, C. (2011). Adenovirus membrane penetration activates the NLRP3 inflammasome. Journal of virology, 85(1), 146-155.
Bonam, S. R., Wang, F., & Muller, S. (2019). Lysosomes as a therapeutic target. Nat Rev Drug Discov, 18(12), 923-948. doi: 10.1038/s41573-019-0036-1
Boyle, K. B., Ellison, C. J., Elliott, P. R., Schuschnig, M., Grimes, K., Dionne, M. S., Randow, F. (2023). TECPR1 conjugates LC3 to damaged endomembranes upon detection of sphingomyelin exposure. The EMBO journal, 42(17), e113012.
Bussi, C., Mangiarotti, A., Vanhille-Campos, C., Aylan, B., Pellegrino, E., Athanasiadi, N., . . . Gutierrez, M. G. (2023). Stress granules plug and stabilize damaged endolysosomal membranes. Nature. doi: 10.1038/s41586-023-06726-w
Carroll, B., & Dunlop, E. A. (2017). The lysosome: a crucial hub for AMPK and mTORC1 signalling. Biochemical Journal, 474(9), 1453-1466.
Chan, F. K.-M., Moriwaki, K., & De Rosa, M. J. (2013). Detection of necrosis by release of lactate dehydrogenase activity. Immune Homeostasis: Methods and Protocols, 65-70.
Chauhan, S., Kumar, S., Jain, A., Ponpuak, M., Mudd, M. H., Kimura, T., . . . Deretic, V. (2016). TRIMs and Galectins Globally Cooperate and TRIM16 and Galectin-3 Co-direct Autophagy in Endomembrane Damage Homeostasis. Dev Cell, 39(1), 13-27. doi: 10.1016/j.devcel.2016.08.003
Chazotte, B. (2011). Labeling lysosomes in live cells with LysoTracker. Cold Spring Harbor Protocols, 2011(2), pdb. prot5571.
Chen, Q. Y., & Costa, M. (2018). PI3K/Akt/mTOR signaling pathway and the biphasic effect of arsenic in carcinogenesis. Molecular Pharmacology, 94(1), 784-792.
Chen, W., Motsinger, M. M., Li, J., Bohannon, K. P., & Hanson, P. I. (2024). Ca2+-sensor ALG-2 engages ESCRTs to enhance lysosomal membrane resilience to osmotic stress. bioRxiv, 2024.2002. 2004.578682.
Chukwurah, E., Farabaugh, K. T., Guan, B. J., Ramakrishnan, P., & Hatzoglou, M. (2021). A tale of two proteins: PACT and PKR and their roles in inflammation. The FEBS Journal, 288(22), 6365-6391.
Coronado, L. M., Nadovich, C. T., & Spadafora, C. (2014). Malarial hemozoin: from target to tool. Biochimica et Biophysica Acta (BBA)-General Subjects, 1840(6), 2032-2041.
Costa-Mattioli, M., & Walter, P. (2020). The integrated stress response: From mechanism to disease. Science, 368(6489). doi: 10.1126/science.aat5314
Cotto, J. J., & Morimoto, R. I. (1999). Stress-induced activation of the heat-shock response: cell and molecular biology of heat-shock factors. Biochem Soc Symp, 64, 105-118. Retrieved from www.ncbi.nlm.nih.gov/pubmed/10207624 Crowley, L. C., Scott, A. P., Marfell, B. J., Boughaba, J. A., Chojnowski, G., & Waterhouse, N. J. (2016). Measuring cell death by propidium iodide uptake and flow cytometry. Cold Spring Harbor Protocols, 2016(7), pdb. prot087163.
Daussy, C. F., & Wodrich, H. (2020). “Repair Me if You Can”: membrane damage, response, and control from the viral perspective. Cells, 9(9), 2042.
Eapen, V. V., Swarup, S., Hoyer, M. J., Paulo, J. A., & Harper, J. W. (2021). Quantitative proteomics reveals the selectivity of ubiquitin-binding autophagy receptors in the turnover of damaged lysosomes by lysophagy. elife, 10, e72328.
Ebstrup, M. L., Sønder, S. L., Fogde, D. L., Heitmann, A. S. B., Dietrich, T. N., Dias, C., . . . Nylandsted, J. (2023). Annexin A7 mediates lysosome repair independently of ESCRT-III. Frontiers in cell and developmental biology, 11.
Emara, M. M., Fujimura, K., Sciaranghella, D., Ivanova, V., Ivanov, P., & Anderson, P. (2012). Hydrogen peroxide induces stress granule formation independent of eIF2α phosphorylation. Biochemical and biophysical research communications, 423(4), 763-769.
Fedorovskiy, A. G., Burakov, A. V., Terenin, I. M., Bykov, D. A., Lashkevich, K. A., Popenko, V. I., . . . Prassolov, V. S. (2023). A solitary stalled 80S ribosome prevents mRNA recruitment to stress granules. Biochemistry (Moscow), 88(11), 1786-1799.
Fehrenbacher, N., & Jäättelä, M. (2005). Lysosomes as targets for cancer therapy. Cancer research, 65(8), 2993-2995.
Flavin, W. P., Bousset, L., Green, Z. C., Chu, Y., Skarpathiotis, S., Chaney, M. J., . . . Campbell, E. M. (2017). Endocytic vesicle rupture is a conserved mechanism of cellular invasion by amyloid proteins. Acta neuropathologica, 134, 629-653.
Freibaum, B. D., Messing, J., Yang, P., Kim, H. J., & Taylor, J. P. (2021). High-fidelity reconstitution of stress granules and nucleoli in mammalian cellular lysate. Journal of Cell Biology, 220(3), e202009079.
Fujimura, K., Sasaki, A. T., & Anderson, P. (2012). Selenite targets eIF4E-binding protein-1 to inhibit translation initiation and induce the assembly of non-canonical stress granules. Nucleic Acids Res, 40(16), 8099-8110. doi: 10.1093/nar/gks566
Gal-Ben-Ari, S., Barrera, I., Ehrlich, M., & Rosenblum, K. (2019). PKR: a kinase to remember. Frontiers in molecular neuroscience, 11, 480.
Gallardo, J., Pérez-Illana, M., Martín-González, N., & San Martín, C. (2021). Adenovirus structure: what is new? International journal of molecular sciences, 22(10), 5240.
Ghosh, S., Dellibovi-Ragheb, T. A., Kerviel, A., Pak, E., Qiu, Q., Fisher, M., . . . Altan-Bonnet, N. (2020). beta-Coronaviruses Use Lysosomes for Egress Instead of the Biosynthetic Secretory Pathway. Cell, 183(6), 1520-1535 e1514. doi: 10.1016/j.cell.2020.10.039
Gorsheneva, N. A., Sopova, J. V., Azarov, V. V., Grizel, A. V., & Rubel, A. A. (2024). Biomolecular Condensates: Structure, Functions, Methods of Research. Biochemistry (Moscow), 89 (Suppl 1), S205-S223.
Greber, U. F., Webster, P., Weber, J., & Helenius, A. (1996). The role of the adenovirus protease on virus entry into cells. The EMBO journal, 15(8), 1766-1777.
Guerra, E. D., Baakdah, F., Georges, E., Bohle, D. S., & Cerruti, M. (2019). What is pure hemozoin? A close look at the surface of the malaria pigment. Journal of inorganic biochemistry, 194, 214-222.
Guillén-Boixet, J., Kopach, A., Holehouse, A. S., Wittmann, S., Jahnel, M., Schlüßler, R., . . . Mateju, D. (2020). RNA-induced conformational switching and clustering of G3BP drive stress granule assembly by condensation. Cell, 181(2), 346-361. e317.
Holz, M. K., Ballif, B. A., Gygi, S. P., & Blenis, J. (2005). mTOR and S6K1 mediate assembly of the translation preinitiation complex through dynamic protein interchange and ordered phosphorylation events. Cell, 123(4), 569-580.
Hornung, V., Bauernfeind, F., Halle, A., Samstad, E. O., Kono, H., Rock, K. L., . . . Latz, E. (2008). Silica crystals and aluminum salts activate the NALP3 inflammasome through phagosomal destabilization. Nature immunology, 9(8), 847-856.
Hui, L., Chen, X., & Geiger, J. D. (2012). Endolysosome involvement in LDL cholesterol-induced Alzheimer's disease-like pathology in primary cultured neurons. Life sciences, 91(23-24), 1159-1168.
Hyman, A. A., Weber, C. A., & Jülicher, F. (2014). Liquid-liquid phase separation in biology. Annual review of cell and developmental biology, 30, 39-58.
Ivanov, P., Kedersha, N., & Anderson, P. (2019). Stress Granules and Processing Bodies in Translational Control. Cold Spring Harb Perspect Biol, 11(5). doi: 10.1101/cshperspect.a032813
Jackson, R. J., Hellen, C. U., & Pestova, T. V. (2010). The mechanism of eukaryotic translation initiation and principles of its regulation. Nature reviews Molecular cell biology, 11(2), 113-127.
Jain, S., Wheeler, J. R., Walters, R. W., Agrawal, A., Barsic, A., & Parker, R. (2016). ATPase-Modulated Stress Granules Contain a Diverse Proteome and Substructure. Cell, 164(3), 487-498. doi: 10.1016/j.cell.2015.12.038
Jammi, N. V., Whitby, L. R., & Beal, P. A. (2003). Small molecule inhibitors of the RNA-dependent protein kinase. Biochemical and biophysical research communications, 308(1), 50-57.
Jia, J., Abudu, Y. P., Claude-Taupin, A., Gu, Y., Kumar, S., Choi, S. W., . . . Deretic, V. (2018). Galectins Control mTOR in Response to Endomembrane Damage. Mol Cell, 70(1), 120-135 e128. doi: 10.1016/j.molcel.2018.03.009
Jia, J., Bissa, B., Brecht, L., Allers, L., Choi, S. W., Gu, Y., . . . Hallows, K. (2020a). AMPK is activated during lysosomal damage via a galectin-ubiquitin signal transduction system. Autophagy, 16(8), 1550-1552.
Jia, J., Bissa, B., Brecht, L., Allers, L., Choi, S. W., Gu, Y., . . . Hallows, K. (2020b). AMPK, a regulator of metabolism and autophagy, is activated by lysosomal damage via a novel galectin-directed ubiquitin signal transduction system. Molecular cell, 77(5), 951-969. e959.
Jia, J., Claude-Taupin, A., Gu, Y., Choi, S. W., Peters, R., Bissa, B., . . . Lidke, K. A. (2020c). Galectin-3 coordinates a cellular system for lysosomal repair and removal. Developmental Cell, 52(1), 69-87. e68.
Jia, J., Claude-Taupin, A., Gu, Y., Choi, S. W., Peters, R., Bissa, B., . . . Lidke, K. A. (2020d). MERIT, a cellular system coordinating lysosomal repair, removal and replacement. Autophagy, 16(8), 1539-1541.
Jia, J., Wang, F., Bhujabal, Z., Peters, R., Mudd, M., Duque, T., . . . Behrends, C. (2022). Stress granules and mTOR are regulated by membrane atg8ylation during lysosomal damage. Journal of Cell Biology, 221(11), e202207091.
Kaur, N., de la Ballina, L. R., Haukaas, H. S., Torgersen, M. L., Radulovic, M., Munson, M. J., . . . Carlsson, S. R. (2023). TECPR1 is activated by damage-induced sphingomyelin exposure to mediate noncanonical autophagy. The EMBO journal, 42(17), e113105.
Kedersha, N., Cho, M. R., Li, W., Yacono, P. W., Chen, S., Gilks, N., . . . Anderson, P. (2000). Dynamic shuttling of TIA-1 accompanies the recruitment of mRNA to mammalian stress granules. J Cell Biol, 151(6), 1257-1268. doi: 10.1083/jcb.151.6.1257
Kedersha, N., Panas, M. D., Achorn, C. A., Lyons, S., Tisdale, S., Hickman, T., . . . Ivanov, P. (2016). G3BP-Caprin1-USP10 complexes mediate stress granule condensation and associate with 40S subunits. Journal of Cell Biology, 212(7).
Kedersha, N. L., Gupta, M., Li, W., Miller, I., & Anderson, P. (1999). RNA-binding proteins TIA-1 and TIAR link the phosphorylation of eIF-2 alpha to the assembly of mammalian stress granules. J Cell Biol, 147(7), 1431-1442. doi: 10.1083/jcb.147.7.1431
Keller, C. C., Kremsner, P. G., Hittner, J. B., Misukonis, M. A., Weinberg, J. B., & Perkins, D. J. (2004). Elevated nitric oxide production in children with malarial anemia: hemozoin-induced nitric oxide synthase type 2 transcripts and nitric oxide in blood mononuclear cells. Infection and immunity, 72(8), 4868-4873.
Khong, A., Matheny, T., Jain, S., Mitchell, S. F., Wheeler, J. R., & Parker, R. (2017). The stress granule transcriptome reveals principles of mRNA accumulation in stress granules. Molecular cell, 68(4), 808-820. e805.
Kumar, P., Nagarajan, A., & Uchil, P. D. (2018). Analysis of cell viability by the lactate dehydrogenase assay. Cold Spring Harb. Protoc, 2018(6), 465-468.
Lamming, D. W., & Bar-Peled, L. (2019). Lysosome: the metabolic signaling hub. Traffic, 20(1), 27-38.
Lawrence, R. E., & Zoncu, R. (2019). The lysosome as a cellular centre for signalling, metabolism and quality control. Nat Cell Biol, 21(2), 133-142. doi: 10.1038/s41556-018-0244-7
Liu, B., Chen, R., Zhang, Y., Huang, J., Luo, Y., Rosthøj, S., . . . Jäättelä, M. (2023). Cationic amphiphilic antihistamines inhibit STAT3 via Ca2+-dependent lysosomal H+ efflux. Cell Reports, 42(2).
Lloyd-Evans, E., & Waller-Evans, H. (2020). Lysosomal Ca2+ homeostasis and signaling in health and disease. Cold Spring Harbor perspectives in biology, 12(6), a035311.
Lu, Y.-N., Kavianpour, S., Zhang, T., Zhang, X., Nguyen, D., Thombre, R., . . . Wang, J. (2021). MARK2 phosphorylates eIF2α in response to proteotoxic stress. PLOS biology, 19(3), e3001096.
Luisoni, S., Suomalainen, M., Boucke, K., Tanner, L. B., Wenk, M. R., Guan, X. L., . . . Greber, U. F. (2015). Co-option of membrane wounding enables virus penetration into cells. Cell host & microbe, 18(1), 75-85.
Maejima, I., Takahashi, A., Omori, H., Kimura, T., Takabatake, Y., Saitoh, T., . . . Yoshimori, T. (2013). Autophagy sequesters damaged lysosomes to control lysosomal biogenesis and kidney injury. EMBO J, 32(17), 2336-2347. doi: 10.1038/emboj.2013.171
Maki, M., Takahara, T., & Shibata, H. (2016). Multifaceted roles of ALG-2 in Ca2+-regulated membrane trafficking. International journal of molecular sciences, 17(9), 1401.
Marceau, F., Bawolak, M.-T., Lodge, R., Bouthillier, J., Gagné-Henley, A., René, C., & Morissette, G. (2012). Cation trapping by cellular acidic compartments: beyond the concept of lysosomotropic drugs. Toxicology and applied pharmacology, 259(1), 1-12.
Martinez, R., Schellenberger, P., Vasishtan, D., Aknin, C., Austin, S., Dacheux, D., . . . Gruenewald, K. (2015). The amphipathic helix of adenovirus capsid protein VI contributes to penton release and postentry sorting. Journal of virology, 89(4), 2121-2135.
McCormick, C., & Khaperskyy, D. A. (2017). Translation inhibition and stress granules in the antiviral immune response. Nat Rev Immunol, 17(10), 647-660. doi: 10.1038/nri.2017.63
Medina, D. L., Di Paola, S., Peluso, I., Armani, A., De Stefani, D., Venditti, R., . . . Ballabio, A. (2015). Lysosomal calcium signalling regulates autophagy through calcineurin and TFEB. Nature cell biology, 17(3), 288-299. doi: 10.1038/ncb3114
Millar, S. R., Huang, J. Q., Schreiber, K. J., Tsai, Y.-C., Won, J., Zhang, J., . . . Youn, J.-Y. (2023). A New Phase of Networking: The Molecular Composition and Regulatory Dynamics of Mammalian Stress Granules. Chemical Reviews.
Montespan, C., Marvin, S. A., Austin, S., Burrage, A. M., Roger, B., Rayne, F., . . . Wodrich, H. (2017). Multi-layered control of Galectin-8 mediated autophagy during adenovirus cell entry through a conserved PPxY motif in the viral capsid. PLOS Pathogens, 13(2), e1006217. doi: 10.1371/journal.ppat.1006217
Moore, J. M., Chaisavaneeyakorn, S., Perkins, D. J., Othoro, C., Otieno, J., Nahlen, B. L., . . . Udhayakumar, V. (2004). Hemozoin differentially regulates proinflammatory cytokine production in human immunodeficiency virus-seropositive and -seronegative women with placental malaria. Infection and immunity, 72(12), 7022-7029.
Mossman, B. T., & Churg, A. (1998). Mechanisms in the pathogenesis of asbestosis and silicosis. Am J Respir Crit Care Med, 157(5 Pt 1), 1666-1680. doi: 10.1164/ajrccm. 157.5.9707141
Nakamura, S., Akayama, S., & Yoshimori, T. (2021). Autophagy-independent function of lipidated LC3 essential for TFEB activation during the lysosomal damage responses. Autophagy, 17(2), 581-583.
Nakamura, S., Shigeyama, S., Minami, S., Shima, T., Akayama, S., Matsuda, T., . . . Namba-Hamano, T. (2020). LC3 lipidation is essential for TFEB activation during the lysosomal damage response to kidney injury. Nature cell biology, 22(10), 1252-1263.
Napolitano, G., & Ballabio, A. (2016). TFEB at a glance. Journal of cell science, 129(13), 2475-2481.
Niekamp, P., Scharte, F., Sokoya, T., Vittadello, L., Kim, Y., Deng, Y., . . . Holthuis, J. C. M. (2022). Ca (2+)-activated sphingomyelin scrambling and turnover mediate ESCRT-independent lysosomal repair. Nat Commun, 13(1), 1875. doi: 10.1038/s41467-022-29481-4
Ogura, M., Kaminishi, T., Shima, T., Torigata, M., Bekku, N., Tabata, K., . . . Hamasaki, M. (2023). Microautophagy regulated by STK38 and GABARAPs is essential to repair lysosomes and prevent aging. EMBO reports, 24(12), e57300.
Pakos-Zebrucka, K., Koryga, I., Mnich, K., Ljujic, M., Samali, A., & Gorman, A. M. (2016). The integrated stress response. EMBO Rep, 17(10), 1374-1395. doi: 10.15252/embr.201642195
Papadopoulos, C., Kirchner, P., Bug, M., Grum, D., Koerver, L., Schulze, N., . . . Meyer, H. (2017). VCP/p97 cooperates with YOD1, UBXD1 and PLAA to drive clearance of ruptured lysosomes by autophagy. EMBO J, 36(2), 135-150. doi: 10.15252/embj.201695148
Papadopoulos, C., & Meyer, H. (2017). Detection and clearance of damaged lysosomes by the endo-lysosomal damage response and lysophagy. Current Biology, 27(24), R1330-R1341.
Patel, R. C., & Sen, G. C. (1998). PACT, a protein activator of the interferon-induced protein kinase, PKR. The EMBO journal, 17(15), 4379-4390.
Patra, S., Patil, S., Klionsky, D. J., & Bhutia, S. K. (2023). Lysosome signaling in cell survival and programmed cell death for cellular homeostasis. Journal of Cellular Physiology, 238(2), 287-305.
Pied, N., Daussy, C. F., Denis, Z., Ragues, J., Faure, M., Iggo, R., . . . Wodrich, H. (2022). TBK1 is part of a galectin 8 dependent membrane damage recognition complex and drives autophagy upon Adenovirus endosomal escape. PLOS Pathog, 18(7), e1010736. doi: 10.1371/journal.ppat.1010736
Pierzyńska-Mach, A., Janowski, P. A., & Dobrucki, J. W. (2014). Evaluation of acridine orange, LysoTracker Red, and quinacrine as fluorescent probes for long-term tracking of acidic vesicles. Cytometry Part A, 85(8), 729-737.
Pisonero-Vaquero, S., & Medina, D. L. (2017). Lysosomotropic drugs: pharmacological tools to study lysosomal function. Current Drug Metabolism, 18(12), 1147-1158. Prentzell, M. T., Rehbein, U., Cadena Sandoval, M., De Meulemeester, A. S.,
Baumeister, R., Brohee, L., . . . Thedieck, K. (2021). G3BPs tether the TSC complex to lysosomes and suppress mTORC1 signaling. Cell, 184(3), 655-674 e627. doi: 10.1016/j.cell.2020.12.024
Protter, D. S., & Parker, R. (2016). Principles and properties of stress granules. Trends in Cell Biology, 26(9), 668-679.
Rabouw, H. H., Langereis, M. A., Anand, A. A., Visser, L. J., de Groot, R. J., Walter, P., & van Kuppeveld, F. J. (2019). Small molecule ISRIB suppresses the integrated stress response within a defined window of activation. Proceedings of the National Academy of Sciences, 116(6), 2097-2102.
Radulovic, M., Schink, K. O., Wenzel, E. M., Nahse, V., Bongiovanni, A., Lafont, F., & Stenmark, H. (2018). ESCRT-mediated lysosome repair precedes lysophagy and promotes cell survival. EMBO J, 37(21). doi: 10.15252/embj.201899753
Riggs, C. L., Kedersha, N., Ivanov, P., & Anderson, P. (2020). Mammalian stress granules and P bodies at a glance. J Cell Sci, 133(16). doi: 10.1242/jcs.242487
Ripin, N., & Parker, R. (2023). Formation, function, and pathology of RNP granules. Cell, 186(22), 4737-4756.
Sadler, A. J., & Williams, B. R. G. (2007). Structure and function of the protein kinase R. Interferon: The 50th Anniversary, 253-292.
Saftig, P., & Puertollano, R. (2021). How lysosomes sense, integrate, and cope with stress. Trends in biochemical sciences, 46(2), 97-112.
Sancak, Y., Bar-Peled, L., Zoncu, R., Markhard, A. L., Nada, S., & Sabatini, D. M. (2010). Ragulator-Rag complex targets mTORC1 to the lysosomal surface and is necessary for its activation by amino acids. Cell, 141(2), 290-303.
Scheffer, L. L., Sreetama, S. C., Sharma, N., Medikayala, S., Brown, K. J., Defour, A., & Jaiswal, J. K. (2014). Mechanism of Ca2+-triggered ESCRT assembly and regulation of cell membrane repair. Nature communications, 5(1), 5646.
Schwarzer, E., Bellomo, G., Giribaldi, G., Ulliers, D., & Arese, P. (2001). Phagocytosis of malarial pigment haemozoin by human monocytes: a confocal microscopy study. Parasitology, 123(2), 125-131.
Schwarzer, E., Turrini, F., Ulliers, D., Giribaldi, G., Ginsburg, H., & Arese, P. (1992). Impairment of macrophage functions after ingestion of Plasmodium falciparum-infected erythrocytes or isolated malarial pigment. The Journal of experimental medicine, 176(4), 1033-1041.
Sidrauski, C., McGeachy, A. M., Ingolia, N. T., & Walter, P. (2015). The small molecule ISRIB reverses the effects of eIF2α phosphorylation on translation and stress granule assembly. elife, 4. doi: 10.7554/eLife.05033
Singh, M., & Patel, R. C. (2012). Increased interaction between PACT molecules in response to stress signals is required for PKR activation. Journal of cellular biochemistry, 113(8), 2754-2764.
Skowyra, M. L., Schlesinger, P. H., Naismith, T. V., & Hanson, P. I. (2018). Triggered recruitment of ESCRT machinery promotes endolysosomal repair. Science, 360(6384), eaar5078.
Srivastava, S. P., Kumar, K. U., & Kaufman, R. J. (1998). Phosphorylation of eukaryotic translation initiation factor 2 mediates apoptosis in response to activation of the double-stranded RNA-dependent protein kinase. J Biol Chem, 273(4), 2416-2423. doi: 10.1074/jbc.273.4.2416
Sun, S., Zhou, X., Corvera, J., Gallick, G. E., Lin, S.-H., & Kuang, J. (2015). ALG-2 activates the MVB sorting function of ALIX through relieving its intramolecular interaction. Cell discovery, 1(1), 1-19.
Tan, J. X., & Finkel, T. (2022). A phosphoinositide signalling pathway mediates rapid lysosomal repair. Nature, 609(7928), 815-821.
Thedieck, K., Holzwarth, B., Prentzell, M. T., Boehlke, C., Klasener, K., Ruf, S., . . . Baumeister, R. (2013). Inhibition of mTORC1 by astrin and stress granules prevents apoptosis in cancer cells. Cell, 154(4), 859-874. doi: 10.1016/j.cell.2013.07.031
Thiele, D. L., & Lipsky, P. E. (1990). Mechanism of L-leucyl-L-leucine methyl ester-mediated killing of cytotoxic lymphocytes: dependence on a lysosomal thiol protease, dipeptidyl peptidase I, that is enriched in these cells. Proceedings of the National Academy of Sciences, 87(1), 83-87.
Thurston, T. L., Wandel, M. P., von Muhlinen, N., Foeglein, A., & Randow, F. (2012). Galectin 8 targets damaged vesicles for autophagy to defend cells against bacterial invasion. Nature, 482(7385), 414-418. doi: 10.1038/nature 10744
Tronel, C., Page, G., Bodard, S., Chalon, S., & Antier, D. (2014). The specific PKR inhibitor C16 prevents apoptosis and IL-1β production in an acute excitotoxic rat model with a neuroinflammatory component. Neurochemistry International, 64, 73-83.
Turakhiya, A., Meyer, S. R., Marincola, G., Böhm, S., Vanselow, J. T., Schlosser, A., . . . Buchberger, A. (2018). ZFAND1 recruits p97 and the 26S proteasome to promote the clearance of arsenite-induced stress granules. Molecular cell, 70(5), 906-919. e907.
Verma, A., Sumi, S., & Seervi, M. (2021). Heat shock proteins-driven stress granule dynamics: yet another avenue for cell survival. Apoptosis, 26(7-8), 371-384.
Vietri, M., Radulovic, M., & Stenmark, H. (2020). The many functions of ESCRTs. Nature reviews Molecular cell biology, 21(1), 25-42.
Wang, Peters, R., Jia, J., Mudd, M., Salemi, M., Allers, L., . . . Trosdal, E. S. (2023). ATG5 provides host protection acting as a switch in the atg8ylation cascade between autophagy and secretion. Developmental Cell.
Wang, F., Gómez-Sintes, R., & Boya, P. (2018). Lysosomal membrane permeabilization and cell death. Traffic, 19(12), 918-931.
Wang, F., Li, J., Fan, S., Jin, Z., & Huang, C. (2020). Targeting stress granules: A novel therapeutic strategy for human diseases. Pharmacological Research, 161, 105143.
Wang, J., Yu, Y., Lu, K., Yang, M., Li, Y., Zhou, X., & Sun, Z. (2017). Silica nanoparticles induce autophagy dysfunction via lysosomal impairment and inhibition of autophagosome degradation in hepatocytes. International Journal of Nanomedicine, 809-825.
Wang, X., & Proud, C. G. (2008). A novel mechanism for the control of translation initiation by amino acids, mediated by phosphorylation of eukaryotic initiation factor 2B. Molecular and cellular biology, 28(5), 1429-1442.
Weissbuch, I., & Leiserowitz, L. (2008). Interplay between malaria, crystalline hemozoin formation, and antimalarial drug action and design. Chemical Reviews, 108(11), 4899-4914.
Wiethoff, C. M., & Nemerow, G. R. (2015). Adenovirus membrane penetration: tickling the tail of a sleeping dragon. Virology, 479, 591-599.
Wyant, G. A., Abu-Remaileh, M., Frenkel, E. M., Laqtom, N. N., Dharamdasani, V., Lewis, C. A., . . . Sabatini, D. M. (2018). NUFIP1 is a ribosome receptor for starvation-induced ribophagy. Science, 360(6390), 751-758.
Xu, H., & Ren, D. (2015). Lysosomal physiology. Annual review of physiology, 77, 57-80.
Yan, Y., Tao, H., He, J., & Huang, S.-Y. (2020). The HDOCK server for integrated protein-protein docking. Nature protocols, 15(5), 1829-1852.
Yang, C., & Wang, X. (2021). Lysosome biogenesis: Regulation and functions. Journal of Cell Biology, 220(6), e202102001.
Yang, H., & Tan, J. X. (2023). Lysosomal quality control: molecular mechanisms and therapeutic implications. Trends in Cell Biology.
Yang, P., Mathieu, C., Kolaitis, R.-M., Zhang, P., Messing, J., Yurtsever, U., . . . Pan, Q. (2020). G3BP1 is a tunable switch that triggers phase separation to assemble stress granules. Cell, 181(2), 325-345. e328.
Yim, W. W. Y., Yamamoto, H., & Mizushima, N. (2022). Annexins A1 and A2 are recruited to larger lysosomal injuries independently of ESCRTs to promote repair. FEBS letters, 596(8), 991-1003.
Youn, J.-Y., Dyakov, B. J., Zhang, J., Knight, J. D., Vernon, R. M., Forman-Kay, J. D., & Gingras, A.-C. (2019). Properties of stress granule and P-body proteomes. Molecular cell, 76(2), 286-294.
Zhang, C.-S., Jiang, B., Li, M., Zhu, M., Peng, Y., Zhang, Y.-L., . . . Lu, Z. (2014). The lysosomal v-ATPase-Ragulator complex is a common activator for AMPK and mTORC1, acting as a switch between catabolismand anabolism. Cell metabolism, 20(3), 526-540.
Zhang, M. L., Zhang, Z., Niu, X. Z., Ti, H. Y., Zhou, Y. X., Gao, B., . . . Li, H. (2024). Interplay Between Intracellular Transport Dynamics and Liquid-Liquid Phase Separation. Advanced Science, 2308338.
Zhao, Y. G., & Zhang, H. (2020). Phase separation in membrane biology: the interplay between membrane-bound organelles and membraneless condensates. Developmental Cell, 55(1), 30-44.
Zoncu, R., Bar-Peled, L., Efeyan, A., Wang, S., Sancak, Y., & Sabatini, D. M. (2011). mTORC1 senses lysosomal amino acids through an inside-out mechanism that requires the vacuolar H+-ATPase. Science, 334(6056), 678-683.SEQUENCES

SEQUENCES
(NUFIP2)
SEQ ID NO: 1
MEEKPGQPQPQHHHSHHHPHHHPQQQQQQPHHHHHYYFYNHSHNHHHHHH
HQQPHQYLQHGAEGSPKAQPKPLKHEQKHTLQQHQETPKKKTGYGELNGN
AGEREISLKNLSSDEATNPIFSRVLNGNQQVVDTSLKQTVKANTFGKAGI
KTKNFIQKNSMDKKNGKSYENKSGENQSVDKSDTIPIPNGVVTNNSGYIT
NGYMGKGADNDGSGSESGYTTPKKRKARRNSAKGCENLNIVQDKIMQQET
SVPTLKQGLETFKPDYSEQKGNRVDGSKPIWKYETGPGGTSRGKPAVGDM
LRKSSDSKPGVSSKKFDDRPKGKHASAVASKEDSWTLFKPPPVFPVDNSS
AKIVPKISYASKVKENLNKTIQNSSVSPTSSSSSSSSTGETQTQSSSRLS
QVPMSALKSVTSANFSNGPVLAGTDGNVYPPGGQPLLTTAANTLTPISSG
TDSVLQDMSLTSAAVEQIKTSLFIYPSNMQTMLLSTAQVDLPSQTDQQNL
GDIFQNQWGLSFINEPSAGPETVTGKSSEHKVMEVTFQGEYPATLVSQGA
EIIPSGTEHPVFPKAYELEKRTSPQVLGSILKSGTTSESGALSLEPSHIG
DLQKADTSSQGALVFLSKDYEIESQNPLASPTNTLLGSAKEQRYQRGLER
NDSWGSFDLRAAIVYHTKEMESIWNLQKQDPKRIITYNEAMDSPDQ
SEQ ID NO: 2
GYTTPKKRKARR
SEQ ID NO: 3
AGGAAAGCUAGGCGCAAUA
SEQ ID NO: 4
GGGUGAUAUGCUUCGGAAA
SEQ ID NO: 5
AUUAAGCCCUGCGAGAAU
SEQ ID NO: 6
AUGGUGAACUAAACGGUAA
SEQ ID NO: 7
GUGGUGGAGUUGCGCAUUA
SEQ ID NO: 8
AGACAUAGCUCAGACAGUA
SEQ ID NO: 9
GAAGGCGACCGACGAGAUA
SEQ ID NO: 10
GCGAGAACAACGAAUAAAU

1. A method of treating an autophagy modulated disease state and/or condition comprising administering to a patient or subject in need an effective amount of a modulator of NUFIP2 or G3BP1 or a modulator of the expression of NUFIP2 or G3BP1, wherein the disease state is cancer and the modulator is an inhibitor of NUFIP2 or G3BP1.

2. The method according to claim 1 wherein said inhibitor of NUFIP2 or G3BP1 is a siRNA according any one of SEQ ID Nos. 3-10 or SJ-19-0043.

3. The method according to claim 1 wherein said inhibitor of NUFIP2 is a siRNA according to SEQ ID Nos 3-6.

4. The method according to 1 wherein said inhibitor of G3BP1 is a siRNA according to SEQ ID Nos. 7-10.

5. The method according to claim 3 wherein said siRNA is a conjugated siRNA or is presented in a lipid nanoparticle.

6. The method according to claim 5 wherein said siRNA is presented in a lipid nanoparticle.

7. The method according to claim 5 wherein said siRNA is a conjugated siRNA.

8. The method according to claim 7 wherein said conjugated siRNA is a dynamic polyconjugate (DPC), an antibody-SiRNA conjugate or a GalNAc-SiRNA conjugate.

9. The method according to claim 1 wherein said inhibitor is combined with an effective amount of a lysosomotropic agent in the treatment of cancer.

10. The method according to claim 9 wherein said lysosomotropic agent is a lysosomotropic detergent.

11. (canceled)

12. The method according to claim 9 wherein said lysosomotropic amine is sphingosine, O-methyl-serine dodecylamide hydrochloride (MSDH), N-dodecylimidazole or a mixture thereof.

13. The method according to claim 9 wherein said lysosomotropic agent is chloroquine, chlorpromazine, thioridazine, aripiprazole, clomipramine, imipramine, desipramine, seramasine, or a mixture thereof.

14. The method according to claim 9, wherein said lysosomotropic agent is glycyl-L-phenylalanine-2-naphthyl amide (GPN), Leu-Leu-OMe (LLOMe) or a mixture thereof.

15. The method according to claim 1 wherein said cancer is a tumor.

16. The method according to claim 15 wherein said cancer is a carcinoma or a tumor of the central nervous system.

17. The method according to claim 16 wherein said cancer is pancreatic cancer, a glioma, glioblastoma or a neuroblastoma.

18. The method according to claim 9 wherein said cancer is a carcinoma or a tumor of the central nervous system.

19. The method according to claim 18 wherein said cancer is pancreatic cancer, a glioma, glioblastsoma or a neuroblastoma.

20. A method of treating cancer in a patient or subject in need comprising administering to said patient or subject an effective amount of integrated stress response inhibitor (ISRIB) in combination with a lysosomotropic agent and/or a siRNA according to any one or more of SEQ ID Nos: 3-10.

21. (canceled)

22. (canceled)

23. A pharmaceutical composition comprising an effective amount of a siRNA according to SEQ ID Nos 3-10 in combination with a lysosomotropic agent.
